(19) United States
(12) Patent Application Publication
(10) Pub. No.: US 2024/0043467 A1
(43) Pub. Date: Feb. 8, 2024

(54) 5'-SUBSTITUTED NUCLEOSIDE MONOPHOSPHATES, PRODRUGS THEREOF, AND USES RELATED THERETO

(71) Applicant: EMORY UNIVERSITY, Atlanta, GA (US)

(72) Inventors: Eric J. MILLER, Atlanta, GA (US); Madhuri DASARI, Atlanta, GA (US); Stephen C. PELLY, Atlanta, GA (US); Dennis C. LIOTTA, Atlanta, GA (US)

(73) Assignee: EMORY UNIVERSITY, Atlanta, GA (US)

(21) Appl. No.: 18/041,785

(22) PCT Filed: Aug. 31, 2021
(86) PCT No.: PCT/US21/48488

§ 371 Date: Feb. 15, 2023

Related U.S. Application Data
(60) Provisional application No. 63/072,369, filed on Aug. 31, 2020.

(51) Int. Cl. C07H 19/10

(52) U.S. Cl. CPC C07H 19/10

(57) ABSTRACT

Disclosed are 5′-substituted nucleoside monophosphates, which contain 5-fluorouracil or 5-fluorothiouracil as the nucleobase. In general, the 5′-substituted nucleoside monophosphates disclosed herein can inhibit human thymidylate synthase and thereby possess anti-cancer therapeutic effects. The 5′-substitution in these nucleoside monophosphates can prevent metabolic cleavage of the monophosphate group, mediated by enzymes such as 5′-nucleotidases, phospholipase D, etc. This feature can improve metabolic profiles, enhance target specificity, and/or reduce side effects of these compounds, compared to their corresponding unsubstituted analogs. Also disclosed are prodrugs of these 5′-substituted nucleoside monophosphates. After administration, the prodrugs can be metabolized to release their corresponding 5′-substituted nucleoside monophosphates. Methods of treating cancer using the 5′-substituted nucleoside monophosphates and prodrugs thereof are disclosed. An exemplary method involves orally administering a prodrug disclosed herein to a subject in need thereof.

### CROSS-REFERENCE TO PRIORITY APPLICATION

This application claims priority to U.S. Provisional Application No. 63/072,369, filed Aug. 31, 2020, which is incorporated herein by reference in its entirety.

### TECHNICAL FIELD

The present disclosure relates to prodrugs of nucleoside monophosphates and derivatives thereof, and pharmaceutical compositions and uses related thereto.

### BACKGROUND

Although conventional chemotherapy has been successful to some extent, the main drawbacks of chemotherapy are its poor oral bioavailability, high-dose requirements, adverse side effects, and non-specific targeting. Notably, drug metabolism of anticancer agents plays an important role in dictating their therapeutic efficacy. After administration, anticancer agents are typically metabolized through a number of parallel and/or sequential reactions. This process usually generates a range of metabolites of the anticancer agents. These metabolites may have different activities/reactivities, thereby giving rise to the afore-mentioned drawbacks of conventional chemotherapy.

One example is 5-fluorouracil (5-FU). 5-FU is a uracil analog that acts as an antimetabolite against cancer progression. It is approved by the FDA for treating colorectal, breast, pancreatic, stomach, esophageal, and cervical cancers. 5-FU itself is an inactive prodrug. After administration, multiple active/reactive metabolites of 5-FU are generated, including (1) 5-fluorodeoxyuridine monophosphate (FdUMP), which exerts anticancer effects predominantly through inhibition of thymidylate synthase (TS), and (2) various corresponding reactive ribonucleoside and deoxyribonucleoside triphosphates, which can be incorporated into growing strands of RNA and DNA, respectively.

Eighty to eighty-five percent of orally administered 5-FU is metabolized in the gut and liver by dihydropyrimidine dehydrogenase (DPD). Therefore, it has poor oral bioavailability and requires administration by intravenous injection.

A variety of prodrug strategies have been developed to improve oral bioavailability and other pharmacokinetic properties of 5-FU. However, most of these 5-FU prodrugs generate 5-FU as a metabolite, which then goes on to produce the afore-mentioned active/reactive metabolites with different mechanisms of action, thereby rendering toxicity to healthy tissues versus malignant tissues difficult to control.

There is a need for improved anticancer agents, particularly those with better oral bioavailability, improved metabolic profiles, enhanced target specificity, and/or reduced side effects. There is also a need for improved chemotherapy regimens, particularly those with lower doses and/or reduced side effects.

### SUMMARY

Disclosed are 5′-substituted nucleoside monophosphates, which contain 5-fluorouracil or 5-fluorothiouracil as a nucleobase. The 5′-substituted nucleoside monophosphates can inhibit human thymidylate synthase (TS) and possess anti-cancer therapeutic effects.

The 5′-substitution in these compounds can prevent metabolic cleavage of the monophosphate group, mediated by enzymes such as 5′-nucleotidases, phospholipase D (PLD), etc. This feature can improve metabolic profiles, enhance target specificity, and/or reduce side effects of these compounds, compared to their corresponding unsubstituted analogs.

In some examples, the 5′-substituted nucleoside monophosphates have a structure of Formula I or a pharmaceutically acceptable salt thereof:

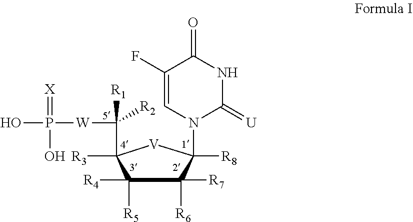

wherein:
(1) R₁ and R₂ are independently selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen,
(2) R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ, or
(3) one of R₁ and R₂ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and the other one of R₁ and R₂ joins with the 4′ carbon and the 5′ carbon to form a 3- or 4-membered carbocycle optionally substituted by one or more Rₐ;
wherein U is O or S;
wherein V is O or S;
wherein W is O or optionally substituted methylene;
wherein X is O or S;
wherein R₃ is absent or selected from hydrogen, deuterium, halogen, cyano, ethynyl, optionally substituted alkyl, optionally O-substituted hydroxyl, and ester; wherein R₄ is hydrogen or deuterium;
wherein R₅ is selected from fluorine, optionally O-substituted hydroxyl, amino, acyl, ester, amide, acylamino, and substituted alkyl containing a substituent selected from fluorine, optionally O-substituted hydroxyl, and amino;
wherein R₆, R₇, and R₈ are independently selected from hydrogen, deuterium, halogen, cyano, ethynyl, optionally substituted alkyl, optionally O-substituted hydroxyl, and ester; and wherein Rₐ is selected from deuterium, halogen, and hydroxyl.

The “ethene moiety” formed by R₁, R₂, and the 5′ carbon is —(C═CH₂—

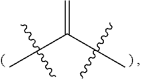 [SMILES: *C(*)=C]

which can be substituted by one or more Rₐ. In this group, the carbon atom with two open valencies is the 5′ carbon; R₁ and R₂ merge together to form ═CH₂, which can be substituted by one or more Rₐ.

In some examples, R₁ and R₂ are independently selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen.

In some examples, R₁ is hydrogen, deuterium, halogen, methyl optionally substituted by one or more Rₐ, or ethenyl optionally substituted by one or more Rₐ. In some examples, R₁ is hydrogen.

In some examples, R₂ is selected from cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ. In some examples, R₂ is methyl, —CF₃, or —CH₂OH.

In some examples, R₁ is hydrogen and R₂ is selected from cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ. In some examples, R₁ is hydrogen and R₂ is methyl, —CF₃, or —CH₂OH.

In some examples, U, V, W, and X are O.

In some examples, R₃, R₄, R₆, R₇, and R₈ are hydrogen and R₅ is hydroxyl.

Exemplary 5′-substituted nucleoside monophosphates include, but are not limited to, the following compounds and their pharmaceutically acceptable salts thereof:
(R)-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)-2-hydroxyethyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)allyl dihydrogen phosphate, and
1-((2S,3S,5R)-5-(5-fluoro-2,4-dioxo-3,4-dihydropyrimidin-1(2H)-yl)-3-hydroxytetrahydrofuran-2-yl)cyclopropyl dihydrogen phosphate.

Also disclosed are prodrugs of the 5′-substituted nucleoside monophosphates. After administration, the prodrugs can be metabolized in vivo to release their corresponding 5′-substituted nucleoside monophosphates.

In some examples, the prodrugs have a structure of Formula II or Formula III, or a pharmaceutically acceptable salt thereof,

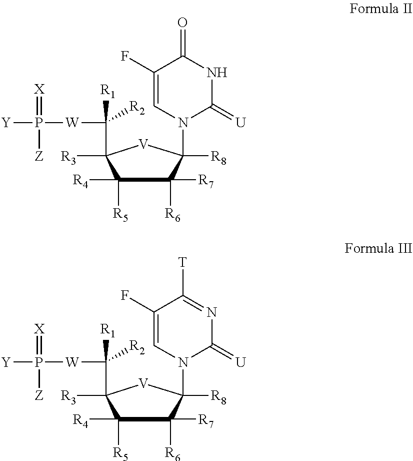

wherein R₁, R₂, R₃, R₄, R₅, R₆, R₇, R₈, U, V, W, and X are defined in Formula I;
wherein Y and Z are independently selected from —O—R₉, —S—R₁₀, and R₁₁

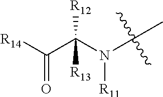

with the proviso that Y and Z are not both hydroxyl;
wherein T is —NR₁₅R₁₆ or —OR₁₇;
wherein R₉ selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, —R_{q1}—R_{q2}—R_{q3}—R_{q4}, and —Rᵣ₁—Rᵣ₂;
wherein R₁₀ is selected from optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₁ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₂ and R₁₃ are independently selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and standard amino acid side chain,
wherein when the standard amino acid side chain is a proline side chain, one of R₁₂ and R₁₃ is hydrogen, and the other one of R₁₂ and R₁₃ forms a pyrrolidine ring together with R₁₁, the nitrogen atom connected to R₁₁, and the carbon atom connected to R₁₂ and R₁₃;
wherein R₁₄ is —NRₛ₁R^{s2} or —ORₜ;
wherein R₁₅ and R₁₆ are independently selected from hydrogen, acyl, ester, thioester, and amide;
wherein R₁₇ is acyl, ester, thioester, or amide;
wherein:
   R_{q1} is absent or a C₁-C₉ alkyl chain (i.e., C₁-C₉ bridging alkylene),
   R_{q2} is absent or is selected from substituted methylene or ethylene, —O—, —S—, —S(═O)—, —S—S—, and —S(O)₂—,
   R_{q3} is a C₂-C₂₀ alkyl chain (i.e., C₂-C₂₀ bridging alkylene), and
   R_{q4} is selected from hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅;
wherein:
   Rᵣ₁ is optionally substituted C₁-C₄ bridging alkylene, and
   Rᵣ₂ is selected from ester, thioester, amide, acylamino, carbonate ester, carbamate, disulfide, optionally substituted (4-acylamino)phenyl, and optionally substituted (4-acyloxy)phenyl; and
wherein:
   Rₛ₁ and Rₛ₂ are independently selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl, and
   Rₜ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl.

In some examples, Y is —O—R₉ and Z is

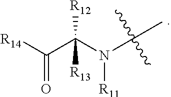

In some examples, R₉ is phenyl or naphthyl, R₁₁ is hydrogen, one of R₁₂ and R₁₃ is hydrogen, the other one of R₁₂ and R₁₃ is methyl, and R₁₄ is —ORₜ, wherein Rₜ is isopropyl, 2-ethylbutyl, or benzyl.

Also disclosed are prodrugs of nucleoside monophosphates that can be unsubstituted at the 5′ position. After administering to a subject, the prodrug can be metabolized to release the corresponding nucleoside monophosphate.

Also disclosed are pharmaceutical formulations containing a 5′-substituted nucleoside monophosphate, as described herein, a prodrug thereof, also as described herein, or a prodrug of a nucleoside monophosphate that is unsubstituted at the 5′ position, also as described herein. Generally, the pharmaceutical formulations further contain a pharmaceutically acceptable excipient. The pharmaceutical formulations can be in the form of a tablet, a capsule, a pill, a caplet, a gel, a cream, a granule, a solution, an emulsion, a suspension, or a nanoparticulate formulation. In some examples, the pharmaceutical formulations are oral formulations.

Also disclosed are methods of treating cancer in a subject in need thereof. The methods typically include administering an effective amount of a 5′-substituted nucleoside monophosphate, as described herein, a prodrug thereof, also as described herein, or a prodrug of a nucleoside monophosphate that is unsubstituted at the 5′ position, also as described herein, to the subject. In some examples, the prodrug of the 5′-substituted nucleoside monophosphate is administered. In some examples, the prodrug of the 5′-substituted nucleoside monophosphate is administered orally.

The cancer to be treated can be breast cancer, head and neck cancer, anal cancer, stomach cancer, skin cancer, colon and rectal cancer, pancreas cancer, esophageal cancer, gastrointestinal cancer, neuroendocrine tumor, thymic cancer, cervical cancer, bladder cancer, or hepatobiliary cancer. In some examples, the cancer is hepatobiliary cancer.

### BRIEF DESCRIPTION OF DRAWINGS

FIG. 1 shows a thermal ellipsoid representation of a representative molecule of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (MD-7-29) in the asymmetric unit.

FIG. 2 shows a thermal ellipsoid representation of the asymmetric unit of 1-((2R,4S,5R)-4-((tert-butyldimethylsilyl)oxy)-5-((S)-1-hydroxyethyl)tetrahydrofuran-2-yl)-5-fluoropyrimidine-2,4(1H,3H)-dione (MD-7-42). There are two molecules of the compound in the asymmetric unit.

FIG. 3 shows a thermal ellipsoid representation of a representative molecule of 1-((2R,4S,5R)-4-(benzyloxy)-5-((S)-2,2,2-trifluoro-1-hydroxyethyl)tetrahydrofuran-2-yl)-5-fluoropyrimidine-2,4(1H,3H)-dione (MD-7-159-1) in the asymmetric unit.

FIG. 4 is a diagram illustrating the availability of space in the binding pocket of hTS for substitutions at the 5′-position of the deoxy ribose ring of 5-fluorodeoxyuridine monophosphate (FdUMP).

FIG. 5 is a graph showing the normalized reaction rate (%) plotted against the log concentration of FdUMP (log([FdUMP])). The normalization was performed using the reaction rate determined in the absence of inhibitor as a reference. The experimental data was pooled from five repeats. FdUMP used in this assay was in the form of a disodium salt before being dissolved in the reaction buffer to make a stock solution.

FIG. 6 is a graph showing the normalized reaction rate (%) plotted against the log concentration of MD-7-105 (log([MD-7-105])). The normalization was performed using the reaction rate determined in the absence of inhibitor as a reference. The experimental data was pooled from five repeats. MD-7-105 used in this assay was in the form of a dilithium salt before being dissolved in the reaction buffer to make a stock solution.

### DETAILED DESCRIPTION

Before the present disclosure is described in greater detail, it is to be understood that this disclosure is not limited to particular examples described, and as such may, of course, vary. It is also to be understood that the terminology used herein is for the purpose of describing particular examples only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications and patents are cited.

Examples of the present disclosure will employ, unless otherwise indicated, techniques of medicine, organic chemistry, medicinal chemistry, biochemistry, molecular biology, pharmacology, and the like, which are within the skill of the art. Such techniques are explained fully in the literature, such as the references cited herein.

The disclosed compounds, mixtures, compositions, and formulations, can be used for, can be used in conjunction with, can be used in preparation for, or are products of the disclosed methods. All methods described herein can be performed in any suitable order unless otherwise indicated or otherwise clearly contradicted by context.

As will be apparent to those of skill in the art upon reading this disclosure, each of the individual examples described and illustrated herein has discrete components and features which may be readily separated from or combined with the features of any of the other several examples without departing from the scope or spirit of the present disclosure. It is understood that when combinations, subsets, interactions, groups, etc. of these compounds, mixtures, compositions, formulations, or components thereof are disclosed, while specific reference of each individual and collective combinations of these materials may not be explicitly disclosed, each is specifically contemplated and described herein. For example, if a compound is disclosed and discussed and a number of modifications or derivations that can be made to a number of molecules including the compound are discussed, each and every combination and permutation of the compound and the modifications/derivations that are possible are specifically contemplated unless specifically indicated to the contrary. Thus, if a class of chemical groups A, B, and C are disclosed as well as a class of chemical groups D, E, and F and an example of a combination, A-D, is disclosed, then even if each combination is not individually recited, each is individually and collectively contemplated. Thus, in this example, each of the combinations A-E, A-F, B-D, B-E, B-F, C-D, C-E, and C-F are specifically contemplated and should be considered disclosed from the disclosure of A, B, and C; D, E, and F; and the example combination A-D. Likewise, any subset or sub-group of the afore-mentioned combinations is also specifically contemplated and disclosed. Thus, for example, the sub-group of A-E, B-F, and C-E is specifically contemplated and should be considered disclosed from the disclosure of A, B, and C; D, E, and F; and the example combination A-D.

Further, each of the compounds, mixtures, compositions, formulations, and components thereof contemplated and disclosed as above can also be specifically and independently included or excluded from any group, sub-group, list, set, etc. of such materials. These concepts apply to all aspects of this application including, but not limited to, steps in methods of making and using the disclosed compounds, compositions, mixtures, and formulations. Thus, if there are a variety of additional steps that can be performed it is understood that each of these additional steps can be performed with any specific example or combination of examples of the disclosed methods, and that each such combination is specifically contemplated and should be considered disclosed.

### I. Definitions

It must be noted that, as used in the specification and the appended claims, the singular forms “a,” “an,” and “the” include plural referents unless the context clearly dictates otherwise. For example, reference to “a compound” or “the compound” may include a plurality of compounds.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

Use of the term “about” is intended to describe values either above or below the stated value in a range of approx. +/−10%; in other examples the values may range in value either above or below the stated value in a range of approx. +/−5%; in other examples the values may range in value either above or below the stated value in a range of approx. +/−2%; in other examples the values may range in value either above or below the stated value in a range of approx. +/−1%. The preceding ranges are intended to be made clear by context, and no further limitation is implied.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

A carbon range (e.g., C₁-C₁₀) is intended to disclose individually every possible carbon value and/or sub-range encompassed within. For example, a carbon range of C₁-C₁₀ discloses C₁, C₂, C₃, C₄, C₅, C₆, C₇, C₈, C₉, and C₁₀, as well as discloses sub-ranges encompassed therein, such as C₂-C₉, C₃-C₈, C₁-C₅, etc.

The terms “derivative” and “derivatives” refer to chemical compounds/moieties with a structure similar to that of a parent compound/moiety but different from it in respect to one or more components, functional groups, atoms, etc. Optionally, the derivatives retain sufficient functional attributes of the parent compound/moiety. Optionally, the derivatives can be formed from the parent compound/moiety by chemical reaction(s). The differences between the derivatives and the parent compound/moiety can include, but are not limited to, replacement of one or more functional groups with one or more different functional groups or introducing or removing one or more substituents of hydrogen atoms. The derivatives may be lacking one or more atoms, a salt, in different hydration/oxidation states, or having one or more atoms within the molecule switched, such as, but not limited to, replacing an oxygen atom with a sulfur atom or replacing an amino group with a hydroxy group. Contemplated derivatives include switching carbocyclic, aromatic rings with heterocyclic rings or switching heterocyclic rings with carbocyclic, aromatic rings, typically of the same or similar ring sizes. The derivatives can also differ from the parent compound/moiety with respect to the protonation state.

The term “alkyl” refers to univalent groups derived from alkanes (i.e., acyclic saturated hydrocarbons) by removal of a hydrogen atom from any carbon atom. Alkyl groups can be linear or branched. Suitable alkyl groups can have one to 30 carbon atoms, i.e., C₁-C₃₀ alkyl. If the alkyl is branched, it is understood that at least three carbon atoms are present.

The term “heteroalkyl” refers to alkyl groups where one or more carbon atoms are replaced with a heteroatom such as, O, N, S, or Si. Optionally, the nitrogen and/or sulphur heteroatom(s) may be oxidized, and the nitrogen heteroatom(s) may be quaternized. Heteroalkyl groups can be linear or branched. Suitable heteroalkyl groups can have one to 30 carbon atoms, i.e., C₁-C₃₀ heteroalkyl. If the heteroalkyl is branched, it is understood that at least one carbon atom and at least one heteroatom are present.

The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear or branched. Suitable alkenyl groups can have two to 30 carbon atoms, i.e., C₂-C₃₀ alkenyl. If the alkenyl is branched, it is understood that at least three carbon atoms are present.

The term “heteroalkenyl” refers to alkenyl groups in which one or more carbon atoms are replaced by a heteroatom such as, O, N, S, or Si. Optionally, the nitrogen and/or sulphur heteroatom(s) may be oxidized, and the nitrogen heteroatom(s) may be quaternized. Typically, the carbon atoms involved in the carbon-carbon double bond(s) are not replaced by any of the heteroatom(s). Heteroalkenyl groups can be linear or branched. Suitable heteroalkenyl groups can have two to 30 carbon atoms, i.e., C₂-C₃₀ heteroalkenyl. If the heteroalkenyl is branched, it is understood that at least two carbon atoms and at least one heteroatom are present.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear or branched. Suitable alkynyl groups can have two to 30 carbon atoms, i.e., C₂-C₃₀ alkynyl. If the alkynyl is branched, it is understood that at least four carbon atoms are present.

The term “heteroalkynyl” refers to alkynyl groups in which one or more carbon atoms are replaced by a heteroatom such as, O, N, S, or Si. Optionally, the nitrogen and/or sulphur heteroatom(s) may be oxidized, and the nitrogen heteroatom(s) may be quaternized. Typically, the carbon atoms involved in the carbon-carbon triple bond(s) are not replaced by any of the heteroatom(s). Heteroalkynyl groups can be linear or branched. Suitable heteroalkynyl groups can have two to 30 carbon atoms, i.e., C₂-C₃₀ heteroalkynyl. If the heteroalkynyl is branched, it is understood that at least two carbon atoms and at least one heteroatom are present.

The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner, in a fused manner, or a combination thereof. Accordingly, in polycyclic aryl groups, the rings can be attached together in a pendant manner, in a fused manner, or a combination thereof. Suitable aryl groups can have six to 50 carbon atoms, i.e., C₆-C₅₀ aryl. The number of “members” of an aryl group refers to the total number of carbon atoms in the ring(s) of the aryl group.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Huckel rule (4n+2). Heteroarenes can be monocyclic or polycyclic. In polycyclic heteroarenes, the rings can be attached together in a pendant manner, in a fused manner, or a combination thereof. Accordingly, in polycyclic heteroaryl groups, the rings can be attached together in a pendant manner, in a fused manner, or a combination thereof. Suitable heteroaryl groups can have three to 50 carbon atoms, i.e., C₃-C₅₀ heteroaryl. The number of “members” of a heteroaryl group refers to the total number of carbon atom(s) and heteroatom(s) in the ring(s) of the heteroaryl group.

“Carbocycle” or “carbocyclyl” refers to non-aromatic, cyclic hydrocarbon groups. They can be saturated or unsaturated (but non-aromatic), monocyclic or polycyclic. In polycyclic carbocyclyls, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. Suitable carbocyclyl groups can have three to 30 carbon atoms, i.e., C₃-C₃₀ carbocyclyl. The number of “members” of a carbocyclyl group refers to the total number of carbon atoms in the ring(s) of the carbocyclyl group.

“Heterocarbocycle” or “heterocarbocyclyl” refers to carbocycles in which one or more carbon atoms are replaced by heteroatom(s) independently selected from elements like nitrogen, oxygen, sulphur, and silicon. Optionally, the nitrogen and/or sulphur heteroatom(s) may be oxidized, and the nitrogen heteroatom(s) may be quaternized. Heterocarbocyclyls may be saturated or unsaturated (but not aromatic), monocyclic or polycyclic. In polycyclic heterocarbocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. Suitable heterocarbocyclyl groups can have two to 30 carbon atoms, i.e., C₂-C₃₀ heterocarbocyclyl. The number of “members” of a heterocarbocyclyl group refers to the total number of carbon atom(s) and heteroatom(s) in the ring(s) of the heterocarbocyclyl group.

As used herein, “heterocycle” or “heterocyclyl” refers to mono- and polycyclic ring systems having one or more heteroatoms independently selected from elements like nitrogen, oxygen, sulfur, and silicon, and containing at least two carbon atoms. Optionally, the nitrogen and/or sulphur heteroatom(s) may be oxidized, and the nitrogen heteroatom(s) may be quaternized. The mono- and polycyclic ring systems may be aromatic, non-aromatic, or a mixture of aromatic and non-aromatic rings. Heterocyclyls include heterocarbocyclyls and heteroaryls. In polycyclic heterocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. Suitable heterocyclyl groups can have two to 30 carbon atoms, i.e., C₂-C₃₀ heterocyclyl. The number of “members” of a heterocyclyl group refers to the total number of carbon atom(s) and heteroatom(s) in the ring(s) of the heterocyclyl group.

The term “substituted,” as used herein, means that the chemical group or moiety contains one or more substituents replacing the hydrogen atom(s) in the original chemical group or moiety. It is understood that any substitution is in accordance with permitted valence of the substituted atom and the substituent, and that the substitution results in a stable compound, e.g., a compound that does not spontaneously undergo transformation such as by rearrangement, cyclization, elimination, etc., under room temperature. Suitable substituents include, but are not limited to, deuterium, halogen, azido, cyano, isocyano, nitrate, nitrosooxy, nitroso, nitro, formyl, carboxyl, carbonate, alkyl, heteroalkyl, alkenyl, heteroalkenyl, alkynyl, heteroalkynyl, carbocyclyl, heterocyclyl, aryl, heteroaryl, azo, acyl, hydroxyl, thiol, sulfinyl, sulfonyl, sulfonate, sulfamoyl, amino, acylamino, amide, silyl, ester, thioester, carbonate ester, carbamate, aminooxy, hydroxyamino, and —SF₅, wherein each substituent may be further substituted by one or more R groups. In some examples, two substituents on the same atom can join together with that atom to form a cyclic moiety, such as a carbocycle or a heterocycle. The R group, in each occurrence, can be independently selected from halogen, alkyl optionally substituted by one or more halogen, heteroalkyl optionally substituted by one or more halogen atoms, alkenyl optionally substituted by one or more halogen, heteroalkenyl optionally substituted by one or more halogen, alkynyl optionally substituted by one or more halogen, heteroalkynyl optionally substituted by one or more halogen, carbocyclyl optionally substituted by one or more halogen, heterocyclyl optionally substituted by one or more halogen, aryl optionally substituted by one or more halogen, heteroaryl optionally substituted by one or more halogen, —OH, —SH, —NH₂, —N₃, —OCN, —NCO, —ONO₂, —CN, —NC, —ONO, —CONH₂, —NO, —NO₂, —ONH₂, —SCN, —SNCS, —SF₅, —CF₃, —CH₂CF₃, —CH₂Cl, —CHCl₂, —CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), —Si(R^{G1})(R^{G2})(R^{G3}), or -GR^{G1} in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2}), —C(═NR^{G2})O—, —C(═NR^{G2})NR^{G3}—, —OC(═NR^{G2})— NR^{G2}C(═NR^{G3})—, —NR^{G2}SO₂—, —C(═NR^{G2})NR^{G3}—, —OC(═NR^{G2}), —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is, independently, a hydrogen atom, a halogen atom, an alkyl group optionally substituted by one or more halogen, a heteroalkyl group optionally substituted by one or more halogen, an alkenyl group optionally substituted by one or more halogen, a heteroalkenyl group optionally substituted by one or more halogen, an alkynyl group optionally substituted by one or more halogen, a heteroalkynyl group optionally substituted by one or more halogen, a carbocyclyl group optionally substituted by one or more halogen, a heterocyclyl group optionally substituted by one or more halogen, an aryl group optionally substituted by one or more halogen, or a heteroaryl group optionally substituted by one or more halogen. When the R group is —Si(R^{G1})(R^{G2})(R^{G3}), two groups from R^{G1}, R^{G2}, and R^{G3} can join together with the Si atom to form a heterocycle. In some examples, two R groups on the same atom can join together with that atom to form a cyclic moiety, such as a carbocycle or a heterocycle.

The term “optionally substituted,” as used herein, means that substitution is optional and therefore it is possible for the designated atom/chemical group/compound to be unsubstituted.

As used herein, the term “halogen” refers to fluorine, chlorine, bromine, and iodine.

As used herein, the term “stereoisomers” refers to compounds made up of the same atoms having the same bond order but having different three-dimensional arrangements of atoms which are not interchangeable. As used herein, the term “enantiomers” refers to two stereoisomers which are non-superimposable mirror images of one another. As used herein, the term “diastereomer” refers to two stereoisomers which are not mirror images but also not superimposable. The terms “racemate” and “racemic mixture” refer to a mixture of enantiomers. The term “chiral center” refers to a carbon atom to which four different groups are attached. Choice of the appropriate chiral column, eluent, and conditions necessary for effective separation of stereoisomers, such as a pair of enantiomers, is well known to one of ordinary skill in the art using standard techniques (e.g., Jacques et al., Enantiomers, Racemates, and Resolutions, John Wiley and Sons, Inc., 1981).

As used herein, “subject” includes, but is not limited to, human or non-human mammals. The term does not denote a particular age or sex. Thus, adult and non-adult subjects, whether male or female, are intended to be covered. Exemplary subjects include human, livestock, and domestic pet. A “patient” refers to a subject afflicted with a disease or disorder, including human and non-human mammal subjects.

As used herein, the terms “prevent” and “preventing” include the prevention of the recurrence, spread or onset. It is not intended that the present disclosure is limited to complete prevention. In some examples, the onset is delayed, or the severity of the disease is reduced.

As used herein, the terms “treat” and “treating” are not limited to the case where the subject (e.g., patient) is cured and the disease is eradicated. Rather, examples of the present disclosure also contemplate treatment that merely reduces symptoms and/or delays disease progression.

### II. Compounds

Disclosed are 5′-substituted nucleoside monophosphates, which contain 5-fluorouracil or 5-fluorothiouracil as the nucleobase. In general, the 5′-substituted nucleoside monophosphates disclosed herein can inhibit human thymidylate synthase (TS) and thereby possess anti-cancer therapeutic effects.

The 5′-substitution in these nucleoside monophosphates can prevent metabolic cleavage of the monophosphate group, mediated by enzymes such as 5′-nucleotidases, phospholipase D (PLD), etc. This feature can improve metabolic profiles, enhance target specificity, and/or reduce side effects of these compounds, compared to their corresponding unsubstituted analogs.

Also disclosed are prodrugs of these 5′-substituted nucleoside monophosphates. After administration, the prodrugs can be metabolized to release their corresponding 5′-substituted nucleoside monophosphates.

Also disclosed are prodrugs of nucleoside monophosphates that are unsubstituted at the 5′ position. After administration to a subject as described herein, the prodrugs can be metabolized to release their corresponding nucleoside monophosphates.

To the extent that chemical formulas described herein contain one or more unspecified chiral centers, the formulas are intended to encompass all stable stereoisomers, enantiomers, and diastereomers. Such compounds can exist as a single enantiomer, a mixture of diastereomers, a racemic mixture, or combinations thereof. It is also understood that the chemical formulas encompass all tautomeric forms.

Optionally, the alkyl groups described herein have 1-30 carbon atoms, i.e., C₁-C₃₀ alkyl. In some forms, the C₁-C₃₀ alkyl can be a linear C₁-C₃₀ alkyl or a branched C₃-C₃₀ alkyl.

Optionally, the alkyl groups have 1-20 carbon atoms, i.e., C₁-C₂₀ alkyl. In some forms, the C₁-C₂₀ alkyl can be a linear C₁-C₂₀ alkyl or a branched C₃-C₂₀ alkyl. Optionally, the alkyl groups have 1-10 carbon atoms, i.e., C₁-C₁₀ alkyl. In some forms, the C₁-C₁₀ alkyl can be a linear C₁-C₁₀ alkyl or a branched C₃-C₁₀ alkyl. Representative straight chain alkyl groups include methyl, ethyl, n-propyl, n-butyl, n-pentyl, n-hexyl, n-heptyl, n-octyl, n-nonyl, and the like. Representative branched alkyl groups include isopropyl, sec-butyl, isobutyl, tert-butyl, isopentyl, and the like.

Optionally, the heteroalkyl groups described herein have 1-30 carbon atoms, i.e., C₁-C₃₀ heteroalkyl. In some forms, the C₁-C₃₀ heteroalkyl can be a linear C₁-C₃₀ heteroalkyl or a branched C₁-C₃₀ heteroalkyl. Optionally, the heteroalkyl groups have 1-20 carbon atoms, i.e., C₁-C₂₀ heteroalkyl. In some forms, the C₁-C₂₀ heteroalkyl can be a linear C₁-C₂₀ heteroalkyl or a branched C₁-C₂M heteroalkyl. Optionally, the heteroalkyl groups have 1-10 carbon atoms, i.e., C₁-C₁₀ heteroalkyl. In some forms, the C₁-C₁₀ heteroalkyl can be a linear C₁-C₁₀ heteroalkyl or a branched C₁-C₁₀ heteroalkyl.

Optionally, the alkenyl groups described herein have 2-30 carbon atoms, i.e., C₂-C₃₀ alkenyl. In some forms, the C₂-C₃₀ alkenyl can be a linear C₂-C₃₀ alkenyl or a branched C₃-C₃₀ alkenyl. Optionally, the alkenyl groups have 2-20 carbon atoms, i.e., C₂-C₂₀ alkenyl. In some forms, the C₂-C₂₀ alkenyl can be a linear C₂-C₂₀ alkenyl or a branched C₃-C₂₀ alkenyl. Optionally, the alkenyl groups have 2-10 carbon atoms, i.e., C₂-C₁₀ alkenyl. In some forms, the C₂-C₁₀ alkenyl can be a linear C₂-C₁₀ alkenyl or a branched C₃-C₁₀ alkenyl. Representative alkenyl groups include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

Optionally, the heteroalkenyl groups described herein have 2-30 carbon atoms, i.e., C₂-C₃₀ heteroalkenyl. In some forms, the C₂-C₃₀ heteroalkenyl can be a linear C₂-C₃₀ heteroalkenyl or a branched C₂-C₃₀ heteroalkenyl. Optionally, the heteroalkenyl groups have 2-20 carbon atoms, i.e., C₂-C₂₀ heteroalkenyl. In some forms, the C₂-C₂₀ heteroalkenyl can be a linear C₂-C₂₀ heteroalkenyl or a branched C₂-C₂₀ heteroalkenyl. Optionally, the heteroalkenyl groups have 2-10 carbon atoms, i.e., C₂-C₁₀ heteroalkenyl. In some forms, the C₂-C₁₀ heteroalkenyl can be a linear C₂-C₁₀ heteroalkenyl or a branched C₂-C₁₀ heteroalkenyl.

Optionally, the alkynyl groups described herein have 2-30 carbon atoms, i.e., C₂-C₃₀ alkynyl. In some forms, the C₂-C₃₀ alkynyl can be a linear C₂-C₃₀ alkynyl or a branched C₄-C₃₀ alkynyl. Optionally, the alkynyl groups have 2-20 carbon atoms, i.e., C₂-C₂₀ alkynyl. In some forms, the C₂-C₂₀ alkynyl can be a linear C₂-C₂₀ alkynyl or a branched C₄-C₂₀ alkynyl. Optionally, the alkynyl groups have 2-10 carbon atoms, i.e., C₂-C₁₀ alkynyl. In some forms, the C₂-C₁₀ alkynyl can be a linear C₂-C₁₀ alkynyl or a branched C₄-C₁₀ alkynyl. Representative alkynyl groups include ethynyl, propynyl, 1-butynyl, 2-butynyl, 1-pentynyl, 2-pentynyl, 3-methyl-1-butynyl, and the like.

Optionally, the heteroalkynyl groups described herein have 2-30 carbon atoms, i.e., C₂-C₃₀ heteroalkynyl. In some forms, the C₂-C₃₀ heteroalkynyl can be a linear C₂-C₃₀ heteroalkynyl or a branched C₂-C₃₀ heteroalkynyl. Optionally, the heteroalkynyl groups have 2-20 carbon atoms, i.e., C₂-C₂₀ heteroalkynyl. In some forms, the C₂-C₂₀ alkenyl can be a linear C₂-C₂₀ heteroalkynyl or a branched C₂-C₂₀ heteroalkynyl. Optionally, the heteroalkynyl groups have 2-10 carbon atoms, i.e., C₂-C₁₀ heteroalkynyl. In some forms, the C₂-C₁₀ heteroalkynyl can be a linear C₂-C₁₀ heteroalkynyl or a branched C₂-C₁₀ heteroalkynyl.

Optionally, the aryl groups described herein have 6-30 carbon atoms, i.e., C₆-C₃₀ aryl. Optionally, the aryl groups have 6-20 carbon atoms, i.e., C₆-C₂₀ aryl. Optionally, the aryl groups have 6-12 carbon atoms, i.e., C₆-C₁₂ aryl. Representative aryl groups include phenyl, naphthyl, and biphenyl.

Optionally, the heteroaryl groups described herein have 3-30 carbon atoms, i.e., C₃-C₃₀ heteroaryl. Optionally, the heteroaryl groups have 3-20 carbon atoms, i.e., C₃-C₂₀ heteroaryl. Optionally, the heteroaryl groups have 3-11 carbon atoms, i.e., C₃-C₁₁ heteroaryl. Representative heteroaryl groups include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

Optionally, the carbocyclyl groups described herein have 3-30 carbon atoms, i.e., C₃-C₃₀ carbocyclyl. Optionally, the carbocyclyl groups described herein have 3-20 carbon atoms, i.e., C₃-C₂₀ carbocyclyl. Optionally, the carbocyclyl groups described herein have 3-12 carbon atoms, i.e., C₃-C₁₂ carbocyclyl. Representative saturated carbocyclyl groups include cyclopropyl, cyclobutyl, cyclopentyl, cyclohexyl, and the like. Representative unsaturated carbocyclyl groups include cyclopentenyl, cyclohexenyl, and the like.

Optionally, the heterocarbocyclyl groups described herein have 2-30 carbon atoms, i.e., C₂-C₃₀ heterocarbocyclyl. Optionally, the heterocarbocyclyl groups described herein have 2-20 carbon atoms, i.e., C₂-C₂₀ heterocarbocyclyl. Optionally, the heterocarbocyclyl groups described herein have 2-11 carbon atoms, i.e., C₂-C₁₁ heterocarbocyclyl. Representative heterocarbocyclyl groups include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, and the like.

In some examples, the optionally O-substituted hydroxyl groups described herein may be —O—R_{b}, wherein R_{b}, in each occurrence, is independently selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “ester” refers to —C(═O)OR_{c1} or —OC(═O)R_{c2}, wherein R_{c1} and R_{c2} are independently selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally and independently substituted by one or more R groups described herein.

As used herein, “amino” refers to —NR_{d1}R_{d2}, wherein R_{f1} and R_{f2} are independently selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally and independently substituted by one or more R groups described herein. When R_{d1} and R_{d2} are hydrogen, the amino group is primary amino.

As used herein, “acyl” refers —C(═O)Rₑ, wherein Rₑ is selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “amide” refers to —C(═O)NR_{f1}R_{f2}, wherein R_{f1} and R_{f2} are independently selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally and independently substituted by one or more R groups described herein. When both R_{f1} and R_{f2} are hydrogen, the amide group is carbamoyl.

As used herein, “acylamino” refers to —NR_{g}[C(═O)Rₕ], wherein R_{g} is selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein, and Rₕ is selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “carbonate ester” refers to —OC(═O)ORᵢ, wherein Rᵢ is selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “carbamate” refers to —OC(═O)NRⱼ₁Rⱼ₂ or —NRₖ[(C═O)OR₁], wherein Rⱼ₁, Rⱼ₂, Rₖ, and Rₗ are independently selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally and independently substituted by one or more R groups described herein.

As used herein, “sulfinyl” refers to —S(═O)Rₘ, wherein Rₘ is selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “sulfonyl” refers to —S(═O)₂Rₙ, wherein Rₙ is selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “thioester” refers to —C(═O)SRₒ₁ or —SC(═O)Rₒ₂, wherein Rₒ₁ and Rₒ₂ are independently selected from alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally and independently substituted by one or more R groups described herein.

As used herein, “disulfide” refers to —S—S—R_{z}, wherein R_{z} is independently selected from hydrogen, alkyl, heteroalkyl, alkenyl, heteroalkenyl, alkynyl, heteroalkynyl, carbocyclyl, heterocyclyl, aryl, and heteroaryl, wherein each of the alkyl, heteroalkyl, alkenyl, heteroalkenyl, alkynyl, heteroalkynyl, carbocyclyl, heterocyclyl, aryl, and heteroaryl can be optionally substituted by one or more R groups described herein.

As used herein, “thiol” refers to the univalent radical —SH.

As used herein, “sulfonate” refers to —SO₃.

As used herein, “sulfamoyl” refers to —S(═O)₂NH₂.

As used herein, “silyl” refers to the univalent radical derived from silane by removal of a hydrogen atom, i.e., —SiH₃.

As used herein, “carbonate” refers to —O(C═O)OH.

As used herein, “aminooxy” refers to —O—NH₂.

As used herein, “hydroxyamino” refers to —NH(OH).

As used herein, “alkoxy” or “alkyloxy” refers to an alkyl group as defined herein with the indicated number of carbon atoms attached through an oxygen bridge. Exemplary alkoxy groups include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy.

As used herein, “alkylamino” refers an alkyl group as defined herein with the indicated number of carbon atoms attached through an amino bridge. An example of an alkylamino is methylamino (i.e., —NH—CH₃).

As used herein, “alkylthio” refers to an alkyl group as defined herein with the indicated number of carbon atoms attached through a sulfur bridge. An example of an alkylthio is methylthio (i.e., —S—CH₃).

As used herein, “alkylene” or “bridging alkylene” refers to divalent functional groups derived from an alkane by removal of hydrogen atoms from two different carbon atoms (for example, ethylene (i.e., —CH₂—CH₂—)), or by removal of two hydrogen atoms from one carbon atom (for example, methylene (i.e., —CH₂—)).

As used herein, “pharmaceutically acceptable salt” refers to the modification of the original compound by making the acid or base salts thereof. Examples of pharmaceutically acceptable salts include, but are not limited to, mineral or organic acid salts of basic residues such as amines, as well as alkali or organic salts of acidic residues such as carboxylic acids or phosphorus acids. For original compounds containing a basic residue, the pharmaceutically acceptable salts can be prepared by treating the original compounds with an appropriate amount of a non-toxic inorganic or organic acid; alternatively, the pharmaceutically acceptable salts can be formed in situ during preparation of the original compounds; alternatively, the pharmaceutically acceptable salts can be prepared via ion-exchange with existing salts of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the pharmaceutically acceptable salts can be prepared by treating the original compounds with an appropriate amount of a non-toxic base; alternatively, the pharmaceutically acceptable salts can be formed in situ during preparation of the original compounds; alternatively, the pharmaceutically acceptable salts can be prepared via ion-exchange with existing salts of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Optionally, the pharmaceutically acceptable salts can be prepared by reacting the free acid or base form of the original compounds with a stoichiometric amount or more of the appropriate base or acid, respectively, in water, in an organic solvent, or in a mixture thereof. Lists of suitable pharmaceutically acceptable salts can be found in Remington's Pharmaceutical Sciences, 20th Ed., Lippincott Williams & Wilkins, Baltimore, M D, 2000; and Handbook of Pharmaceutical Salts: Properties, Selection, and Use, Stahl and Wermuth, Eds., Wiley-VCH, Weinheim, 2002.

In some examples, the pharmaceutical acceptable salts of the disclosed compounds and prodrugs thereof are salts with ammonium hydroxide, i.e., ammonium salts. In some examples, the pharmaceutical acceptable salts of the disclosed compounds and prodrugs thereof are lithium salts.

A. 5′-Substituted Nucleoside Monophosphates

The 5′-substituted nucleoside monophosphates disclosed herein have a structure of Formula I or a pharmaceutically acceptable salt thereof:

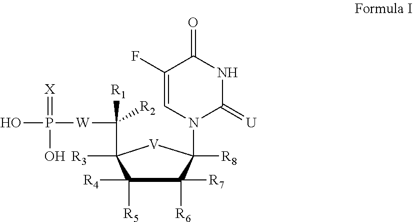

wherein:
(1) R₁ and R₂ are independently selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen,
(2) R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ, or
(3) one of R₁ and R₂ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and the other one of R₁ and R₂ joins with the 4′ carbon and the 5′ carbon to form a 3- or 4-membered carbocycle optionally substituted by one or more Rₐ;
wherein U is O or S;
wherein V is O or S;
wherein W is O or optionally substituted methylene;
wherein X is O or S;
wherein R₃ is absent or selected from hydrogen, deuterium, halogen, cyano, ethynyl, optionally substituted alkyl, optionally O-substituted hydroxyl, and ester;
wherein R₄ is hydrogen or deuterium;
wherein R₅ is selected from fluorine, optionally O-substituted hydroxyl, amino, acyl, ester, amide, acylamino, and substituted alkyl containing a substituent selected from fluorine, optionally O-substituted hydroxyl, and amino;
wherein R₆, R₇, and R₈ are independently selected from hydrogen, deuterium, halogen, cyano, ethynyl, optionally substituted alkyl, optionally O-substituted hydroxyl, and ester; and wherein Rₐ is selected from deuterium, halogen, and hydroxyl.

The “ethene moiety” formed by R₁, R₂, and the 5′ carbon is —(C═CH₂—

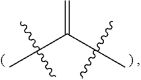 [SMILES: *C(*)=C]

which can be substituted by one or more Rₐ. In this group, the carbon atom with two open valences is the 5′ carbon; R₁ and R₂ merge together to form ═CH₂, which can be substituted by one or more Rₐ. An exemplary compound containing the ethene moiety is 1-((2S,3S,5R)-5-(5-fluoro-2,4-dioxo-3,4-dihydropyrimidin-1(2H)-yl)-3-hydroxytetrahydrofuran-2-yl)vinyl dihydrogen phosphate.

The substituted or optionally substituted groups described in Formula I can have one or more substituents independently selected from deuterium, halogen, azido, cyano, isocyano, nitrate, nitrosooxy, nitroso, nitro, formyl, carboxyl, carbonate, alkyl, heteroalkyl, alkenyl, heteroalkenyl, alkynyl, heteroalkynyl, carbocyclyl, heterocyclyl, aryl, heteroaryl, azo, acyl, hydroxyl, thiol, sulfinyl, sulfonyl, sulfonate, sulfamoyl, amino, acylamino, amide, silyl, ester, thioester, carbonate ester, carbamate, aminooxy, hydroxyamino, and —SF₅, wherein each substituent may be further substituted by one or more R groups. In some examples, two substituents on the same atom can join together with that atom to form a cyclic moiety, such as a carbocycle or a heterocycle.

The R group, in each occurrence, can be independently selected from halogen, alkyl optionally substituted by one or more halogen, heteroalkyl optionally substituted by one or more halogen, alkenyl optionally substituted by one or more halogen, heteroalkenyl optionally substituted by one or more halogen, alkynyl optionally substituted by one or more halogen, heteroalkynyl optionally substituted by one or more halogen, carbocyclyl optionally substituted by one or more halogen, heterocyclyl optionally substituted by one or more halogen, aryl optionally substituted by one or more halogen, heteroaryl optionally substituted by one or more halogen, —OH, —SH, —NH₂, —N₃, —OCN, —NCO, —ONO₂, —CN, —NC, —ONO, —CONH₂, —NO, —NO₂, —ONH₂, —SCN, —SNCS, —SF₅, —CF₃, —CH₂CF₃, —CH₂Cl, —CHCl₂, —CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), —Si(R^{G1})(R^{G2})(R^{G3}), or -GR^{G1} in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2}), —C(═NR^{G2})O—, —C(═NR^{G1})NR—, —OC(═NR^{G2})— NR^{G2}C(═NR^{G3})—, —NR^{G2}SO₂—, —C(═NR^{G2})NR^{G3}—, —OC(═NR^{G1}), —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is, independently, a hydrogen atom, a halogen atom, an alkyl group optionally substituted by one or more halogen, a heteroalkyl group optionally substituted by one or more halogen, an alkenyl group optionally substituted by one or more halogen, a heteroalkenyl group optionally substituted by one or more halogen, an alkynyl group optionally substituted by one or more halogen, a heteroalkynyl group optionally substituted by one or more halogen, a carbocyclyl group optionally substituted by one or more halogen, a heterocyclyl group optionally substituted by one or more halogen, an aryl group optionally substituted by one or more halogen, or a heteroaryl group optionally substituted by one or more halogen. When the R group is —Si(R^{G1})(R^{G2})(R^{G3}), two groups from R^{G1}, R^{G2}, and R^{G3} can join together with the Si atom to form a heterocycle.

In some examples, two R groups on the same atom can join together with that atom to form a cyclic moiety, such as a carbocycle or a heterocycle.

In some examples, the substituted or optionally substituted groups described in Formula I can have one or more substituents independently selected from alkyl, heteroalkyl, alkenyl, heteroalkenyl, alkynyl, heteroalkynyl, carbocyclyl, heterocyclyl, aryl, and heteroaryl, wherein each substitutent may be further substituted by one or more R groups. In some examples, two substituents on the same atom can join together with that atom to form a cyclic moiety, such as a carbocycle or a heterocycle.

In some examples, the substituted or optionally substituted groups described in Formula I can have one or more substituents independently selected from deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(tert-butyl)silyl, methyl(methyl)(isobutyl)silyl), formyl, carboxyl, mercapto, sulfamoyl, alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), alkyl (such as methyl, ethyl, isopropyl, tert-butyl), heteroalkyl, alkenyl, heteroalkenyl, alkynyl, heteroalkynyl, carbocyclyl, heterocyclyl, aryl, heteroaryl, and —SF₅.

In some examples, the substituted groups or optionally substituted groups described in Formula I can have one or more substituents independently selected from deuterium, halogen, nitro, cyano, hydroxyl, trifluoromethoxy, trifluoromethyl, trimethylsilyl, amino, formyl, carboxyl, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, carbocyclyl, aryl, and heterocyclyl.

1. Substituents at the 5′ Position

### Group I

Optionally, R₁ and R₂ are independently selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen.

In some examples, R₁ is hydrogen, deuterium, halogen, methyl optionally substituted by one or more Rₐ, or ethenyl optionally substituted by one or more Rₐ. In some examples, R₁ is hydrogen, methyl, or ethenyl. In some examples, R₁ is hydrogen.

In some examples, R₁ is cyano, carboxyl, ethyl or propyl optionally substituted by one or more Rₐ, ethynyl optionally substituted by Rₐ, 3- or 4-membered carbocyclyl optionally substituted by one or more Rₐ, or 3- or 4-membered heterocyclyl optionally substituted by one or more Rₐ.

In some examples, R₂ is cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, or 3- or 4-membered heterocyclyl optionally substituted by one or more Rₐ. In some examples, R₂ is methyl optionally substituted by one or more Rₐ, ethyl optionally substituted by one or more Rₐ, ethenyl optionally substituted by one or more Rₐ, or 3- or 4-membered carbocyclyl optionally substituted by one or more Rₐ. In some examples, R₂ is methyl, —CF₃, —CHF₂, —CH₂OH, ethenyl, cyclopropyl, or cyclobutyl. In some examples, R₂ is methyl, —CF₃, or —CH₂OH. In some examples, R₂ is methyl. In some examples, R₂ is —CF₃.

In some examples, R₂ is hydrogen, deuterium, or halogen. In some examples, R₂ is hydrogen.

In some examples, R₁ is hydrogen, and R₂ is selected from cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ. In some examples, R₁ is hydrogen, and R₂ is methyl optionally substituted by one or more Rₐ, ethyl optionally substituted by one or more Rₐ, ethenyl optionally substituted by one or more Rₐ, or 3- or 4-membered carbocyclyl optionally substituted by one or more Rₐ. In some examples, R₁ is hydrogen, and R₂ is methyl, —CF₃, —CHF₂, —CH₂OH, ethenyl, cyclopropyl, or cyclobutyl. In some examples, R₁ is hydrogen and R₂ is methyl, —CF₃, or —CH₂OH. In some examples, R₁ is hydrogen and R₂ is methyl. In some examples, R₁ is hydrogen and R₂ is —CF₃.

### Group II

Optionally, R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ.

In some examples, R₁ and R₂ join with the 5′ carbon to form a cyclopropane moiety optionally substituted by one or more Rₐ, a cyclobutene moiety optionally substituted by one or more Rₐ, or an ethene moiety optionally substituted by one or more Rₐ.

In some examples, R₁ and R₂ join with the 5′ carbon to form a cyclopropane moiety, a cyclobutene moiety, or an ethene moiety.

### Group III

Optionally, one of R₁ and R₂ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and the other one of R₁ and R₂ joins with the 4′ carbon and the 5′ carbon to form a 3- or 4-membered carbocycle optionally substituted by one or more Rₐ.

In some examples, R₁ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and R₂ joins with the 4′ carbon and the 5′ carbon to form a 3- or 4-membered carbocycle optionally substituted by one or more Rₐ. In some examples, R₁ is hydrogen. In some examples, R₂ joins with the 4′ carbon and the 5′ carbon to form a 3- or 4-membered carbocycle. In some examples, R₁ is hydrogen, and R₂ joins with the 4′ carbon and the 5′ carbon to form a 3- or 4-membered carbocycle.

In some examples, R₂ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and R₁ joins with the 4′ carbon and the 5′ carbon to form a 3- or 4-membered carbocycle optionally substituted by one or more Rₐ. In some examples, R₂ is hydrogen. In some examples, R₁ joins with the 4′ carbon and the 5′ carbon to form a 3- or 4-membered carbocycle. In some examples, R₂ is hydrogen, and R₁ joins with the 4′ carbon and the 5′ carbon to form a 3- or 4-membered carbocycle.

2. U, V, W, and X

In some examples, U is O. In some examples, U is S.

In some examples, V is O. In some examples, V is S.

In some examples, W is O. In some examples, W is optionally substituted methylene. In some examples, W is methylene (i.e., —CH₂—).

In some examples, X is O. In some examples, X is S.

In some examples, U, V, W, and X are O. In some examples, V, W, and X are O, and U is S. In some examples, U, W, and X are O, and V is S. In some examples, U, V, and X are O, and W is methylene. In some examples, U, V, and W are O, and X is S.

3. R₃ to R₈

R₃ is absent or selected from hydrogen, deuterium, halogen, cyano, ethynyl, optionally substituted alkyl, optionally O-substituted hydroxyl, and ester.

In some examples, R₃ is absent (e.g., for Group III described above).

In some examples, R₃ is selected from hydrogen, deuterium, halogen, cyano, ethynyl, optionally substituted alkyl, optionally O-substituted hydroxyl, and ester. In some examples, R₃ is selected from hydrogen, deuterium, halogen, cyano, ethynyl, optionally substituted alkyl, —OR_{b}, and —OC(═O)R_{c2}. R_{b} and R_{c2} are the same as described above. In some examples, R₃ is hydrogen.

R₄ is hydrogen or deuterium. In some examples, R₄ is hydrogen.

R₅ is selected from fluorine, optionally O-substituted hydroxyl, amino, acyl, ester, amide, acylamino, and substituted alkyl containing a substituent selected from fluorine, optionally O-substituted hydroxyl, and amino. In some examples, R₅ is selected from fluorine, substituted alkyl containing one or more fluorine substituents, —OR_{b}, —RₚOR_{b}, —OC(═O)R_{c2}, —C(═O)OR_{c1}, —NR_{d1}R_{d2}, —C(═O)Rₑ, —C(═O)NR_{f1}R_{f2}, —RₚNR_{d1}R_{d2}, and —NR_{g}C(═O)Rₕ. R_{b}, R_{c1}, R_{c2}, R_{d1}, R_{d2}, Rₑ, R_{f1}, R_{f2}, R_{g}, and Rₕ are the same as described above. Rₚ is an optionally substituted bridging alkylene. In some examples, R₅ is hydroxyl.

R₆, R₇, and R₈ are independently selected from hydrogen, deuterium, halogen, cyano, ethynyl, optionally substituted alkyl, optionally O-substituted hydroxyl, and ester. In some examples, R₆, R₇, and R₈ are independently selected from hydrogen, deuterium, halogen, cyano, ethynyl, optionally substituted alkyl, —OR_{b}, and —OC(═O)R_{c2}. R_{b} and R_{c2} are the same as described above.

In some examples, R₆ and R₇ are independently selected from hydrogen, deuterium, fluorine, and hydroxyl, with the proviso that R₆ and R₇ are not both hydroxyl. In some examples, R₆ is hydrogen. In some examples, R₆ is hydroxyl. In some examples, R₇ is hydrogen. In some examples, R₆ and R₇ are hydrogen. In some examples, R₆ is hydroxyl and R₇ is hydrogen.

In some examples, R₈ is hydrogen.

In some examples, R₃, R₄, R₆, R₇, and R₈ are hydrogen, and R₅ is hydroxyl. In some examples, R₃, R₄, R₇, and R₈ are hydrogen, and R₅ and R₆ are hydroxyl.

4. Exemplary Compounds

In some examples, the 5′-substituted nucleoside monophosphates have a structure of Formula Ia or a pharmaceutically acceptable salt thereof:

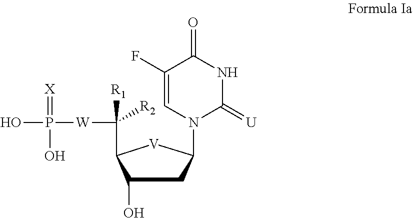

wherein R₁, R₂, U, V, W, and X are the same as described above.

In some examples, R₁ is hydrogen and R₂ is selected from cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ. In some examples, R₁ is hydrogen and R₂ is methyl optionally substituted by one or more Rₐ, ethyl optionally substituted by one or more Rₐ, ethenyl optionally substituted by one or more Rₐ, or 3- or 4-membered carbocyclyl optionally substituted by one or more Rₐ. In some examples, R₁ is hydrogen and R₂ is methyl, —CF₃, —CHF₂, —CH₂OH, ethenyl, cyclopropyl, or cyclobutyl. In some examples, R₁ is hydrogen and R₂ is methyl, —CF₃, or —CH₂OH. In some examples, R₁ is hydrogen and R₂ is methyl or —CF₃.

In some examples, R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ. In some examples, R₁ and R₂ join with the 5′ carbon to form a cyclopropane moiety optionally substituted by one or more Rₐ, a cyclobutene moiety optionally substituted by one or more Rₐ, or an ethene moiety optionally substituted by one or more Rₐ. In some examples, R₁ and R₂ join with the 5′ carbon to form a cyclopropane moiety, a cyclobutene moiety, or an ethene moiety.

In some examples, R₁ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and R₂ joins with the 4′ carbon and the 5′ carbon to form a 3- or 4-membered carbocycle optionally substituted by one or more Rₐ. In some examples, R₁ is hydrogen, and R₂ joins with the 4′ carbon and the 5′ carbon to form a 3- or 4-membered carbocycle.

In some examples, the 5′-substituted nucleoside monophosphates have a structure of Formula Ia′ or a pharmaceutically acceptable salt thereof:

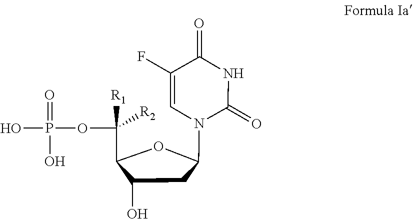

wherein R₁ and R₂ are the same as described above.

In some examples, R₁ is hydrogen and R₂ is selected from cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ. In some examples, R₁ is hydrogen and R₂ is methyl optionally substituted by one or more Rₐ, ethyl optionally substituted by one or more Rₐ, ethenyl optionally substituted by one or more Rₐ, or 3- or 4-membered carbocyclyl optionally substituted by one or more Rₐ. In some examples, R₁ is hydrogen and R₂ is methyl, —CF₃, —CHF₂, —CH₂OH, ethenyl, cyclopropyl, or cyclobutyl. In some examples, R₁ is hydrogen and R₂ is methyl, —CF₃, or —CH₂OH. In some examples, R₁ is hydrogen and R₂ is methyl or —CF₃.

In some examples, R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ. In some examples, R₁ and R₂ join with the 5′ carbon to form a cyclopropane moiety optionally substituted by one or more Rₐ, a cyclobutene moiety optionally substituted by one or more Rₐ, or an ethene moiety optionally substituted by one or more Rₐ. In some examples, R₁ and R₂ join with the 5′ carbon to form a cyclopropane moiety, a cyclobutene moiety, or an ethene moiety.

In some examples, R₁ is selected from hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and R₂ joins with the 4′ carbon and the 5′ carbon to form a 3- or 4-membered carbocycle optionally substituted by one or more Rₐ. In some examples, R₁ is hydrogen, and R₂ joins with the 4′ carbon and the 5′ carbon to form a 3- or 4-membered carbocycle.

Exemplary 5′-substituted nucleoside monophosphates include, but are not limited to, the following compounds and pharmaceutically acceptable salts (such as ammonium and lithium salts) thereof:

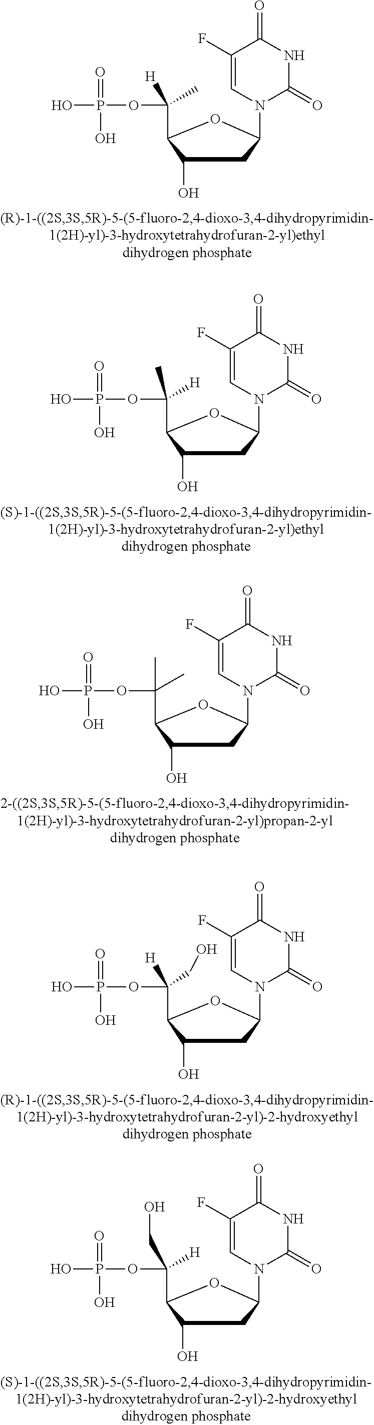

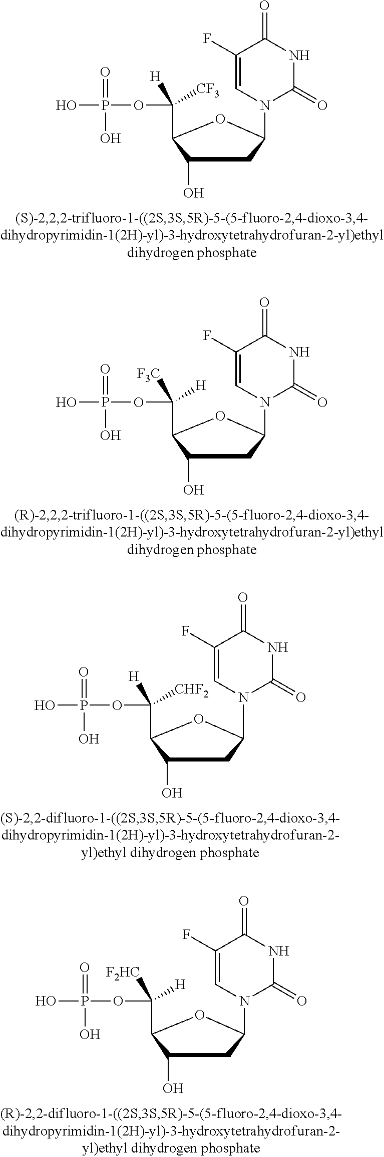

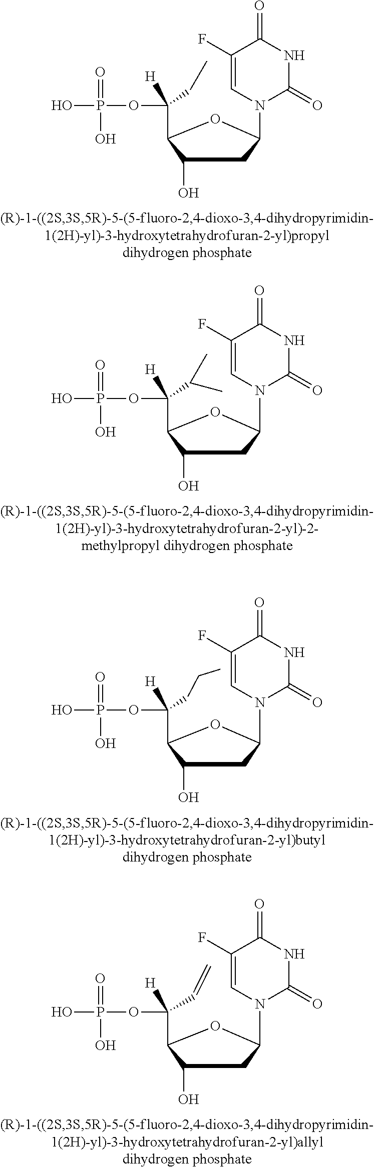

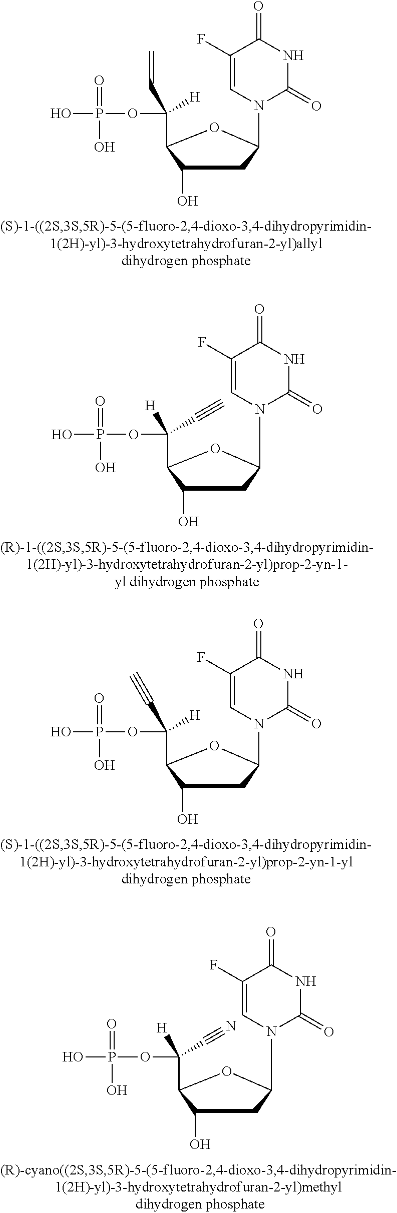

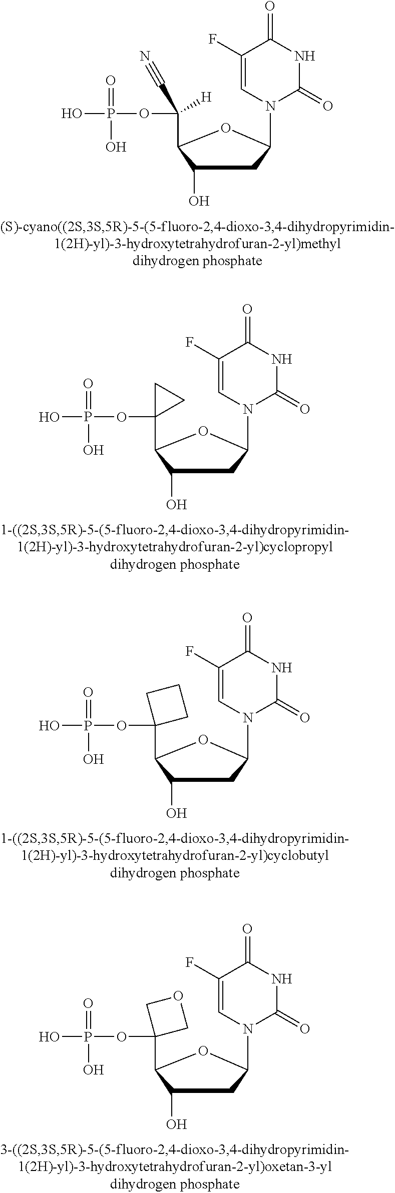

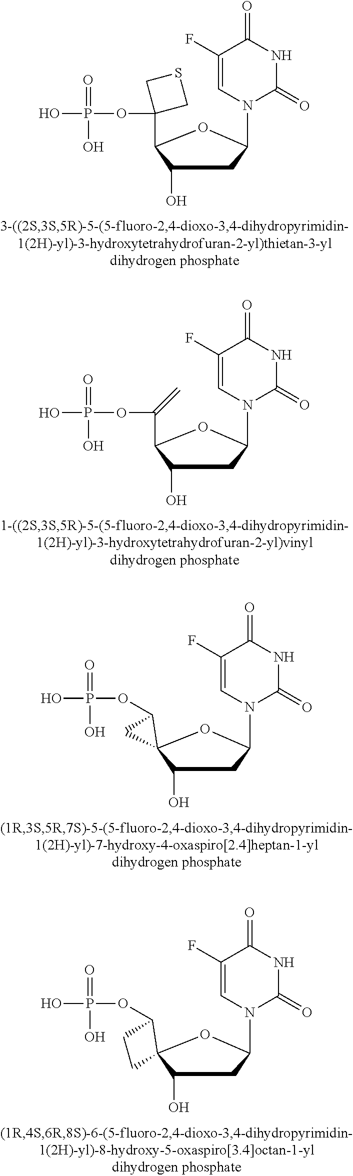

In some examples, the 5′-substituted nucleoside monophosphates are selected from the following compounds and pharmaceutically acceptable salts (such as ammonium and lithium salts) thereof:

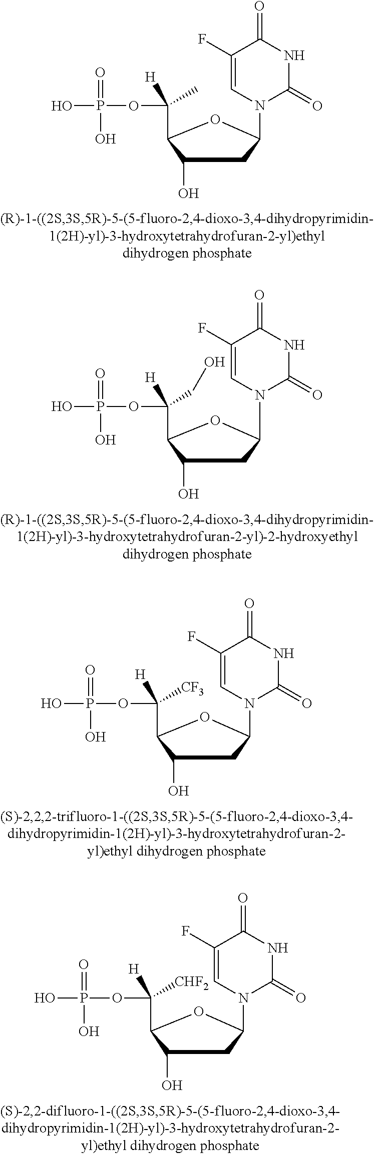

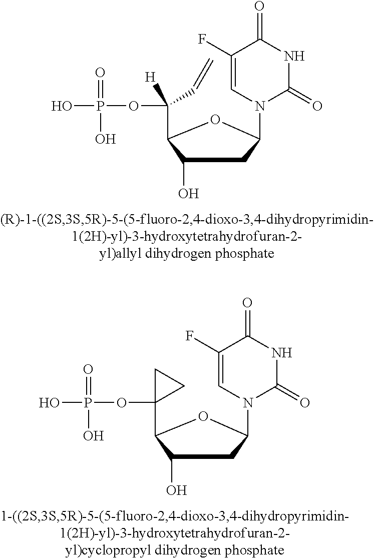

B. Prodrugs of 5′-Substituted Nucleoside Monophosphates

In general, prodrugs of the 5′-substituted nucleoside monophosphates have a structure of Formula II or Formula III, or a pharmaceutically acceptable salt thereof:

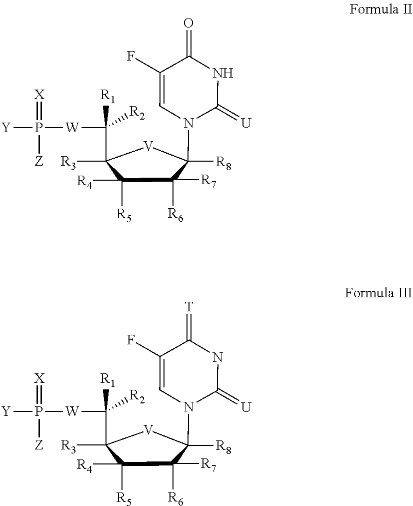

wherein R₁, R₂, R₃, R₄, R₅, R₆, R₇, R₈, U, V, W, and X are as described above;
wherein Y and Z are independently selected from —O—R₉, —S—R₁₀, and R₁ with the proviso that Y and Z are not both hydroxyl;

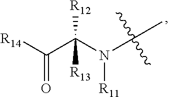

wherein T is —NR₁₅R₁₆ or —OR₁₇;
wherein R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, —R_{q1}—R_{q2}—R_{q3}—R_{q4}, and —Rᵣ₁—Rᵣ₂;
wherein R₁₀ is selected from optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₁ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₂ and R₁₃ are independently selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and standard amino acid side chain,
wherein when the standard amino acid side chain is a proline side chain, one of R₁₂ and R₁₃ is hydrogen, and the other one of R₁₂ and R₁₃ forms a pyrrolidine ring together with R₁₁, the nitrogen atom connected to R₁₁, and the carbon atom connected to R₁₂ and R₁₃;
wherein R₁₄ is —NRₛ₁Rₛ₂ or —ORₜ;
wherein R₁₅ and R₁₆ are independently selected from hydrogen, acyl, ester, thioester, and amide;
wherein R₁₇ is acyl, ester, thioester, or amide;
wherein:
   R_{q1} is absent or a C₁-C₉ alkyl chain (i.e., C₁-C₉ bridging alkylene),
   R_{q2} is absent or is selected from substituted methylene or ethylene, —O—, —S—, —S(═O)—, —S—S—, and —S(O)₂—,
   R_{q3} is a C₂-C₂₀ alkyl chain (i.e., C₂-C₂₀ bridging alkylene), and
   R_{q4} is selected from hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅;
wherein:
   Rᵣ₁ is optionally substituted bridging C₁-C₄ alkylene or -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5, and
   Rᵣ₂ is selected from ester, thioester, amide, acylamino, carbonate ester, carbomate, disulfide, optionally substituted (4-acylamino)phenyl, and optionally substituted (4-acyloxy)phenyl; and
wherein:
   Rₛ₁ and Rₛ₂ are independently selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl, and
   Rₜ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl.

In some examples, the prodrugs have a structure of Formula IIa, Formula Ib, Formula IIIa, Formula IIIb, or a pharmaceutically acceptable salt thereof,

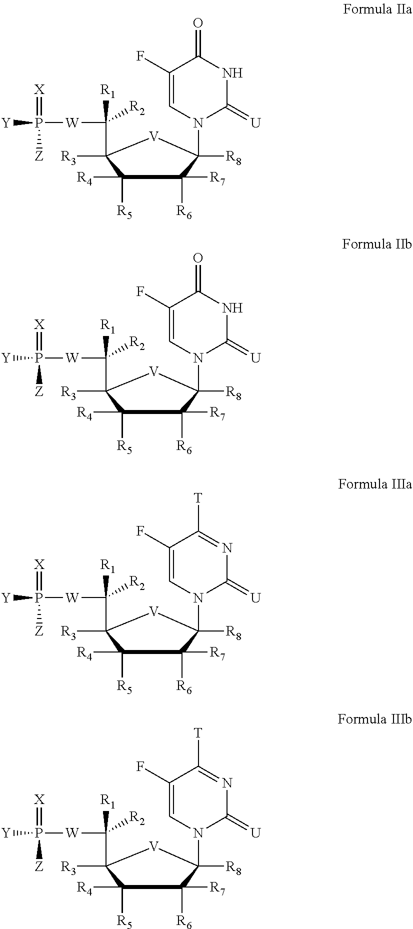

wherein R₁, R₂, R₃, R₄, R₅, R₆, R₇, R₈, T, U, V, W, X, Y and Z are as described above.

In some examples, the prodrugs have a structure of Formula IIa′, Formula IIb′, Formula IIIa′, Formula IIIb′, or a pharmaceutically acceptable salt thereof,

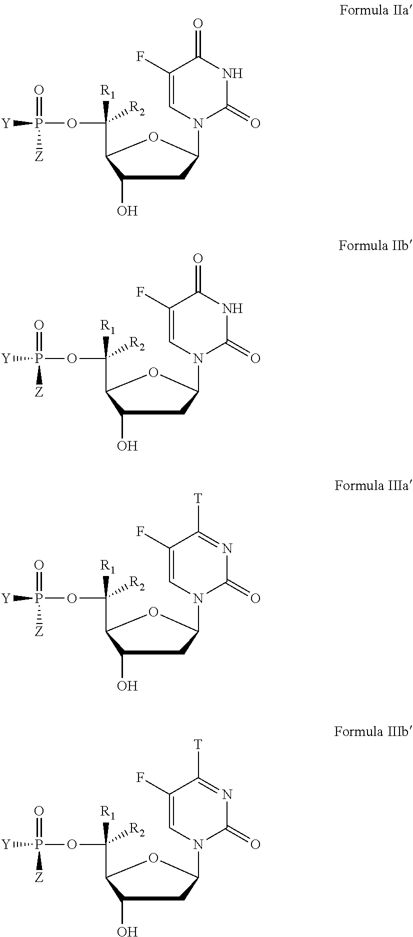

wherein R₁, R₂, T, Y, and Z are the same as described above.

In some examples, the prodrugs have a structure of Formula II, such as Formulas IIa, IIb, IIa′, and IIb′.

In some examples, the prodrugs have a structure of Formula III, such as Formulas IIIa, IIIb, IIIa′, and IIIb′. In some examples, T is —NR₁₅R₁₆. In some examples, R₁₅ and R₁₆ are independently selected from hydrogen, —C(═O)R_{c}, —C(═O)OR_{c1}, —C(═O)SRₒ₁, and —C(═O)NR_{f1}R_{f2}, wherein R_{c1}, Rₑ, R_{f1}, R_{f2}, and Rₒ₁ are the same as described above. In some examples, R₁₅ is hydrogen and R₁₆ is an ester, e.g., —C(═O)OR_{c1}. In some examples, R₁₅ is hydrogen and R₁₆ is —C(═O)OR_{c1}, wherein R_{c1} is optionally substituted C₁-C₁₂ alkyl or optionally substituted C₁-C₁₀ alkyl. In some examples, R₁₅ is hydrogen and R₁₆ is —C(═O)OR_{c1}, wherein R_{c1} is C₂-C₉ alkyl or C₂-C₈ alkyl. In some examples, R₁₅ is hydrogen and R₁₆ is —C(═O)OCH₂CH₂CH₂CH₃ or —C(═O)OCH₂CH₂CH₂CH₂CH₃. In some examples, both R₁₅ and R₁₆ are hydrogen. In some examples, T is —OR₁₇. In some examples, R₁₇ is selected from —C(═O)Rₑ, —C(═O)OR_{c1}, —C(═O)SRₒ₁, and —C(═O)NR_{f1}R_{f2}, wherein R_{c1}, Rₑ, R_{f1}, R_{f2}, and Rₒ₁ are the same as described above.

In some examples, Rᵣ₁ is optionally substituted C₁-C₄ bridging alkylene, such as optionally substituted, linear, C₁-C₄ bridging alkylene, e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—. In some examples, Rᵣ₁ is —CH₂CH₂— or —CH₂CH₂CH₂CH₂—.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5. In some examples, Q is —CH₄, h as

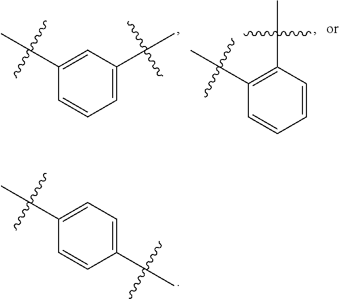 [SMILES: *c1cccc(*)c1 ; *c1ccccc1* ; *c1ccc(*)cc1]

In some examples, Q is —CH₂—C₆H₄—, such as

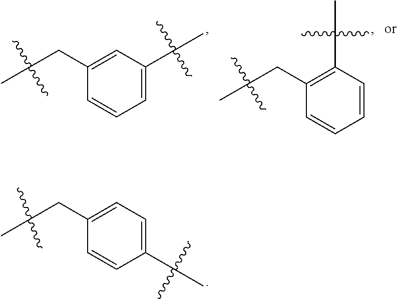 [SMILES: *Cc1cccc(*)c1 ; *Cc1ccccc1* ; *Cc1ccc(*)cc1]

Rᵣ₂ is selected from ester, thioester, amide, acylamino, carbonate ester, carbomate, disulfide, optionally substituted (4-acylamino)phenyl, and optionally substituted (4-acyloxy)phenyl. In some examples, Rᵣ₂ is selected from —C(═O)OR_{c1}, —OC(═O)R_{c2}, —C(═O)SRₒ₁, —SC(═O)Rₒ₂, —C(═O)NR_{f1}R_{f2}, —NR_{g}[C(═O)Rₕ], —OC(═O)ORᵢ, —OC(═O)NRⱼ₁Rⱼ₂, —NRₖ[(C═O)ORₗ], —S—S—R_{z}, optionally substituted (4-acylamino)phenyl, and optionally substituted (4-acyloxy)phenyl, wherein R_{c1}, R_{c2}, R_{f1}, R_{f2}, R_{g}, Rₕ, Rᵢ, Rⱼ₁, Rⱼ₂, Rₖ, Rₗ, Rₒ₁, Rₒ₂, and R_{z} are the same as described above. In some examples, R_{c1}, R_{c2}, R_{f1}, R_{f2}, Rₕ, Rᵢ, Rⱼ₁, Rⱼ₂, Rₖ, Rₗ, Rₒ₁, Rₒ₂, and R_{z} are independently selected from optionally substituted aryl (such as phenyl or naphthyl) and optionally substituted alkyl (such as benzyl, isopropyl, and 2-ethylbutyl); in this case, “optionally substituted” refers to optionally substituted by one or more R groups described herein. In some examples, R_{c1}, R_{c2}, R_{f1}, R_{f2}, Rₕ, Rᵢ, Rⱼ₁, Rⱼ₂, Rₖ, Rₗ, Rₒ₁, Rₒ₂, and R_{z} are independently phenyl or naphthyl. In some examples, R_{c1}, R_{c2}, R_{f1}, R_{f2}, Rₕ, Rᵢ, Rⱼ₁, Rⱼ₂, Rₖ, Rₗ, Rₒ₁, Rₒ₂, and R_{z} are independently benzyl, isopropyl, or 2-ethylbutyl. In some examples, R_{g} and Rₖ are independently hydrogen or optionally substituted alkyl such as methyl; in this case, “optionally substituted” refers to optionally substituted by one or more R groups described herein. In some examples, R_{g} and Rₖ are hydrogen. In some examples, R_{g} and Rₖ are methyl. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl. In some examples, R_{z} is a C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₅-C₂₂ alkyl or a linear C₁₁-C₂₀ alkyl.

In some examples, Rᵣ₂ is —S—S—R_{z}. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₁₁-C₂₀ alkyl or a linear C₅-C₂₂ alkyl.

In some examples, Rᵣ₂ is —NR_{g}[C(═O)Rₕ]; in some examples, R_{g} is hydrogen or methyl. In some examples, Rᵣ₂ is —OC(═O)ORᵢ. In some examples, Rᵣ₂ is optionally substituted (4-acylamino)phenyl or optionally substituted (4-acyloxy)phenyl; in some examples, Rᵣ₂ is (4-acylamino)phenyl and (4-acyloxy)phenyl.

Exemplary —Rᵣ₁—Rᵣ₂ includes, but is not limited to, —CH₂—OC(═O)ORᵢ, —CH₂CH₂CH₂—NHC(═O)ORₕ, —CH₂CH₂CH₂—S—S—R_{z}, —CH₂CH₂CH₂CH₂—S—S—R_{z}, —CH₂CH₂CH(CH₃)—S—S—R_{z}, —CH₂CH₂CH₂CH(CH₃)—S—S—R_{z}, —CH₂CH₂CH₂—NH[C(═O)Rₕ],

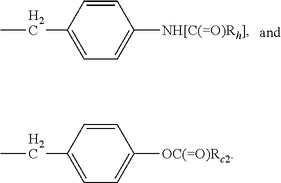

In some examples, Rᵣ₁ is optionally substituted C₁-C₄ bridging alkylene, and Rᵣ₂ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is optionally substituted, linear, C₁-C₄ bridging alkylene (e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—), and Rᵣ₂ is —S—S—R_{z}, wherein R_{z} is alkyl, optionally substituted by one or more R groups described herein. In some examples, Rᵣ₁ is —CH₂CH₂— or —CH₂CH₂CH₂CH₂—, and Rᵣ₂ is —S—S—R_{z}, wherein R_{z} is a linear C₂-C₂₂ alkyl or a linear C₁-C₂₀ alkyl.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5; and Rᵣ₂ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is -Q-CH₂—, wherein Q is —C₆H₄— (such as

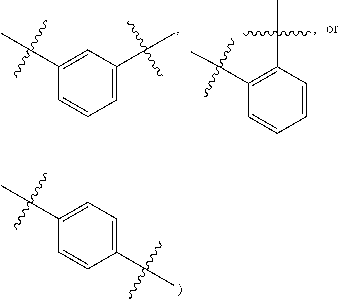 [SMILES: *c1cccc(*)c1 ; *c1ccccc1* ; *c1ccc(*)cc1]

or —CH₂—C₆H₄— (such as

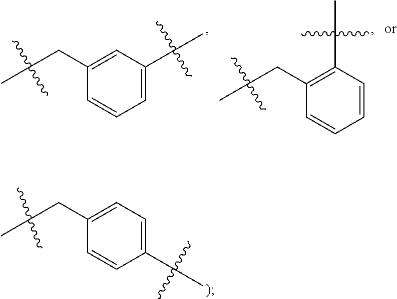 [SMILES: *Cc1cccc(*)c1 ; *Cc1ccccc1* ; *Cc1ccc(*)cc1]

and Rᵣ₂ is —S—S—R_{z}, wherein R_{z} is a linear C₂-C₂₂ alkyl or a linear C₁-C₂₀ alkyl.

As used herein, “standard amino acid” refers to the following twenty amino acids: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Unless the context indicates otherwise, the substituted or optionally substituted groups described in Formulas II, III, IIa, IIb, IIIa, IIIb, IIa′, IIb′, IIIa′, and IIIb′ can have one or more substituents independently selected from deuterium, halogen, azido, cyano, isocyano, nitrate, nitrosooxy, nitroso, nitro, formyl, carboxyl, carbonate, alkyl, heteroalkyl, alkenyl, heteroalkenyl, alkynyl, heteroalkynyl, carbocyclyl, heterocyclyl, aryl, heteroaryl, azo, acyl, hydroxyl, thiol, sulfinyl, sulfonyl, sulfonate, sulfamoyl, amino, acylamino, amide, silyl, ester, thioester, carbonate ester, carbamate, aminooxy, hydroxyamino, and —SF₅, wherein each substituent may be further substituted by one or more R groups as described herein. In some examples, two substituents on the same atom can join together with that atom to form a cyclic moiety, such as a carbocycle or a heterocycle.

For example, the Si-substituted silyl in Formulas II, III, IIa, IIb, IIIa, IIIb, IIa′, IIb′, IIIa′, and IIIb′ can have one, two, or three substituents independently selected from those described above. When there are multiple substituents, two of the substituents can join together with the Si atom to form a heterocycle. In some examples, the substituent(s) is independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. In some examples, the Si-substituted silyl has three substituents, independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. Exemplary Si-substituted silyl groups include, but are not limited to, the following:

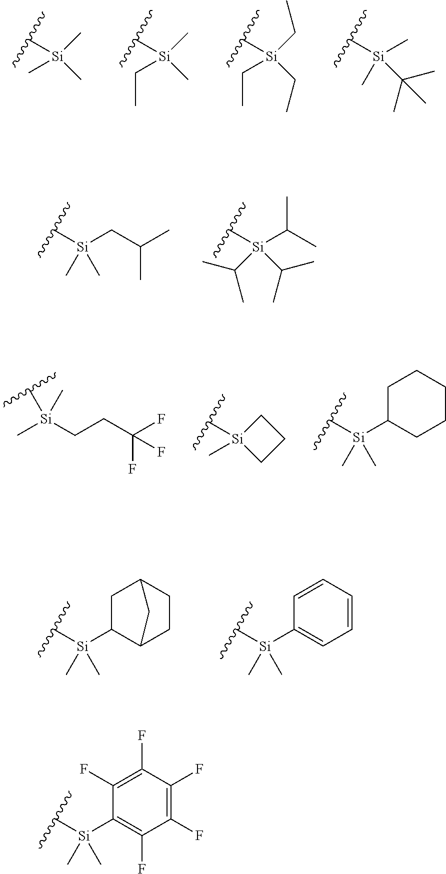

In some examples, the substituted or optionally substituted groups described in Formulas II, III, IIa, IIb, IIIa, IIIb, IIa′, IIb′, IIIa′, and IIIb′ can have one or more substituents independently selected from alkyl, heteroalkyl, alkenyl, heteroalkenyl, alkynyl, heteroalkynyl, carbocyclyl, heterocyclyl, aryl, and heteroaryl, wherein each substitutent may be further substituted by one or more R groups. In some examples, two substituents on the same atom can join together with that atom to form a cyclic moiety, such as a carbocycle or a heterocycle.

In some examples, the substituted or optionally substituted groups described in Formulas II, III, IIa, IIb, IIIa, IIIb, IIa′, IIb′, IIIa′, and IIIb′ can have one or more substituents independently selected from deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(tert-butyl)silyl, methyl(methyl)(isobutyl)silyl), formyl, carboxyl, mercapto, sulfamoyl, alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), alkyl (such as methyl, ethyl, isopropyl, tert-butyl), heteroalkyl, alkenyl, heteroalkenyl, alkynyl, heteroalkynyl, carbocyclyl, heterocyclyl, aryl, heteroaryl, and —SF₅.

In some examples, the substituted groups or optionally substituted groups described in Formulas II, III, IIa, IIb, IIIa, IIIb, IIa′, IIb′, IIIa′, and IIIb′ can have one or more substituents independently selected from deuterium, halogen, nitro, cyano, hydroxyl, trifluoromethoxy, trifluoromethyl, trimethylsilyl, amino, formyl, carboxyl, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, carbocyclyl, aryl, and heterocyclyl.

1. ProTide Prodrugs

In some examples, the prodrugs are ProTide prodrugs. These prodrugs have a structure according to one or more of Formulas II, IIa, IIa′, IIb, IIb′, IIIa, IIIa′, IIIb or IIIb′, or a pharmaceutically acceptable salt thereof, wherein Y is —O—R₉ or —S—R₁₀, and Z is

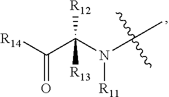

and wherein the other groups in the foregoing formulas are the same as described above.

In some examples, Y is —O—R₉. In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂, wherein Rᵣ₁ and Rᵣ₂ are the same as described above.

In some examples, R₉ is selected from optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₉ is optionally substituted aryl, such as phenyl or naphthyl. In some examples, R₉ is phenyl. In some examples, R₉ is naphthyl. In some examples, R₉ is optionally substituted alkyl, such as benzyl, isopropyl, and 2-ethylbutyl.

In some examples, Y is —S—R₁₀. In some examples, R₁₀ is optionally substituted aryl, such as phenyl or naphthyl. In some examples, R₁₀ is phenyl. In some examples, R₁₀ is naphthyl. In some examples, R₁₀ is optionally substituted alkyl, such as benzyl, isopropyl, and 2-ethylbutyl.

In some examples, R₁₁ is hydrogen.

In some examples, one of R₁₂ and R₁₃ is hydrogen.

In some examples, one of R₁₂ and R₁₃ is a standard amino acid side chain. In some examples, one of R₁₂ and R₁₃ is an alanine side chain, i.e., methyl.

In some examples, R₁₂ is hydrogen, and R₁₃ is a standard amino acid side chain. In some examples, R₁₂ is hydrogen, and R₁₃ is an alanine side chain, i.e., methyl. In some examples, R₁₂ is hydrogen, and R₁₃ is a proline side chain, wherein R₁₃ forms a pyrrolidine ring together with R₁₁, the nitrogen atom connected to R₁₁, and the carbon atom connected to R₁₂ and R₁₃.

In some examples, R₁₃ is hydrogen, and R₁₂ is a standard amino acid side chain. In some examples, R₁₃ is hydrogen, and R₁₂ is an alanine side chain, i.e., methyl. In some examples, R₁₃ is hydrogen, and R₁₂ is a proline side chain, wherein R₁₂ forms a pyrrolidine ring together with R₁₁, the nitrogen atom connected to R₁₁, and the carbon atom connected to R₁₂ and R₁₃. In some examples, R₁₄ is —NRₛ₁Rₛ₂. Rₛ₁ and Rₛ₂ can be independently selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl.

In some examples, R₁₄ is —ORₜ. Rican be selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₁ is isopropyl, 2-ethylbutyl, or benzyl. In some examples, R₁ is isopropyl. In some examples, Rt is 2-ethylbutyl. In some examples, Rt is benzyl.

In some examples, Y is —O—R₉, and Z is

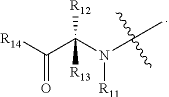

In some examples, R₉ is phenyl or naphthyl, R₁₁ is hydrogen, one of R₁₂ and R₁₃ is hydrogen, the other one of R₁₂ and R₁₃ is methyl, and R₁₄ is —ORₜ, wherein Rₜ is isopropyl, 2-ethylbutyl, or benzyl.

In some examples, the ProTide prodrugs have a structure selected from the following structures or pharmaceutically acceptable salts (such as ammonium and lithium salts) thereof:

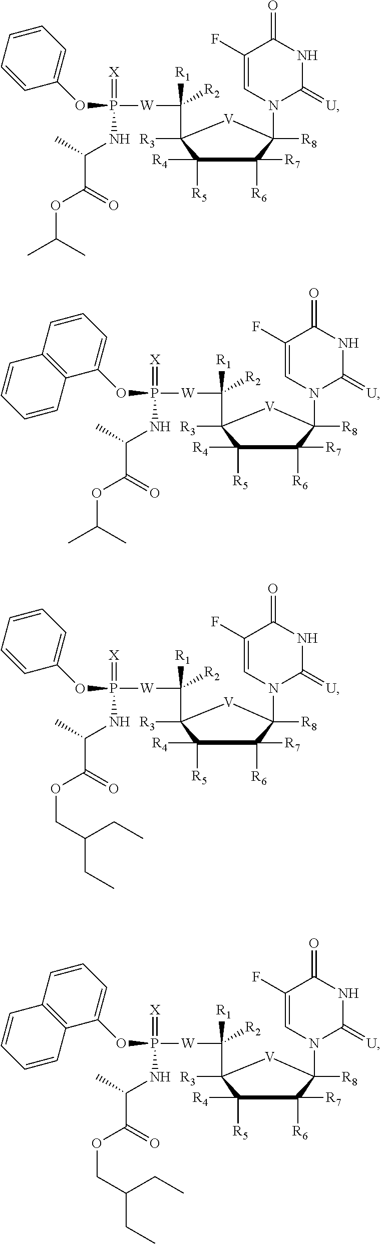

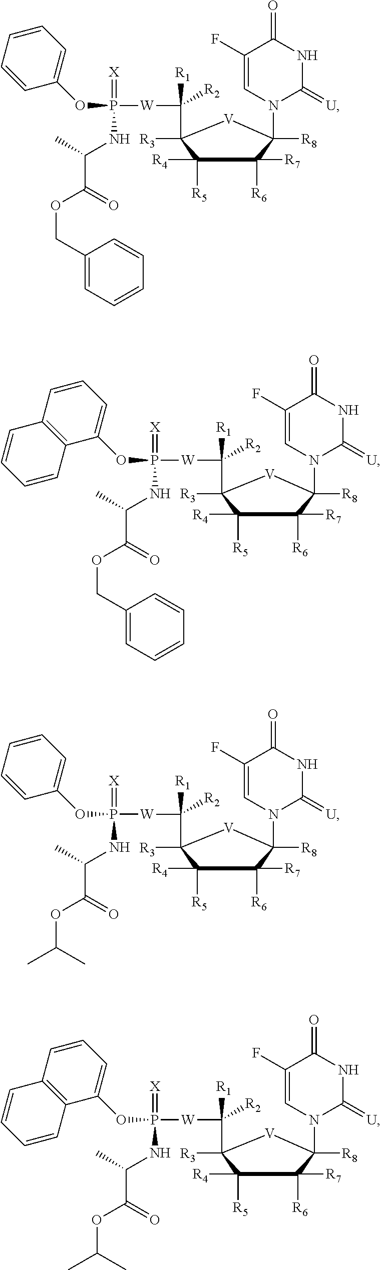

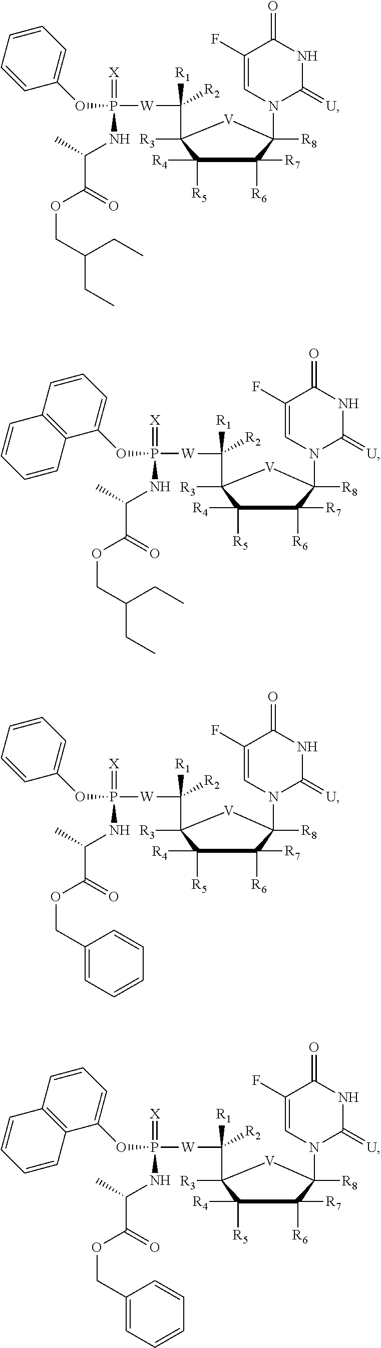

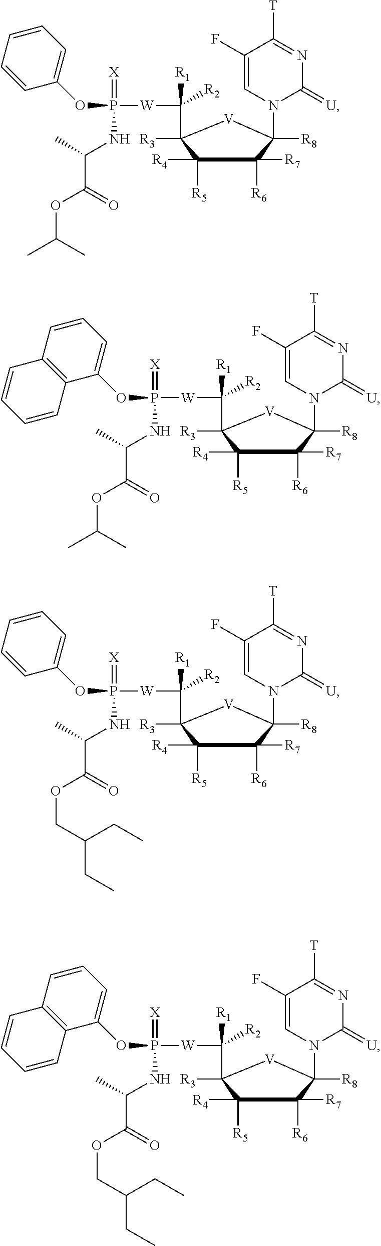

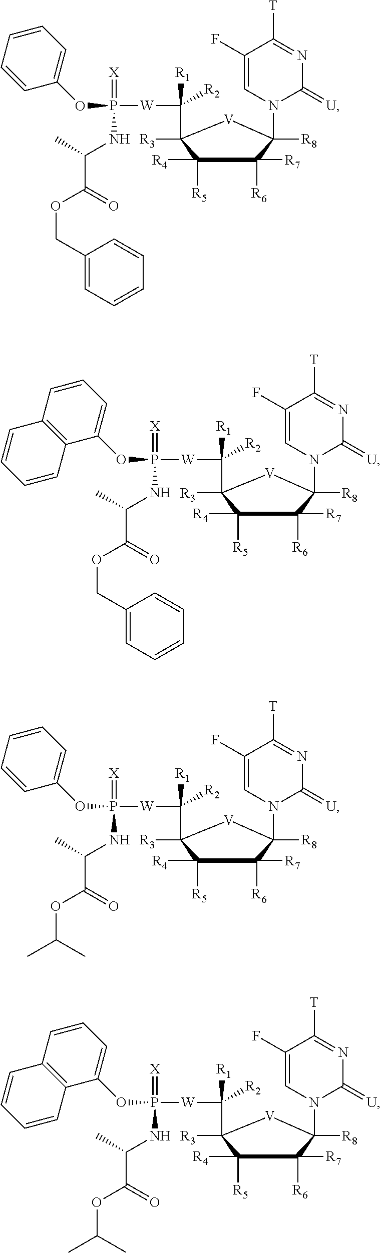

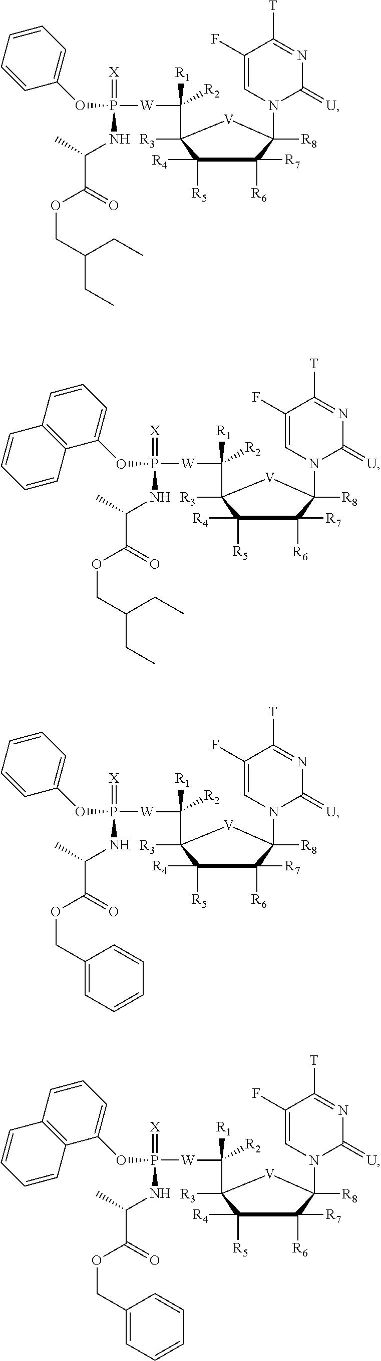

wherein R₁, R₂, R₃, R₄, R₅, R₆, R₇, R₈, T, U, V, W, and X are the same as described above.

In some examples, the ProTide prodrugs have a structure selected from the following structures or pharmaceutically acceptable salts (such as ammonium and lithium salts) thereof:

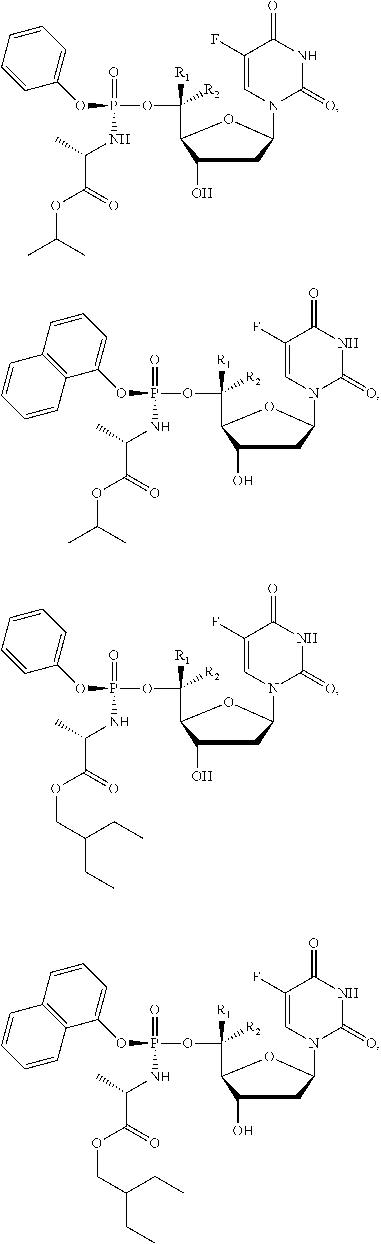

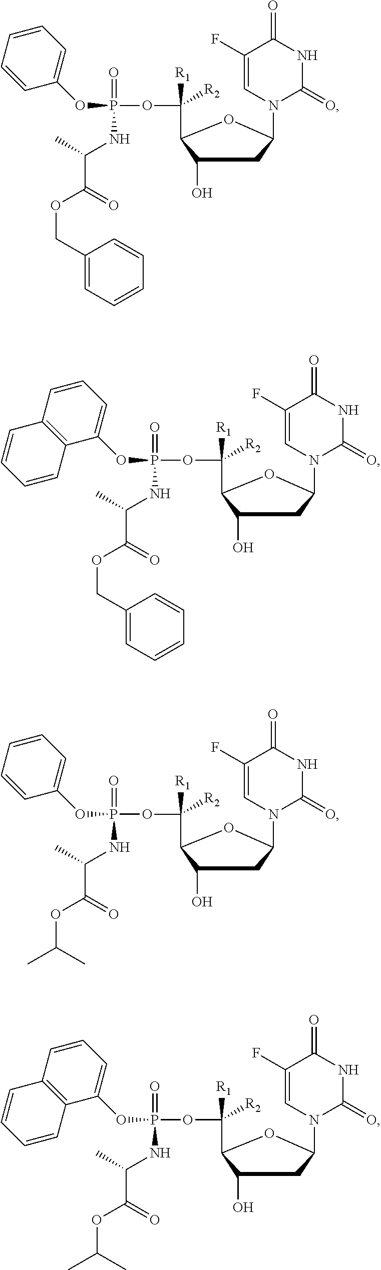

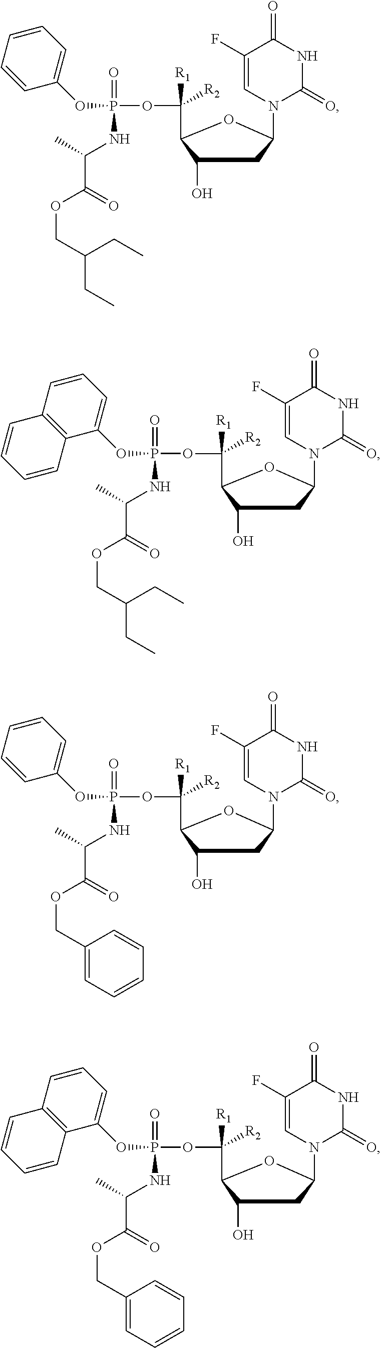

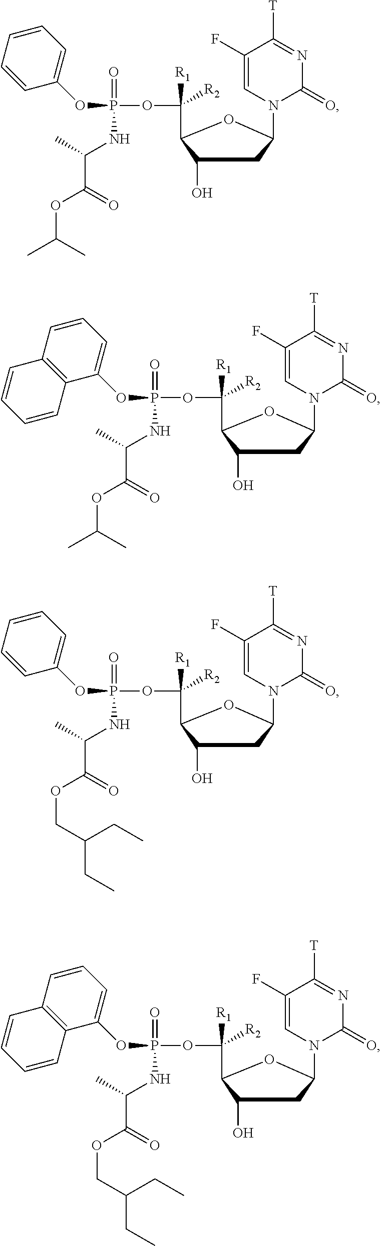

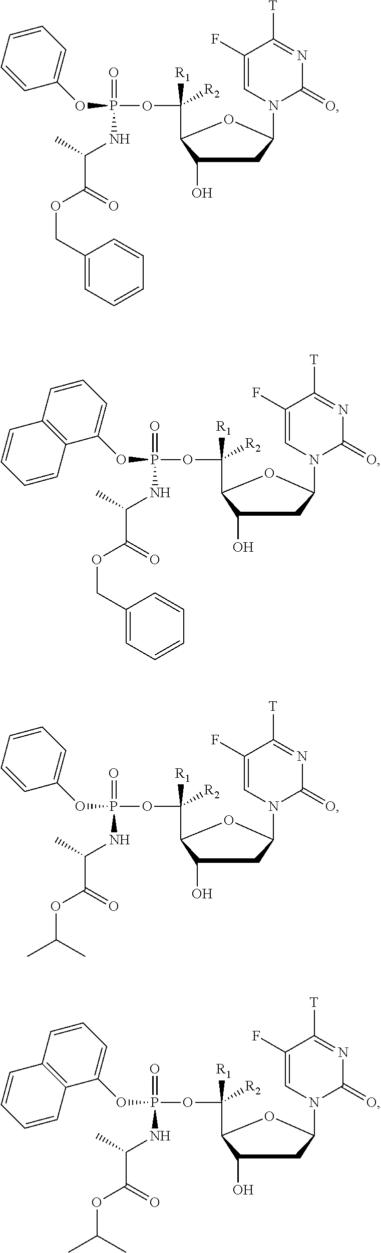

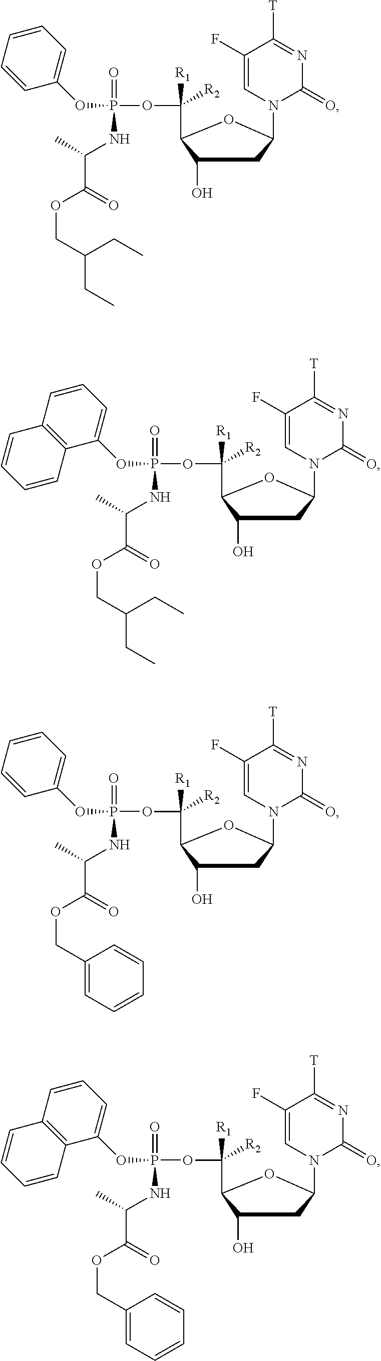

wherein T, R₁ and R₂ are the same as described above.

Exemplary ProTide prodrugs include, but are not limited to, the following structures and pharmaceutically acceptable salts thereof:

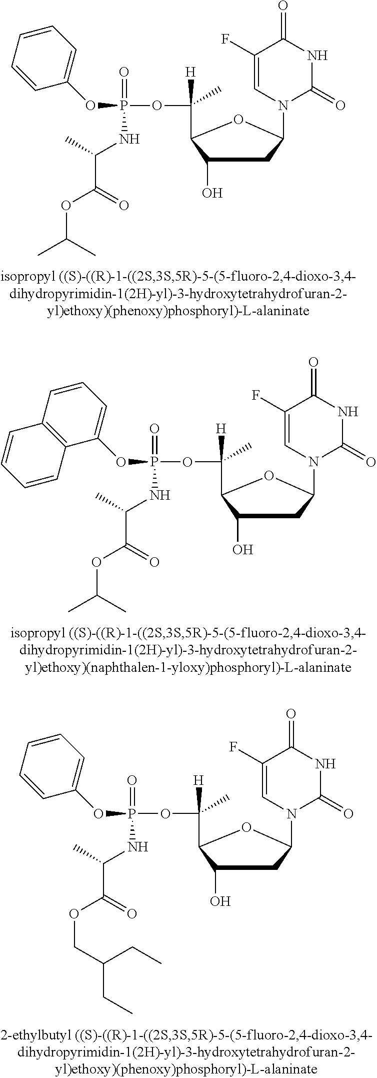

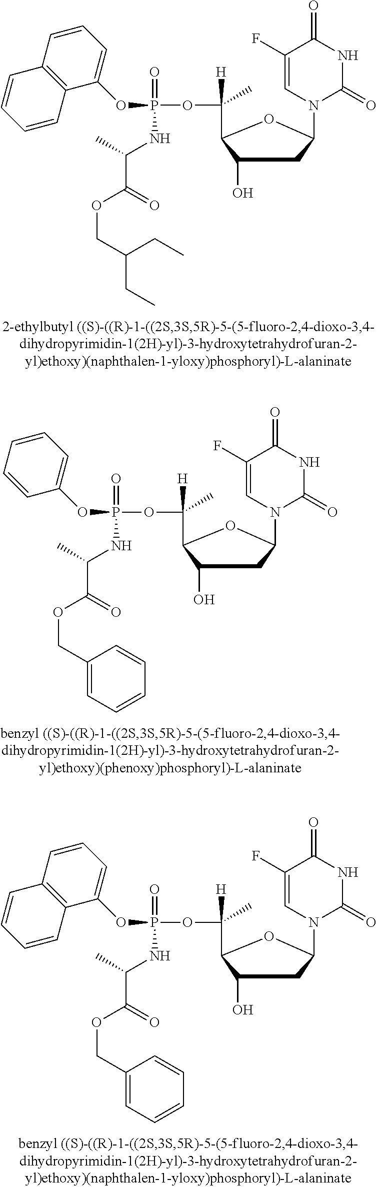

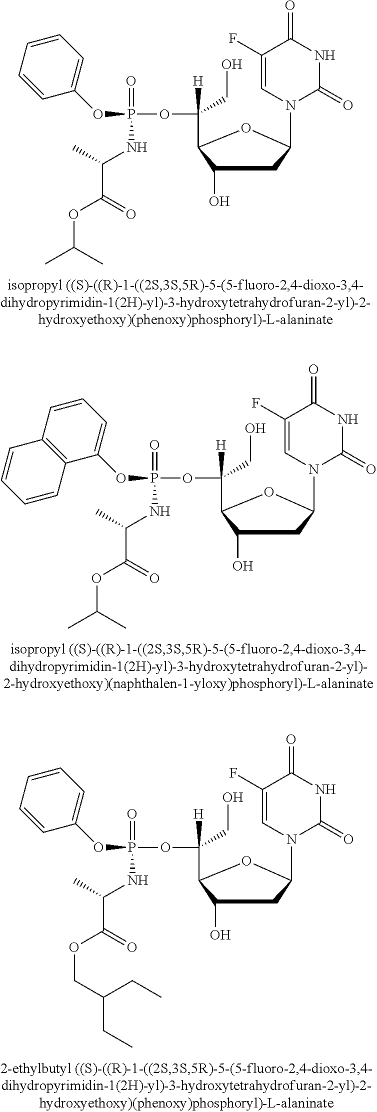

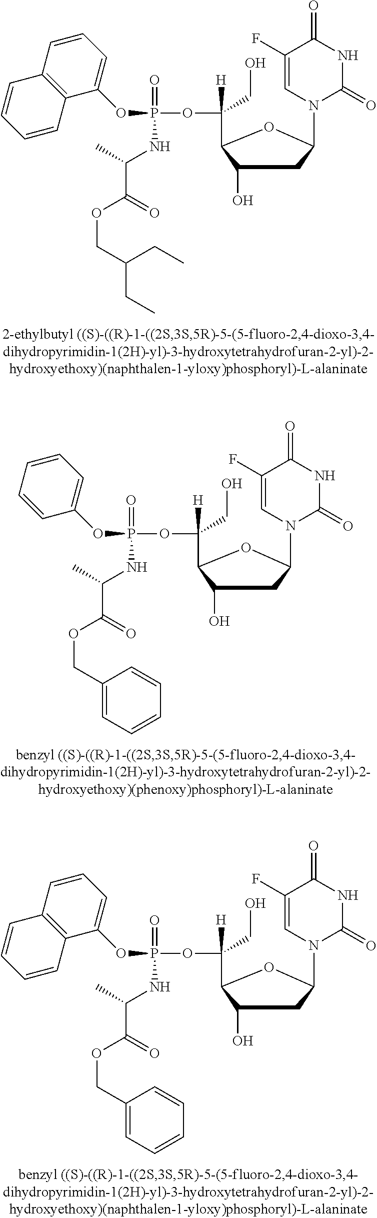

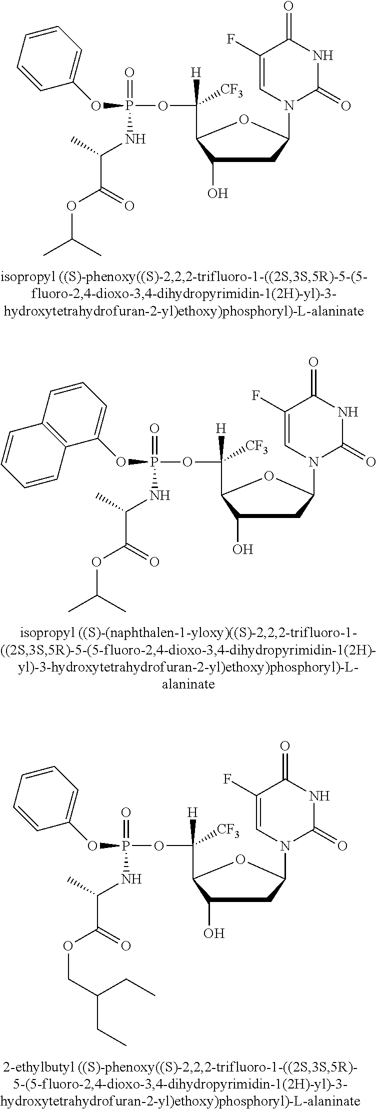

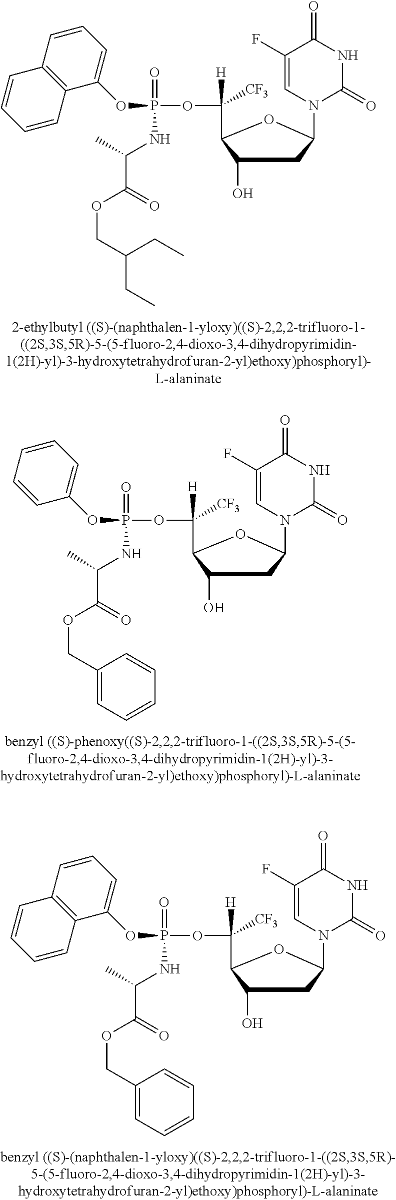

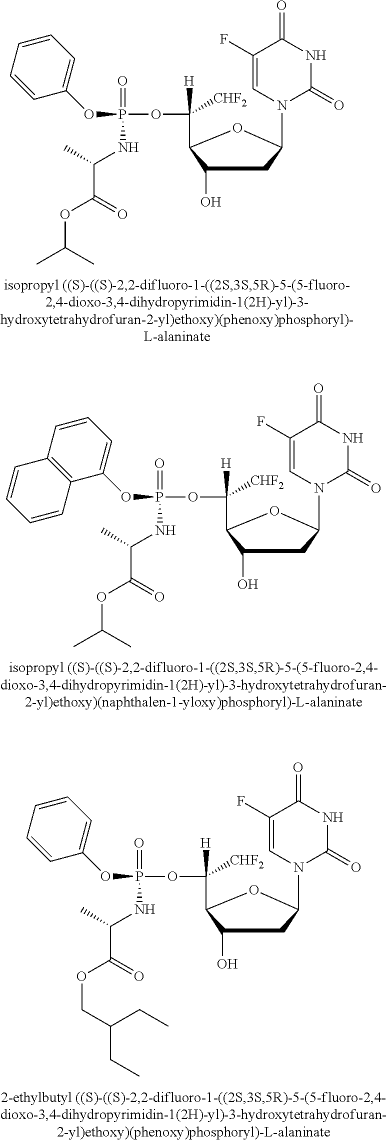

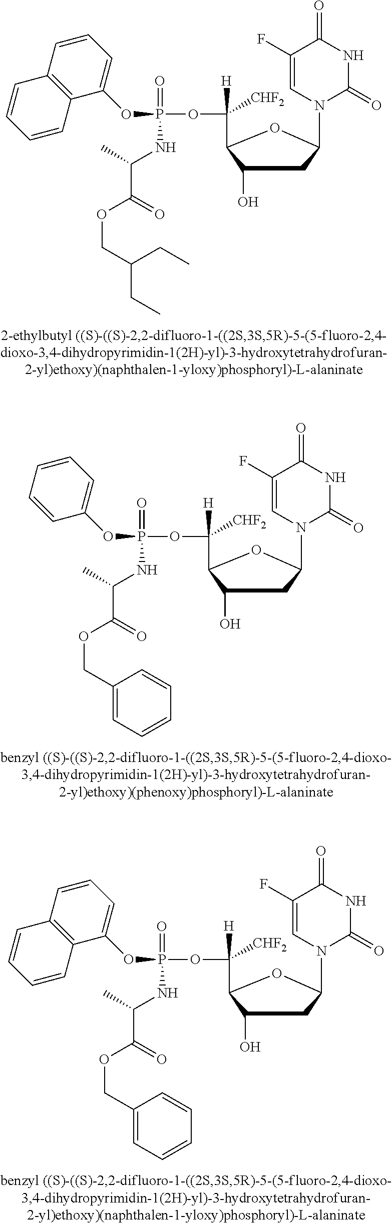

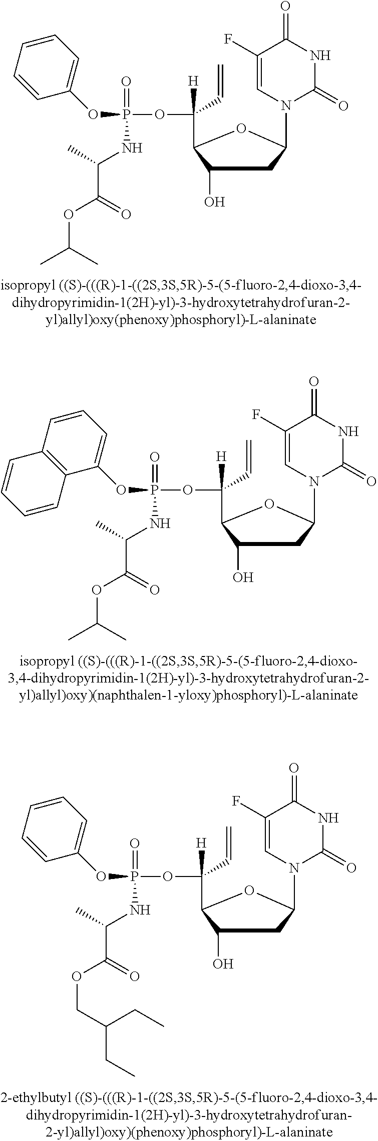

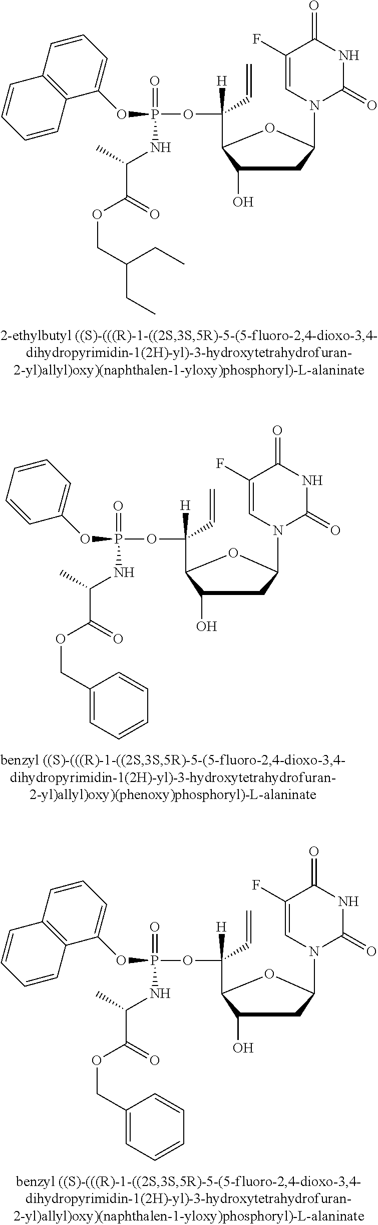

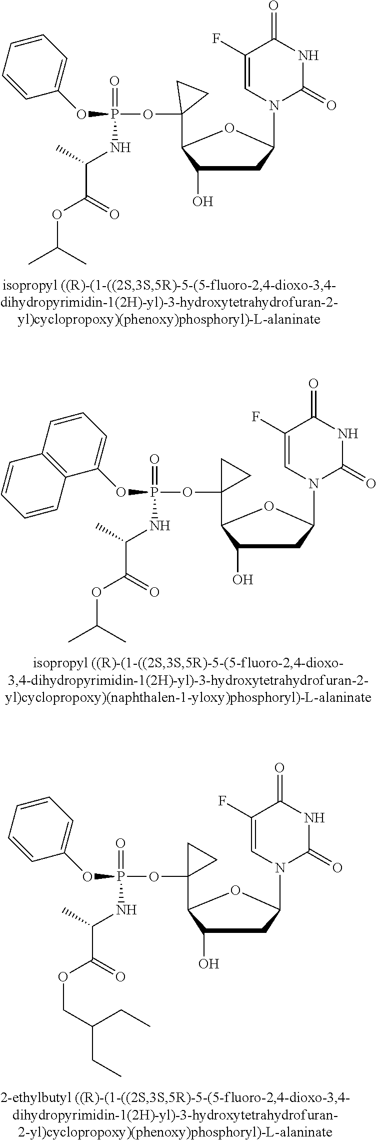

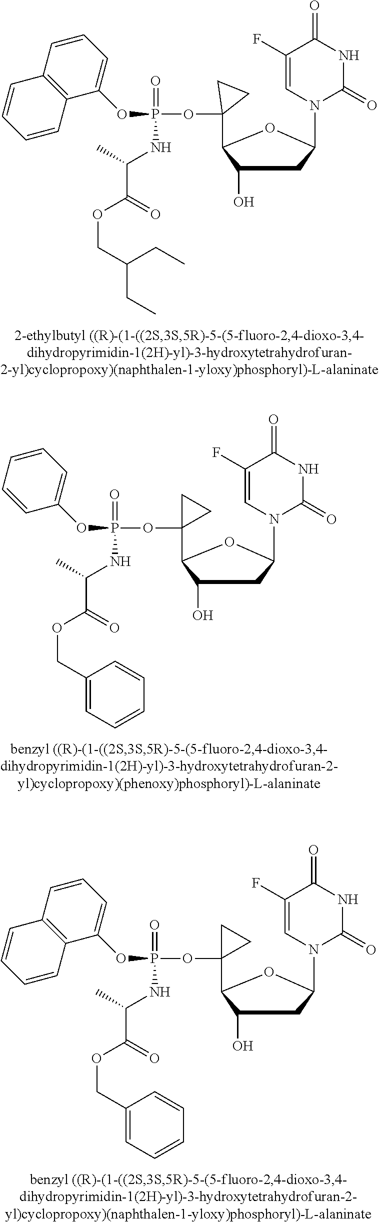

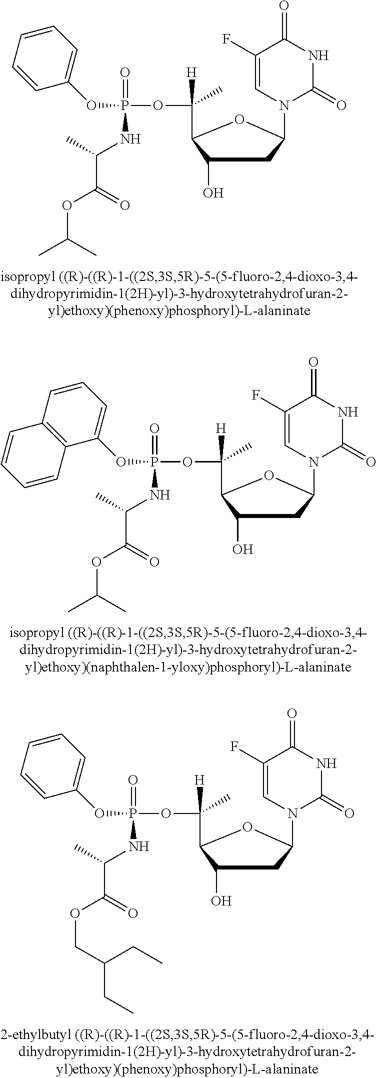

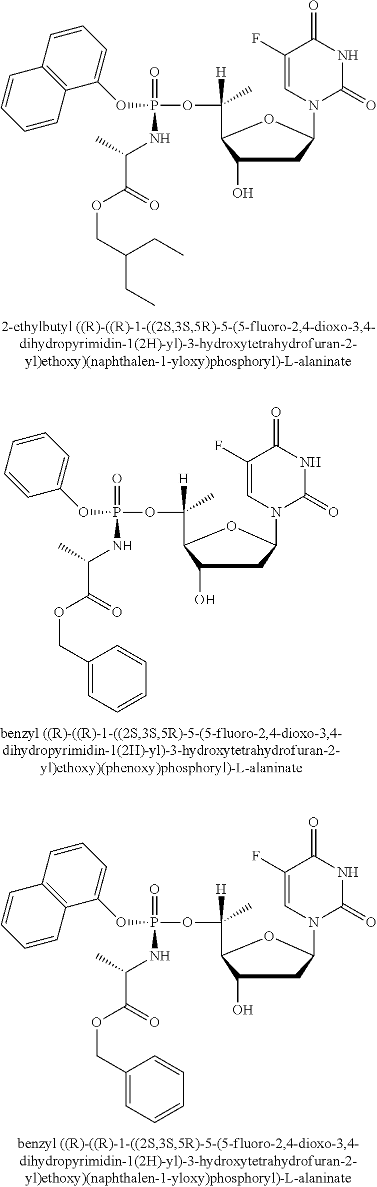

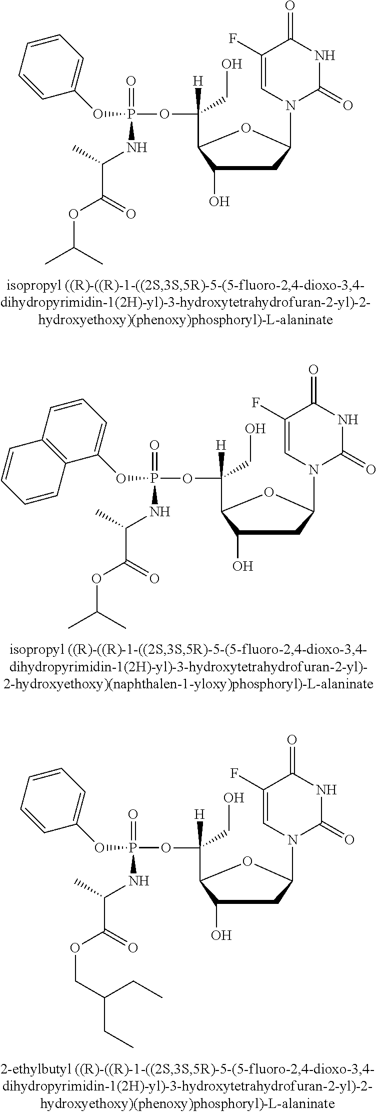

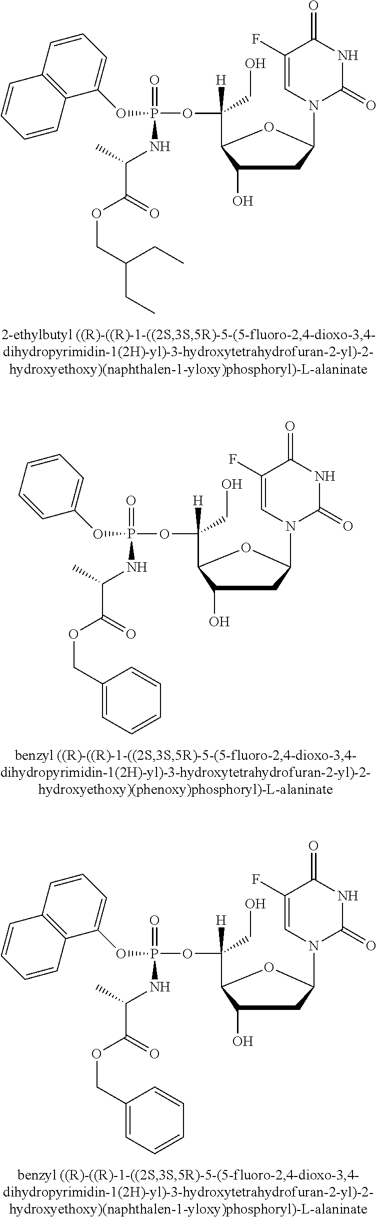

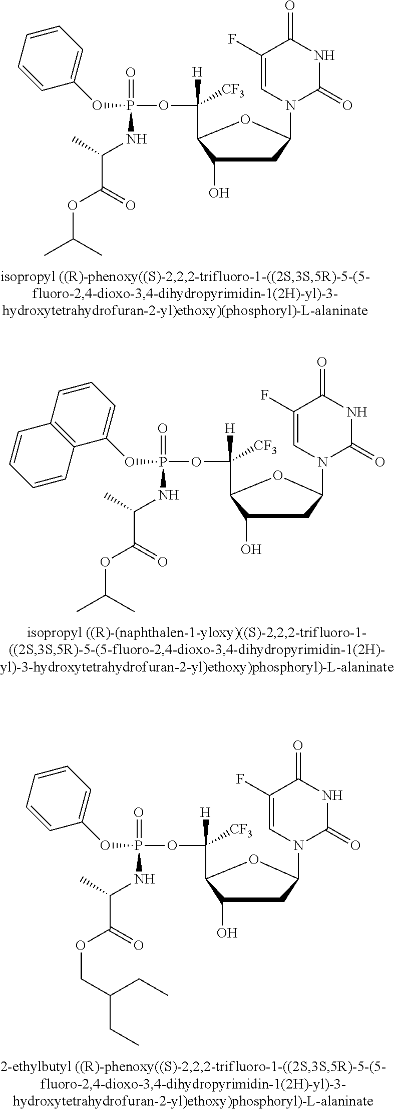

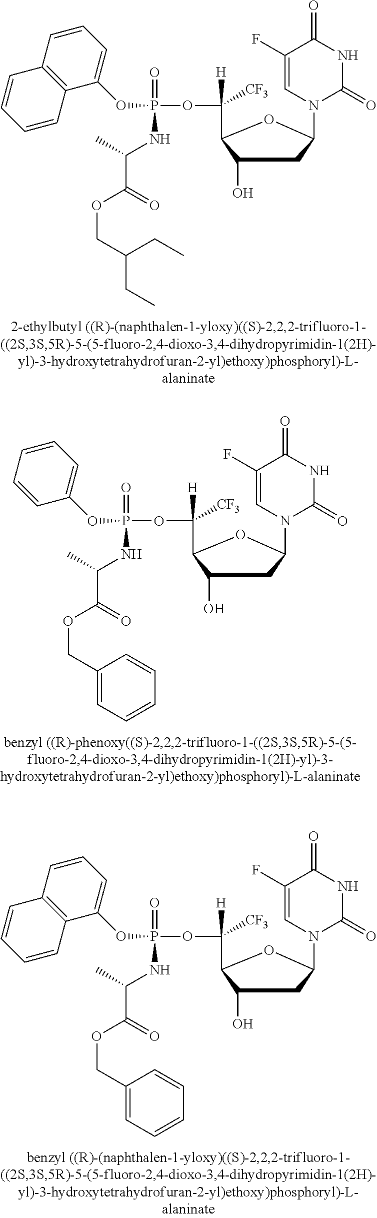

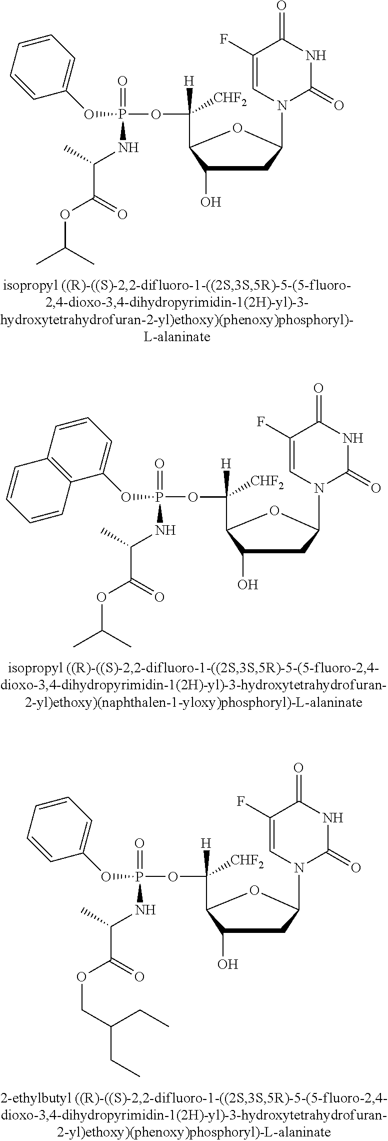

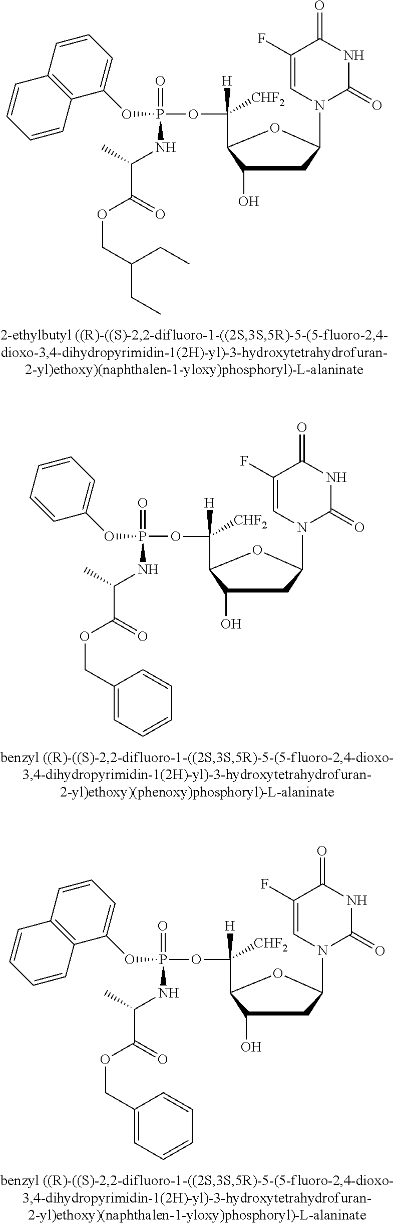

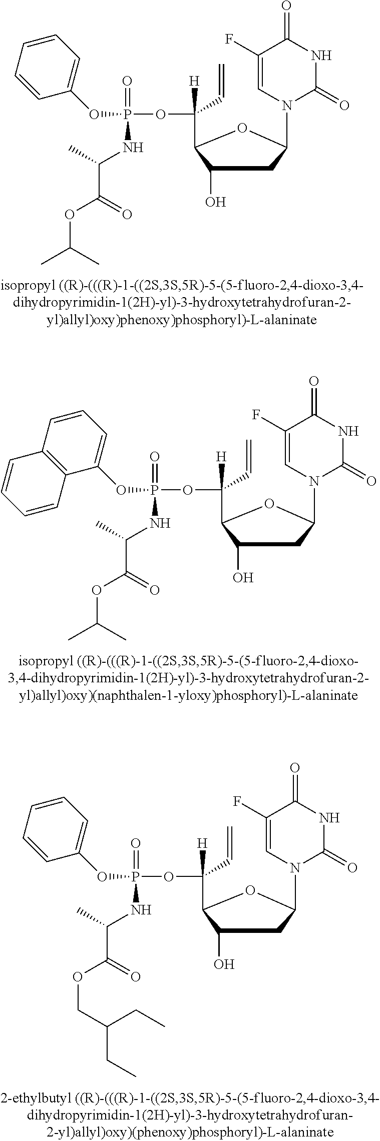

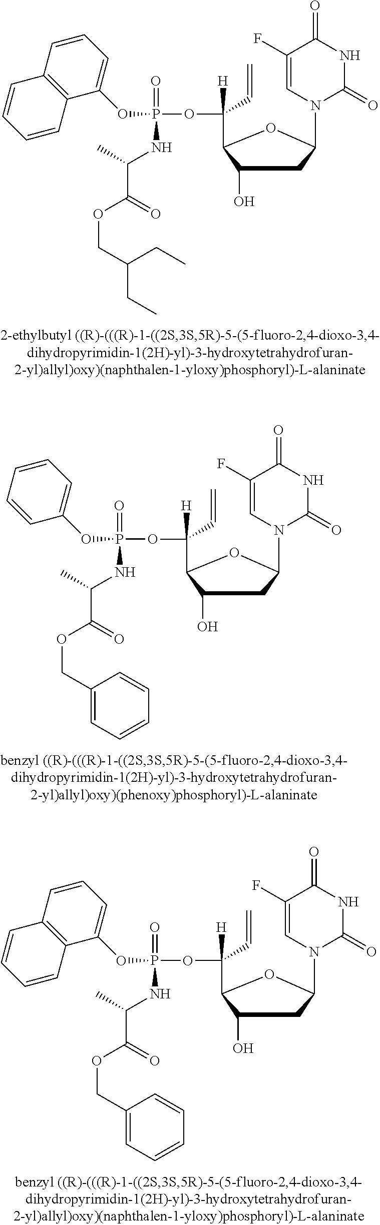

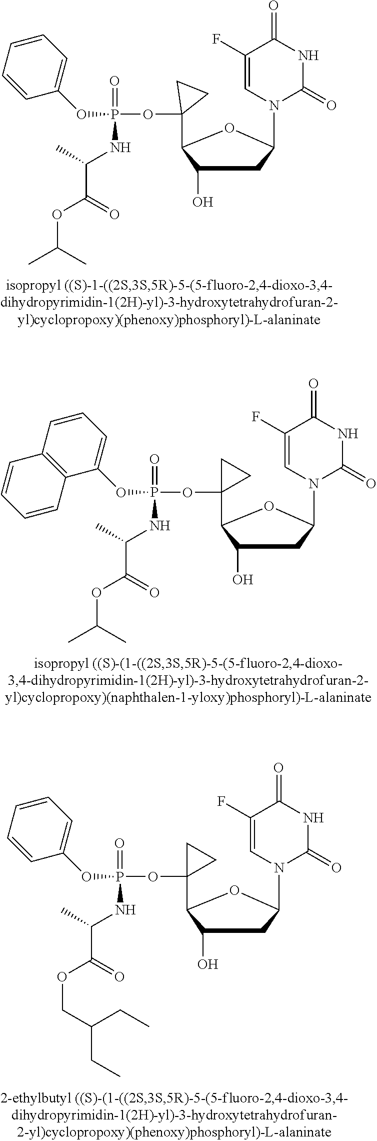

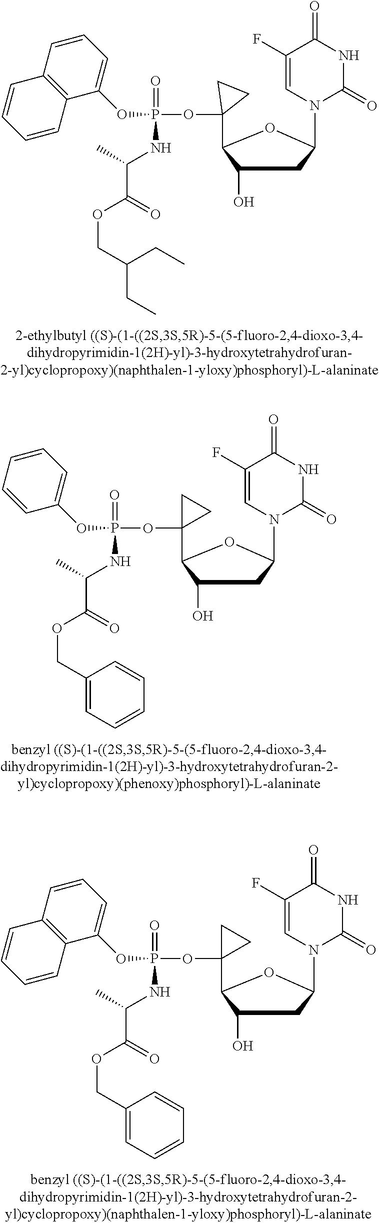

2. Lipid-Derived Prodrugs

In some examples, the prodrugs are lipid-derived prodrugs. These prodrugs have a structure of Formulas II, IIa, IIa′, IIb, IIb′, IIIa, IIIa′, IIIb or IIIb′, or a pharmaceutically acceptable salt thereof, wherein Y is —O—R₉ or —S—R₁₀ and Z is —O—R_{q1}—R_{q2}—R_{q3}—R_{q4}, and wherein the other groups in the foregoing formulas are the same as described above.

In some examples, Y is —O—R₉. In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂, wherein Rᵣ₁ and Rᵣ₂ are the same as described above.

In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₉ is hydrogen. In some examples, R₉ is optionally substituted alkyl, such as benzyl, isopropyl, and 2-ethylbutyl. In some examples, R₉ is optionally substituted aryl, such as phenyl and naphthyl.

In some examples, Y is —S—R₁₀. In some examples, R₁₀ is optionally substituted alkyl, such as benzyl, isopropyl, and 2-ethylbutyl. In some examples, R₁₀ is optionally substituted aryl, such as phenyl and naphthyl.

In some examples, when R_{q2} is —O—, R_{q4} is not hydrogen, methyl, or ethyl.

In some examples, when both R_{q1} and R_{q2} are absent, R_{q4} is not hydrogen, methyl, or ethyl.

In some examples, when R_{q1} is absent, R_{q2} is also absent.

In some examples, both R_{q1} and R_{q2} are present, i.e., Z is —O—R_{q1}—R_{q2}—R_{q3}—R_{q4}.

In some examples, both R_{q1} and R_{q2} are absent, i.e., Z is —O—R_{q3}—R_{q4}.

In some examples, R_{q1} is present, and R_{q2} is absent, i.e., Z is —O—R_{q1}—R_{q3}—R_{q4}.

R_{q1} is absent or a C₁-C₉ alkyl chain (i.e., C₁-C₉ bridging alkylene). In some examples, R_{q1} is absent. In some examples, R_{q1} is a C₁-C₉ alkyl chain (i.e., C₁-C₉ bridging alkylene). In some examples, R_{q1} is a linear C₁-C₉ alkyl chain (i.e., linear C₁-C₉ bridging alkylene), such as methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), and nonylene (—CH₂CH₂CH₂CH₂CH₂CH₂CH₂CH₂CH₂—).

In some examples, R_{q1} is ethylene (—CH₂CH₂—), propylene (—CH₂CH₂CH₂—), or nonylene (—CH₂CH₂CH₂CH₂CH₂CH₂CH₂CH₂CH₂—). In some examples, R_{q1} is ethylene (—CH₂CH₂—) or propylene (—CH₂CH₂CH₂—).

R_{q2} is absent or is selected from substituted methylene or ethylene, —O—, —S—, —S(═O)—, —S—S—, and —S(O)₂—. In some examples, R_{q2} is absent. In some examples, R_{q2} is selected from substituted methylene or ethylene, —O—, —S—, —S(═O)—, —S—S—, and —S(O)₂—. In some examples, the substituted methylene or ethylene contains one or more halogen substituents. In some examples, the one or more halogen substituents are fluorine.

In some examples, R_{q2} is substituted methylene, such as —CF₂—. In some examples, R_{q2} is —O—. In some examples, R_{q2} is —S—. In some examples, R_{q2} is —S—S—.

R_{q3} is a C₂-C₂₀ alkyl chain (i.e., C₂-C₂₀ bridging alkylene). In some examples, R_{q3} is a linear C₂-C₂₀ alkyl chain (i.e., linear C₂-C₂₀ bridging alkylene), such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—].

In some examples, R_{q3} is a linear C₂-C₇ alkyl chain (i.e., linear bridging C₂-C₇ alkylene). In some examples, R_{q3} is a linear C₅-C₂₀ alkyl chain (i.e., linear C₅-C₂₀ bridging alkylene).

R_{q4} is selected from hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅.

Examples of R_{q4} include, but are not limited to, hydrogen, —CD₃, —CF₃, —CD₂CD₃, —CF₂CF₃, —S-Ph, —O-Ph, —C≡CH, —C≡CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

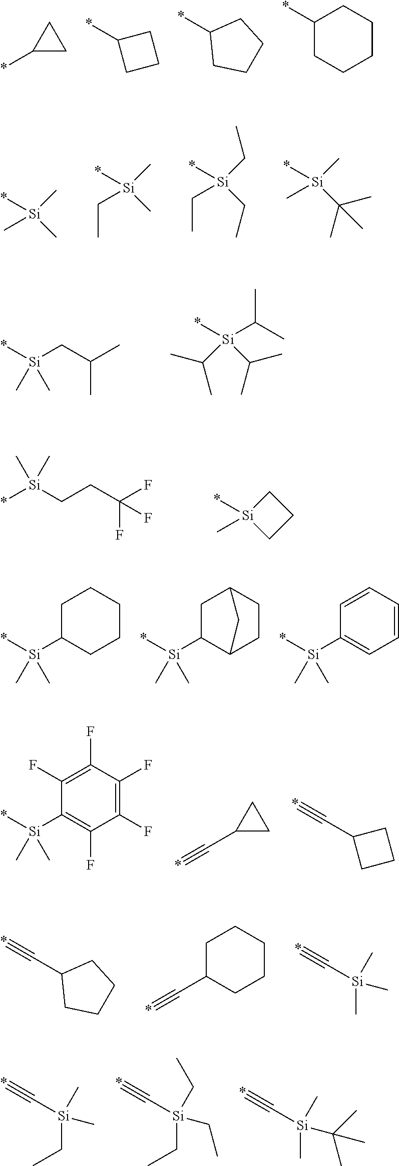

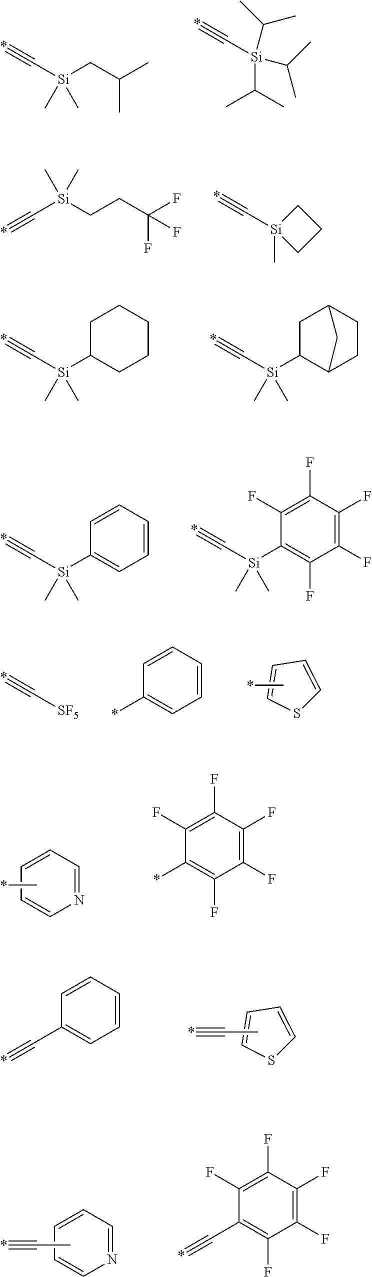

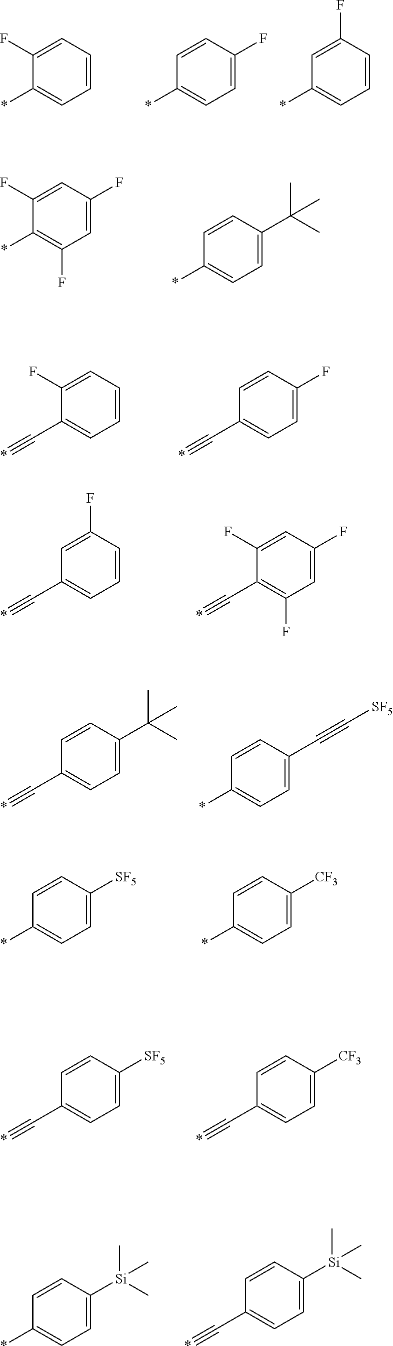

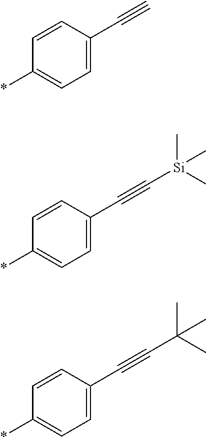

wherein * indicates the point of attachment to R_{q3}.

In some examples, R_{q4} is hydrogen, methyl or ethyl. In some examples, R₄ is hydrogen.

In some examples, R₄ is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅.

In some examples, R_{q4} is substituted methyl or ethyl, having one or more substituents.

In some examples, the one or more substituents are independently selected from deuterium, halogen, and alkyl. In some examples, R_{q4} is selected from —CD₃, —CF₃, —C(CH₃)₃, —CD₂CD₃, and —CF₂CF₃. In some examples, R_{q4} is —CF₃.

In some examples, R_{q4} is optionally substituted C₂-C₃ alkenyl or alkynyl, which may have one or more substituents. For example, R_{q4} can be optionally substituted C₂-C₃ alkynyl, such as optionally substituted ethynyl and optionally substituted propynyl (including optionally substituted 1-propynyl and optionally substituted 2-propynyl). In some examples, the one or more substitutions are independently selected from deuterium, halogen (such as fluorine), alkyl, heteroalkyl, alkenyl, heteroalkenyl, alkynyl, heteroalkynyl, carbocyclyl (such as cyclopropyl, cyclobutyl, cyclopentyl, and cyclohexyl), heterocyclyl, aryl (such as phenyl), heteroaryl (such as pyridinyl and thiophenyl), silyl, and —SF₅, wherein each substituent may be further substituted by one or more R groups as described herein. In some examples, R_{q4} is selected from —C≡CH, —C≡CCD₃, —C≡CCH₂F, —C≡CCHF₂, —C≡CCF₃, —C≡CSi(CH₃)₃, —C≡CC(CH₃)₃, —C≡CSF₅, as well as the following:

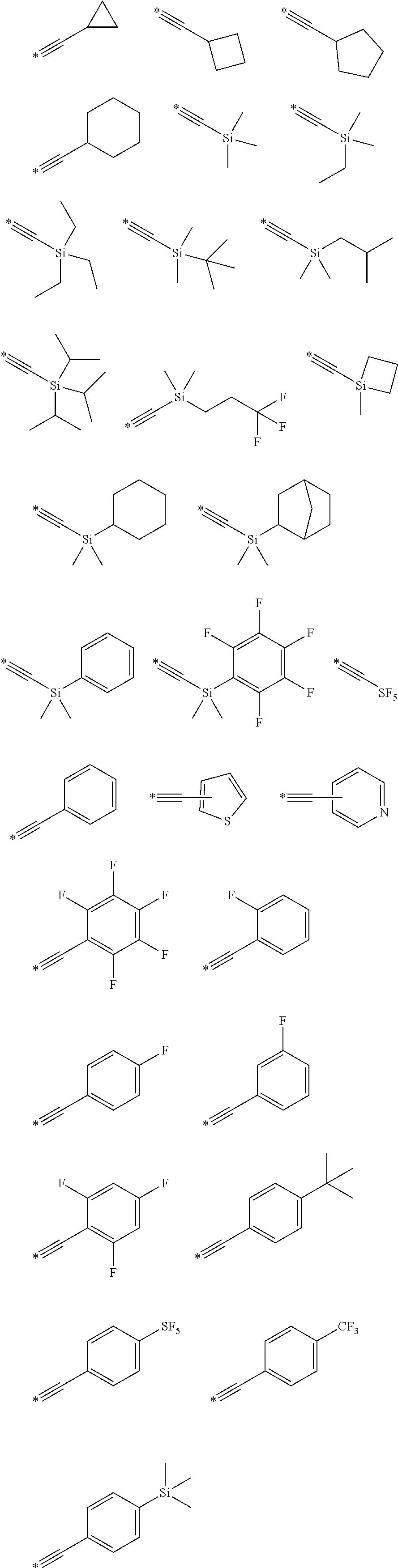

wherein * indicates the point of attachment to R_{q3}. In some examples, R_{q4} is —C≡CSi(CH₃)₃.

In some examples, R_{q4} is optionally substituted carbocyclyl, optionally substituted heterocyclyl, optionally substituted aryl, or optionally substituted heteroaryl. In some examples, the substituent(s) is independently selected from halogen, alkyl, heteroalkyl, silyl, and —SF₅, wherein each substituent may be further substituted by one or more R groups as described herein. Exemplary substituents of R_{q4} include, but are not limited to, fluorine, trifluoromethyl, ethynyl, 2-pentafluorosulfanylethynyl, 2-trimethylsilylethynyl, 2-(tert-butyl)ethynyl, tert-butyl, trimethylsilyl, and —SF₅. In some examples, R_{q4} is optionally substituted carbocyclyl, such as optionally substituted cyclopropyl, optionally substituted cyclobutyl, optionally substituted cyclopentyl, and optionally substituted cyclohexyl. In some examples, R_{q4} is optionally substituted heterocyclyl. In some examples, R_{q4} is optionally substituted aryl, such as optionally substituted phenyl (e.g., phenyl, 2-flurophenyl, 3-flurophenyl, 4-fluorophenyl, 2,4,6-triflurophenyl, 2,3,4,5,6-pentaflurophenyl, 4-(tert-butyl)phenyl, 4-(pentafluorosulfanyl)phenyl, 4-(trifluoromethyl)phenyl, 4-ethynylphenyl, 4-(2-pentafluorosulfanylethynyl)phenyl, 4-(2-trimethylsilylethynyl)phenyl, and 4-(2-(tert-butyl)ethynyl)phenyl. In some examples, R₄ is optionally substituted heteroaryl, such as optionally substituted pyridinyl and optionally substituted thiophenyl. In some examples, R_{q4} is selected from the following:

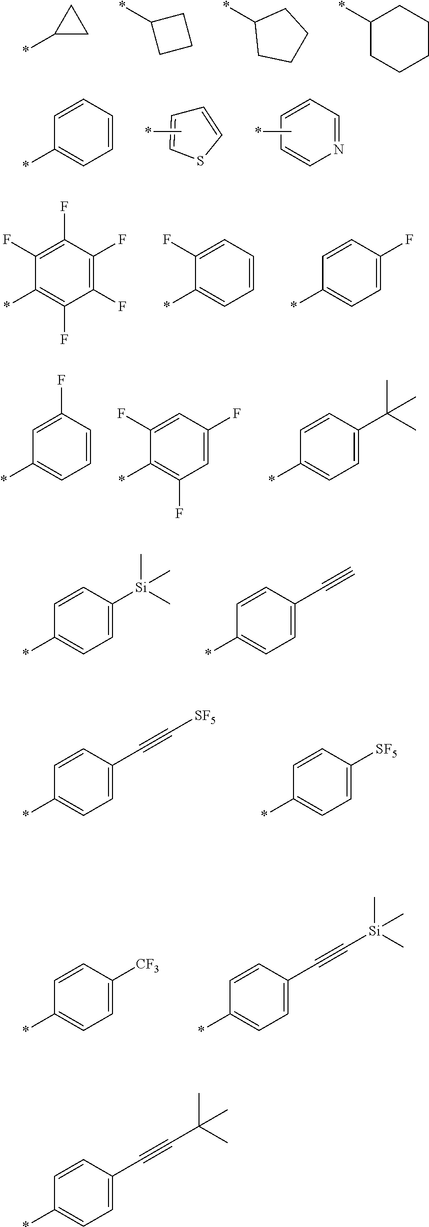

wherein * indicates the point of attachment to R_{q3}. In some examples, R_{q4} is cyclohexyl. In some examples, R_{q4} is 4-fluorophenyl.

In some examples, R_{q4} is Si-substituted silyl, having one or more substituents. When there are multiple substituents, two of the substituents can join together with the Si atom to form a cyclic moiety, such as a heterocycle. In some examples, the one or more substituents are independently selected from alkyl (such as methyl, ethyl, propyl, isopropyl, tert-butyl, and isobutyl), heteroalkyl, carbocyclyl (such as cyclohexyl and bicyclo[2.2.1]heptyl), heterocyclyl, aryl (such as phenyl), and heteroaryl, wherein each substituent may be further substituted by one or more R groups as described herein. In some examples, the Si-substituted silyl has three substituents, independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. In some examples, R_{q4} is selected from the following:

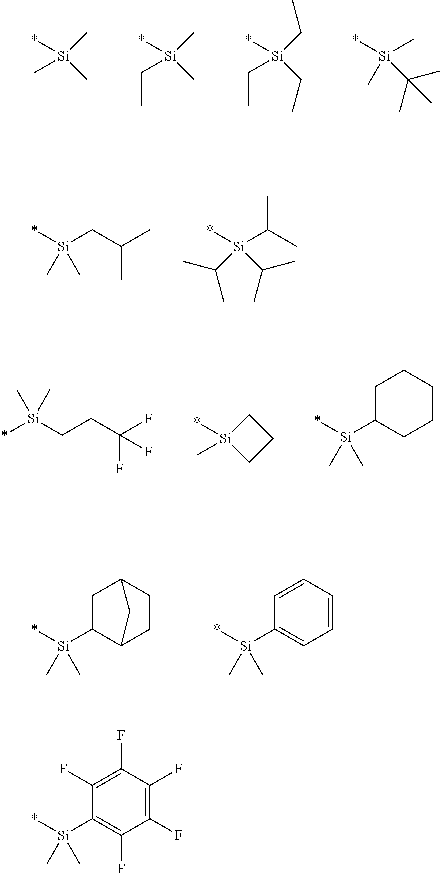

wherein * indicates the point of attachment to R_{q3}. In some examples, R_{q4} is trimethylsilyl.

In some examples, R_{q4} is S-substituted thiol, having one substituent. In some examples, the substituent is selected from alkyl, heteroalkyl, alkenyl, heteroalkenyl, alkynyl, heteroalkynyl, carbocyclyl, heterocyclyl, aryl (such as phenyl), and heteroaryl, which may be further substituted by one or more R groups as described herein. In some examples, the substituent is aryl, which may be further substituted by one or more R groups. In some examples, R_{q4} is —S-Ph.

In some examples, R₄ is O-substituted hydroxyl, having one substituent. In some examples, the substituent is selected from alkyl, heteroalkyl, alkenyl, heteroalkenyl, alkynyl, heteroalkynyl, carbocyclyl, heterocyclyl, aryl (such as phenyl), and heteroaryl, which may be further substituted by one or more R groups as described herein. In some examples, the substituent is aryl, which may be further substituted by one or more R groups. In some examples, R_{q4} is —O-Ph.

In some examples, R_{q4} is —SF₅.

In some examples, both R_{q1} and R₂ are present, i.e., Z is —O—R_{q1}—R_{q2}—R_{q3}—R_{q4}. In some examples, R_{q1} is a linear C₁-C₉ bridging alkylene (such as —CH₂CH₂— or —CH₂CH₂CH₂—); R_{q2} is —CF₂—, —O—, or —S—; R_{q3} is a linear C₂-C₂₀ bridging alkylene (such as a linear C₅-C₂₀ bridging alkylene); and R_{q4} is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅. In some examples, R_{q1} is —CH₂CH₂— or —CH₂CH₂CH₂—; R_{q2} is —CF₂—, —O—, or —S—; R_{q3} is a linear C₅-C₂₀ bridging alkylene; and R_{q4} is selected from —CD₃, —CF₃, —CD₂CD₃, —CF₂CF₃, —S-Ph, —O-Ph, —C≡CH, —C-CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

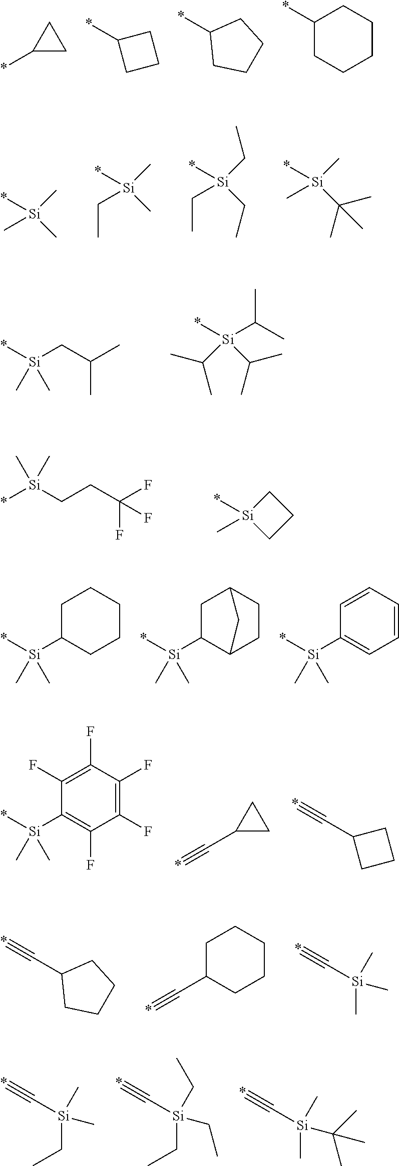

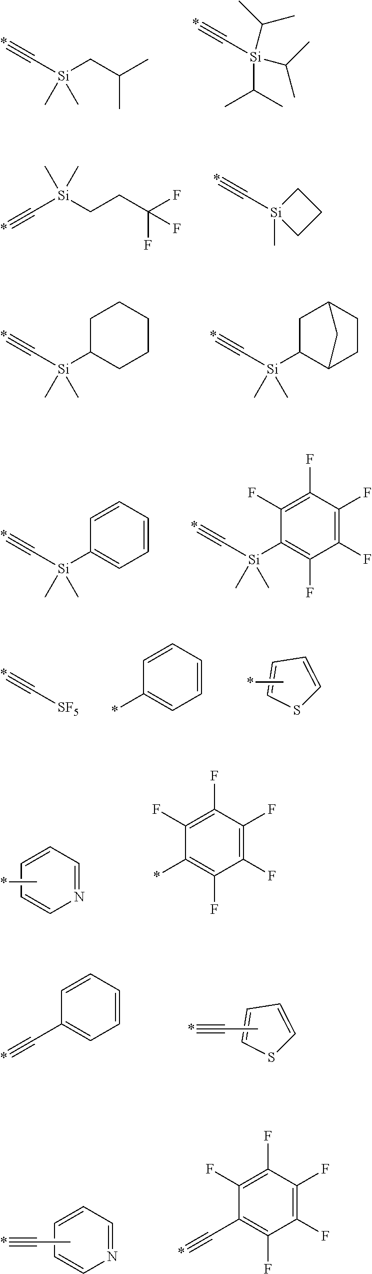

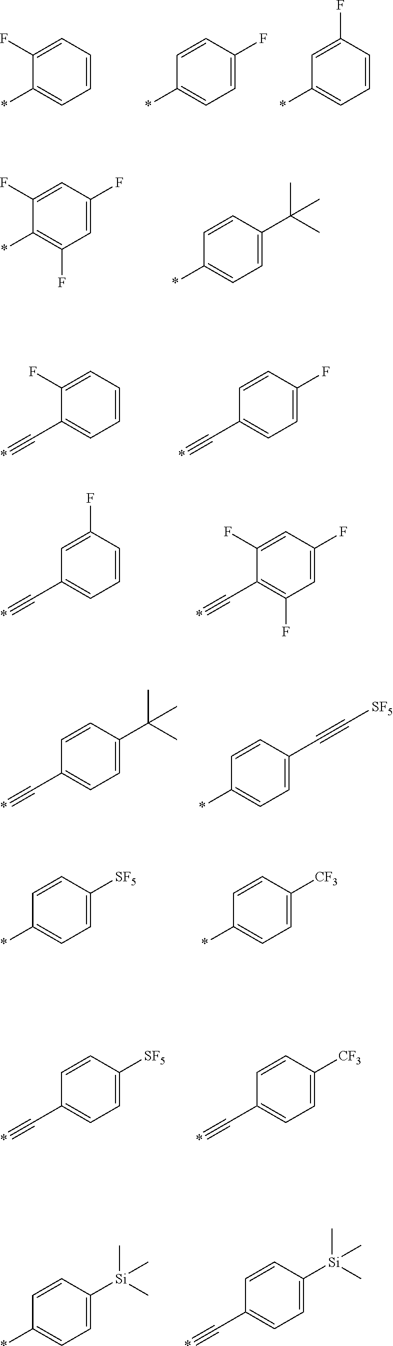

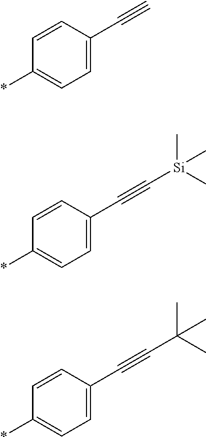

wherein * indicates the point of attachment to R_{q3}.

In some examples, both R_{q1} and R_{q2} are present, i.e., Z is —O—R_{q1}—R_{q2}—R_{q3}—R_{q4}, wherein R_{q1} is —CH₂CH₂— or —CH₂CH₂CH₂—, R_{q2} is —O— or —S—, R_{q3} is a linear C₅-C₂₀ bridging alkylene or a linear C₁₁-C₁₈ bridging alkylene, and R₄ is —CF₃

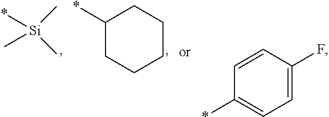

wherein * indicates the point of attachment to R₃.

In some examples, both R_{q1} and R_{q2} are present, i.e., Z is —O—R_{q1}—R_{q2}—R_{q3}—R_{q4}, wherein R_{q1} is —CH₂CH₂—, R_{q2} is —O— or —S—, R_{q3} is a linear C₅-C₂₀ bridging alkylene or a linear C₁₃-C₁₇ bridging alkylene, and R₄ is —CF₃

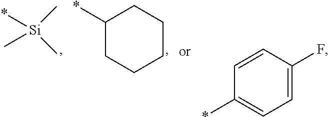

wherein * indicates the point of attachment to R_{q3}.

In some examples, both R_{q1} and R_{q2} are present, i.e., Z is —O—R_{q1}—R_{q2}—R_{q3}—R_{q4}, wherein R_{q1} is a linear C₁-C₉ bridging alkylene (such as —CH₂CH₂— or —CH₂CH₂CH₂—), R_{q2} is —CF₂—, —O—, —S—S—, or —S—, R_{q3} is a linear C₂-C₂₀ bridging alkylene (such as a linear C₅-C₂₀ bridging alkylene or a linear C₂-C₇ bridging alkylene), and R₄ is hydrogen, methyl, or ethyl.

In some examples, both R_{q1} and R_{q2} are present, i.e., Z is —O—R_{Q}—R_{q2}—R_{q3}—R_{q4}, wherein R_{q1} is —CH₂CH₂— or —CH₂CH₂CH₂—, R_{q2} is —O— or —S—, R_{q3} is a linear C₅-C₂₀ bridging alkylene or a linear C₁₅-C₁₉ bridging alkylene, and R₄ is hydrogen.

In some examples, both R_{q1} and R_{q2} are absent, i.e., Z is —O—R_{q3}—R_{q4}. In some examples, R_{q3} is a linear C₂-C₂₀ bridging alkylene (such as a linear C₅-C₂₀ alkylene); and R₄ is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅. In some examples, R_{q3} is a linear C₅-C₂₀ bridging alkylene; and R₄ is selected from —CD₃, —CF₃, —CD₂CD₃, —CF₂CF₃, —S-Ph, —O-Ph, —C≡CH, —C≡CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

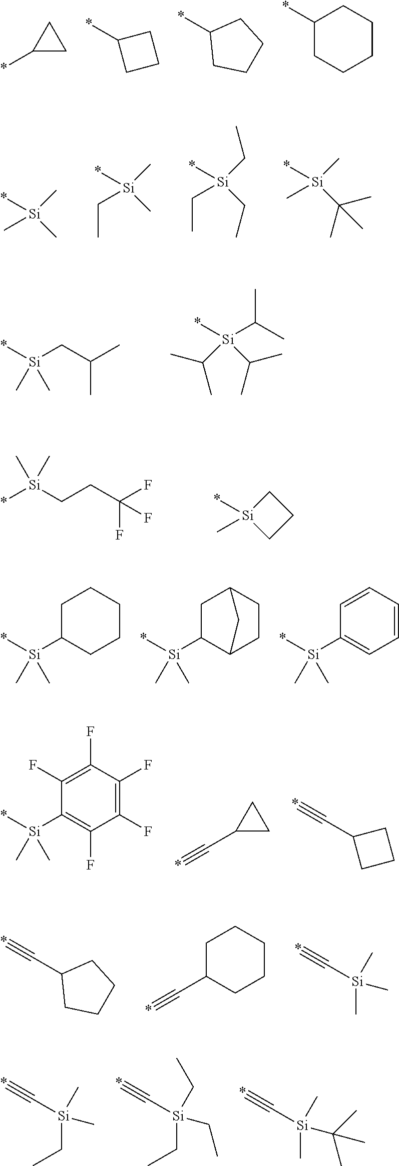

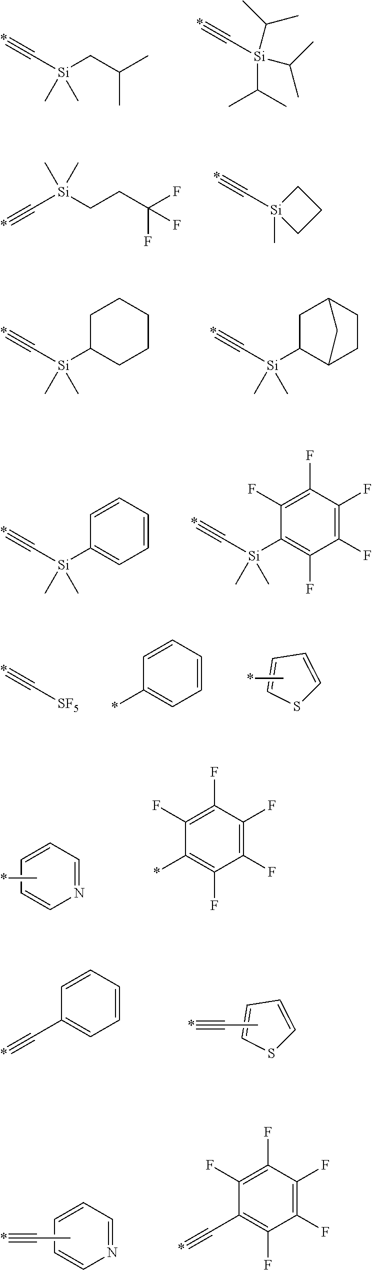

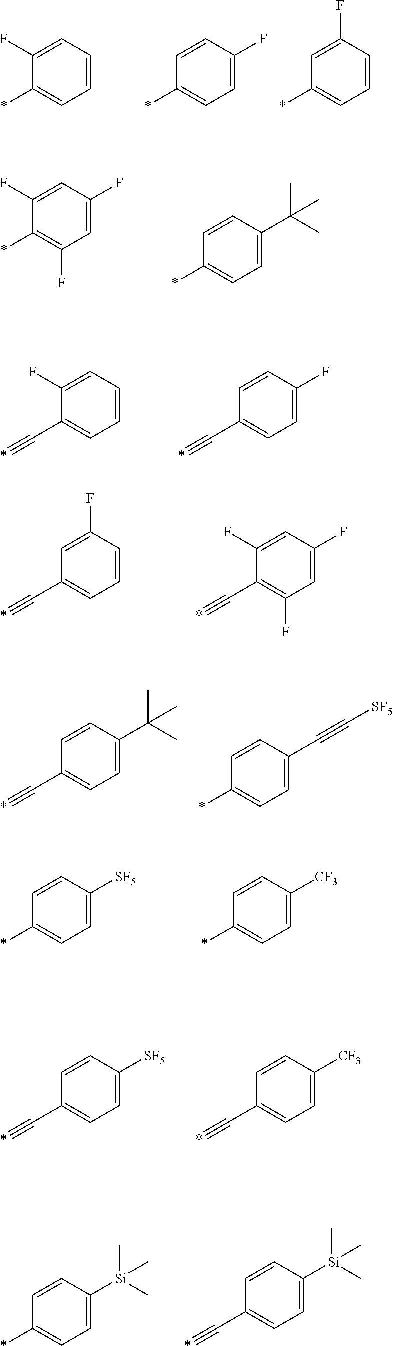

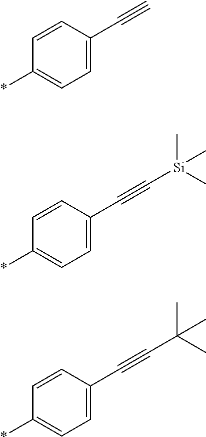

wherein * indicates the point of attachment to Y.

In some examples, both R_{q1} and R_{q2} are absent, i.e., Z is —O—R_{q3}—R_{q4}, wherein R_{q3} is a linear C₂-C₂₀ bridging alkylene (such as a linear C₅-C₂₀ alkylene) and R₄ is selected from hydrogen, methyl or ethyl.

In some examples, Z is selected from:

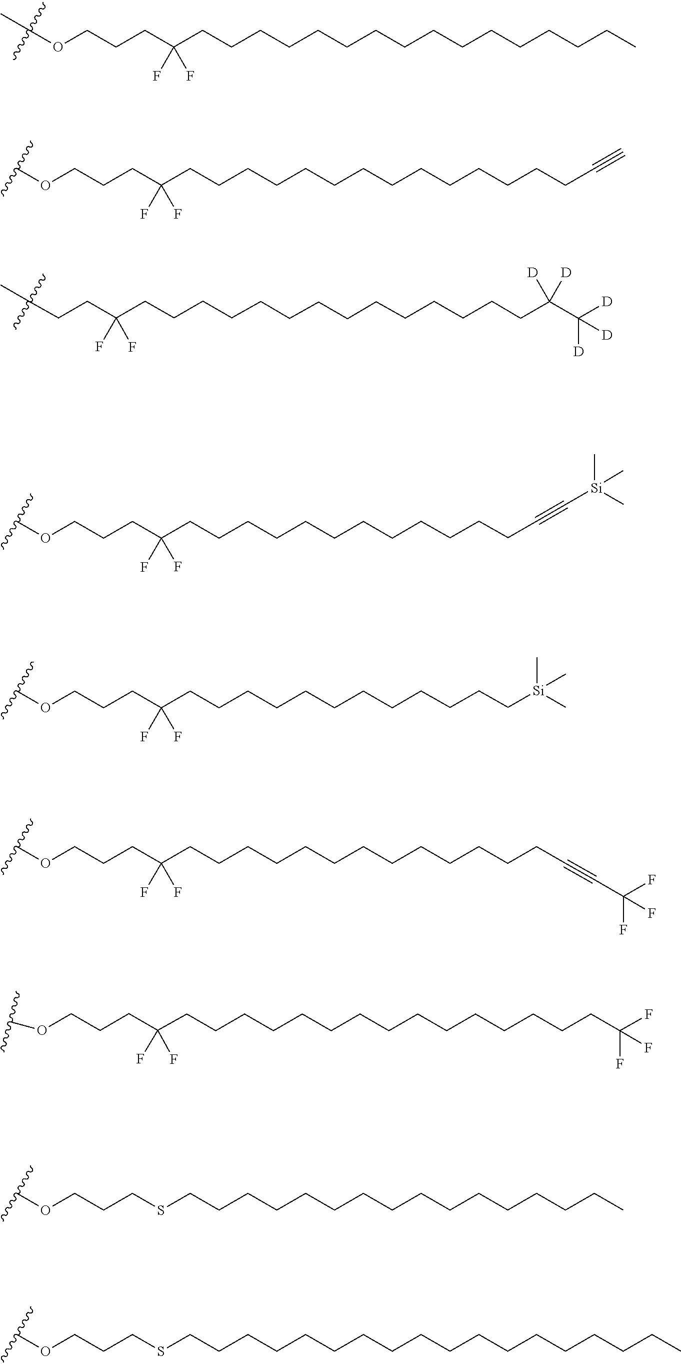

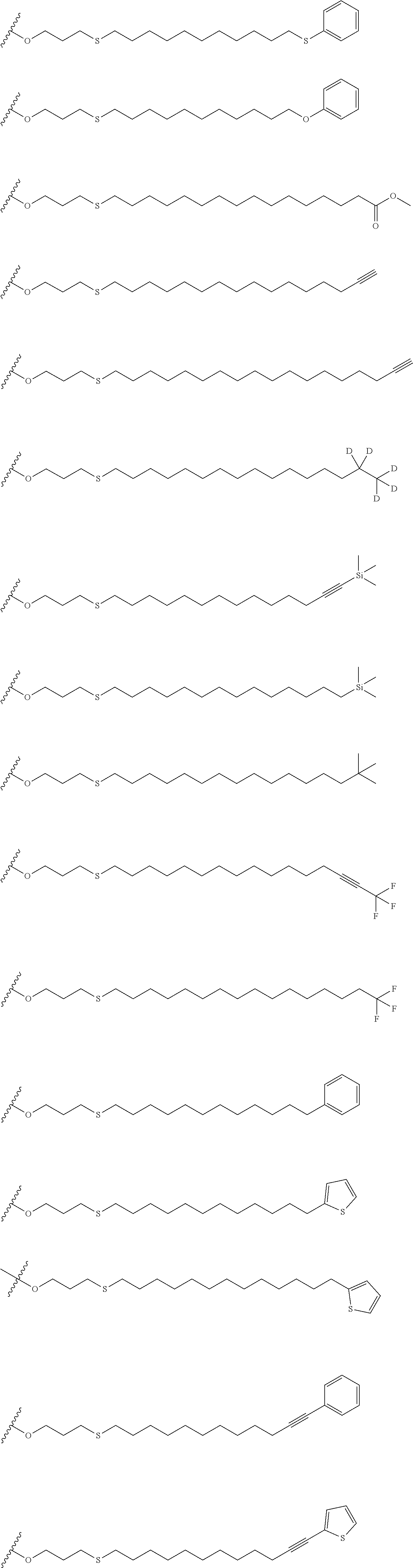

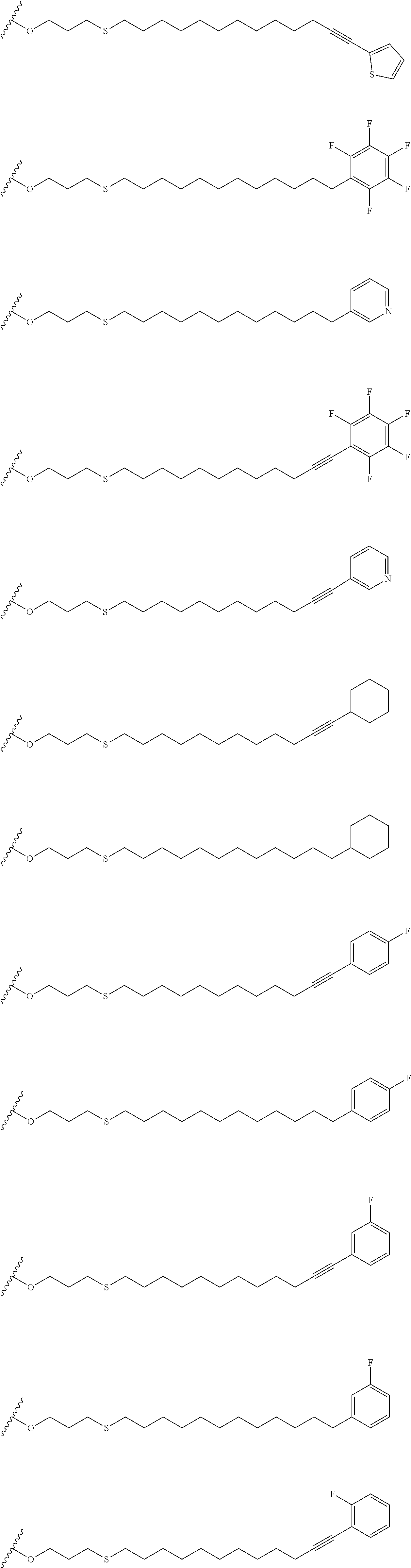

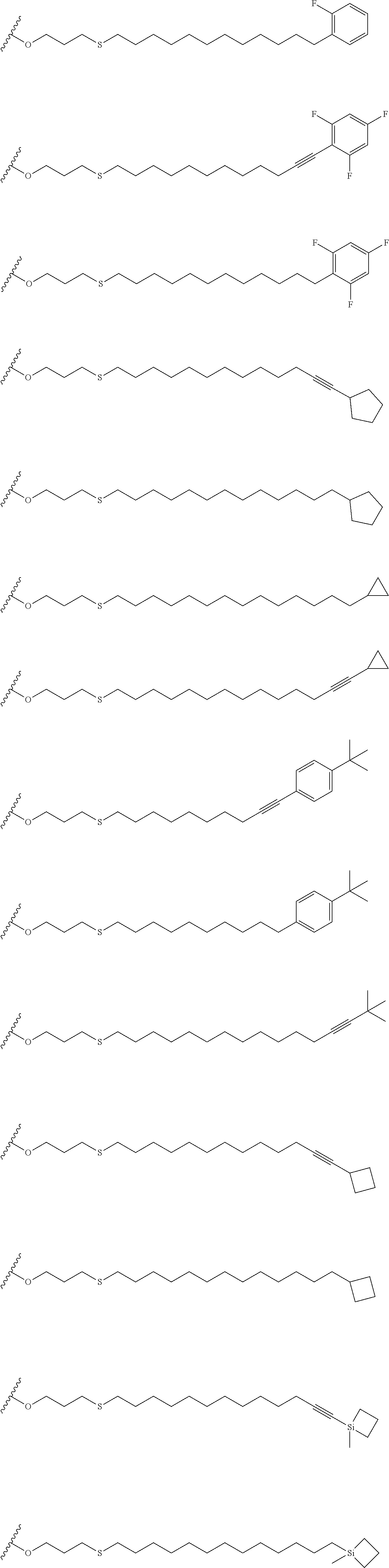

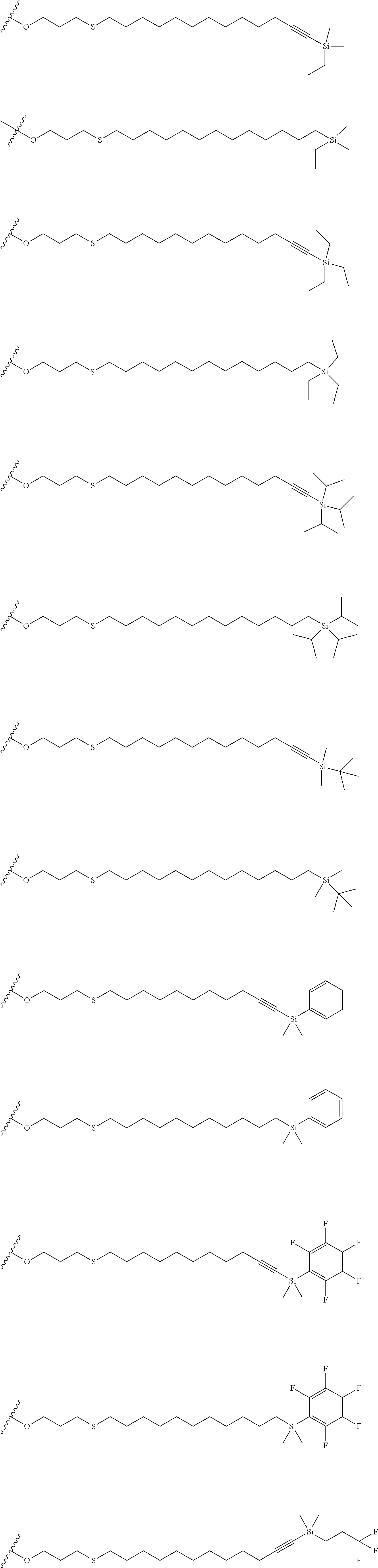

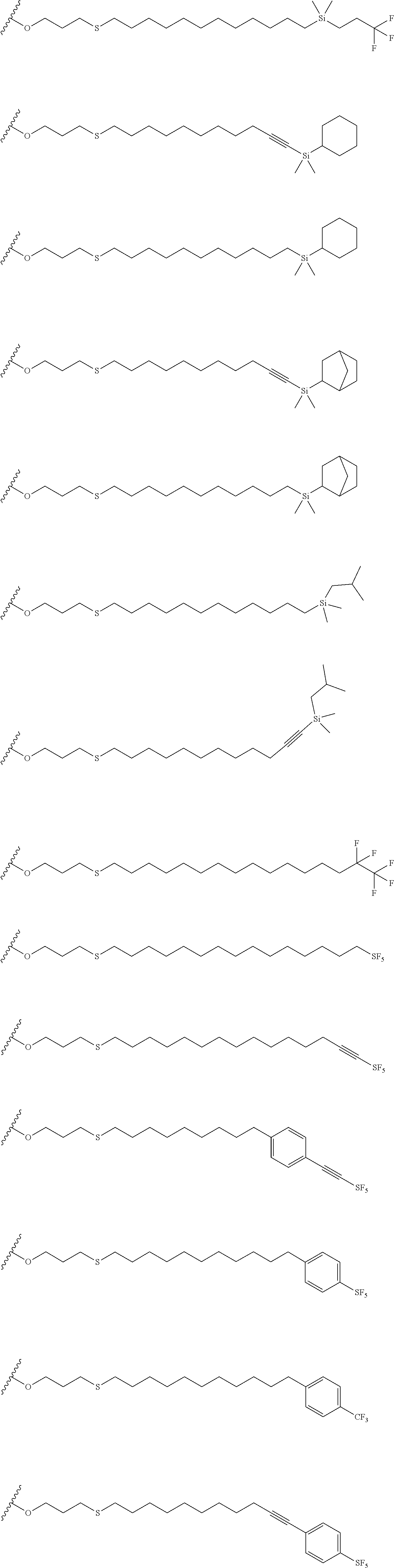

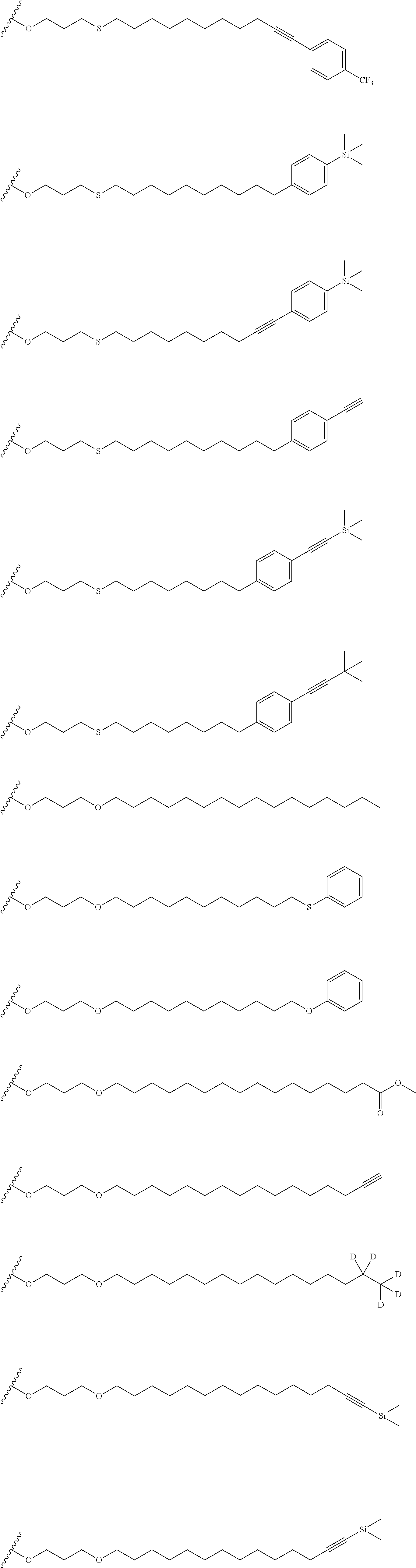

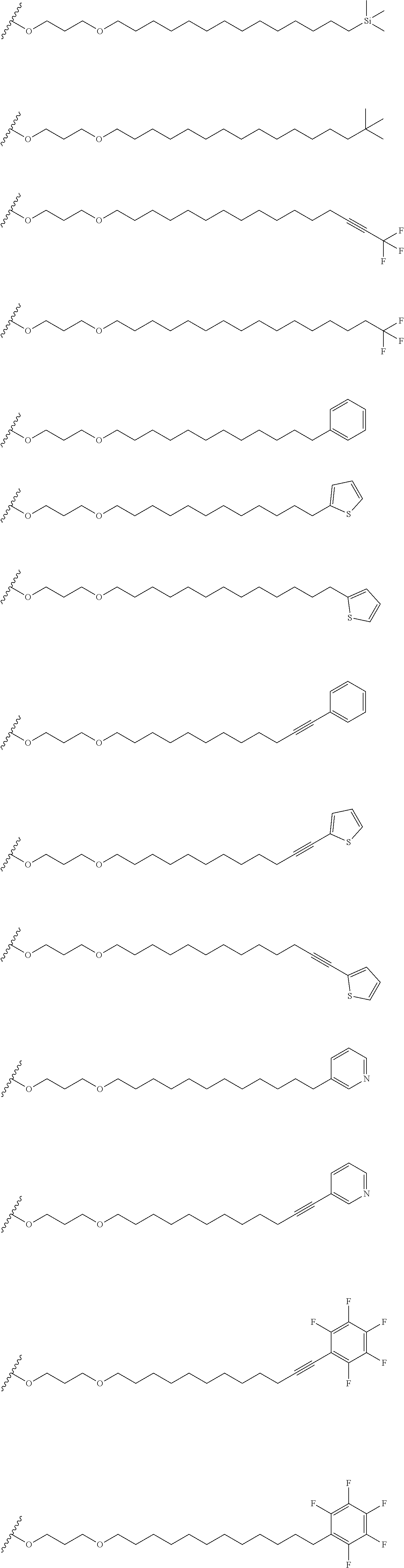

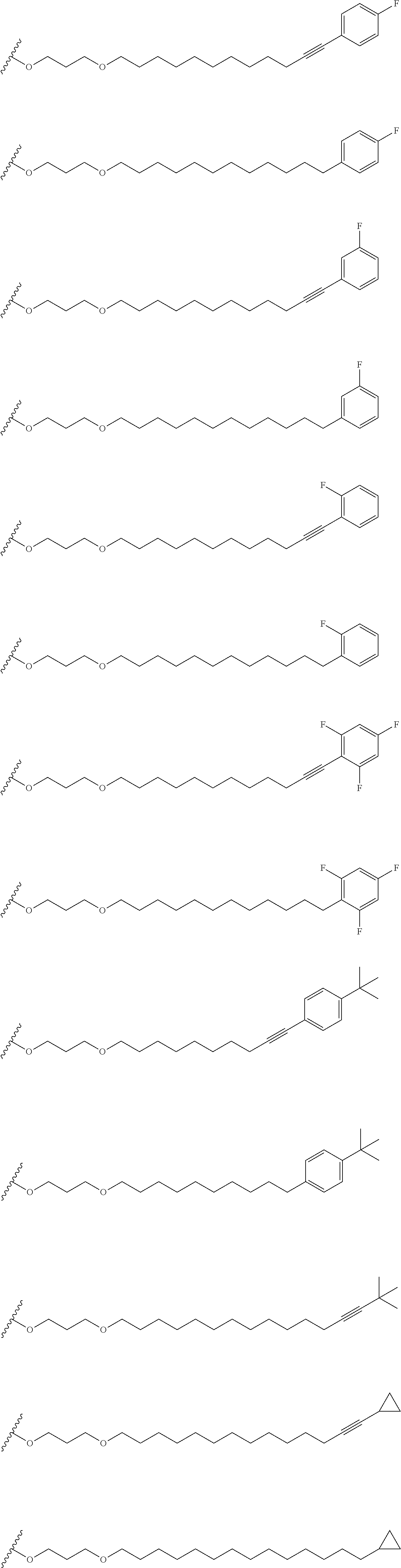

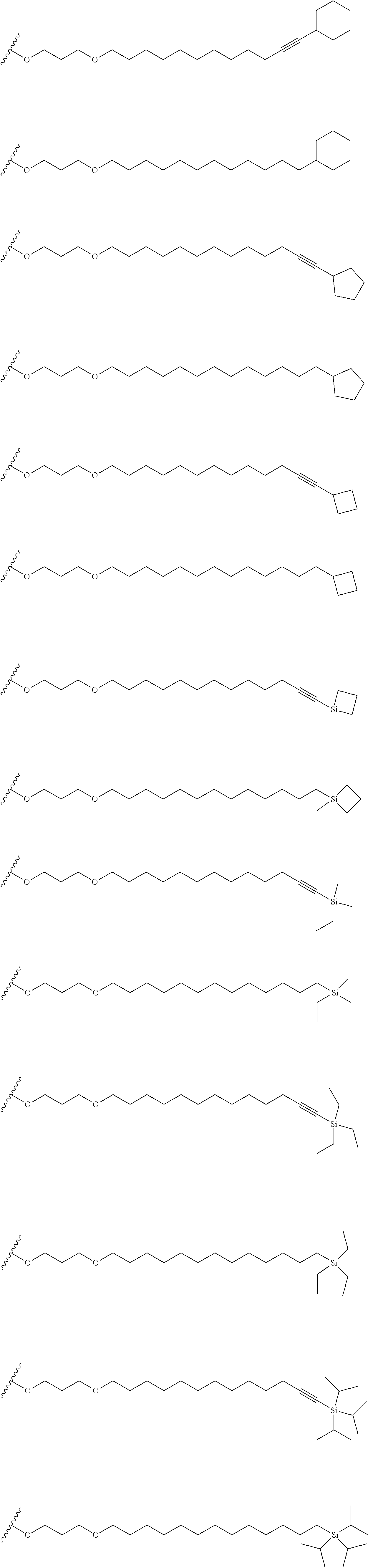

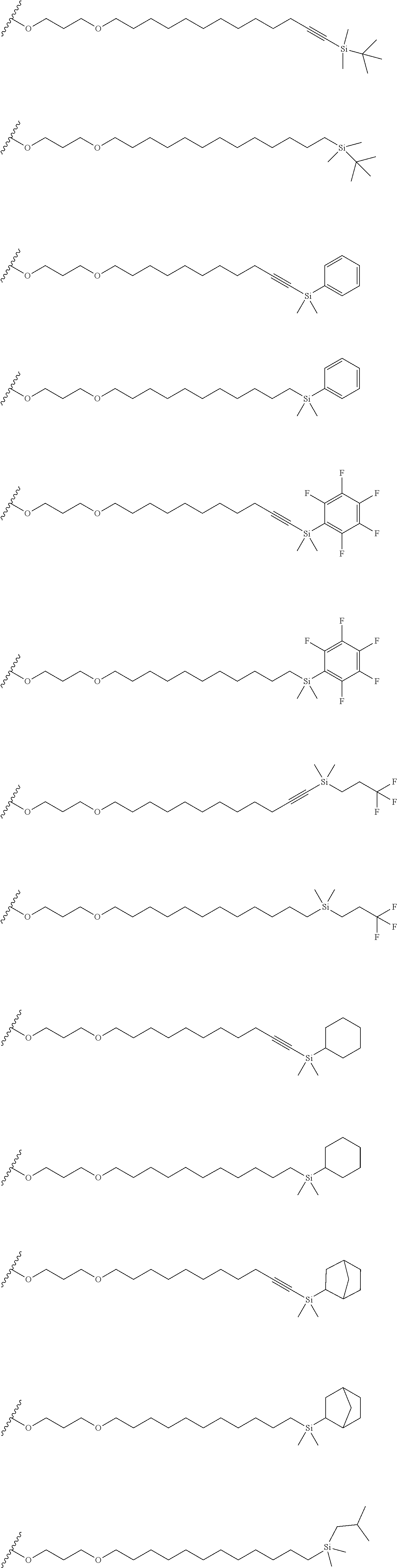

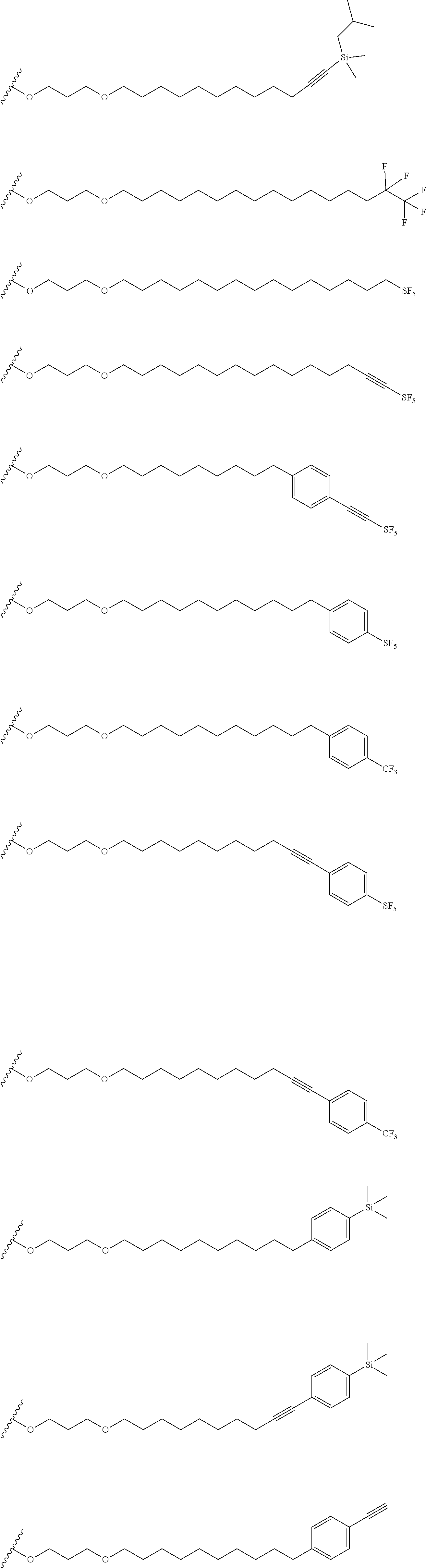

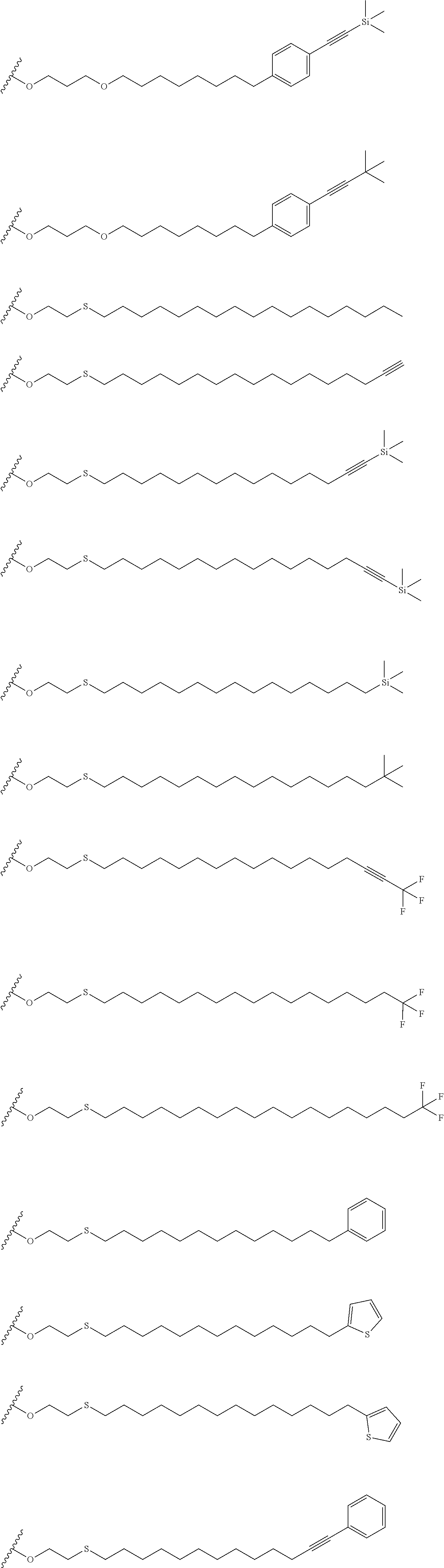

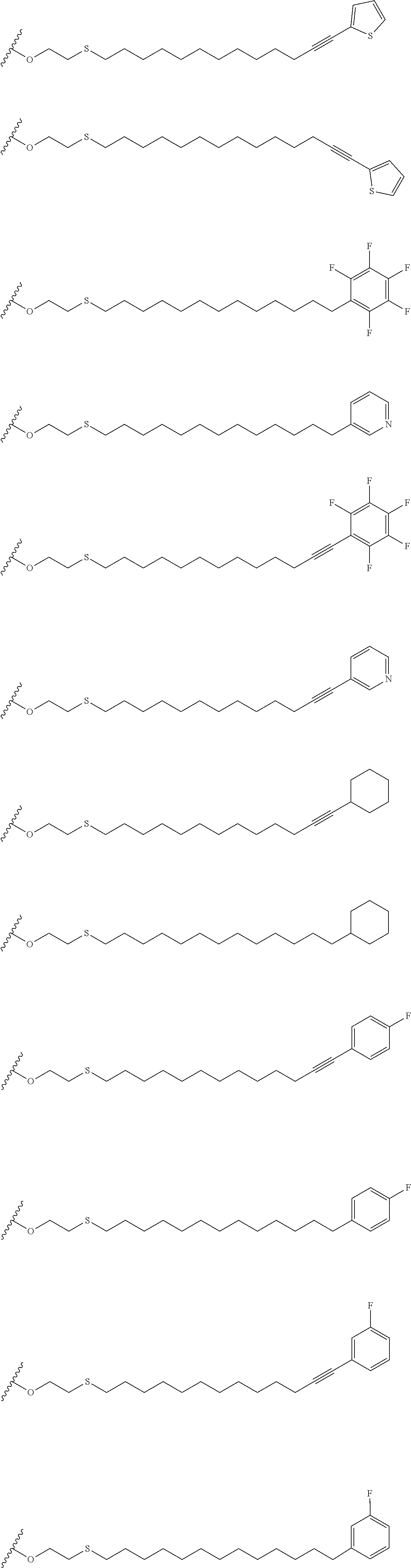

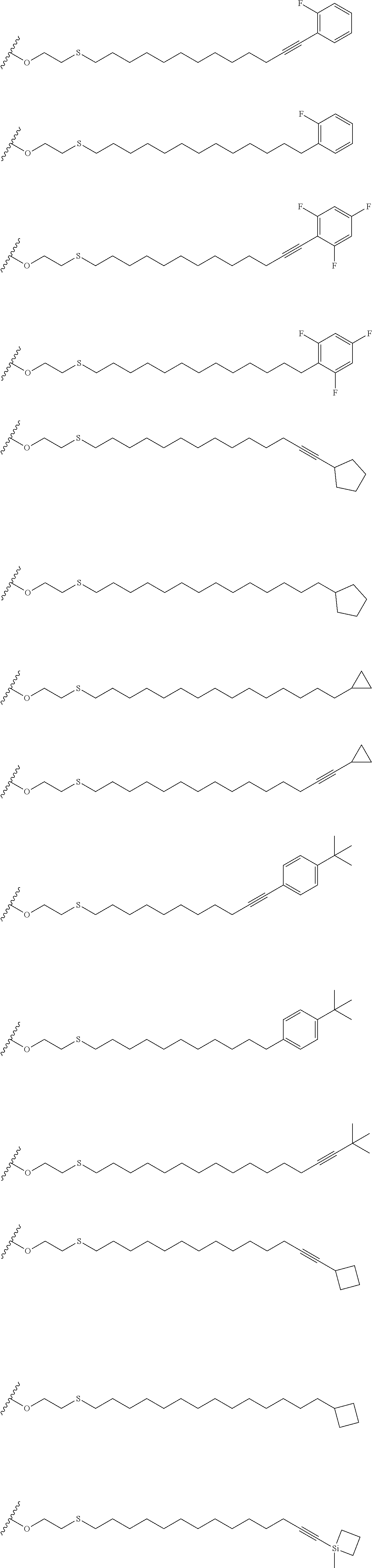

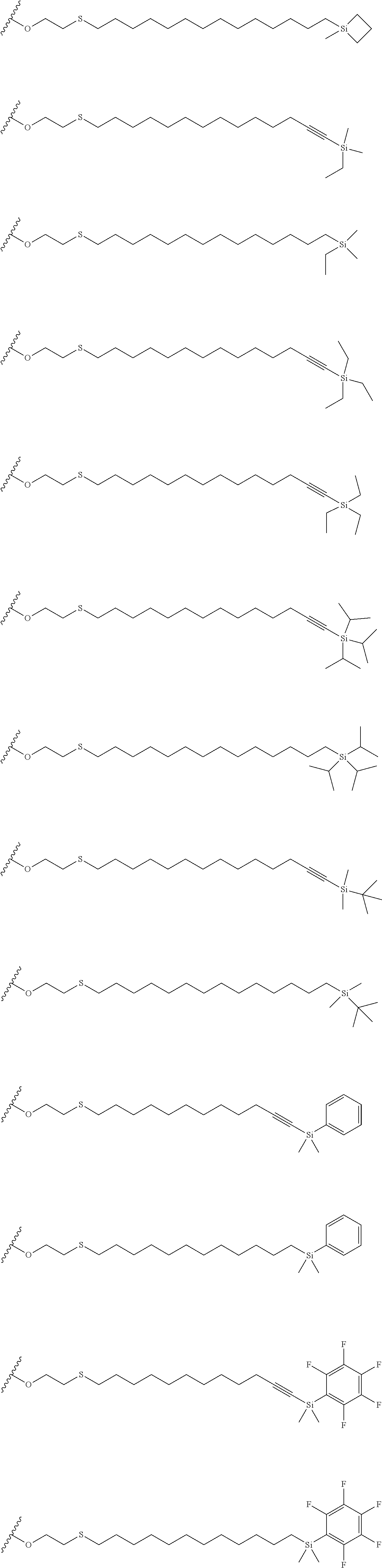

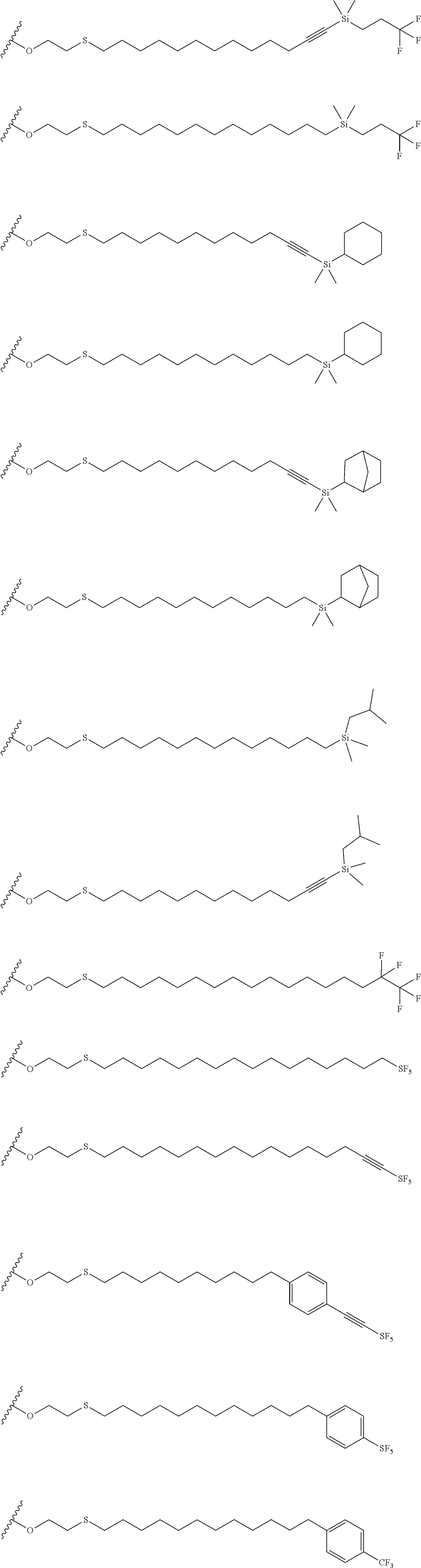

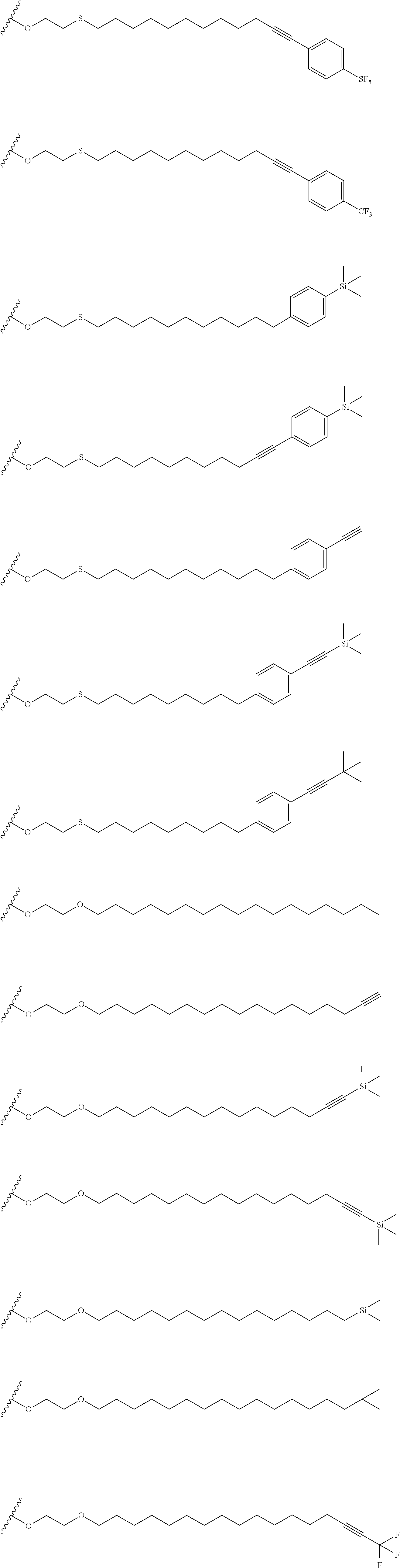

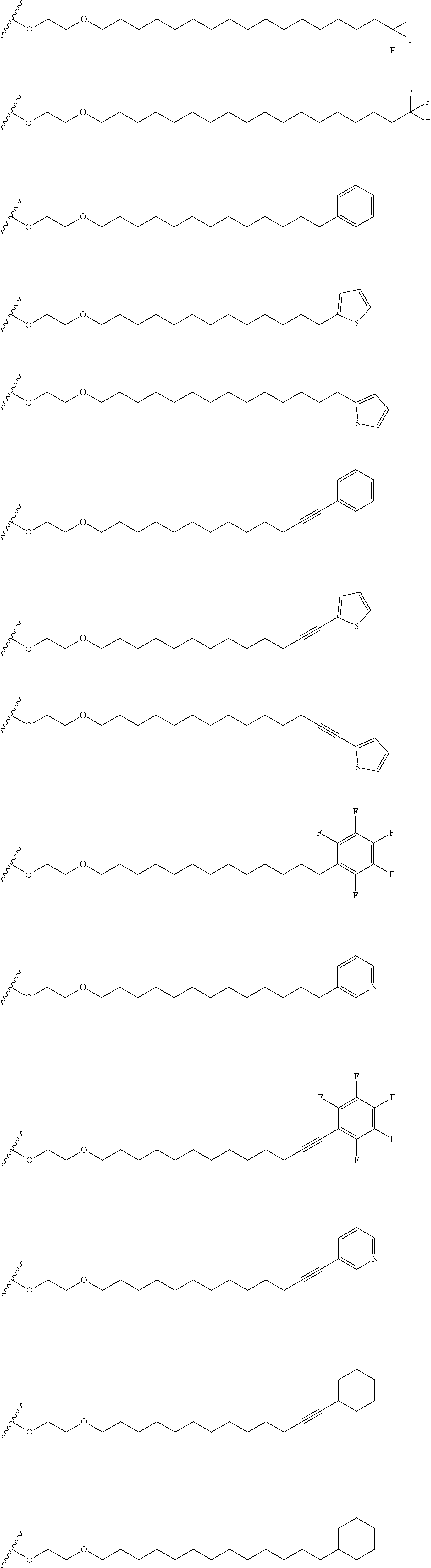

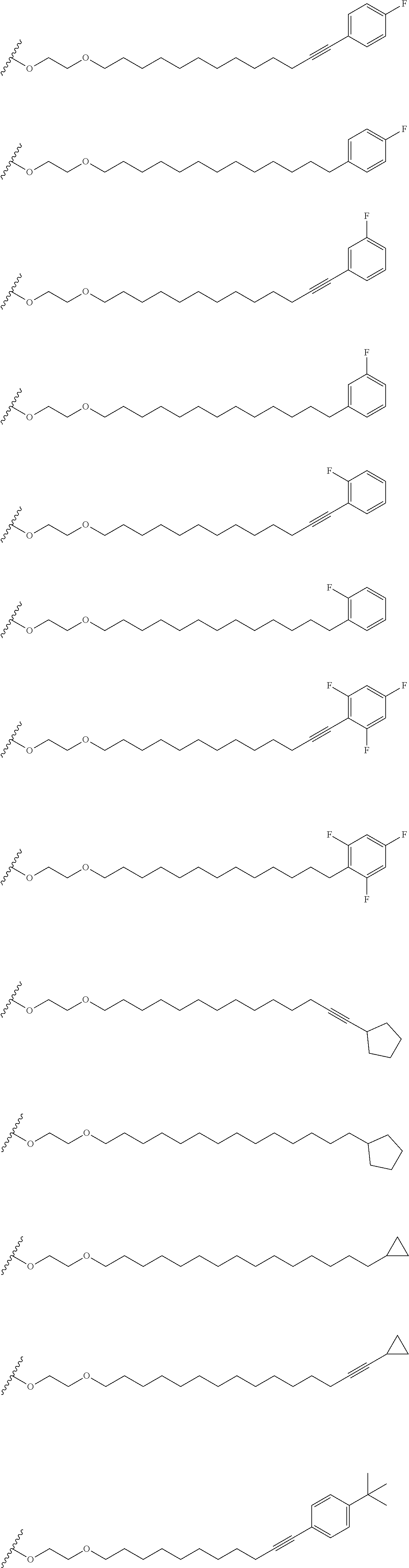

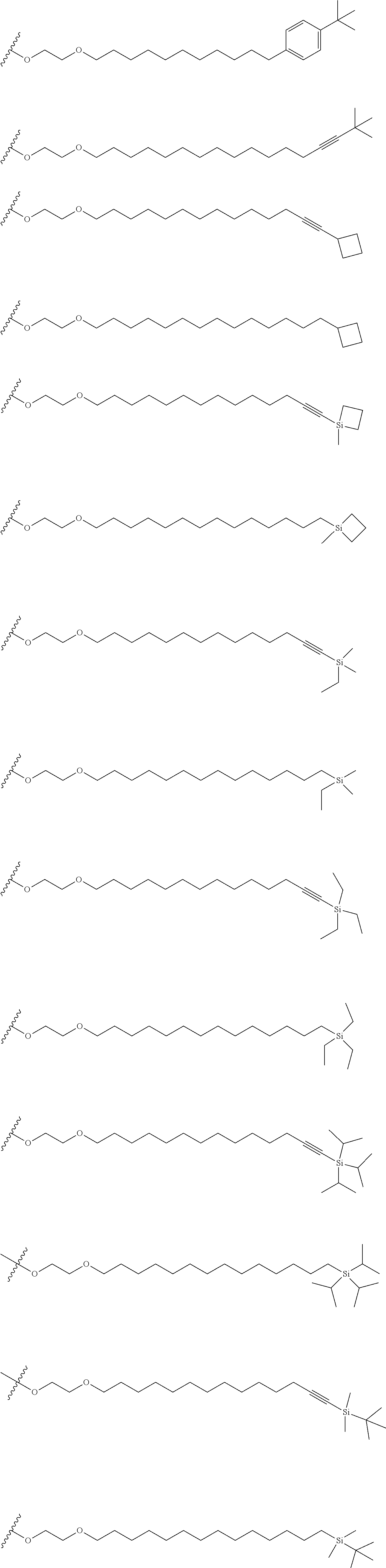

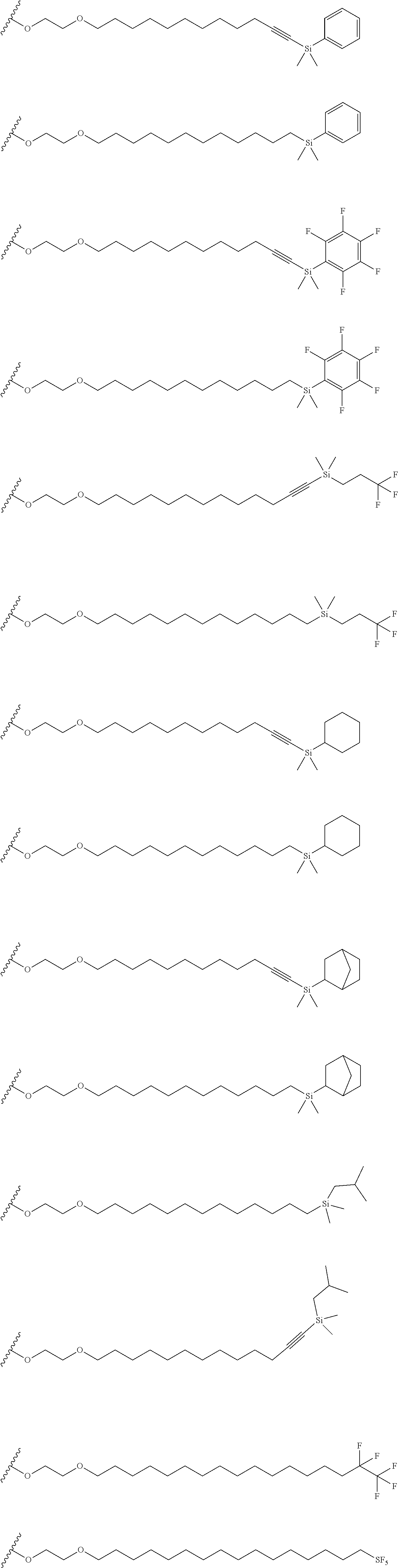

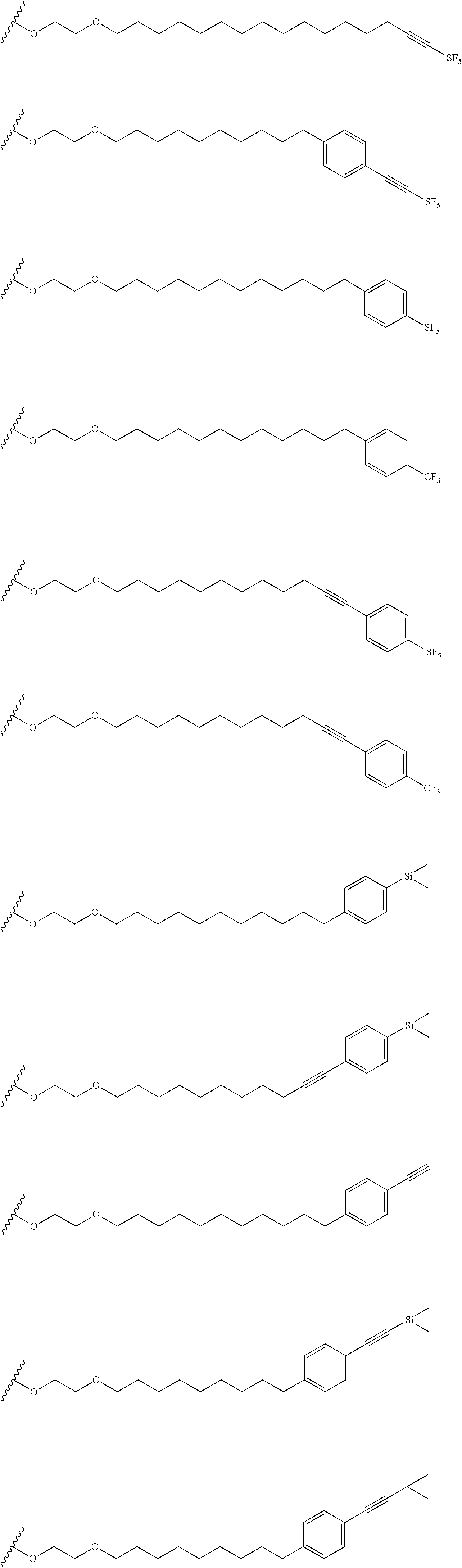

In some examples, Z is selected from:

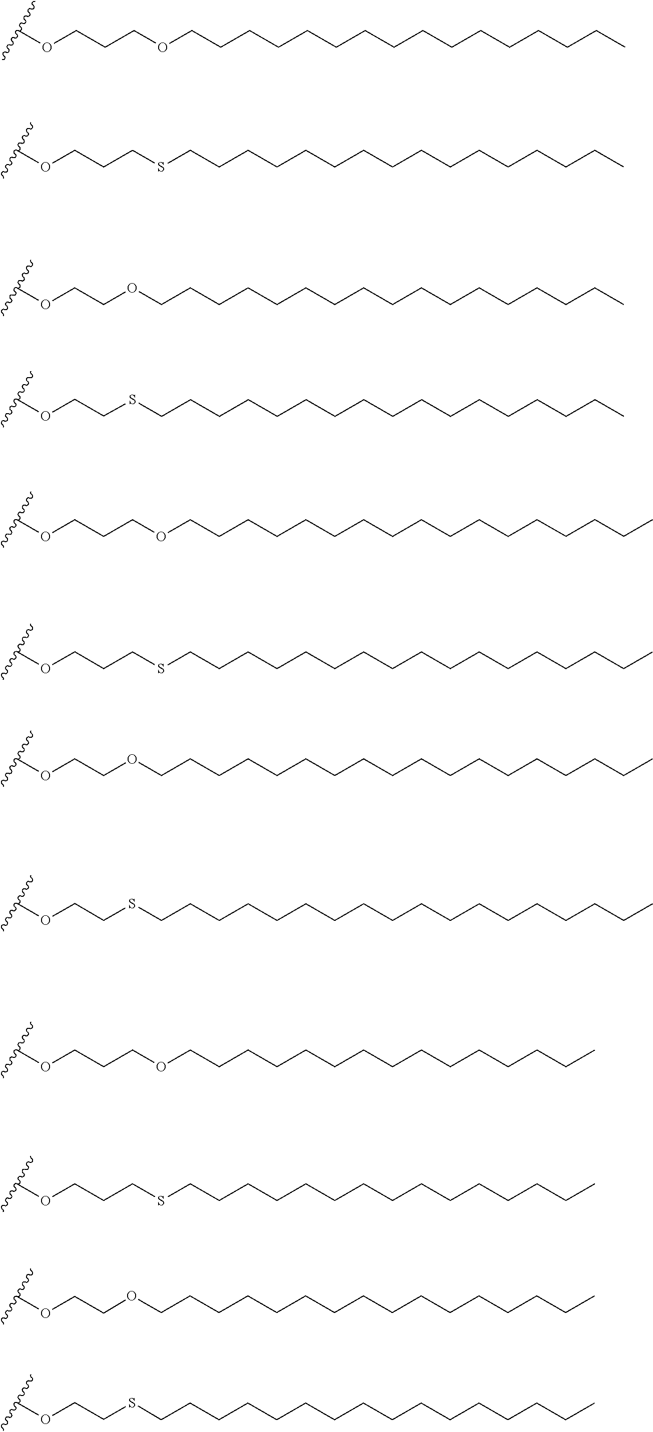

In some examples, Z is selected from:

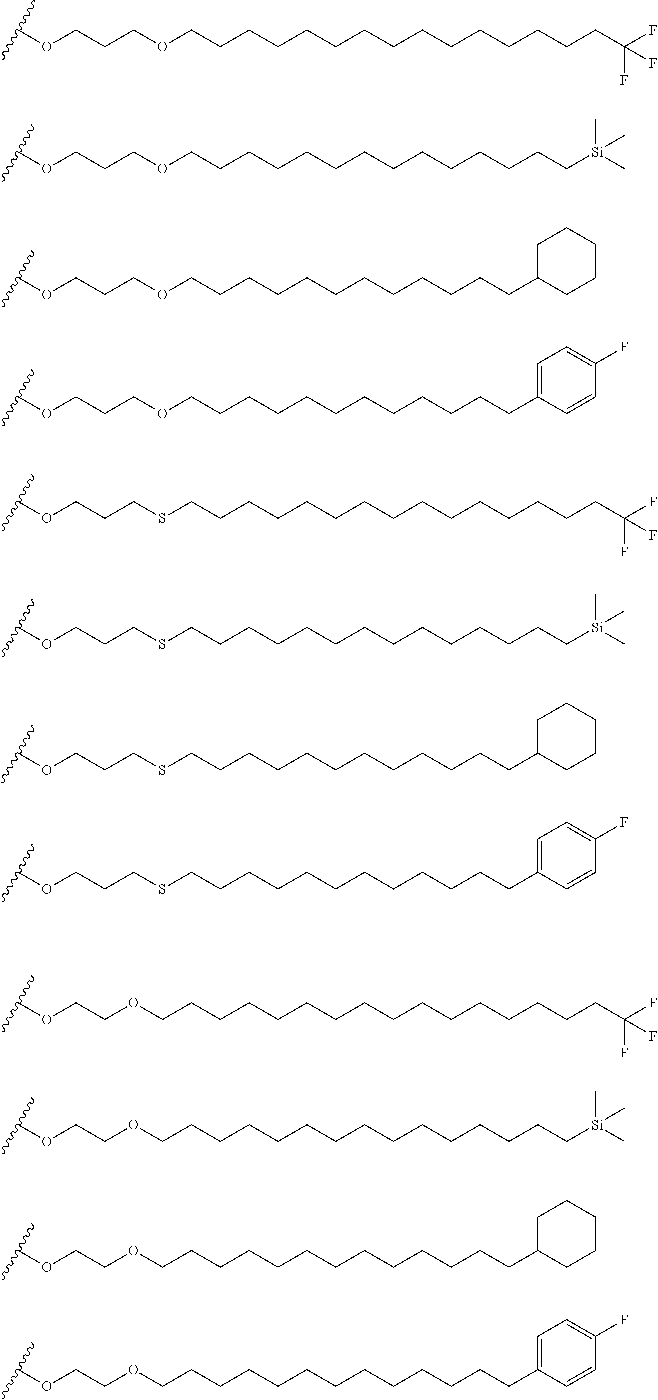

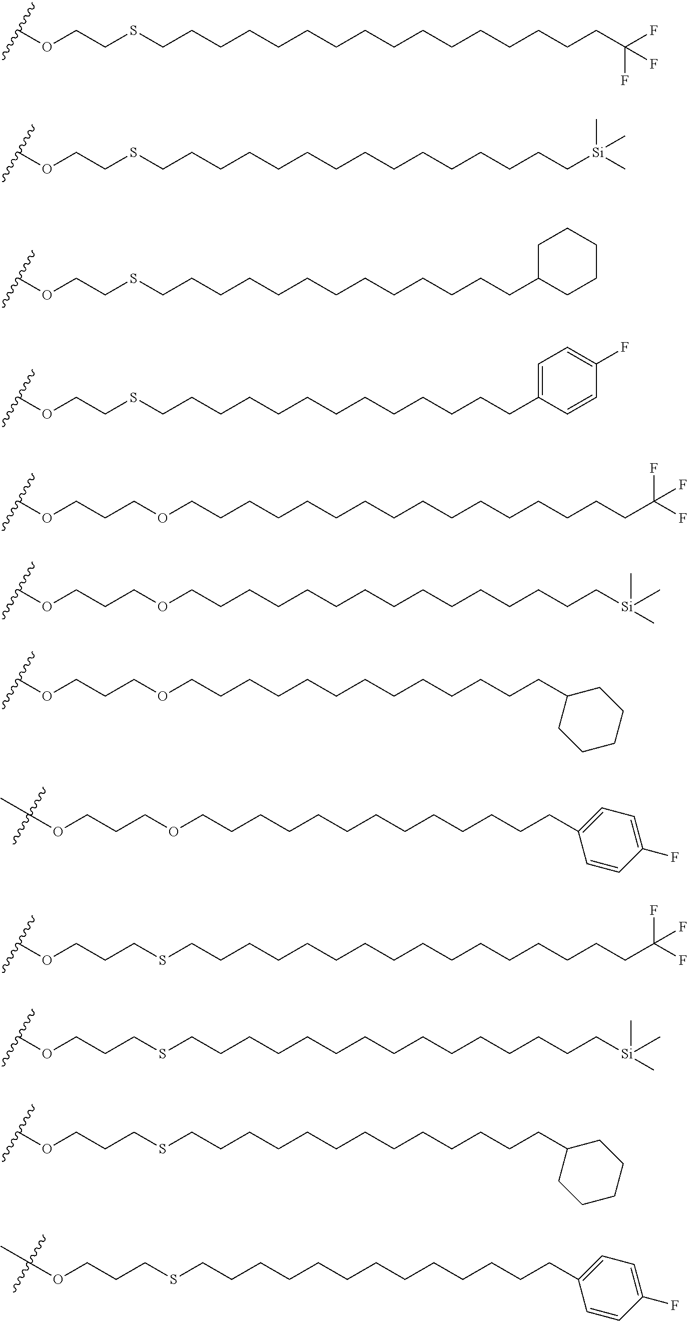

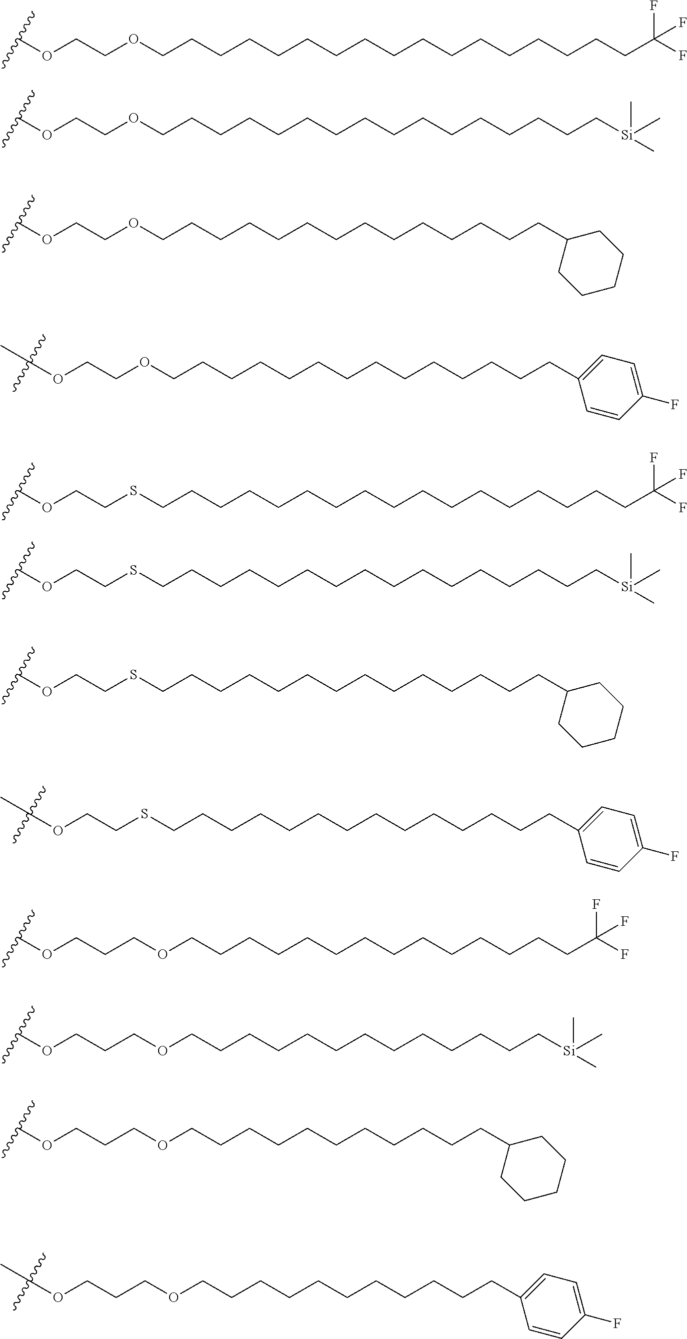

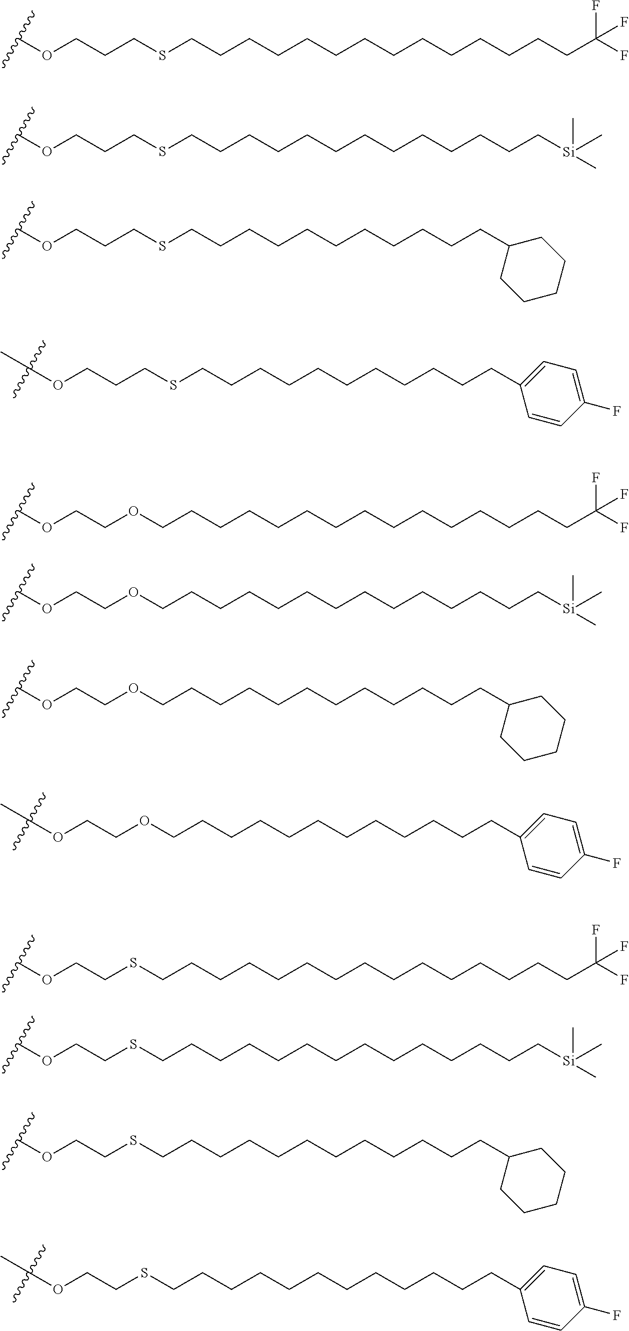

In some examples, the lipid-derived prodrugs have a structure selected from the following structures or pharmaceutically acceptable salts (such as ammonium and lithium salts) thereof:

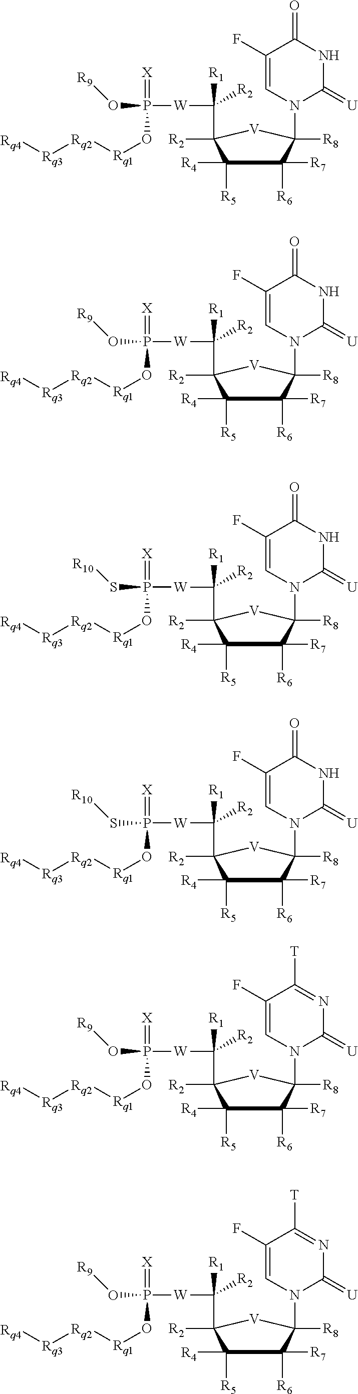

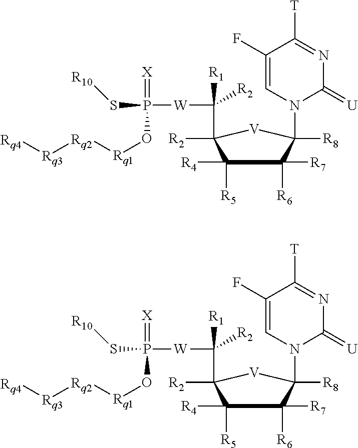

In some examples, the lipid-derived prodrugs have a structure selected from the following structures or pharmaceutically acceptable salts (such as ammonium and lithium salts) thereof:

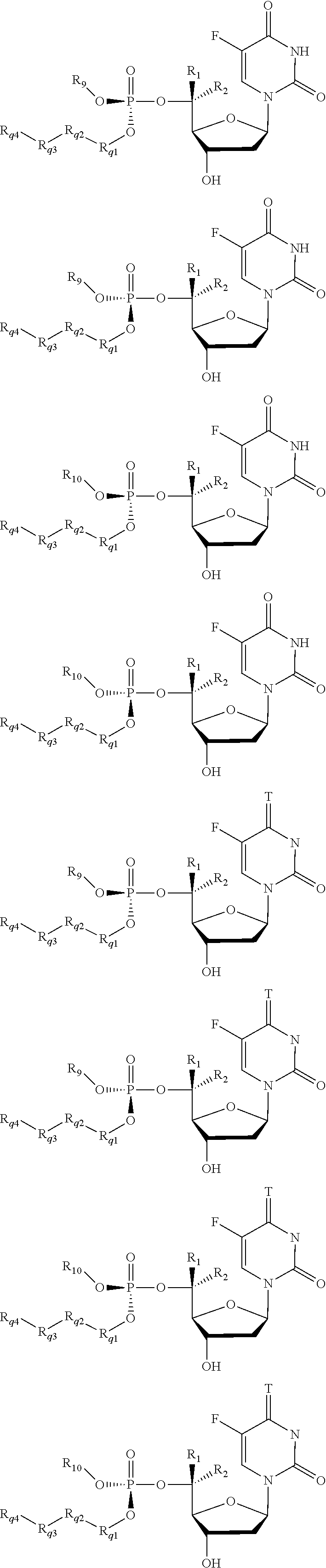

Exemplary lipid-derived prodrugs include, but are not limited to, the following structures and pharmaceutically acceptable salts (such as ammonium and lithium salts) thereof:

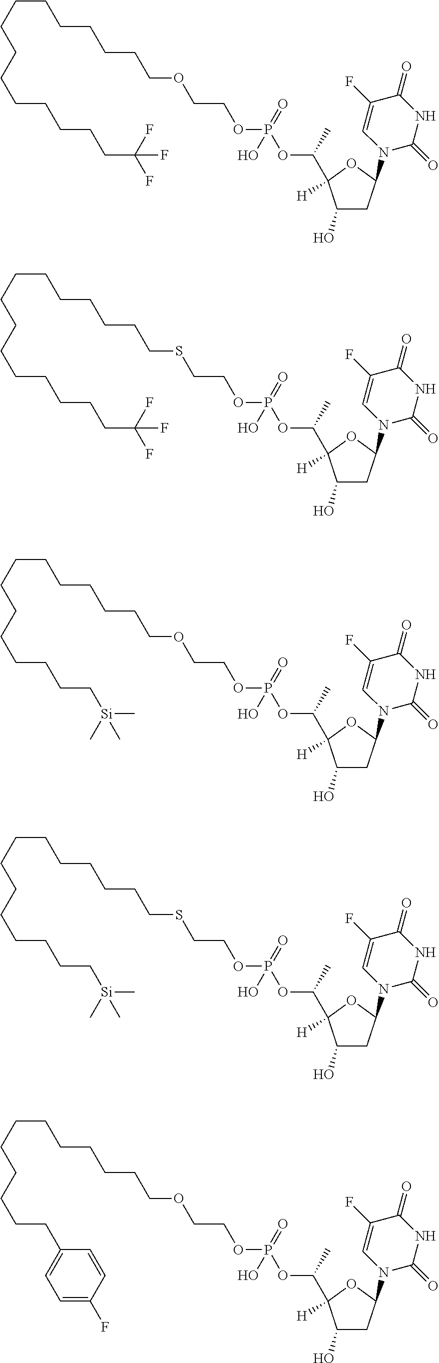 [SMILES: C[C@@H](OP(=O)(O)OCCOCCCCCCCCCCCCCCCC(F)(F)F)[C@H]1O[C@@H](n2cc(F)c(=O)[nH]c2=O)C[C@@H]1O ; C[C@@H](OP(=O)(O)OCCSCCCCCCCCCCCCCCCC(F)(F)F)[C@H]1O[C@@H](n2cc(F)c(=O)[nH]c2=O)C[C@@H]1O ; C[C@@H](OP(=O)(O)OCCOCCCCCCCCCCCCCC[Si](C)(C)C)[C@H]1O[C@@H](n2cc(F)c(=O)[nH]c2=O)C[C@@H]1O ; C[C@@H](OP(=O)(O)OCCSCCCCCCCCCCCCCC[Si](C)(C)C)[C@H]1O[C@@H](n2cc(F)c(=O)[nH]c2=O)C[C@@H]1O ; C[C@@H](OP(=O)(O)OCCOCCCCCCCCCCCCc1ccc(F)cc1)[C@H]1O[C@@H](n2cc(F)c(=O)[nH]c2=O)C[C@@H]1O]

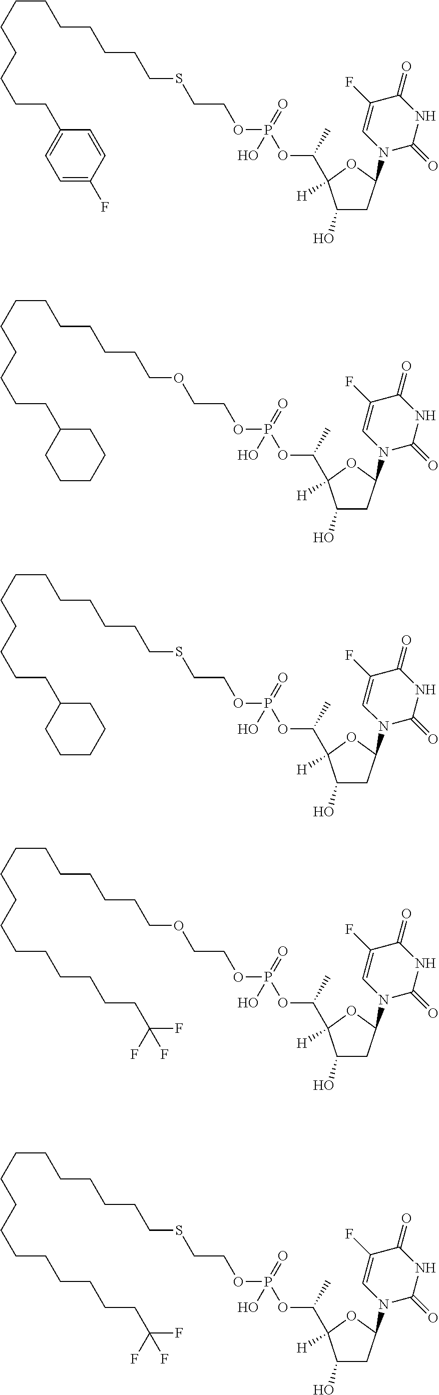 [SMILES: C[C@@H](OP(=O)(O)OCCSCCCCCCCCCCCCc1ccc(F)cc1)[C@H]1O[C@@H](n2cc(F)c(=O)[nH]c2=O)C[C@@H]1O ; C[C@@H](OP(=O)(O)OCCOCCCCCCCCCCCCC1CCCCC1)[C@H]1O[C@@H](n2cc(F)c(=O)[nH]c2=O)C[C@@H]1O ; C[C@@H](OP(=O)(O)OCCSCCCCCCCCCCCCC1CCCCC1)[C@H]1O[C@@H](n2cc(F)c(=O)[nH]c2=O)C[C@@H]1O ; C[C@@H](OP(=O)(O)OCCOCCCCCCCCCCCCCCCCC(F)(F)F)[C@H]1O[C@@H](n2cc(F)c(=O)[nH]c2=O)C[C@@H]1O ; C[C@@H](OP(=O)(O)OCCSCCCCCCCCCCCCCCCCC(F)(F)F)[C@H]1O[C@@H](n2cc(F)c(=O)[nH]c2=O)C[C@@H]1O]

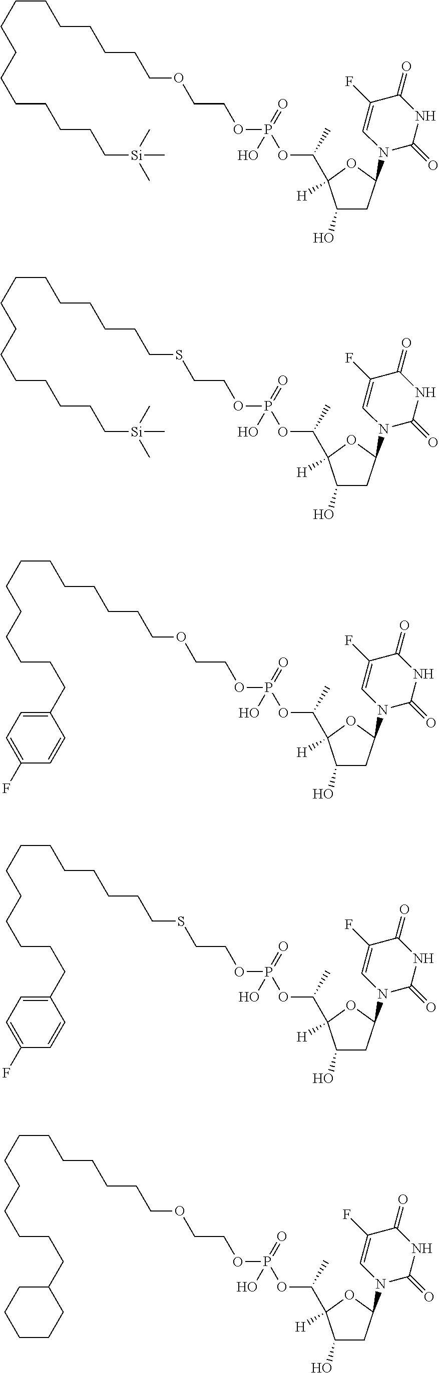 [SMILES: C[C@@H](OP(=O)(O)OCCOCCCCCCCCCCCCCCC[Si](C)(C)C)[C@H]1O[C@@H](n2cc(F)c(=O)[nH]c2=O)C[C@@H]1O ; C[C@@H](OP(=O)(O)OCCSCCCCCCCCCCCCCCC[Si](C)(C)C)[C@H]1O[C@@H](n2cc(F)c(=O)[nH]c2=O)C[C@@H]1O ; C[C@@H](OP(=O)(O)OCCOCCCCCCCCCCCCCc1ccc(F)cc1)[C@H]1O[C@@H](n2cc(F)c(=O)[nH]c2=O)C[C@@H]1O ; C[C@@H](OP(=O)(O)OCCSCCCCCCCCCCCCCc1ccc(F)cc1)[C@H]1O[C@@H](n2cc(F)c(=O)[nH]c2=O)C[C@@H]1O ; C[C@@H](OP(=O)(O)OCCOCCCCCCCCCCCCCC1CCCCC1)[C@H]1O[C@@H](n2cc(F)c(=O)[nH]c2=O)C[C@@H]1O]

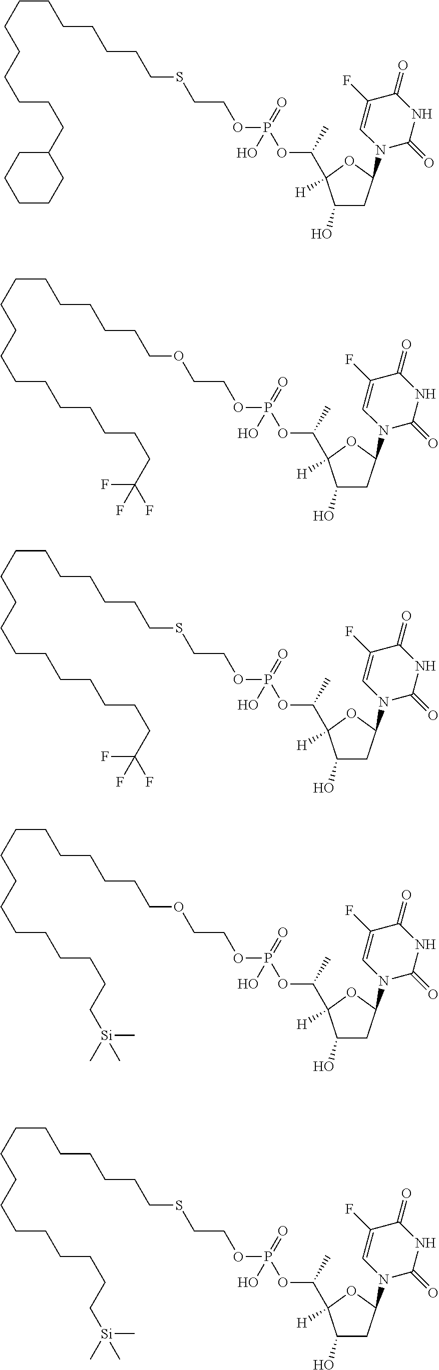 [SMILES: C[C@@H](OP(=O)(O)OCCSCCCCCCCCCCCCCC1CCCCC1)[C@H]1O[C@@H](n2cc(F)c(=O)[nH]c2=O)C[C@@H]1O ; C[C@@H](OP(=O)(O)OCCOCCCCCCCCCCCCCCCCCC(F)(F)F)[C@H]1O[C@@H](n2cc(F)c(=O)[nH]c2=O)C[C@@H]1O ; C[C@@H](OP(=O)(O)OCCSCCCCCCCCCCCCCCCCCC(F)(F)F)[C@H]1O[C@@H](n2cc(F)c(=O)[nH]c2=O)C[C@@H]1O ; C[C@@H](OP(=O)(O)OCCOCCCCCCCCCCCCCCCC[Si](C)(C)C)[C@H]1O[C@@H](n2cc(F)c(=O)[nH]c2=O)C[C@@H]1O ; C[C@@H](OP(=O)(O)OCCSCCCCCCCCCCCCCCCC[Si](C)(C)C)[C@H]1O[C@@H](n2cc(F)c(=O)[nH]c2=O)C[C@@H]1O]

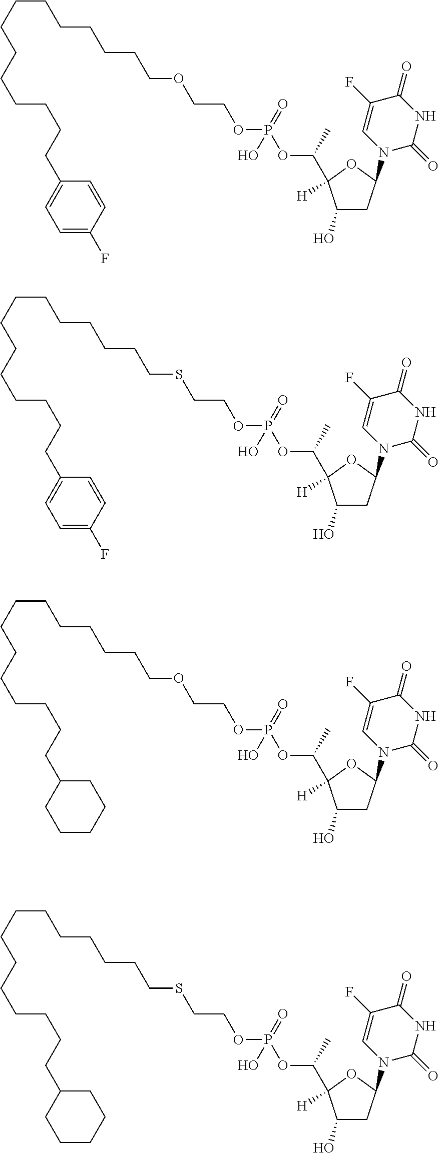 [SMILES: C[C@@H](OP(=O)(O)OCCOCCCCCCCCCCCCCCc1ccc(F)cc1)[C@H]1O[C@@H](n2cc(F)c(=O)[nH]c2=O)C[C@@H]1O ; C[C@@H](OP(=O)(O)OCCSCCCCCCCCCCCCCCc1ccc(F)cc1)[C@H]1O[C@@H](n2cc(F)c(=O)[nH]c2=O)C[C@@H]1O ; C[C@@H](OP(=O)(O)OCCOCCCCCCCCCCCCCCC1CCCCC1)[C@H]1O[C@@H](n2cc(F)c(=O)[nH]c2=O)C[C@@H]1O ; C[C@@H](OP(=O)(O)OCCSCCCCCCCCCCCCCCC1CCCCC1)[C@H]1O[C@@H](n2cc(F)c(=O)[nH]c2=O)C[C@@H]1O]

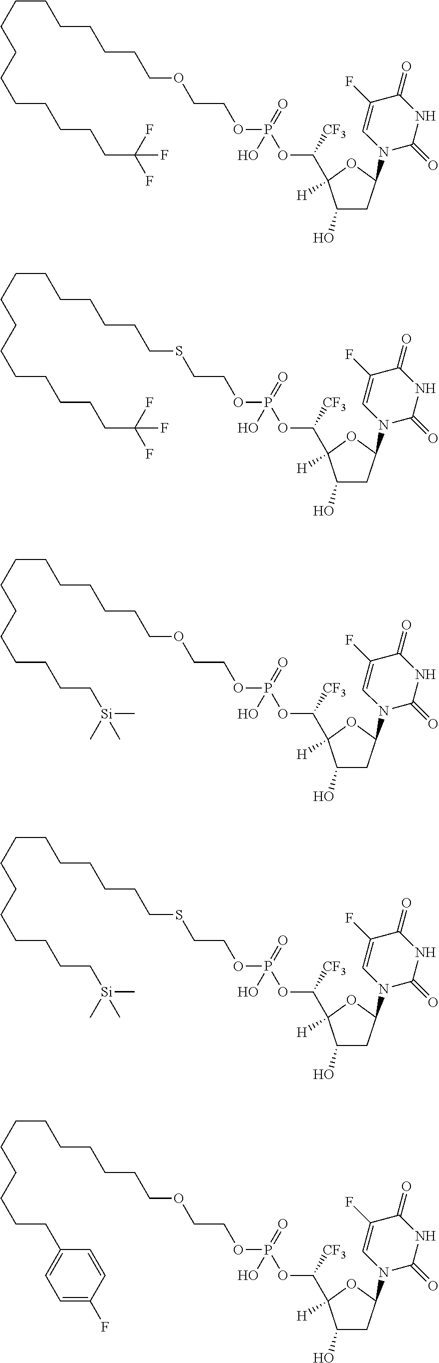 [SMILES: O=c1[nH]c(=O)n([C@H]2C[C@H](O)[C@@H]([C@H](OP(=O)(O)OCCOCCCCCCCCCCCCCCCC(F)(F)F)C(F)(F)F)O2)cc1F ; O=c1[nH]c(=O)n([C@H]2C[C@H](O)[C@@H]([C@H](OP(=O)(O)OCCSCCCCCCCCCCCCCCCC(F)(F)F)C(F)(F)F)O2)cc1F ; C[Si](C)(C)CCCCCCCCCCCCCCOCCOP(=O)(O)O[C@@H]([C@H]1O[C@@H](n2cc(F)c(=O)[nH]c2=O)C[C@@H]1O)C(F)(F)F ; C[Si](C)(C)CCCCCCCCCCCCCCSCCOP(=O)(O)O[C@@H]([C@H]1O[C@@H](n2cc(F)c(=O)[nH]c2=O)C[C@@H]1O)C(F)(F)F ; O=c1[nH]c(=O)n([C@H]2C[C@H](O)[C@@H]([C@H](OP(=O)(O)OCCOCCCCCCCCCCCCc3ccc(F)cc3)C(F)(F)F)O2)cc1F]

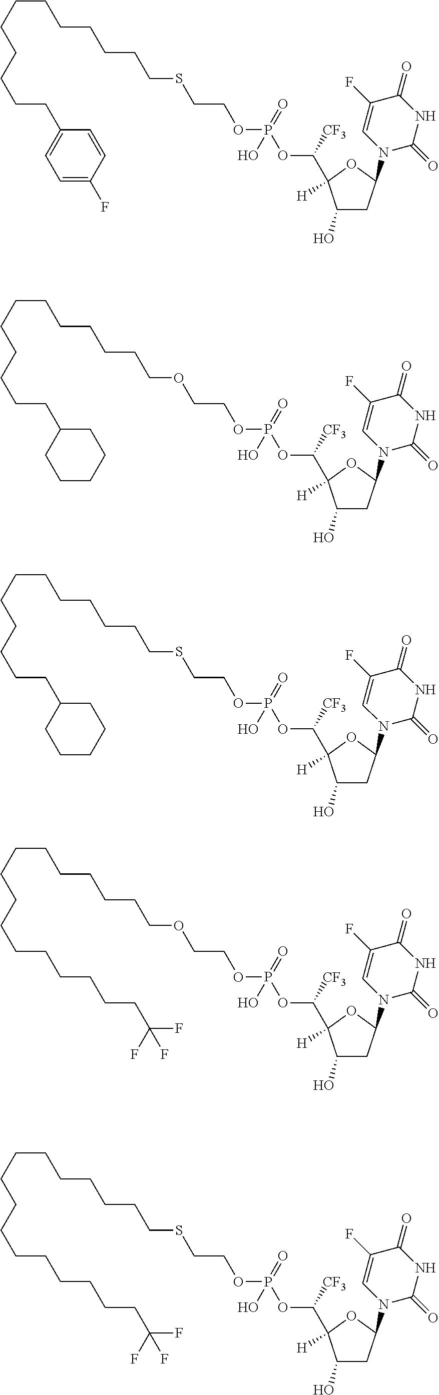 [SMILES: O=c1[nH]c(=O)n([C@H]2C[C@H](O)[C@@H]([C@H](OP(=O)(O)OCCSCCCCCCCCCCCCc3ccc(F)cc3)C(F)(F)F)O2)cc1F ; O=c1[nH]c(=O)n([C@H]2C[C@H](O)[C@@H]([C@H](OP(=O)(O)OCCOCCCCCCCCCCCCC3CCCCC3)C(F)(F)F)O2)cc1F ; O=c1[nH]c(=O)n([C@H]2C[C@H](O)[C@@H]([C@H](OP(=O)(O)OCCSCCCCCCCCCCCCC3CCCCC3)C(F)(F)F)O2)cc1F ; O=c1[nH]c(=O)n([C@H]2C[C@H](O)[C@@H]([C@H](OP(=O)(O)OCCOCCCCCCCCCCCCCCCCC(F)(F)F)C(F)(F)F)O2)cc1F ; O=c1[nH]c(=O)n([C@H]2C[C@H](O)[C@@H]([C@H](OP(=O)(O)OCCSCCCCCCCCCCCCCCCCC(F)(F)F)C(F)(F)F)O2)cc1F]

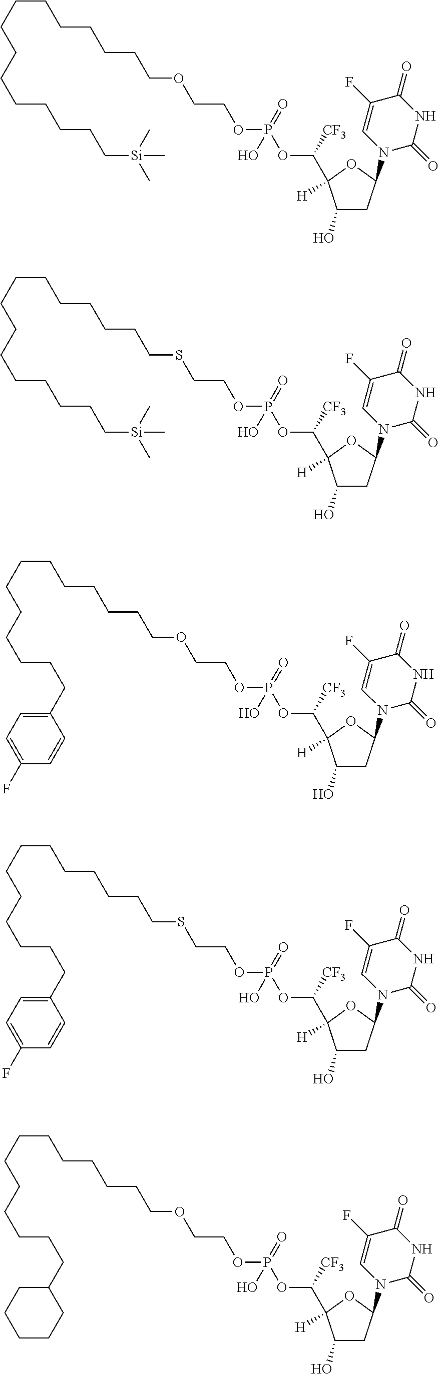 [SMILES: C[Si](C)(C)CCCCCCCCCCCCCCCOCCOP(=O)(O)O[C@@H]([C@H]1O[C@@H](n2cc(F)c(=O)[nH]c2=O)C[C@@H]1O)C(F)(F)F ; C[Si](C)(C)CCCCCCCCCCCCCCCSCCOP(=O)(O)O[C@@H]([C@H]1O[C@@H](n2cc(F)c(=O)[nH]c2=O)C[C@@H]1O)C(F)(F)F ; O=c1[nH]c(=O)n([C@H]2C[C@H](O)[C@@H]([C@H](OP(=O)(O)OCCOCCCCCCCCCCCCCc3ccc(F)cc3)C(F)(F)F)O2)cc1F ; O=c1[nH]c(=O)n([C@H]2C[C@H](O)[C@@H]([C@H](OP(=O)(O)OCCSCCCCCCCCCCCCCc3ccc(F)cc3)C(F)(F)F)O2)cc1F ; O=c1[nH]c(=O)n([C@H]2C[C@H](O)[C@@H]([C@H](OP(=O)(O)OCCOCCCCCCCCCCCCCC3CCCCC3)C(F)(F)F)O2)cc1F]

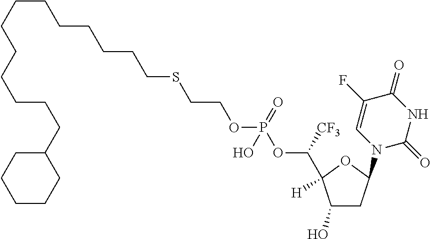 [SMILES: O=c1[nH]c(=O)n([C@H]2C[C@H](O)[C@@H]([C@H](OP(=O)(O)OCCSCCCCCCCCCCCCCC3CCCCC3)C(F)(F)F)O2)cc1F]

3. Additional Prodrugs

In some examples, the prodrugs have a structure of Formulas II, IIa, IIa′, IIb, IIb′, IIIa, IIIa′, IIIb or IIIb′, or a pharmaceutically acceptable salt thereof, wherein Y and Z are independently selected from —O—R₉ and —S—R₁₀, and wherein the other groups in the foregoing formulas are the same as described above.

In some examples, both Y and Z are independently —O—R₉. In some examples, Y and Z are the same. In some examples, Y and Z are different.

In some examples, Y is —O—R₉ and Z is —S—R₁₀.

In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂, wherein Rᵣ₁ and Rᵣ₂ are the same as described above.

In some examples, R₉ is optionally substituted aryl, such as phenyl or naphthyl. In some examples, R₉ is phenyl. In some examples, R₉ is naphthyl. In some examples, R₉ is optionally substituted alkyl, such as benzyl, isopropyl, and 2-ethylbutyl.

In some examples, R₉ is and —Rᵣ₁—Rᵣ₂. Exemplary —Rᵣ₁—Rᵣ₂ includes, but is not limited to, —CH₂—OC(═O)ORᵢ, —CH₂CH₂CH₂—NHC(═O)ORₕ, —CH₂CH₂CH₂—S—S—R_{z}, —CH₂CH₂CH₂CH₂—S—S—R_{z}, —CH₂CH₂CH(CH₃)—S—S—R_{z}, —CH₂CH₂CH₂CH(CH₃)—S—S—R_{z}, —CH₂CH₂CH₂—NH[C(═O)Rₕ],

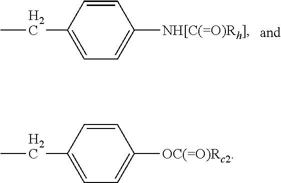

In some examples, R₁₀ is optionally substituted aryl, such as phenyl or naphthyl. In some examples, R₁₀ is phenyl. In some examples, R₁₀ is naphthyl. In some examples, R₁₀ is optionally substituted alkyl, such as benzyl, isopropyl, and 2-ethylbutyl.

In some examples, both Y and Z are the same —O—R₉, wherein R₉ is optionally substituted aryl (such as phenyl and naphthyl), optionally substituted alkyl (such as benzyl, isopropyl, and 2-ethylbutyl), or —Rᵣ₁—Rᵣ₂.

### Exemplary Structures

Exemplary prodrugs have a structure of Formulas II, IIa, IIa′, IIb, IIb′, IIIa, IIIa′, IIIb or IIIb′, or a pharmaceutically acceptable salt thereof, wherein Y is —O—R₉ or —S—R₁₀ and Z is —O—Rᵣ₁—Rᵣ₂, and wherein the other groups in the foregoing formulas are the same as described above.

In some examples, Y is —O—R₉. In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂.

In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₉ is hydrogen. In some examples, R₉ is optionally substituted alkyl, such as benzyl, isopropyl, and 2-ethylbutyl. In some examples, R₉ is optionally substituted aryl, such as phenyl and naphthyl.

In some examples, Y is —S—R₁₀. In some examples, R₁₀ is optionally substituted alkyl, such as benzyl, isopropyl, and 2-ethylbutyl. In some examples, R₁₀ is optionally substituted aryl, such as phenyl and naphthyl.

In some examples, Rᵣ₁ is independently optionally substituted C₁-C₄ bridging alkylene, such as optionally substituted, linear, C₁-C₄ bridging alkylene, e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—. In some examples, Rᵣ₁ is —CH₂CH₂— or —CH₂CH₂CH₂CH₂—.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5. In some examples, Q is —C₆H₄—, such as

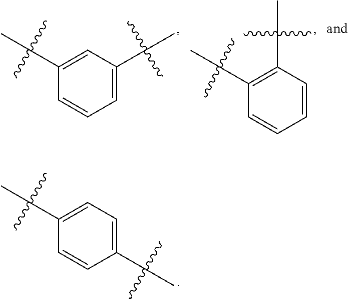 [SMILES: *c1cccc(*)c1 ; *c1ccccc1* ; *c1ccc(*)cc1]

In some examples, Q is —CH₂—C₆H₄—, such as,

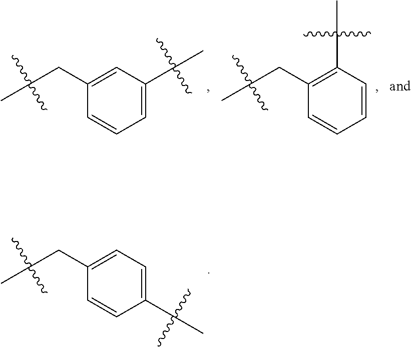 [SMILES: *Cc1cccc(*)c1 ; *Cc1ccccc1* ; *Cc1ccc(*)cc1]

Rᵣ₂ is selected from ester, thioester, amide, acylamino, carbonate ester, carbomate, disulfide, optionally substituted (4-acylamino)phenyl, and optionally substituted (4-acyloxy)phenyl. In some examples, Rᵣ₂ is selected from —C(═O)OR_{c1}, —OC(═O)R_{c2}, —C(═O)SRₒ₁, —SC(═O)Rₒ₂, —C(═O)NR_{f1}R_{f2}, —NR_{g}[C(═O)Rₕ], —OC(═O)ORᵢ, —OC(═O)NRⱼ₁Rⱼ₂, —NRₖ[(C═O)ORᵢ], —S—S—R_{z}, optionally substituted (4-acylamino)phenyl, and optionally substituted (4-acyloxy)phenyl, wherein R_{c1}, R_{c2}, Rₙ, Rₐ, R_{g}, Rₕ, Rᵢ, Rⱼ₁, Rⱼ₂, Rₖ, Rₗ, Rₒ₁, Rₒ₂, and R_{z} are the same as described above. In some examples, R_{c1}, R_{c2}, R_{f1}, R_{f2}, Rₕ, Rᵢ, Rⱼ₁, Rⱼ₂, Rₖ, Rₗ, Rₒ₁, Rₒ₂, and R_{z} are independently selected from optionally substituted aryl (such as phenyl or naphthyl) and optionally substituted alkyl (such as benzyl, isopropyl, and 2-ethylbutyl); in this case, “optionally substituted” refers to optionally substituted by one or more R groups described herein. In some examples, R_{c1}, R_{c2}, Rₙ, Rₐ, Rₕ, Rᵢ, Rⱼ₁, Rⱼ₂, Rₖ, Rₗ, Rₒ₁, Rₒ₂, and R_{z} are independently phenyl or naphthyl. In some examples, R_{c1}, R_{c2}, Rₙ, Rₐ, Rₕ, Rᵢ, Rⱼ₁, Rⱼ₂, Rₖ, Rₗ, Rₒ₁, Rₒ₂, and R_{z} are independently benzyl, isopropyl, or 2-ethylbutyl. In some examples, R_{g} and Rₖ are independently hydrogen or optionally substituted alkyl such as methyl; in this case, “optionally substituted” refers to optionally substituted by one or more R groups described herein. In some examples, R_{g} and Rₖ are hydrogen. In some examples, R_{g} and Rₖ are methyl. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or a C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₁₁-C₂₀ alkyl or a linear C₅-C₂₂ alkyl.

In some examples, Rᵣ₂ is —S—S—R_{z}. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or a C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₁₁-C₂₀ alkyl or a linear C₅-C₂₂ alkyl.

In some examples, Rᵣ₂ is —NR_{g}[C(═O)Rₕ]; in some examples, R_{g} is hydrogen or methyl. In some examples, Rᵣ₂ is —OC(═O)ORᵢ. In some examples, Rᵣ₂ is optionally substituted (4-acylamino)phenyl or optionally substituted (4-acyloxy)phenyl; in some examples, Rᵣ₂ is (4-acylamino)phenyl and (4-acyloxy)phenyl.

Exemplary —Rᵣ₁—Rᵣ₂ includes, but is not limited to, —CHOC(═O)ORᵢ, —CH₂CH₂CH₂—NHC(═O)ORₕ, —CH₂CH₂CH₂—S—S—R_{z}, —CH₂CH₂CH₂CH₂—S—S—Rₕ, —CH₂CH₂CH(CH₃)—S—S—R_{z}, —CH₂CH₂CH₂CH(CH₃)—S—S—R_{z}, —CH₂CH₂CH₂—NH[C(═O)Rₕ],

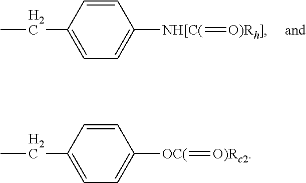

In some examples, Rᵣ₁ is optionally substituted C₁-C₄ bridging alkyene, and Rᵣ₂ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is optionally substituted, linear, C₁-C₄ bridging alkylene (e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—), and Rᵣ₂ is —S—S—R_{z}, wherein R_{z} is alkyl, optionally substituted by one or more R groups described herein. In some examples, Rᵣ₁ is —CH₂CH₂— or —CH₂CH₂CH₂CH₂—, and Rₐ is —S—S—R_{z}, wherein R_{z} is a linear C₁-C₂₀ alkyl or a linear C₂-C₂₂ alkyl.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5; and Rₐ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is -Q-CH₂—, wherein Q is —C₆H₄— (such as

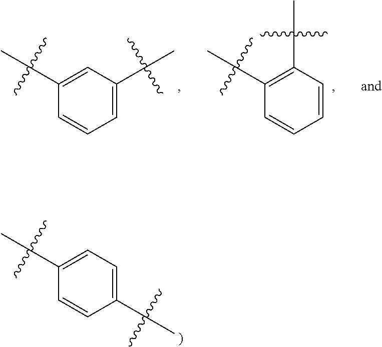 [SMILES: *c1cccc(*)c1 ; *c1ccccc1* ; *c1ccc(*)cc1]

or —CH₂—C₆H₄— (such as

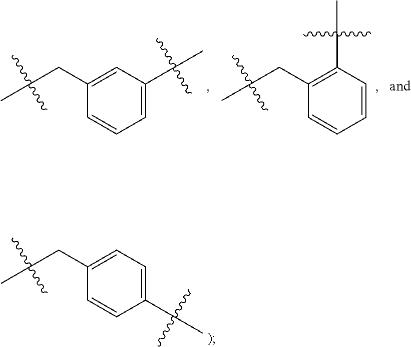 [SMILES: *Cc1cccc(*)c1 ; *Cc1ccccc1* ; *Cc1ccc(*)cc1]

and Rᵣ₂ is —S—S—R_{z}, wherein R_{z} is a linear C₁-C₂₀ alkyl or a linear C₂-C₂₂ alkyl.

When Y is also —O—Rᵣ₁—Rᵣ₂, Y may be the same as or different from Z. In other words, each occurrence of Rᵣ₁ or Rₐ is independent.

C. Prodrugs of Other Nucleoside Monophosphates

Also disclosed are prodrugs of nucleoside monophophases that are unsubstituted at the 5′ position. These prodrugs can have a structure of Formula IV or Formula V, or a pharmaceutically acceptable salt thereof:

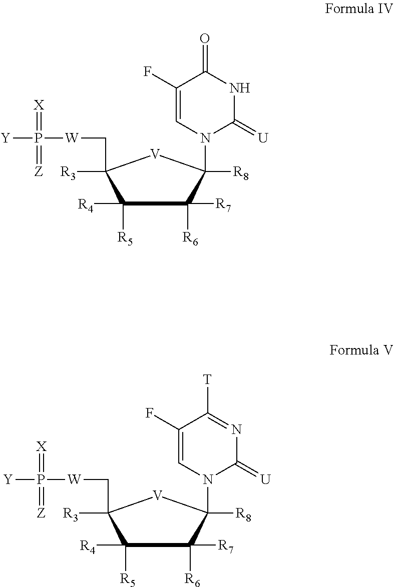

wherein R₁, R₂, R₃, R₄, R₅, R₆, R₇, R₈, U, V, W, and X are described in the sections above for Formulas I, II, and III;
wherein Y is —O—R₉ or —S—R₁₀ and Z is —O—R_{q1}—R_{q2}—R_{q3}—R_{q4} or —O—Rᵣ₁—Rᵣ₂;
wherein T is —NR₁₅R₁₆ or —OR₁₇;
wherein R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, —R_{q1}—R_{q2}—R_{q3}—R_{q4}, and —Rᵣ₁—Rᵣ₂;
wherein R₁₀ is selected from optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₅ and R₁₆ are independently selected from hydrogen, acyl, ester, thioester, and amide;
wherein R₁₇ is acyl, ester, thioester, or amide;
wherein:
   R_{q1} is absent or a C₁-C₉ alkyl chain (i.e., C₁-C₉ bridging alkylene),
   R_{q2} is absent or is selected from substituted methylene or ethylene, —O—, —S—, —S(═O)—, —S—S—, and —S(O)₂—,
   R_{q3} is a C₂-C₂₀ alkyl chain (i.e., C₂-C₂₀ bridging alkylene), and
   R_{q4} is selected from hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅; and
wherein:
   Rᵣ₁ is optionally substituted bridging C₁-C₄ alkylene or -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5, and
   Rᵣ₂ is selected from ester, thioester, amide, acylamino, carbonate ester, carbomate, disulfide, optionally substituted (4-acylamino)phenyl, and optionally substituted (4-acyloxy)phenyl.

In some examples, the prodrugs have a structure of Formula IVa, Formula IVb, Formula Va, Formula Vb, or a pharmaceutically acceptable salt thereof,

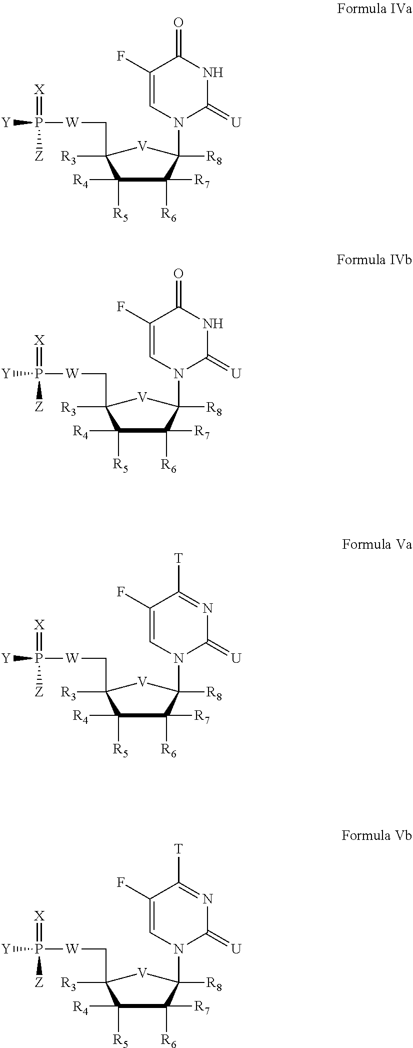

In some examples, the prodrugs have a structure of Formula IVa′, Formula IVb′, Formula Va′, Formula Vb′, or a pharmaceutically acceptable salt thereof,

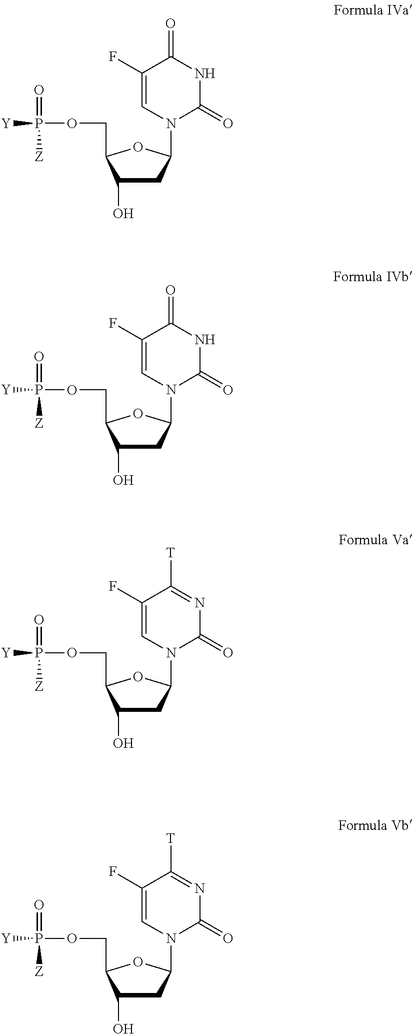

In some examples, the prodrugs have a structure of Formula IVa″, Formula IVb″ Formula Va″, Formula Vb″, or a pharmaceutically acceptable salt thereof,

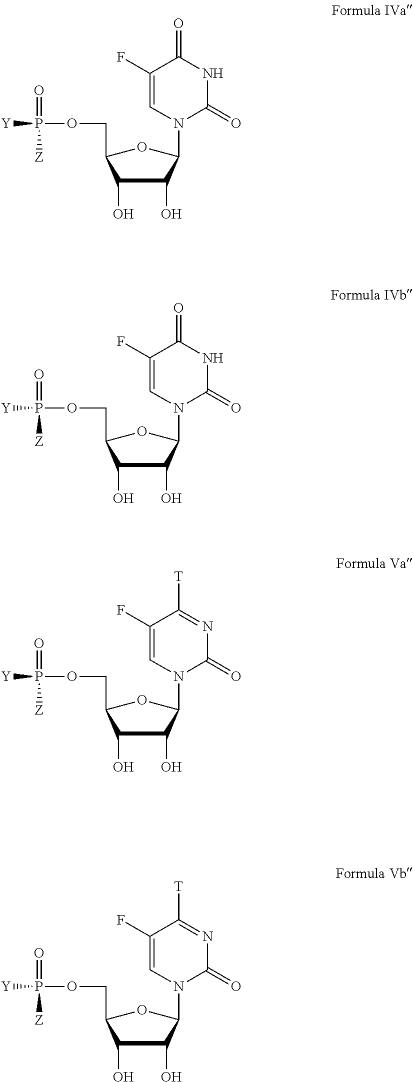

In some examples, the prodrugs have a structure of Formula IV, such as Formulas IVa, IVb, IVa′, IVb′, IVa″, and IVb″.

In some examples, the prodrugs have a structure of Formula V, such as Formulas Va, Vb, Va′, Vb′, Va″, and Vb″. In some examples, T is —NR₁₅R₁₆. In some examples, R₁₅ and R₁₆ are independently selected from hydrogen, —C(═O)Rₑ, —C(═O)OR_{c1}, —C(═O)SRₒ₁, and —C(═O)NR_{f1}R_{f2}, wherein R_{c1}, Rₑ, R_{f1}, R_{f2}, and Rₒ₁ are the same as described above. In some examples, R₁₅ is hydrogen and R₁₆ is an ester, e.g., —C(═O)OR_{c1}. In some examples, R₁₅ is hydrogen and R₁₆ is —C(═O)OR_{c1}, wherein R_{c1} is optionally substituted C₁-C₁₂ alkyl or optionally substituted C₁-C₁₀ alkyl. In some examples, R₁₅ is hydrogen and R₁₆ is —C(═O)OR_{c1}, wherein R_{c1} is C₂-C₉ alkyl or C₂-C₈ alkyl. In some examples, R₁₅ is hydrogen and R₁₆ is —C(═O)OCH₂CH₂CH₂CH₃ or —C(═O)OCH₂CH₂CH₂CH₂CH₃. In some examples, both R₁₅ and R₁₆ are hydrogen. In some examples, T is —OR₁₇. In some examples, R₁₇ is selected from —C(═O)Rₑ, —C(═O)OR_{c1}, —C(═O)SRₒ₁, and —C(═O)NR_{f1}R_{f2}, wherein R_{c1}, Rₑ, R_{f1}, R_{f2}, and Rₒ₁ are the same as described above.

In some examples, wherein Z is —O—R_{q1}—R₂—R_{q3}—R_{q4}. In some examples, Z is —O—Rᵣ₁—Rᵣ₂.

In some examples, Rᵣ₁ is optionally substituted C₁-C₄ bridging alkylene, such as optionally substituted, linear, C₁-C₄ bridging alkylene, e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—. In some examples, Rᵣ₁ is —CH₂CH₂— or —CH₂CH₂CH₂CH₂—.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5. In some examples, Q is —C₆H₄—, such as

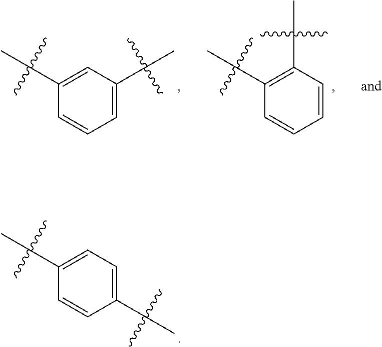 [SMILES: *c1cccc(*)c1 ; *c1ccccc1* ; *c1ccc(*)cc1]

In some examples, Q is —CH₂—C₆H₄—, such as

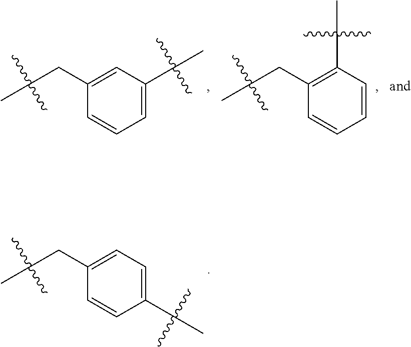 [SMILES: *Cc1cccc(*)c1 ; *Cc1ccccc1* ; *Cc1ccc(*)cc1]

Rᵣ₂ is selected from ester, thioester, amide, acylamino, carbonate ester, carbomate, disulfide, optionally substituted (4-acylamino)phenyl, and optionally substituted (4-acyloxy)phenyl. In some examples, Rᵣ₂ is selected from —C(═O)OR_{c1}, —OC(═O)Rₑ2, —C(═O)SRₒ₁, —SC(═O)Rₒ₂, —C(═O)NR_{f1}R_{f2}, —NR_{g}[C(═O)Rₕ], —OC(═O)ORᵢ, —OC(═O)NRⱼ₁Rⱼ₂, —NRₖ[(C═O)ORᵢ], —S—S—R_{z}, optionally substituted (4-acylamino)phenyl, and optionally substituted (4-acyloxy)phenyl, wherein R_{c1}, R_{c2}, R_{f1}, R_{f2}, R_{g}, Rₕ, Rₗ, Rⱼ₁, Rⱼ₂, Rₖ, Rₗ, Rₒ₁, Rₒ₂, and R_{z} are the same as described above. In some examples, Rei, Rₑ2, R_{f1}, R_{f2}, Rₕ, Rᵢ, Rⱼ₁, Rⱼ₂, Rₖ, Rₗ, Rₒ₁, Rₒ₂, and R_{z} are independently selected from optionally substituted aryl (such as phenyl or naphthyl) and optionally substituted alkyl (such as benzyl, isopropyl, and 2-ethylbutyl); in this case, “optionally substituted” refers to optionally substituted by one or more R groups described herein. In some examples, R_{c1}, R_{c2}, Rₙ, Rₐ, Rₕ, Rᵢ, Rⱼ₁, Rⱼ₂, Rₖ, Rₗ, Rₒ₁, Rₒ₂, and R_{z} are independently phenyl or naphthyl. In some examples, R_{c1}, R_{c2}, Rₙ, Rₐ, Rₕ, Rᵢ, Rⱼ₁, Rⱼ₂, Rₖ, Rₗ, Rₒ₁, Rₒ₂, and R_{z} are independently benzyl, isopropyl, or 2-ethylbutyl. In some examples, R_{g} and Rₖ are independently hydrogen or optionally substituted alkyl such as methyl; in this case, “optionally substituted” refers to optionally substituted by one or more R groups described herein. In some examples, R_{g} and Rₖ are hydrogen. In some examples, R_{g} and Rₖ are methyl. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₂-C₂₂ alkyl or a C₁-C₂₀ alkyl. In some examples, R_{z} is a linear C₂-C₂₂ alkyl or a linear C₁-C₂₀ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₈-C₂₂ alkyl or a linear C₁₁-C₂₀ alkyl.

In some examples, Rᵣ₂ is —S—S—R_{z}. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or a C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₈-C₂₂ alkyl or a linear C₁₁-C₂₀ alkyl.

In some examples, Rᵣ₂ is —NR_{g}[C(═O)Rₕ]; in some examples, R_{g} is hydrogen or methyl. In some examples, Rᵣ₂ is —OC(═O)ORᵢ. In some examples, Rᵣ₂ is optionally substituted (4-acylamino)phenyl or optionally substituted (4-acyloxy)phenyl; in some examples, Rᵣ₂ is (4-acylamino)phenyl and (4-acyloxy)phenyl.

Exemplary —Rᵣ₁—Rᵣ₂ includes, but is not limited to, —CH₂—OC(═O)ORᵢ, —CH₂CH₂CH₂—NHC(═O)ORₕ, —CH₂CH₂CH₂—S—S—R_{z}, —CH₂CH₂CH₂CH₂—S—S—R_{z}, —CH₂CH₂CH(CH₃)—S—S—R_{z}, —CH₂CH₂CH₂CH(CH₃)—S—S—R_{z}, —CH₂CH₂CH₂—NH[C(═O)Rₕ],

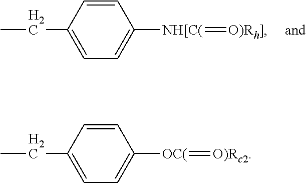

In some examples, Rᵣ₁ is optionally substituted C₁-C₄ bridging alkylene, and Rₐ is —S—S—R_{z}, wherein R_{z} is alkyl, alkenyl, alkynyl, carbocyclyl, heterocyclyl, aryl, or heteroaryl, optionally substituted by one or more R groups described herein. In some examples, R₁ is optionally substituted, linear, C₁-C₄ bridging alkylene (e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—), and Rₐ is —S—S—R_{z}, wherein R_{z} is alkyl, optionally substituted by one or more R groups described herein. In some examples, R₁ is —CH₂CH₂— or —CH₂CH₂CH₂CH₂—, and R₂ is —S—S—R_{z}, wherein R_{z} is a linear C₁-C₂₀ alkyl or a linear C₂-C₂₂ alkyl.

In some examples, R₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5; and Rₐ is —S—S—R_{z}, wherein R_{z} is alkyl, alkenyl, alkynyl, carbocyclyl, heterocyclyl, aryl, or heteroaryl, optionally substituted by one or more R groups described herein. In some examples, R₁ is -Q-CH₂—, wherein Q is —C₆H₄— (such as

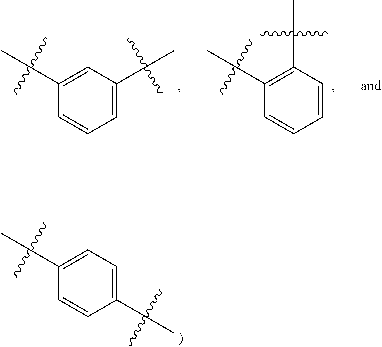 [SMILES: *c1cccc(*)c1 ; *c1ccccc1* ; *c1ccc(*)cc1]

or —CH₂—C₆H₄-(such as

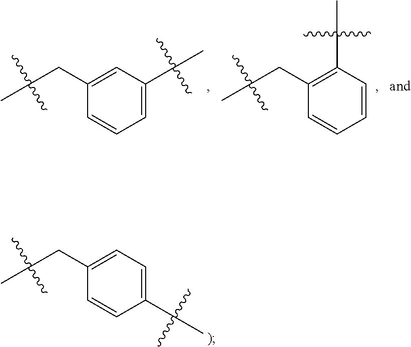 [SMILES: *Cc1cccc(*)c1 ; *Cc1ccccc1* ; *Cc1ccc(*)cc1]

and Rᵣ₂ is —S—S—R_{z}, wherein R_{z} is a linear C₁-C₂₀ alkyl or a linear C₂-C₂₂ alkyl.

Unless the context indicates otherwise, the substituted or optionally substituted groups described in Formulas IV, V, IVa, IVb, Va, Vb, IVa′, IVb′, Va′, Vb′, IVa″, IVb″, Va″, and Vb″ can have one or more substituents independently selected from deuterium, halogen, azido, cyano, isocyano, nitrate, nitrosooxy, nitroso, nitro, formyl, carboxyl, carbonate, alkyl, heteroalkyl, alkenyl, heteroalkenyl, alkynyl, heteroalkynyl, carbocyclyl, heterocyclyl, aryl, heteroaryl, azo, acyl, hydroxyl, thiol, sulfinyl, sulfonyl, sulfonate, sulfamoyl, amino, acylamino, amide, silyl, ester, thioester, carbonate ester, carbamate, aminooxy, hydroxyamino, and —SF₅, wherein each substituent may be further substituted by one or more R groups as described herein. In some examples, two substituents on the same atom can join together with that atom to form a cyclic moiety, such as a carbocycle or a heterocycle.

For example, the Si-substituted silyl in Formulas IV, V, IVa, IVb, Va, Vb, IVa′, IVb′, Va′, Vb′, IVa″, IVb″, Va″, and Vb″ can have one, two, or three substituents independently selected from those described above. When there are multiple substituents, two of the substituents can join together with the Si atom to form a heterocycle. In some examples, the substituent(s) is independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. In some examples, the Si-substituted silyl has three substituents, independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. Exemplary Si-substituted silyl groups include, but are not limited to, the following:

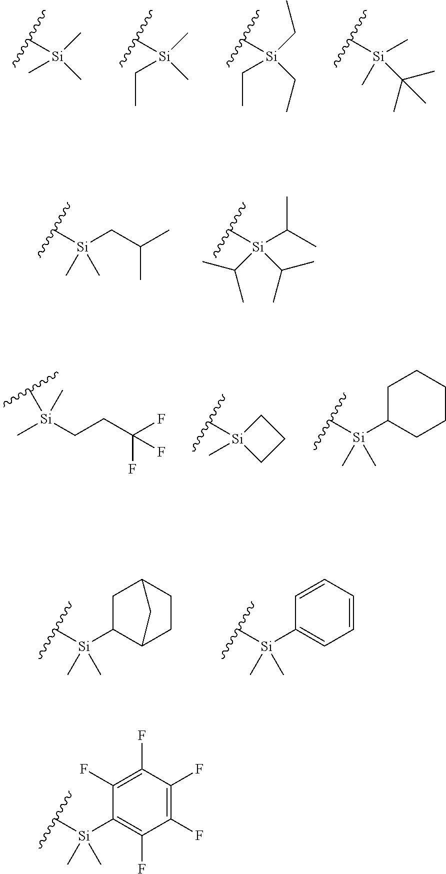

In some examples, the substituted or optionally substituted groups described in Formulas IV, V, IVa, IVb, Va, Vb, IVa′, IVb′, Va′, Vb′, IVa″, IVb″, Va″, and Vb″ can have one or more substituents independently selected from alkyl, heteroalkyl, alkenyl, heteroalkenyl, alkynyl, heteroalkynyl, carbocyclyl, heterocyclyl, aryl, and heteroaryl, wherein each substitutent may be further substituted by one or more R groups. In some examples, two substituents on the same atom can join together with that atom to form a cyclic moiety, such as a carbocycle or a heterocycle.

In some examples, the substituted or optionally substituted groups described in Formulas IV, V, IVa, IVb, Va, Vb, IVa′, IVb′, Va′, Vb′, IVa″, IVb″, Va″, and Vb″ can have one or more substituents independently selected from deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(tert-butyl)silyl, methyl(methyl)(isobutyl)silyl), formyl, carboxyl, mercapto, sulfamoyl, alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), alkyl (such as methyl, ethyl, isopropyl, tert-butyl), heteroalkyl, alkenyl, heteroalkenyl, alkynyl, heteroalkynyl, carbocyclyl, heterocyclyl, aryl, heteroaryl, and —SF₅.

In some examples, the substituted groups or optionally substituted groups described in Formulas IV, V, IVa, IVb, Va, Vb, IVa′, IVb′, Va′, Vb′, IVa″, IVb″, Va″, and Vb″ can have one or more substituents independently selected from deuterium, halogen, nitro, cyano, hydroxyl, trifluoromethoxy, trifluoromethyl, trimethylsilyl, amino, formyl, carboxyl, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, carbocyclyl, aryl, and heterocyclyl.

1. Lipid-Derived Prodrugs

In some examples, the prodrugs are lipid-derived prodrugs. These prodrugs have a structure of Formulas IV, V, IVa, IVb, Va, Vb, IVa′, IVb′, Va′, Vb′, IVa″, IVb″, Va″, or Vb″, or a pharmaceutically acceptable salt thereof, wherein Y is —O—R₉ or —S—R₁₀ and Z is —O—R_{q1}—R_{q2}—R_{q3}—R_{q4}, and wherein the other groups in the foregoing formulas are the same as described above.

In some examples, Y is —O—R₉. In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂.

In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₉ is hydrogen. In some examples, R₉ is optionally substituted alkyl, such as benzyl, isopropyl, and 2-ethylbutyl. In some examples, R₉ is optionally substituted aryl, such as phenyl and naphthyl.

In some examples, Y is —S—R₁₀. In some examples, R₁₀ is optionally substituted alkyl, such as benzyl, isopropyl, and 2-ethylbutyl. In some examples, R₁₀ is optionally substituted aryl, such as phenyl and naphthyl.

In some examples, when R_{q2} is —O—, R_{q4} is not hydrogen, methyl, or ethyl.

In some examples, when both R_{q1} and R_{q2} are absent, R_{q4} is not hydrogen, methyl, or ethyl.

In some examples, when R_{q1} is absent, R_{q2} is also absent.

In some examples, both R_{q1} and R_{q2} are present, i.e., Z is —O—R_{q1}—R_{q2}—R_{q3}—R_{q4}.

In some examples, both R_{q1} and R_{q2} are absent, i.e., Z is —O—R_{q3}—R_{q4}.

In some examples, R_{q1} is present, and R_{q2} is absent, i.e., Z is —O—R_{q1}—R_{q3}—R_{q4}.

R_{q1} is absent or a C₁-C₉ alkyl chain (i.e., C₁-C₉ bridging alkylene). In some examples, R_{q1} is absent. In some examples, R_{q1} is a C₁-C₉ alkyl chain (i.e., C₁-C₉ bridging alkylene). In some examples, R_{q1} is a linear C₁-C₉ alkyl chain (i.e., linear C₁-C₉ bridging alkylene), such as methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), and nonylene (—CH₂CH₂CH₂CH₂CH₂CH₂CH₂CH₂CH₂—).

In some examples, R_{q1} is ethylene (—CH₂CH₂—), propylene (—CH₂CH₂CH₂—), or nonylene (—CH₂CH₂CH₂CH₂CH₂CH₂CH₂CH₂CH₂—). In some examples, R_{q1} is ethylene (—CH₂CH₂—) or propylene (—CH₂CH₂CH₂—).

R_{q2} is absent or is selected from substituted methylene or ethylene, —O—, —S—, —S(═O)—, —S—S—, and —S(O)₂—. In some examples, R₂ is absent. In some examples, R_{q2} is selected from substituted methylene or ethylene, —O—, —S—, —S(═O)—, —S—S—, and —S(O)₂—. In some examples, the substituted methylene or ethylene contains one or more halogen substituents. In some examples, the one or more halogen substituents are fluorine.

In some examples, R_{q2} is substituted methylene, such as —CF₂—. In some examples, R_{q2} is —O—. In some examples, R_{q2} is —S—. In some examples, R_{q2} is —S—S—.

R_{q3} is a C₂-C₂₀ alkyl chain (i.e., C₂-C₂₀ bridging alkylene). In some examples, R_{q3} is a linear C₂-C₂₀ alkyl chain (i.e., linear C₂-C₂₀ bridging alkylene), such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉-], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—].

In some examples, R_{q3} is a linear C₂-C₇ alkyl chain (i.e., linear bridging C₂-C₇ alkylene). In some examples, R₃ is a linear C₈-C₂₀ alkyl chain (i.e., linear C₈-C₂₀ bridging alkylene).

R_{q4} is selected from hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅.

Examples of R_{q4} include, but are not limited to, hydrogen, —CD₃, —CF₃, —CD₂CD₃, —CF₂CF₃, —S-Ph, —O-Ph, —C≡CH, —C≡CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

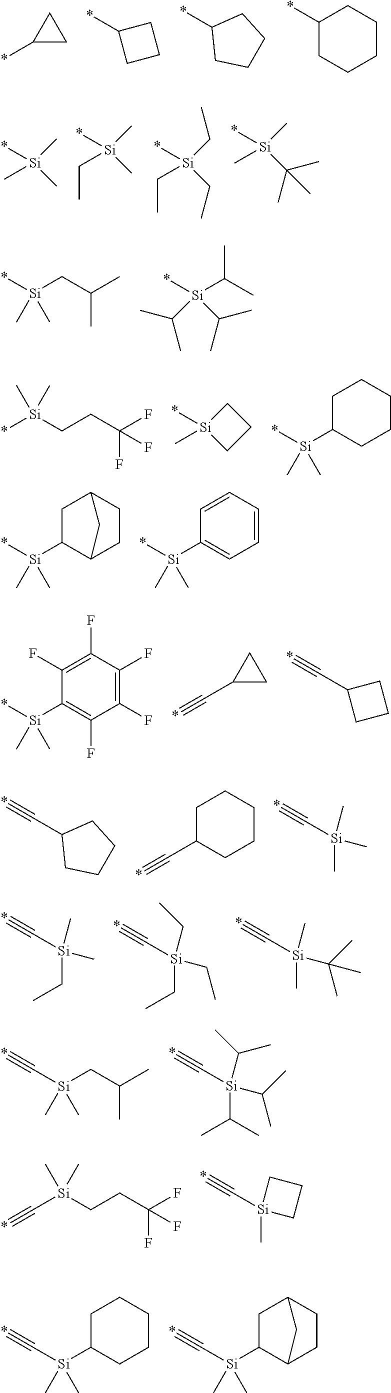

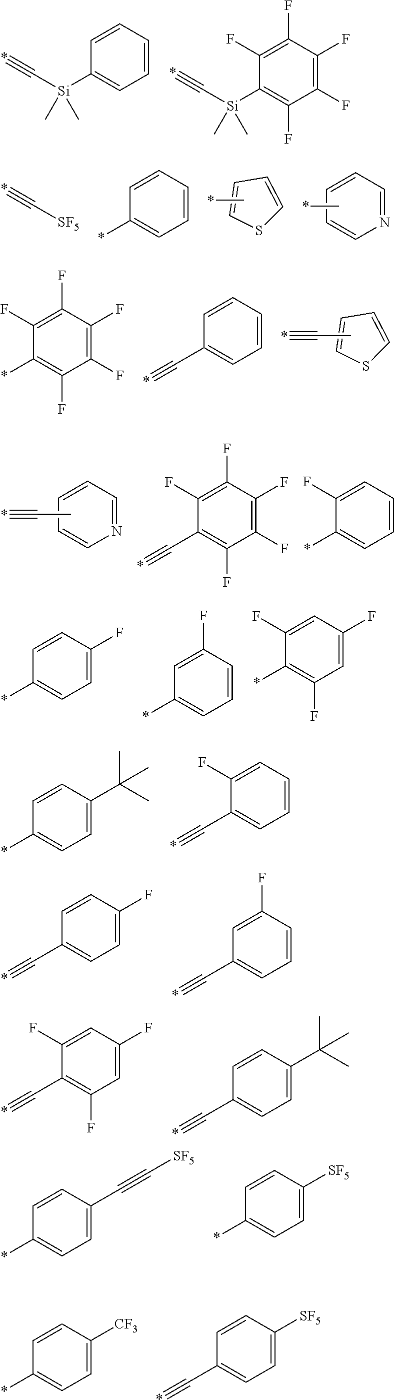

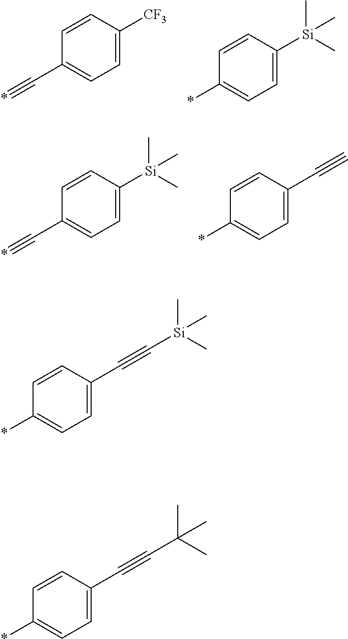

wherein * indicates the point of attachment to R_{q3}.

In some examples, R_{q4} is hydrogen, methyl or ethyl. In some examples, R_{q4} is hydrogen.

In some examples, R_{q4} is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅.

In some examples, R_{q4} is substituted methyl or ethyl, having one or more substituents. In some examples, the one or more substituents are independently selected from deuterium, halogen, and alkyl. In some examples, R_{q4} is selected from —CD₃, —CF₃, —C(CH₃)₃, —CD₂CD₃, and —CF₂CF₃. In some examples, R_{q4} is —CF₃.

In some examples, R_{q4} is optionally substituted C₂-C₃ alkenyl or alkynyl, which may have one or more substituents. For example, R_{q4} can be optionally substituted C₂-C₃ alkynyl, such as optionally substituted ethynyl and optionally substituted propynyl (including optionally substituted 1-propynyl and optionally substituted 2-propynyl). In some examples, the one or more substitutions are independently selected from deuterium, halogen (such as fluorine), alkyl, heteroalkyl, alkenyl, heteroalkenyl, alkynyl, heteroalkynyl, carbocyclyl (such as cyclopropyl, cyclobutyl, cyclopentyl, and cyclohexyl), heterocyclyl, aryl (such as phenyl), heteroaryl (such as pyridinyl and thiophenyl), silyl, and —SF₅, wherein each substituent may be further substituted by one or more R groups as described herein. In some examples, R_{q4} is selected from —C≡CH, —C≡CCD₃, —C≡CCH₂F, —C≡CCHF₂, —C≡CCF₃, —C≡CSi(CH₃)₃, —C≡CC(CH₃)₃, —C≡CSF₅, as well as the following:

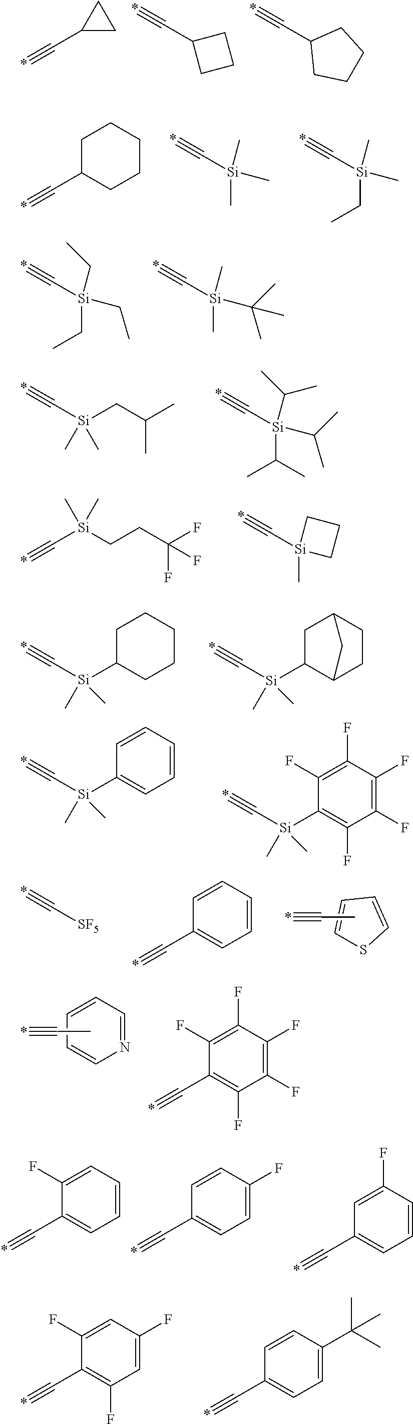

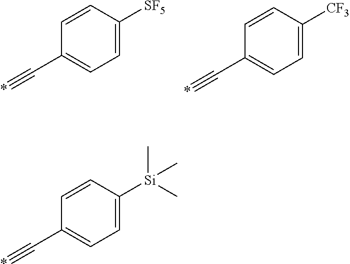

wherein * indicates the point of attachment to R_{q3}. In some examples, R_{q4} is —C≡CSi(CH₃)₃.

In some examples, R_{q4} is optionally substituted carbocyclyl, optionally substituted heterocyclyl, optionally substituted aryl, or optionally substituted heteroaryl. In some examples, the substituent(s) is independently selected from halogen, alkyl, heteroalkyl, silyl, and —SF₅, wherein each substituent may be further substituted by one or more R groups as described herein. Exemplary substituents of R_{q4} include, but are not limited to, fluorine, trifluoromethyl, ethynyl, 2-pentafluorosulfanylethynyl, 2-trimethylsilylethynyl, 2-(tert-butyl)ethynyl, tert-butyl, trimethylsilyl, and —SF₅. In some examples, R_{q4} is optionally substituted carbocyclyl, such as optionally substituted cyclopropyl, optionally substituted cyclobutyl, optionally substituted cyclopentyl, and optionally substituted cyclohexyl. In some examples, R_{q4} is optionally substituted heterocyclyl. In some examples, R_{q4} is optionally substituted aryl, such as optionally substituted phenyl (e.g., phenyl, 2-flurophenyl, 3-flurophenyl, 4-fluorophenyl, 2,4,6-triflurophenyl, 2,3,4,5,6-pentaflurophenyl, 4-(tert-butyl)phenyl, 4-(pentafluorosulfanyl)phenyl, 4-(trifluoromethyl)phenyl, 4-ethynylphenyl, 4-(2-pentafluorosulfanylethynyl)phenyl, 4-(2-trimethylsilylethynyl)phenyl, and 4-(2-(tert-butyl)ethynyl)phenyl. In some examples, R_{q4} is optionally substituted heteroaryl, such as optionally substituted pyridinyl and optionally substituted thiophenyl. In some examples, R_{q4} is selected from the following:

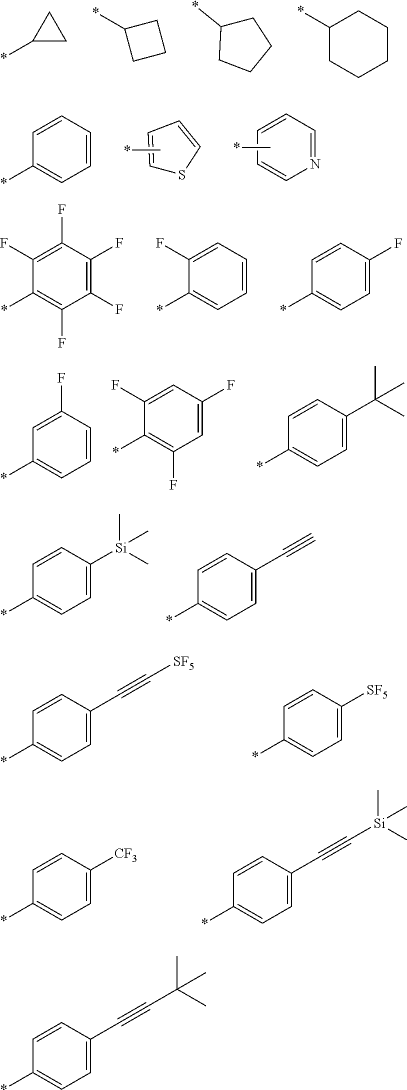

wherein * indicates the point of attachment to R_{q3}. In some examples, R_{q4} is cyclohexyl. In some examples, R_{q4} is 4-fluorophenyl.

In some examples, R_{q4} is Si-substituted silyl, having one or more substituents. When there are multiple substituents, two of the substituents can join together with the Si atom to form a cyclic moiety, such as a heterocycle. In some examples, the one or more substituents are independently selected from alkyl (such as methyl, ethyl, propyl, isopropyl, tert-butyl, and isobutyl), heteroalkyl, carbocyclyl (such as cyclohexyl and bicyclo[2.2.1]heptyl), heterocyclyl, aryl (such as phenyl), and heteroaryl, wherein each substituent may be further substituted by one or more R groups as described herein. In some examples, the Si-substituted silyl has three substituents, independently selected from alkyl, heteroalkyl, alkenyl, heteroalkenyl, alkynyl, heteroalkynyl, carbocyclyl, heterocyclyl, aryl, and heteroaryl, wherein each substituent may be further substituted by one or more R groups. In some examples, R_{q4} is selected from the following:

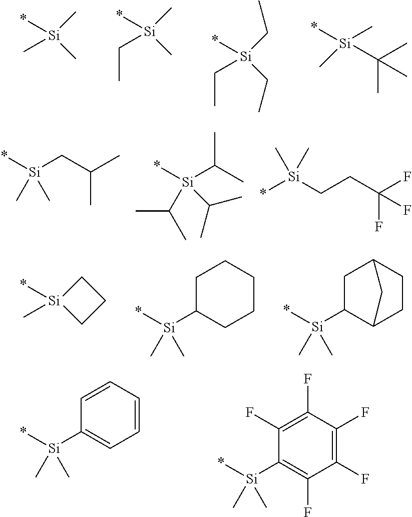

wherein * indicates the point of attachment to R_{q3}. In some examples, R_{q4} is trimethylsilyl.

In some examples, R_{q4} is S-substituted thiol, having one substituent. In some examples, the substituent is selected from alkyl, heteroalkyl, alkenyl, heteroalkenyl, alkynyl, heteroalkynyl, carbocyclyl, heterocyclyl, aryl (such as phenyl), and heteroaryl, which may be further substituted by one or more R groups as described herein. In some examples, the substituent is aryl, which may be further substituted by one or more R groups. In some examples, R_{q4} is —S-Ph.

In some examples, R_{q4} is O-substituted hydroxyl, having one substituent. In some examples, the substituent is selected from alkyl, heteroalkyl, alkenyl, heteroalkenyl, alkynyl, heteroalkynyl, carbocyclyl, heterocyclyl, aryl (such as phenyl), and heteroaryl, which may be further substituted by one or more R groups as described herein. In some examples, the substituent is aryl, which may be further substituted by one or more R groups. In some examples, R_{q4} is —O-Ph.

In some examples, R_{q4} is —SF₅.

In some examples, both R_{q1} and R_{q2} are present, i.e., Z is —O—R_{q1}—R_{q2}—R_{q3}—R_{q4}. In some examples, R_{q1} is a linear C₁-C₉ bridging alkylene (such as —CH₂CH₂— or —CH₂CH₂CH₂—); R_{q2} is —CF₂—, —O—, or —S—; R_{q3} is a linear C₂-C₂₀ bridging alkylene (such as a linear C₈-C₂₀ bridging alkylene); and R_{q4} is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅. In some examples, R_{q1} is —CH₂CH₂— or —CH₂CH₂CH₂—; R_{q2} is —CF₂—, —O—, or —S—; R_{q3} is a linear C₈-C₂₀ bridging alkylene; and R_{q4} is selected from —CD₃, —CF₃, —CD₂CD₃, —CF₂CF₃, —S-Ph, —O-Ph, —C≡CH, —C≡CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

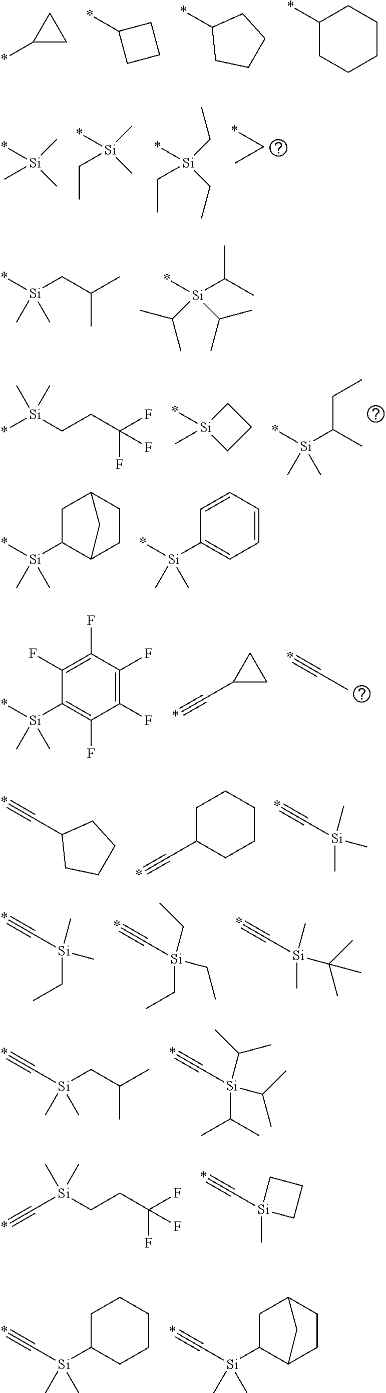

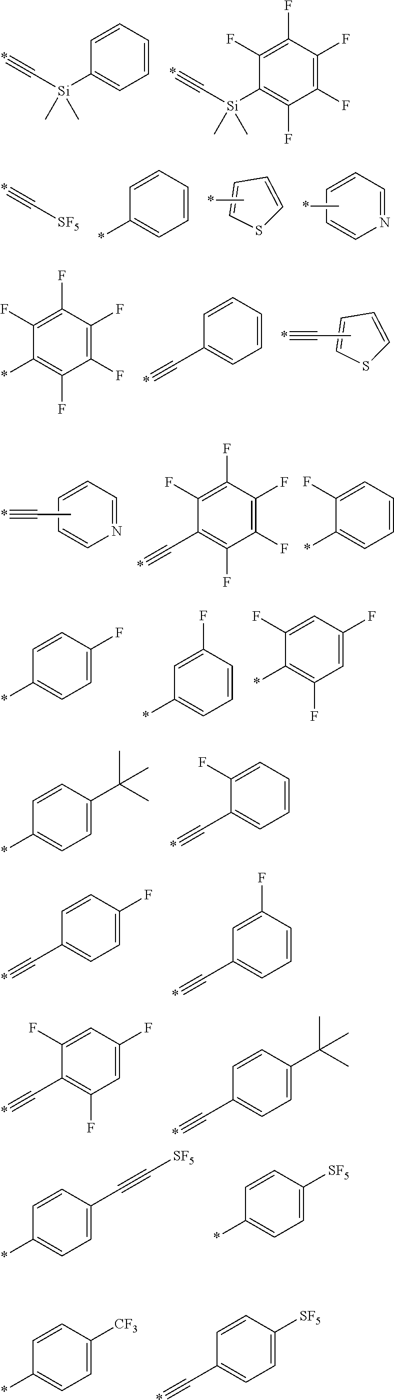

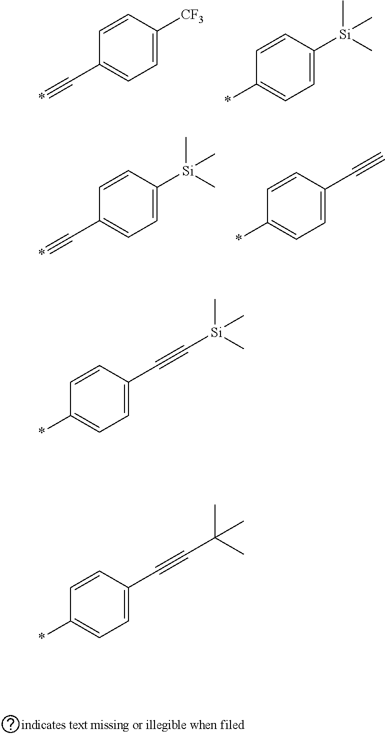

wherein * indicates the point of attachment to R_{q3}.

In some examples, both R_{q1} and R_{q2} are present, i.e., Z is —O—R_{q1}—R_{q2}—R_{q3}—R_{q4}, wherein R_{q1} is —CH₂CH₂— or —CH₂CH₂CH₂—, R_{q2} is —O— or —S—, R_{q3} is a linear C₈-C₂₀ bridging alkylene or a linear C₁₁-C₁₈ bridging alkylene, and R₄ is —CF₃,

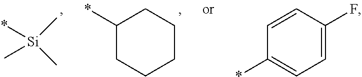

wherein * indicates the point of attachment to R_{q3}.

In some examples, both R_{q1} and R_{q2} are present, i.e., Z is —O—R_{q1}—R_{q2}—R_{q3}—R_{q4}, wherein R_{q1} is —CH₂CH₂—, R_{q2} is —O— or —S—, R_{q3} is a linear C₈-C₂₀ bridging alkylene or a linear C₁₃-C₁₇ bridging alkylene, and R_{q4} is —CF₃,

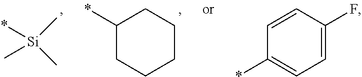

wherein * indicates the point of attachment to R_{q3}.

In some examples, both R_{q1} and R_{q2} are present, i.e., Z is —O—R_{q1}—R_{q2}—R_{q3}—R_{q4}, wherein R_{q1} is a linear C₁-C₉ bridging alkylene (such as —CH₂CH₂— or —CH₂CH₂CH₂—), R_{q2} is —CF₂—, —O—, —S—S— or —S—, R_{q3} is a linear C₂-C₂₀ bridging alkylene (such as a linear C₈-C₂₀ bridging alkylene or a linear C₂-C₇ bridging alkylene), and R_{q4} is hydrogen, methyl, or ethyl.

In some examples, both R_{q1} and R_{q2} are present, i.e., Z is —O—R_{q1}—R_{q2}—R_{q3}—R_{q4}, wherein R_{q1} is —CH₂CH₂— or —CH₂CH₂CH₂—, R_{q2} is —O— or —S—, R_{q3} is a linear C₈-C₂₀ bridging alkylene or a linear C₁₅-C₁₉ bridging alkylene, and R_{q4} is hydrogen.

In some examples, both R_{q1} and R_{q2} are absent, i.e., Z is —O—R_{q3}—R_{q4}. In some examples, R_{q3} is a linear C₂-C₂₀ bridging alkylene (such as a linear C₈-C₂₀ alkylene); and R_{q4} is selected from substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅. In some examples, R_{q3} is a linear C₈-C₂₀ bridging alkylene; and R_{q4} is selected from —CD₃, —CF₃, —CD₂CD₃, —CF₂CF₃, —S-Ph, —O-Ph, —C≡CH, —C≡CCD₃, —CH₂FC≡C, —CHF₂C≡C, —C≡CSi(CH₃)₃, —C≡CC(CH₃)₃, —C≡CCF₃, —C≡CSF₅, —Si(CH₃)₃, —C(CH₃)₃, —C(O)OCH₃, —SF₅, as well as the following:

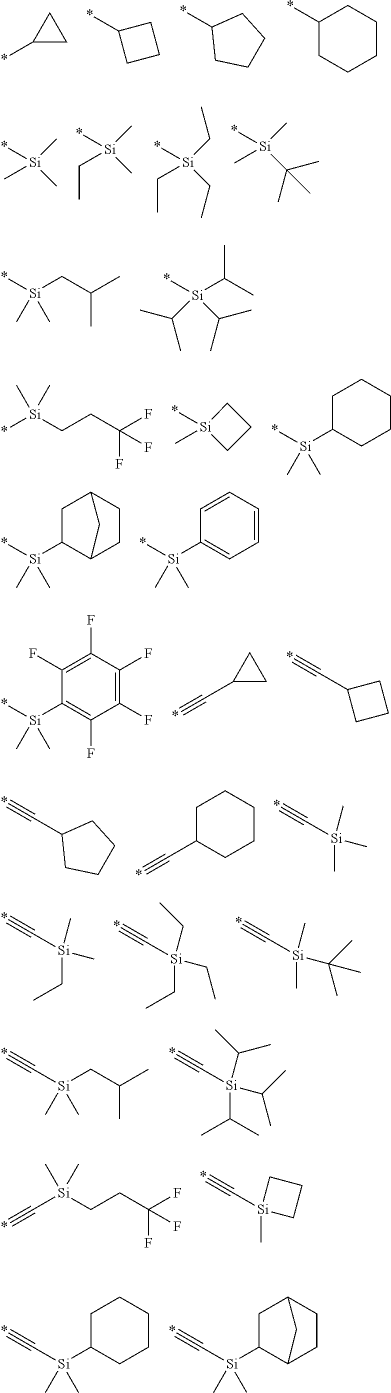

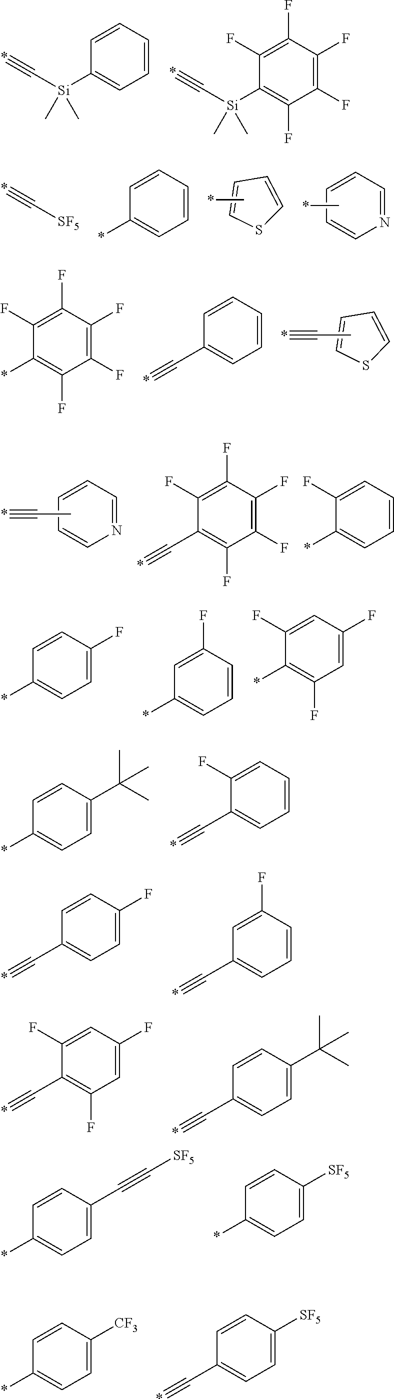

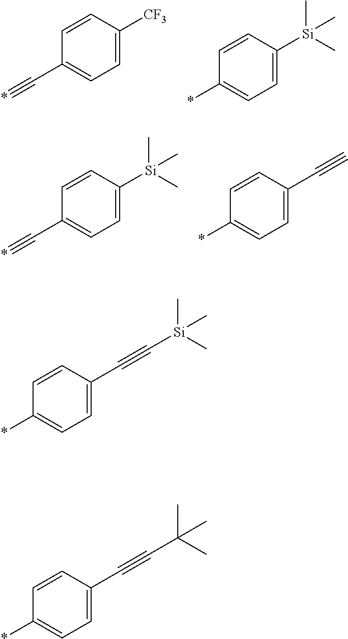

wherein * indicates the point of attachment to Y.

In some examples, both R_{q1} and R_{q2} are absent, i.e., Z is —O—R_{q3}—R_{q4}, wherein R_{q3} is a linear C₂-C₂₀ bridging alkylene (such as a linear C₈-C₂₀ alkylene) and R₄ is selected from hydrogen, methyl or ethyl.

In some examples, Z is selected from:

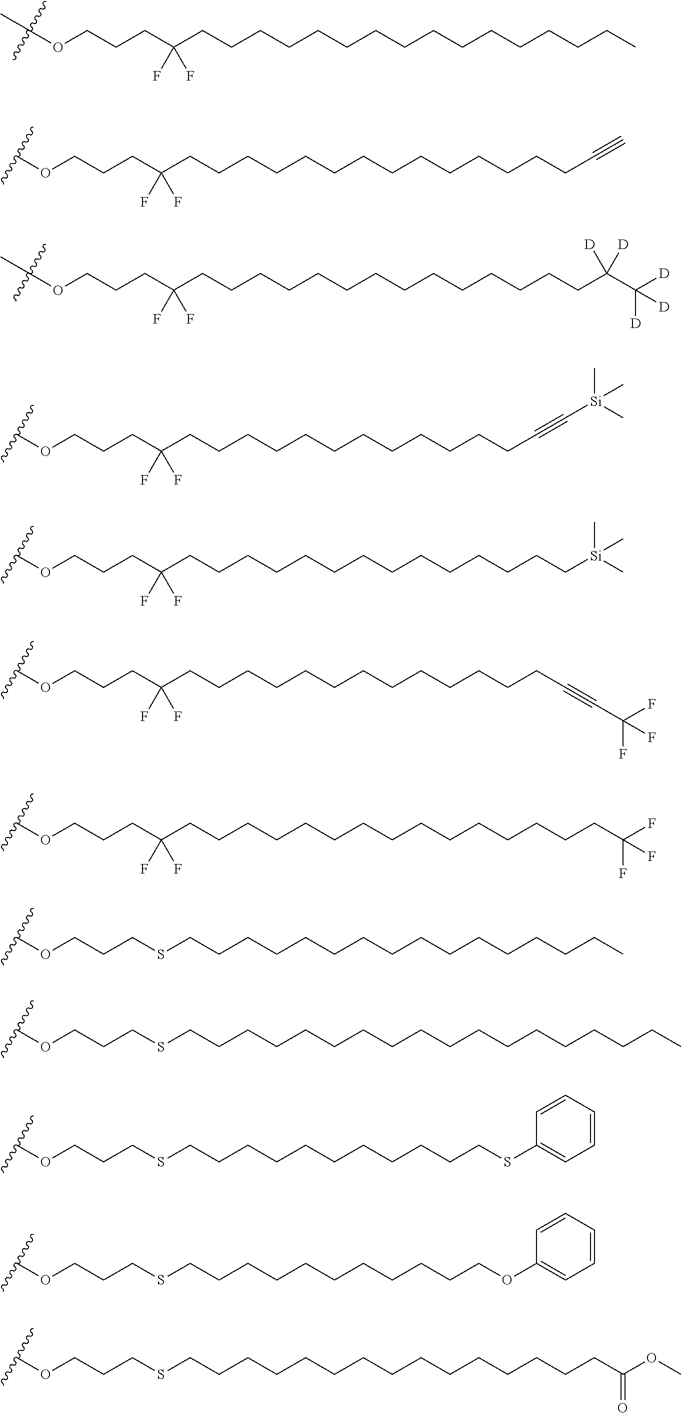

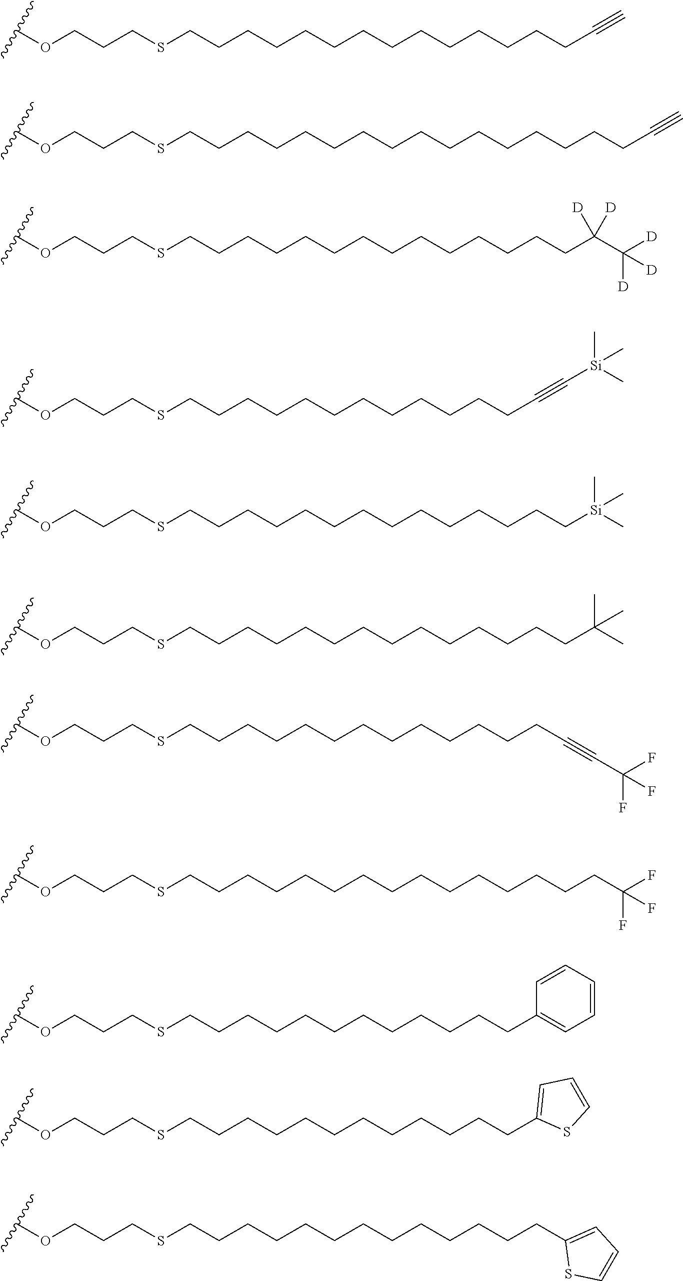

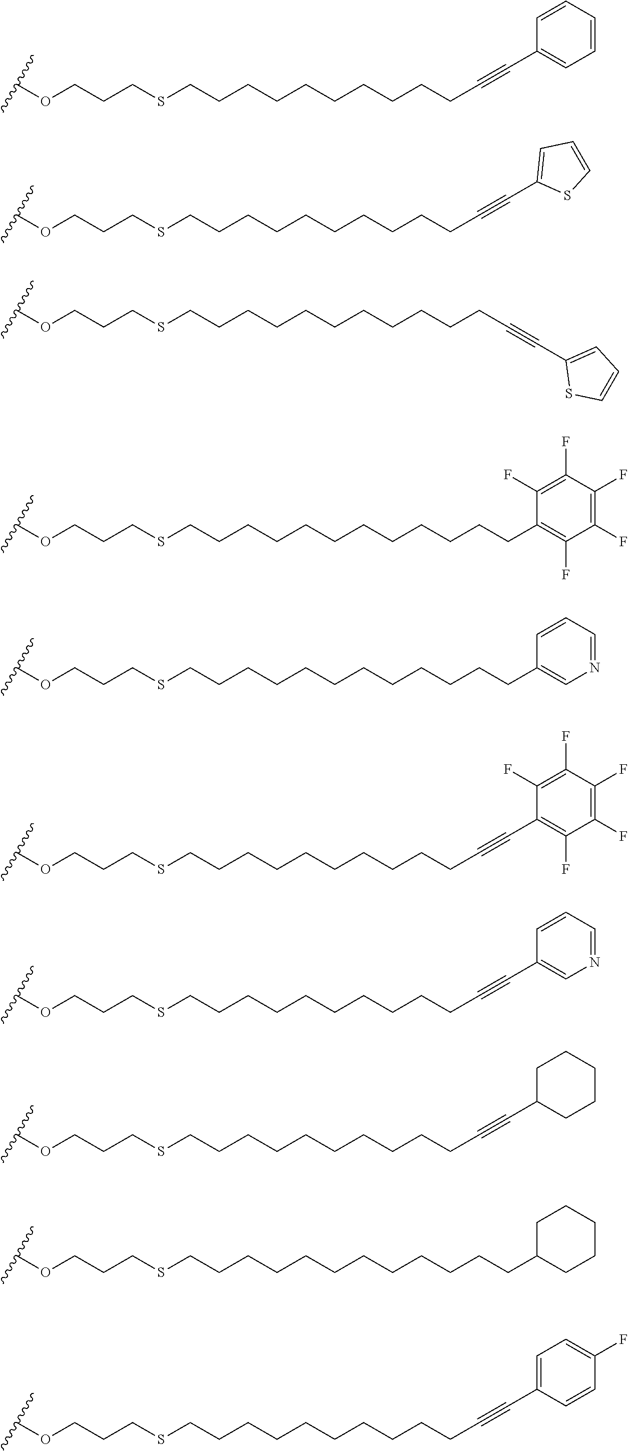

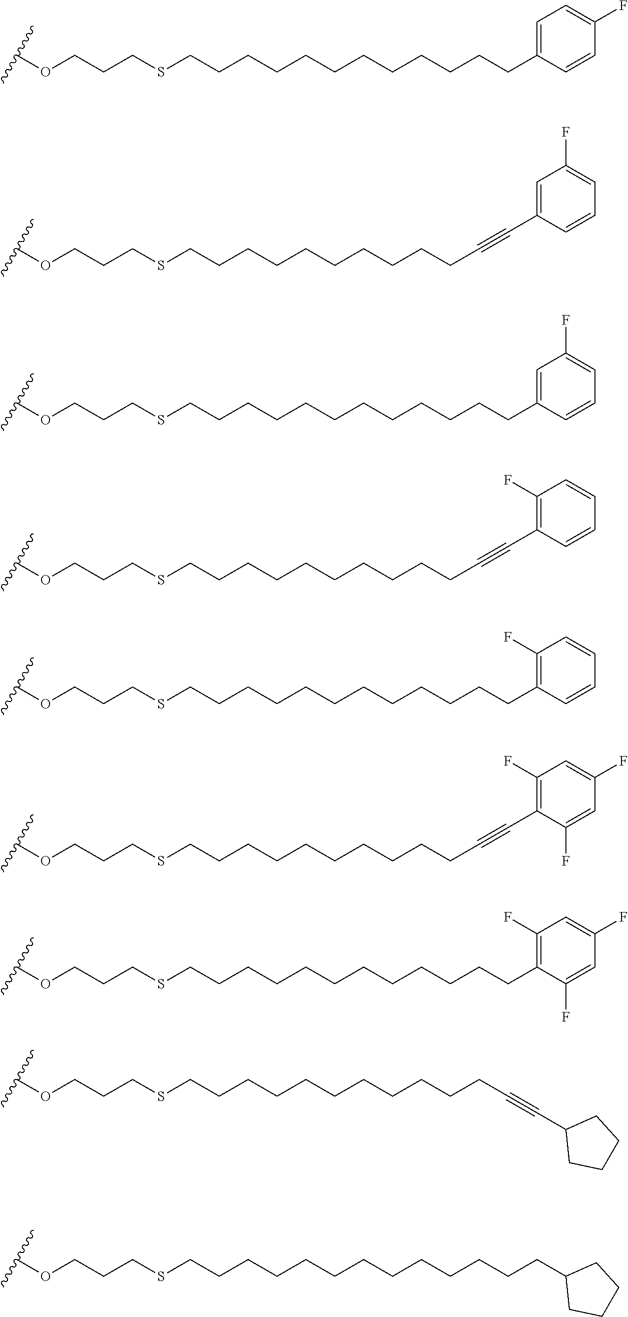

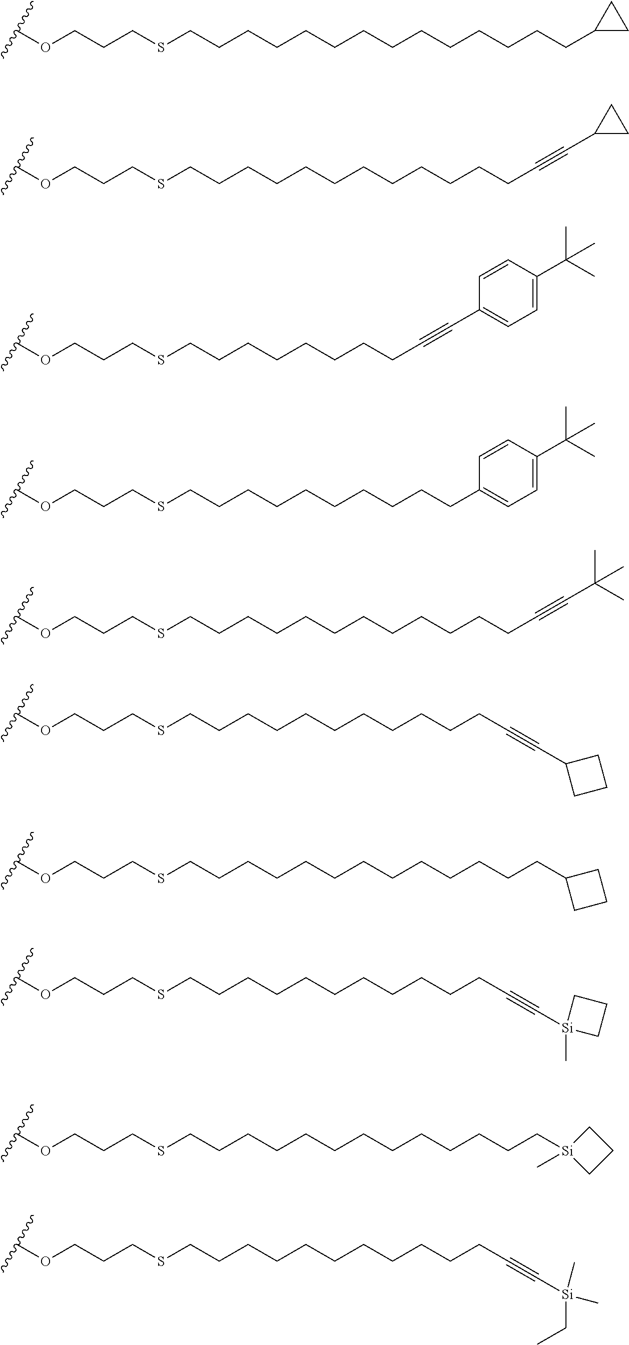

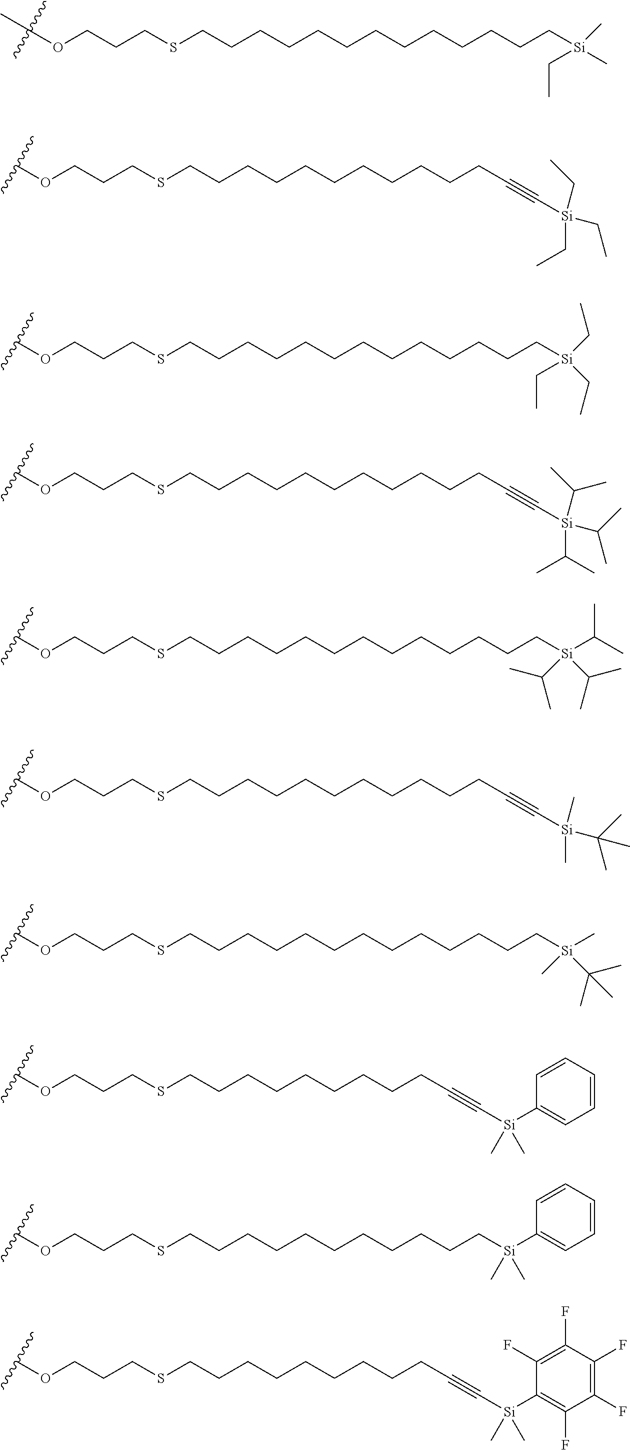

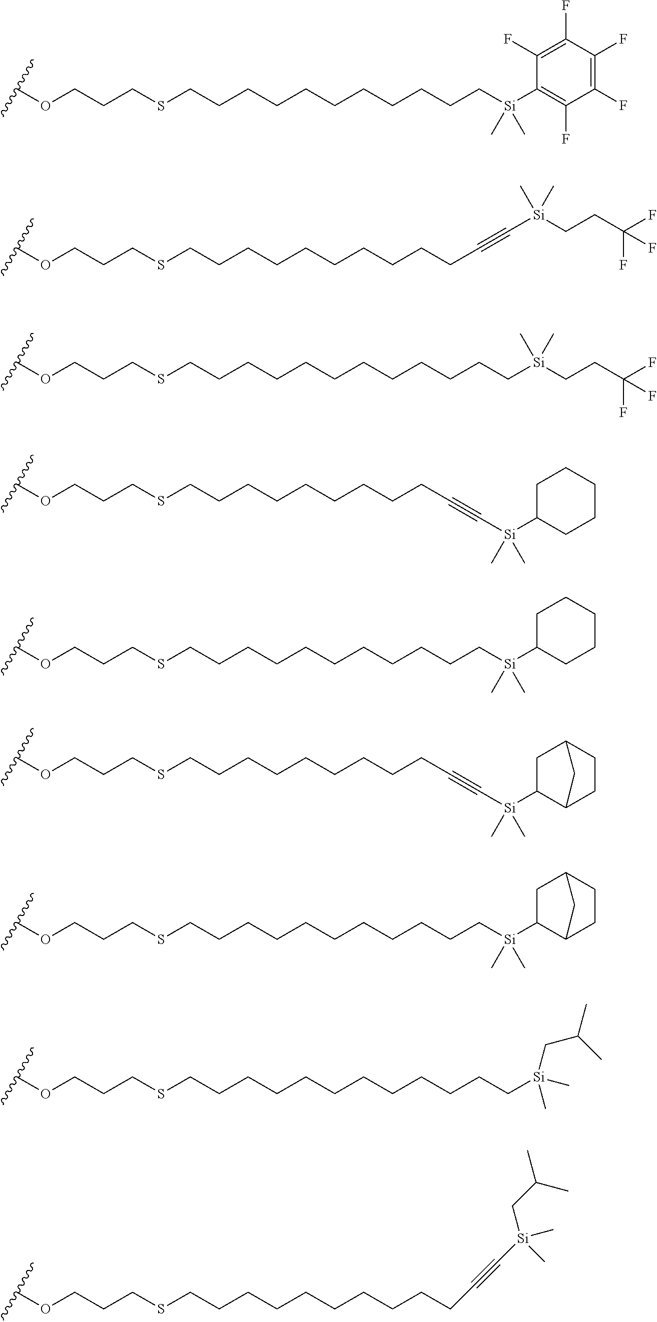

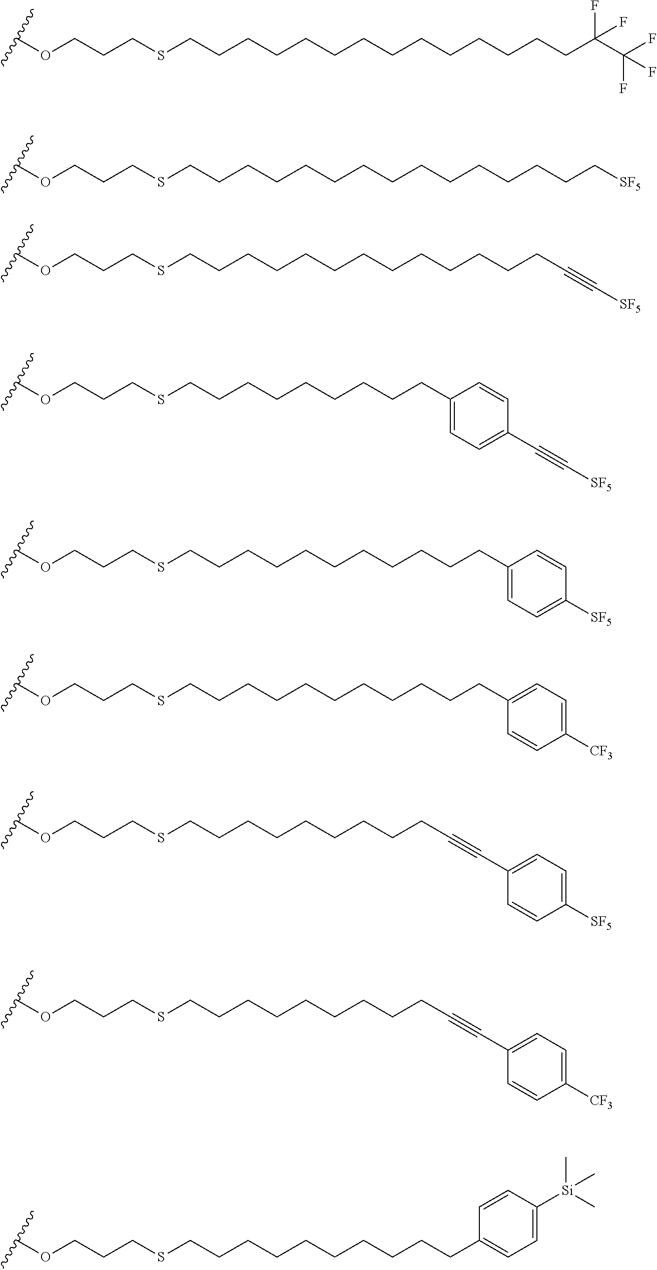

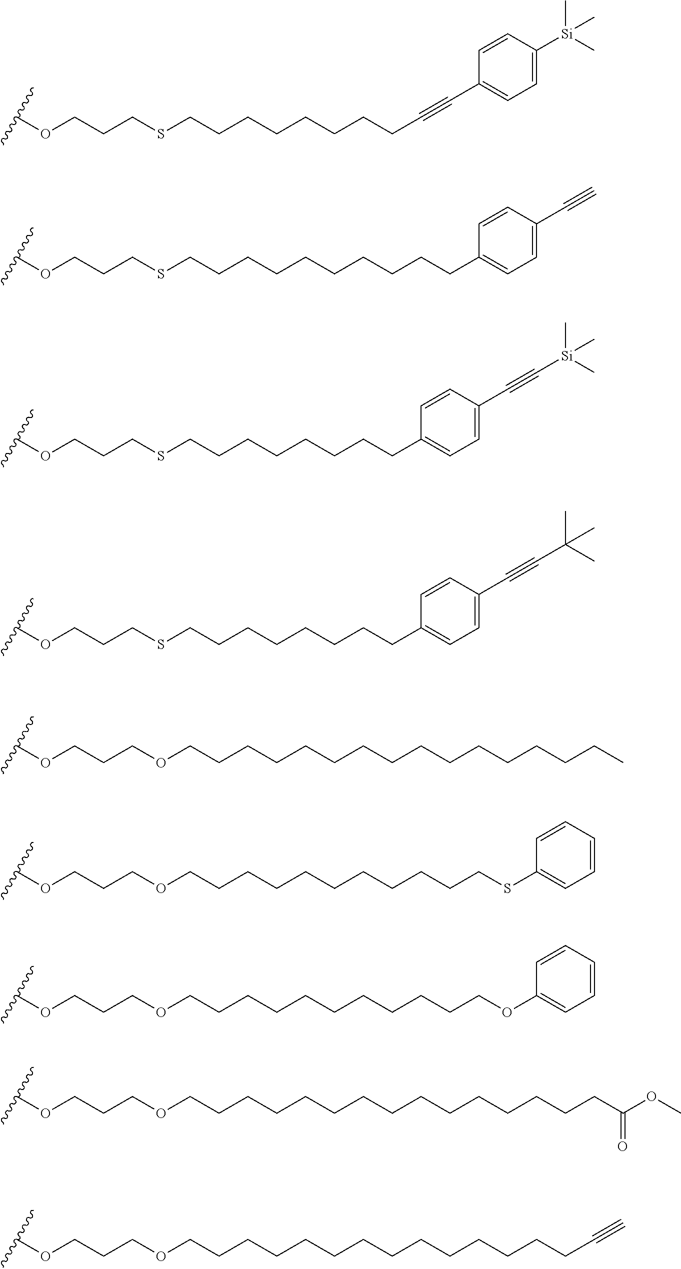

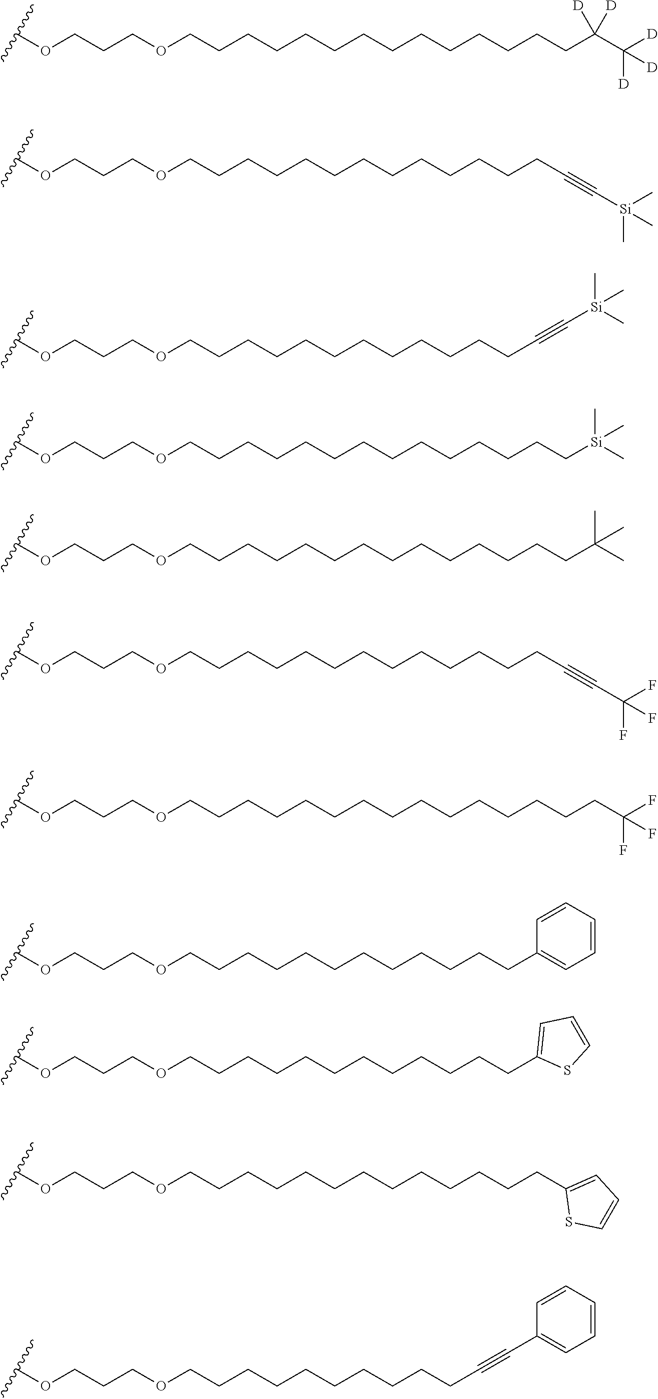

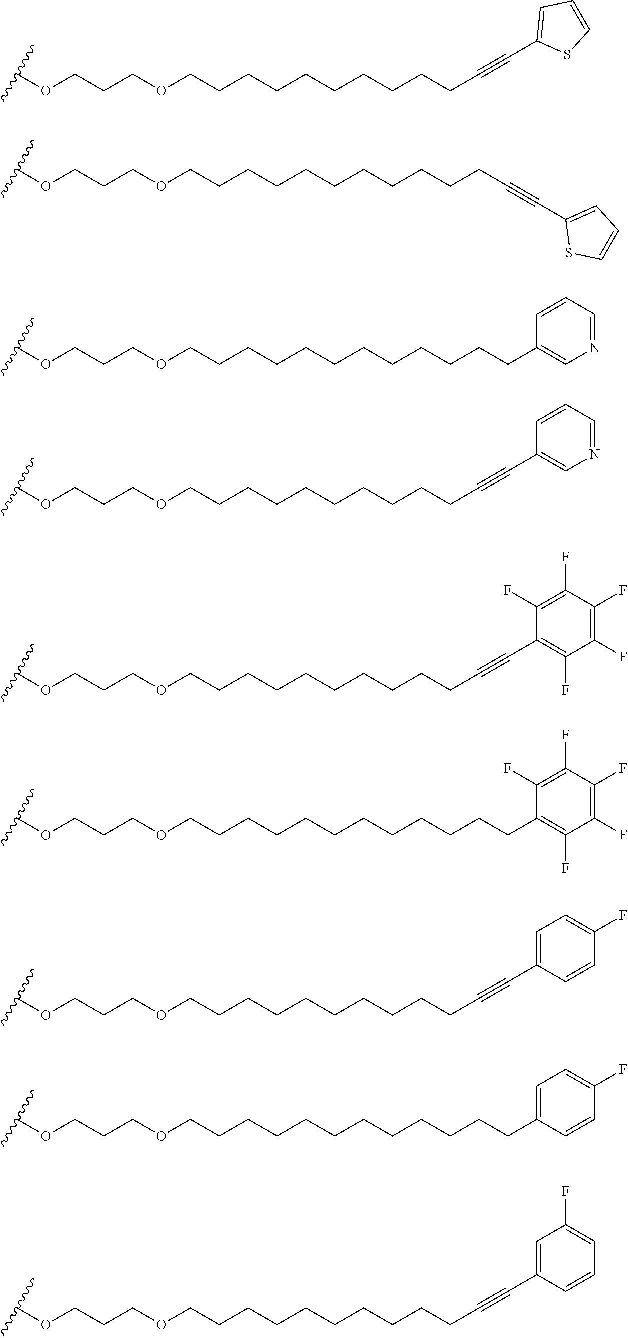

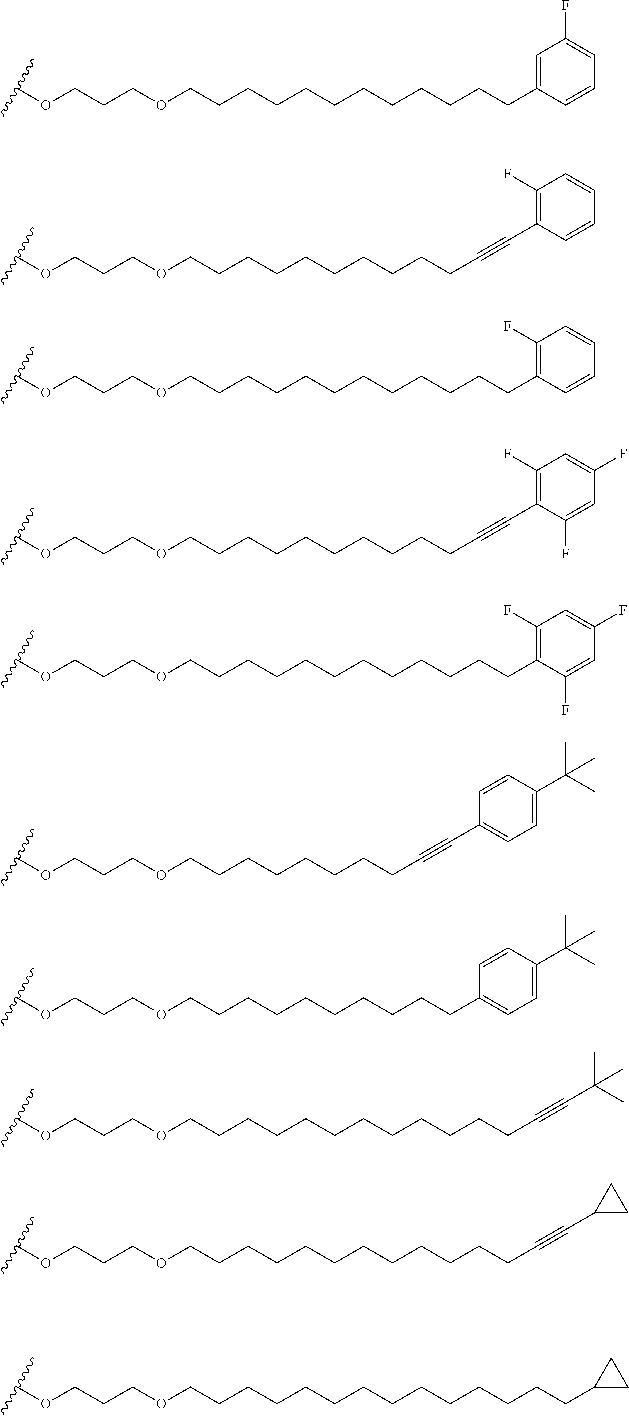

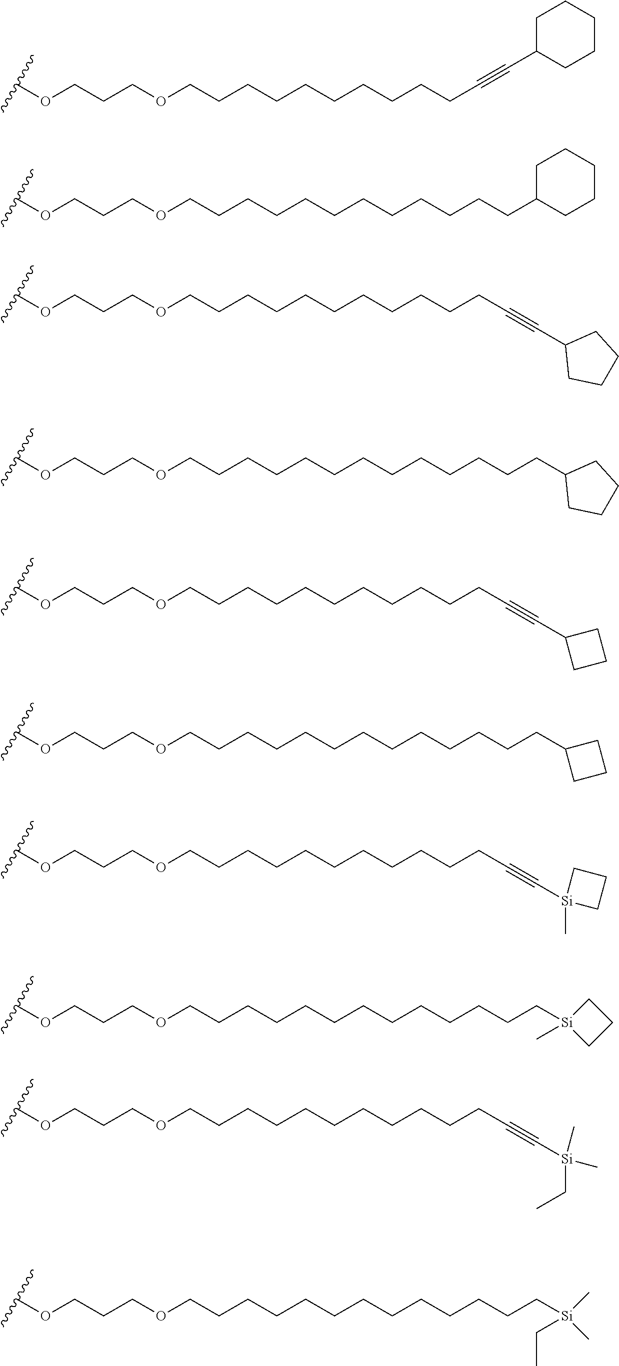

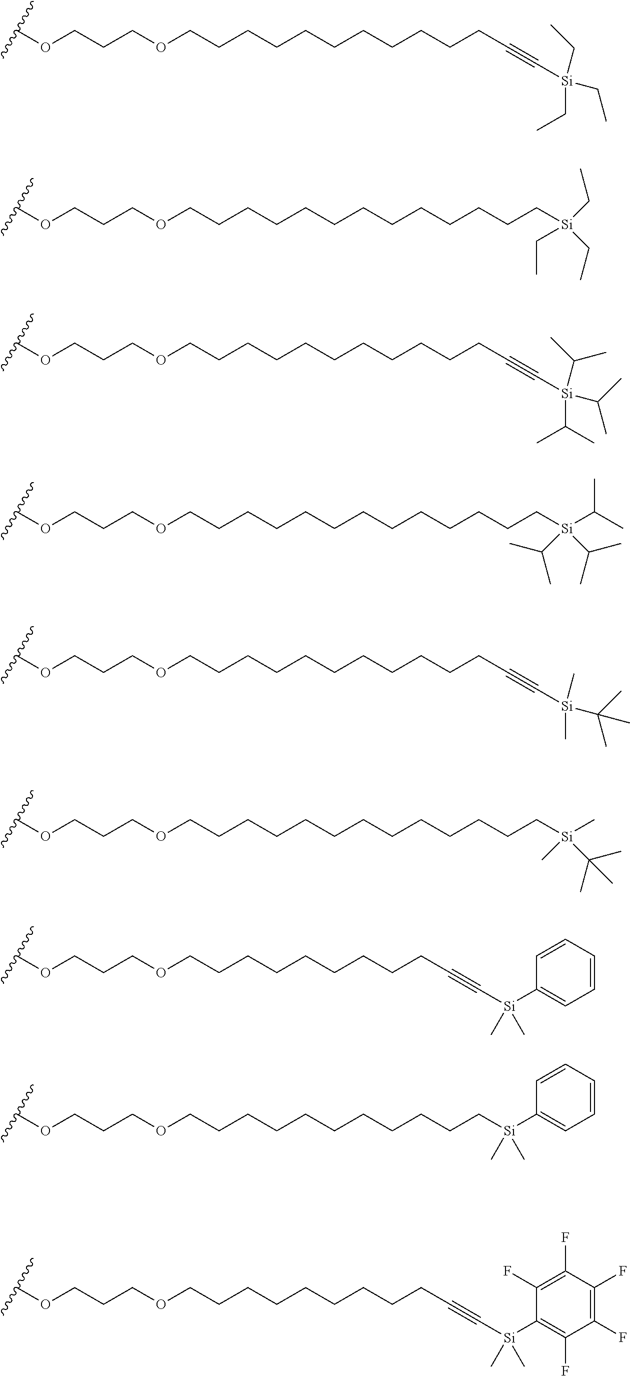

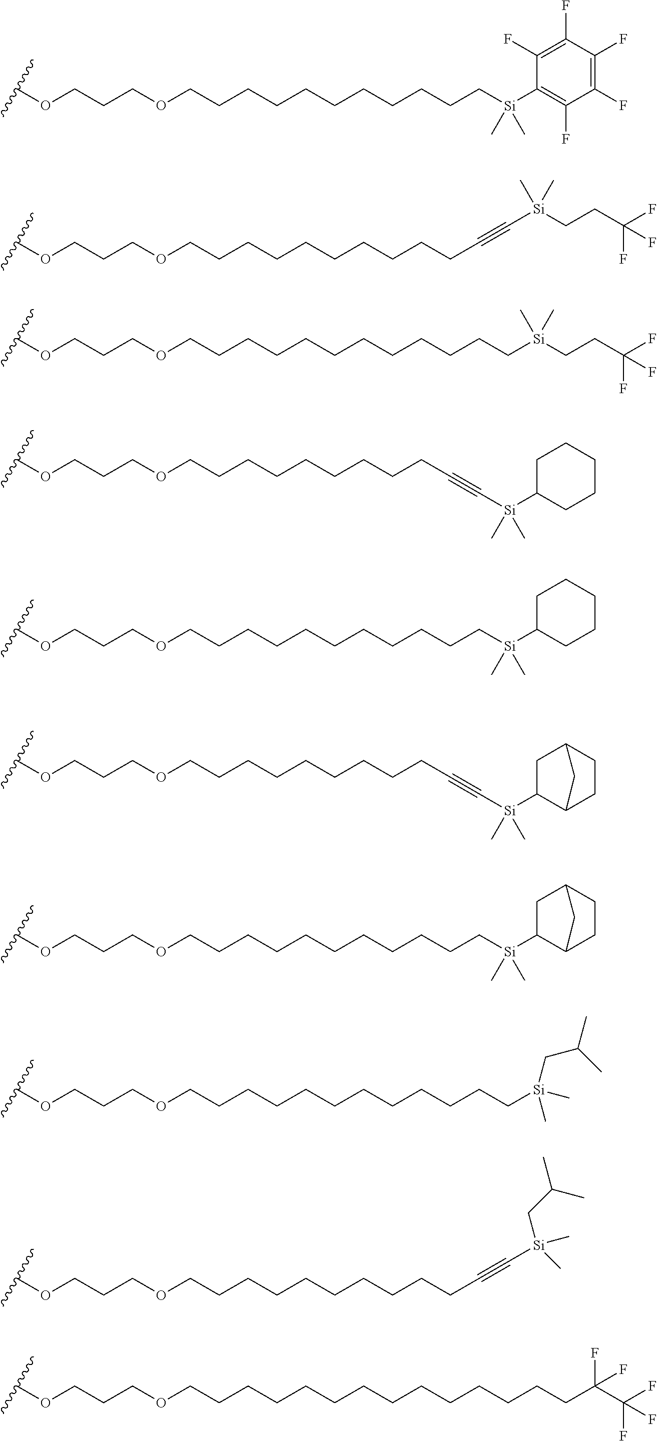

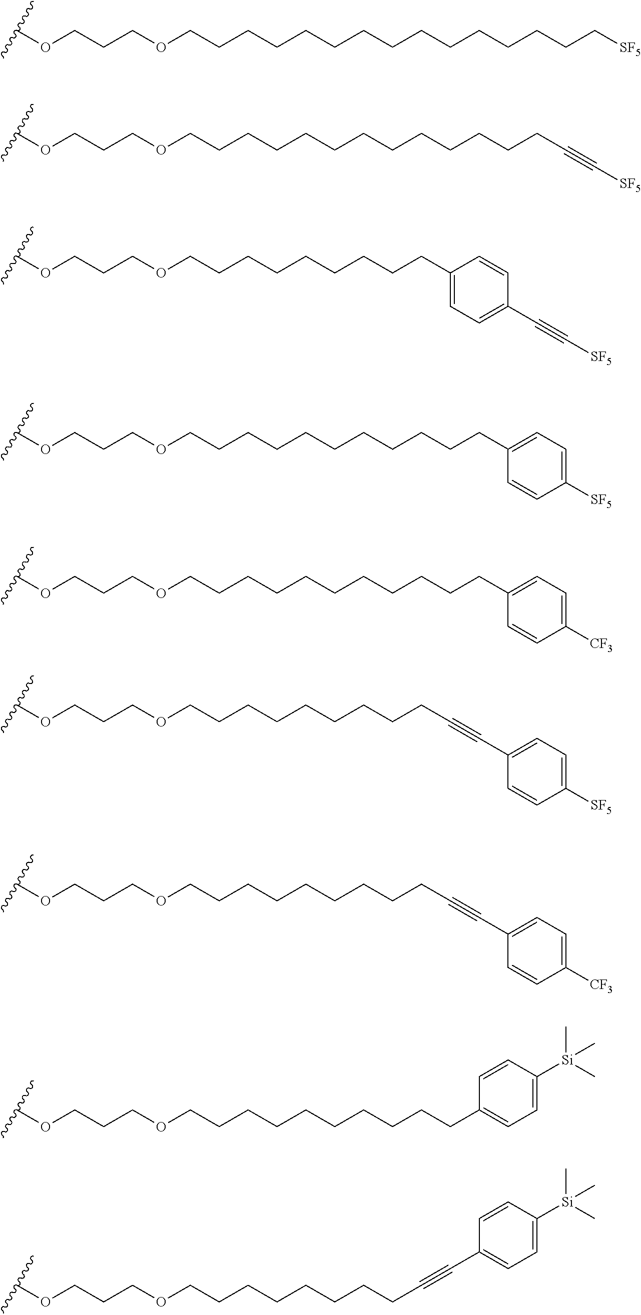

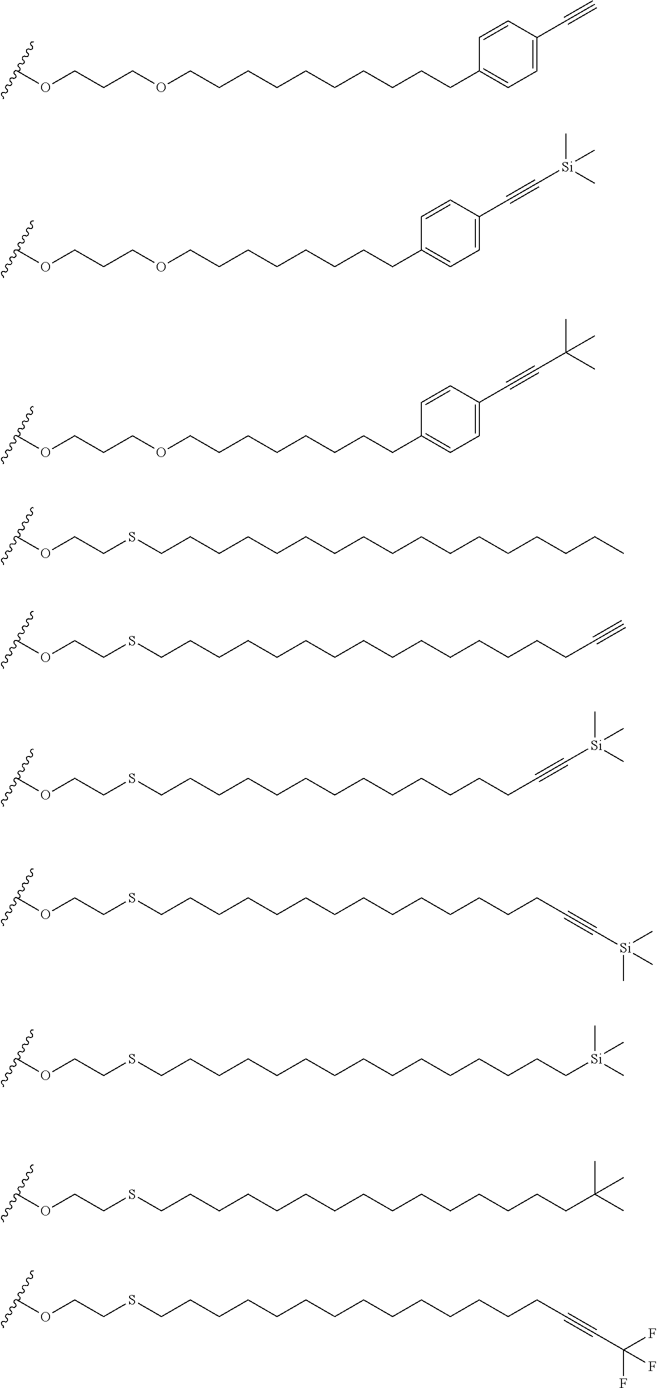

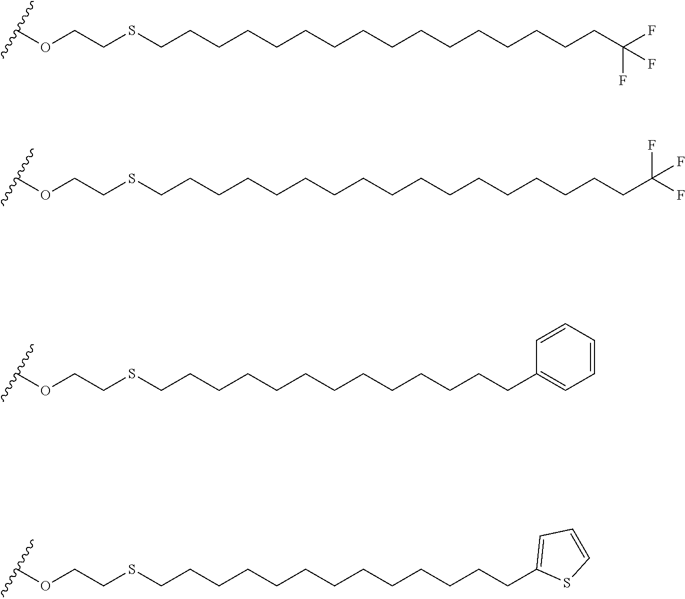 [SMILES: *OCCSCCCCCCCCCCCCCCCCC(F)(F)F ; *OCCSCCCCCCCCCCCCCCCCCC(F)(F)F ; *OCCSCCCCCCCCCCCCCc1ccccc1 ; *OCCSCCCCCCCCCCCCCc1cccs1]

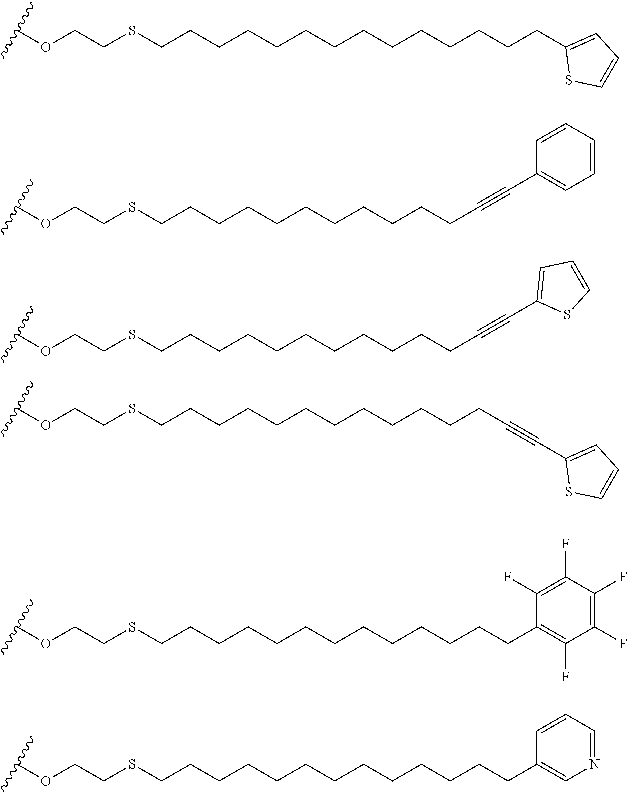

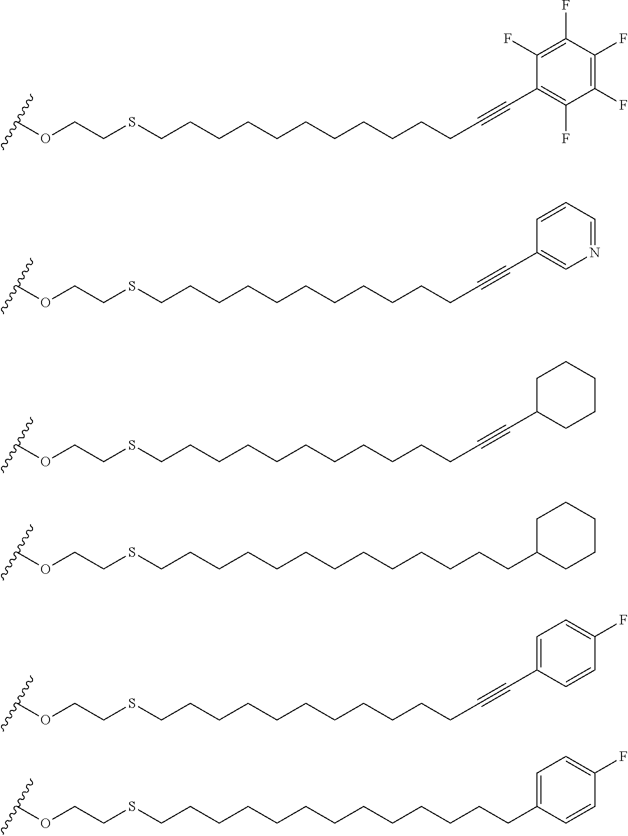 [SMILES: *OCCSCCCCCCCCCCCC#Cc1c(F)c(F)c(F)c(F)c1F ; *OCCSCCCCCCCCCCCC#Cc1cccnc1 ; *OCCSCCCCCCCCCCCC#CC1CCCCC1 ; *OCCSCCCCCCCCCCCCCC1CCCCC1 ; *OCCSCCCCCCCCCCCC#Cc1ccc(F)cc1 ; *OCCSCCCCCCCCCCCCCc1ccc(F)cc1]

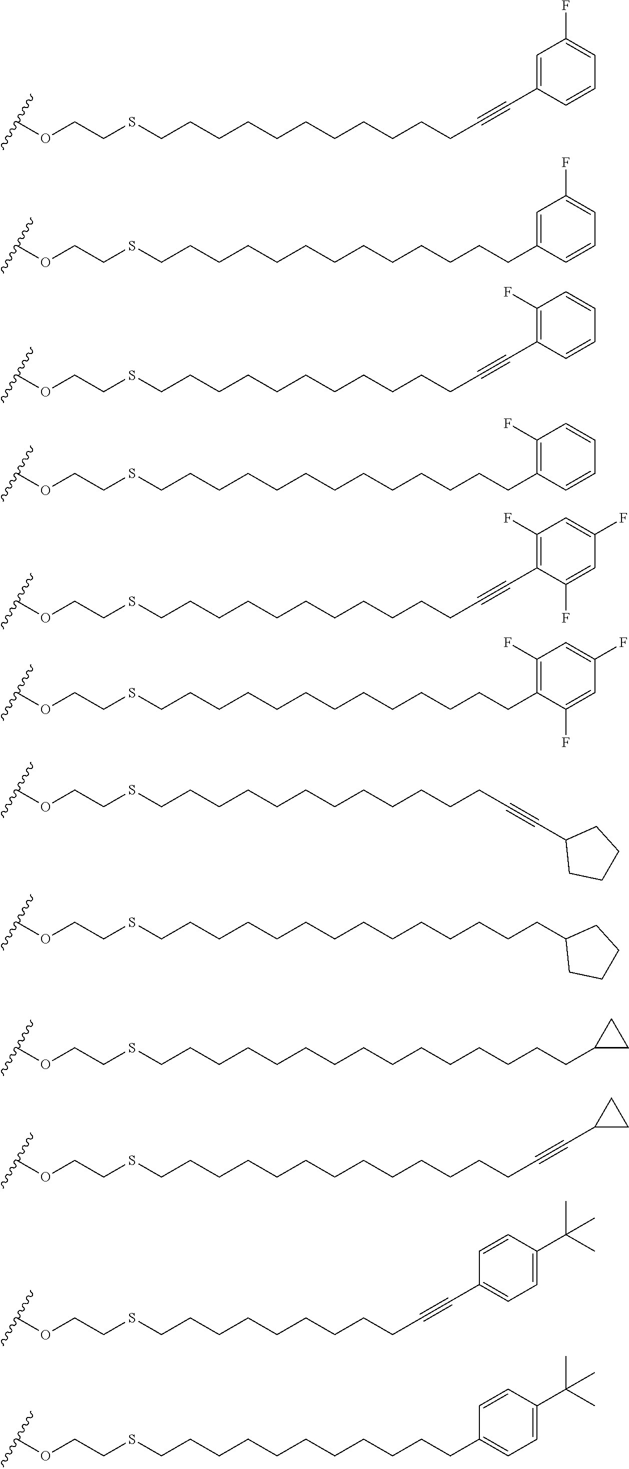

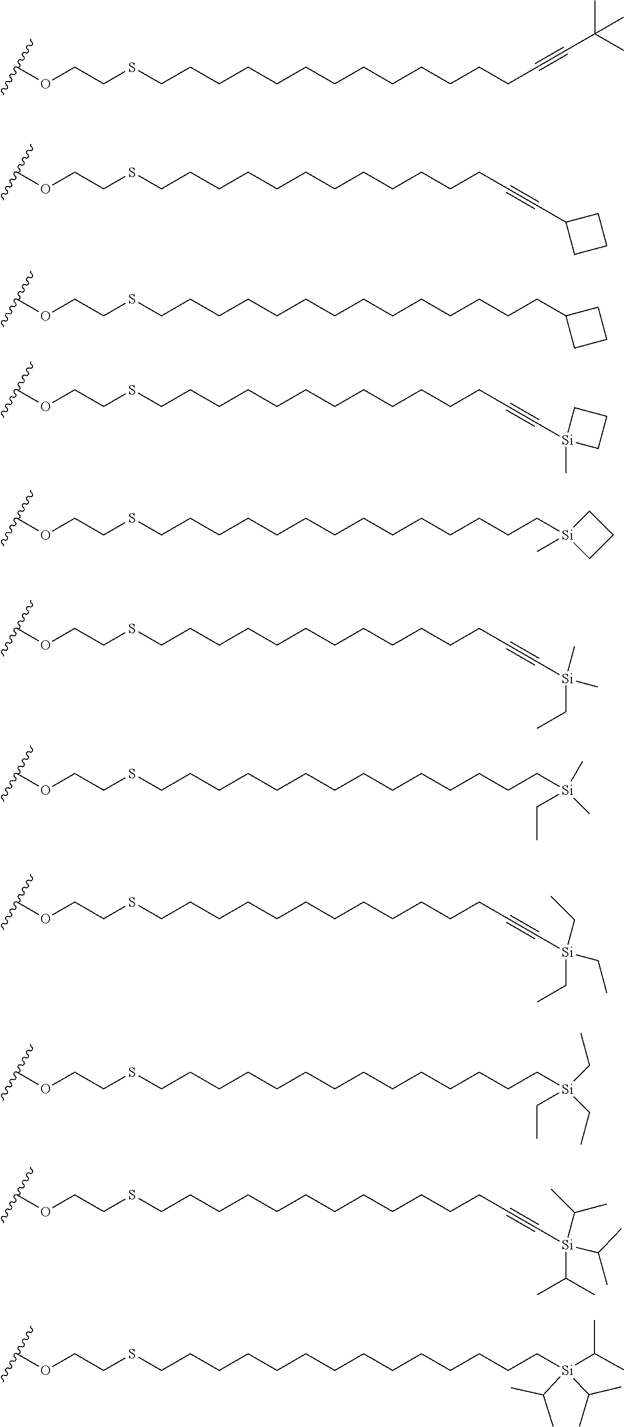

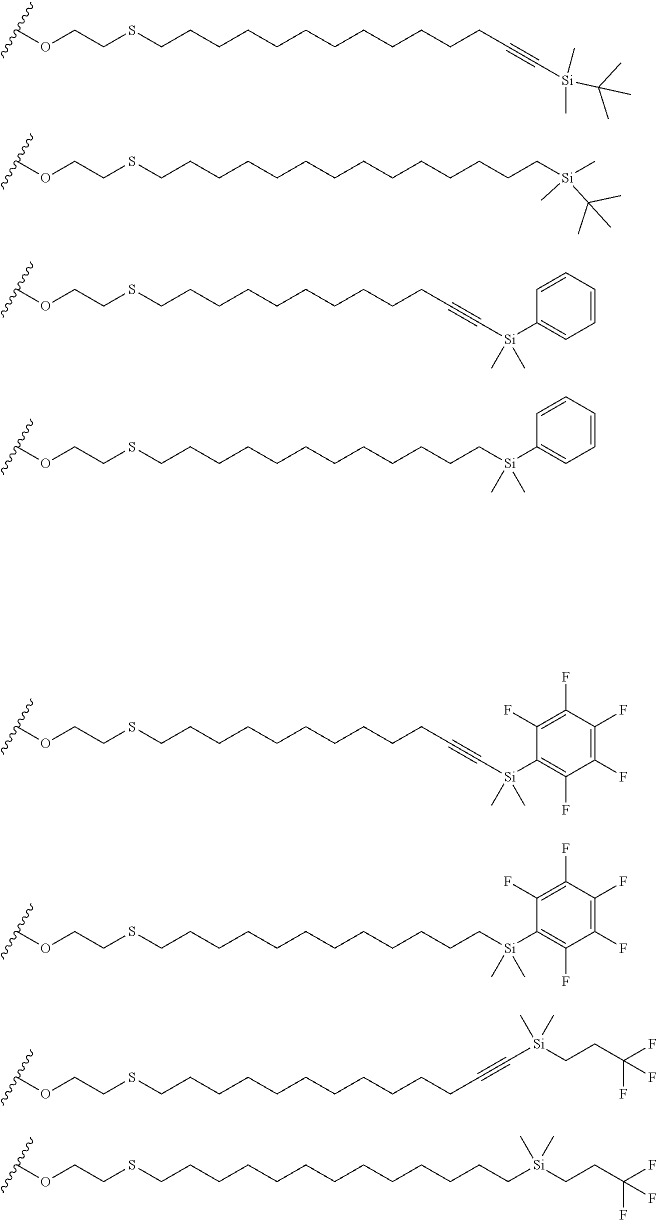

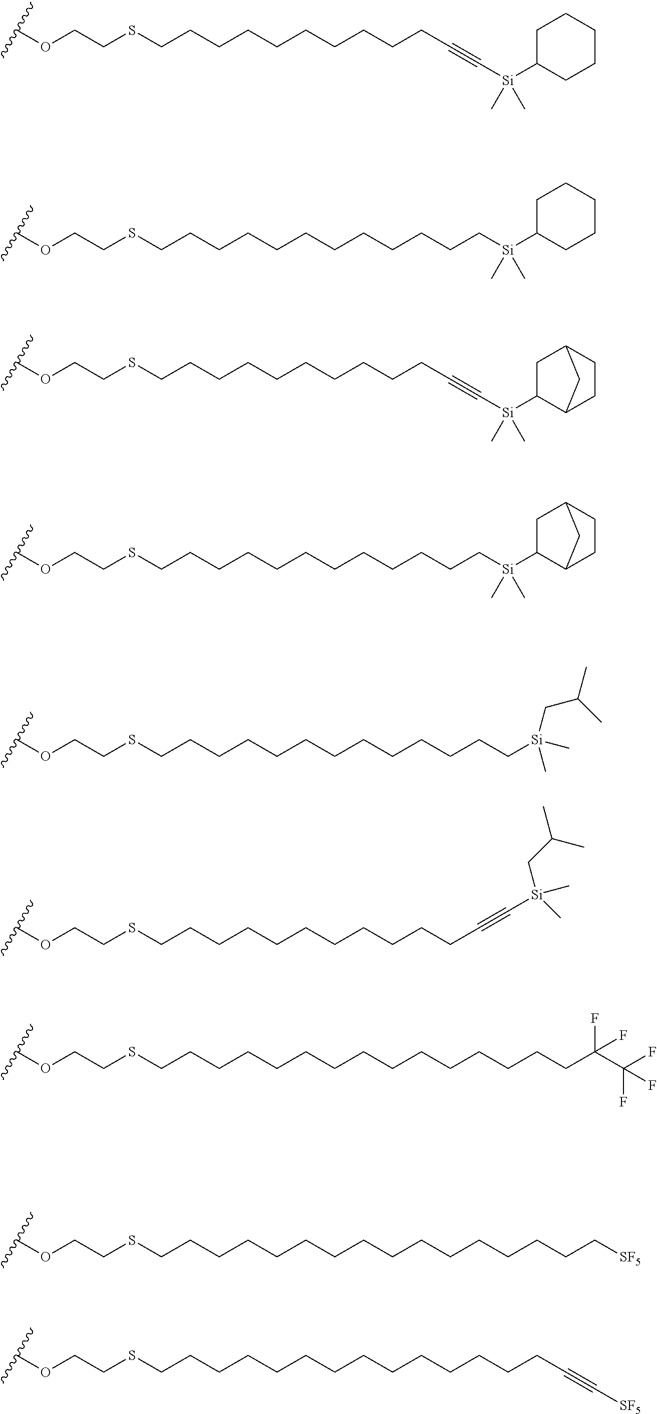

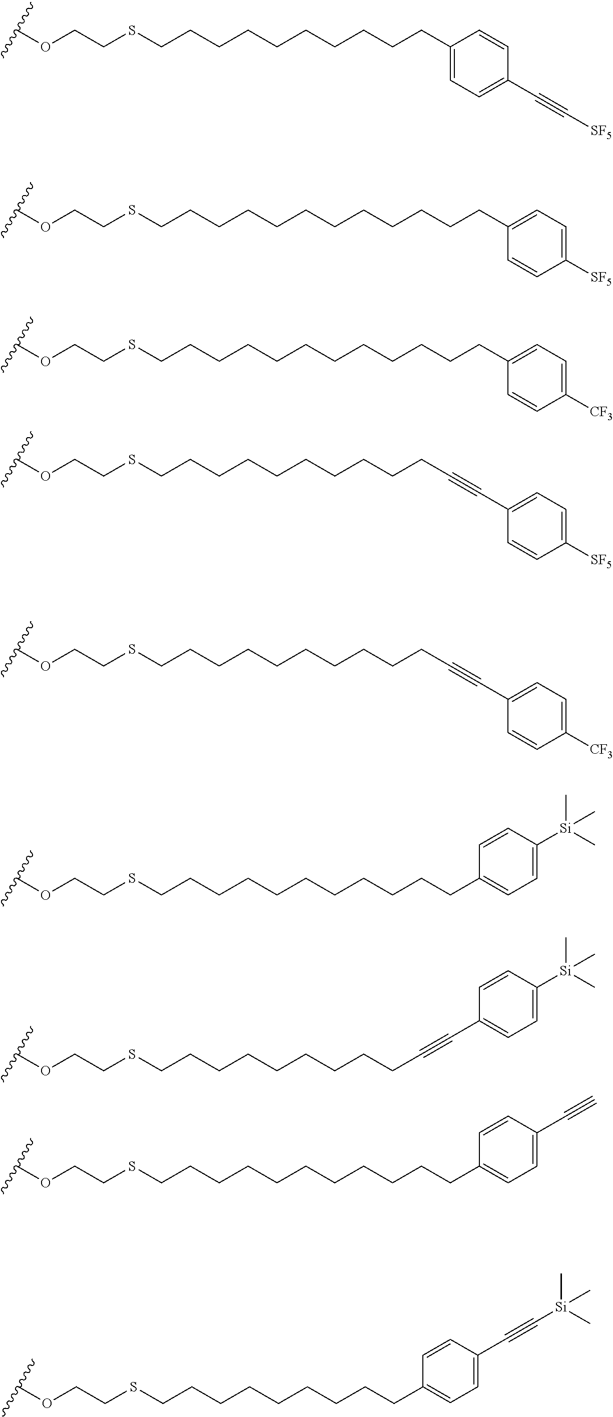

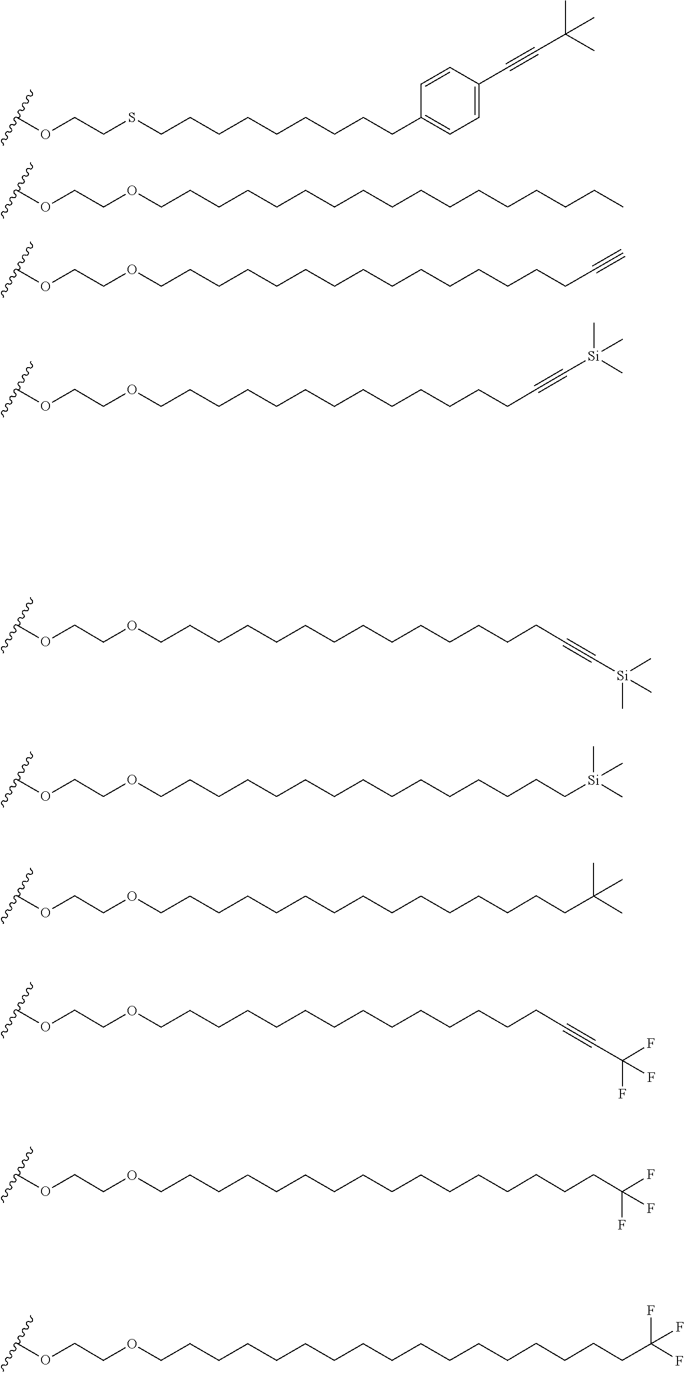

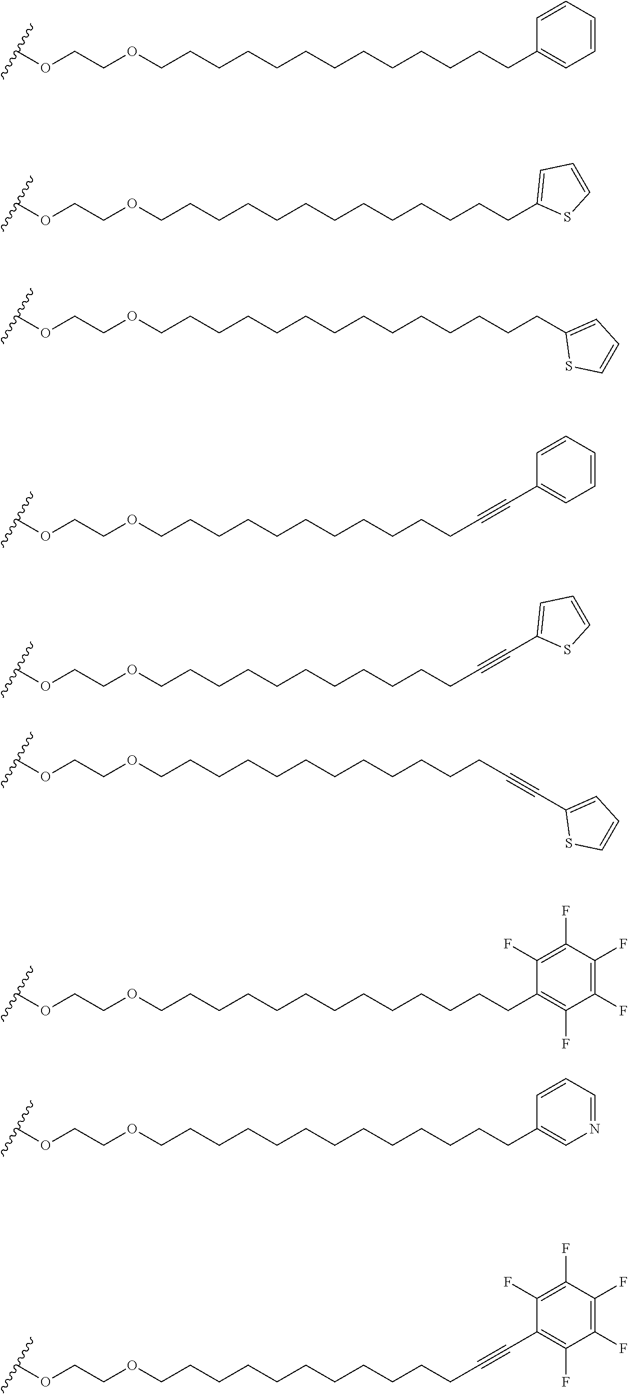

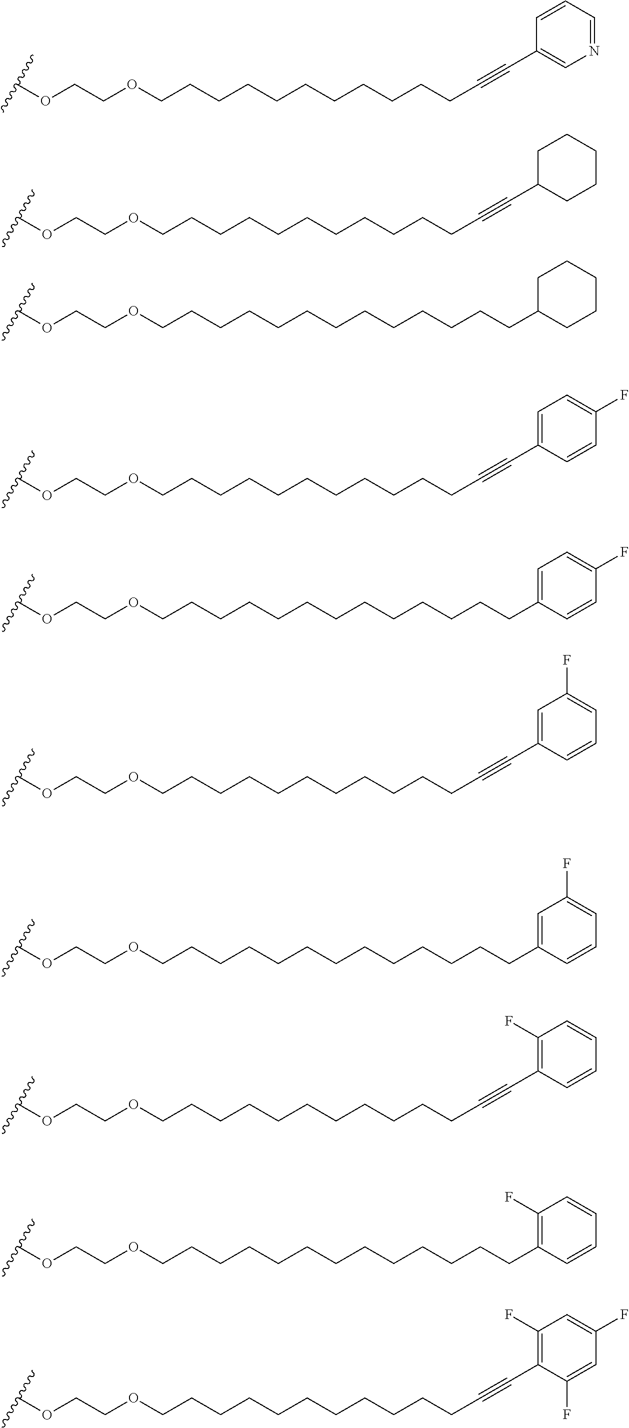

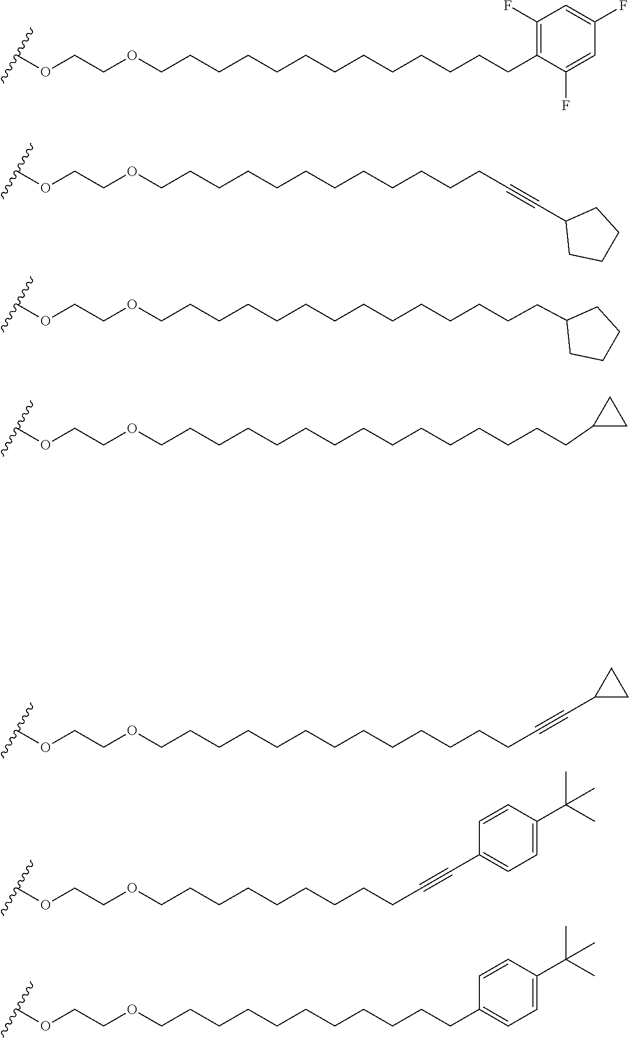

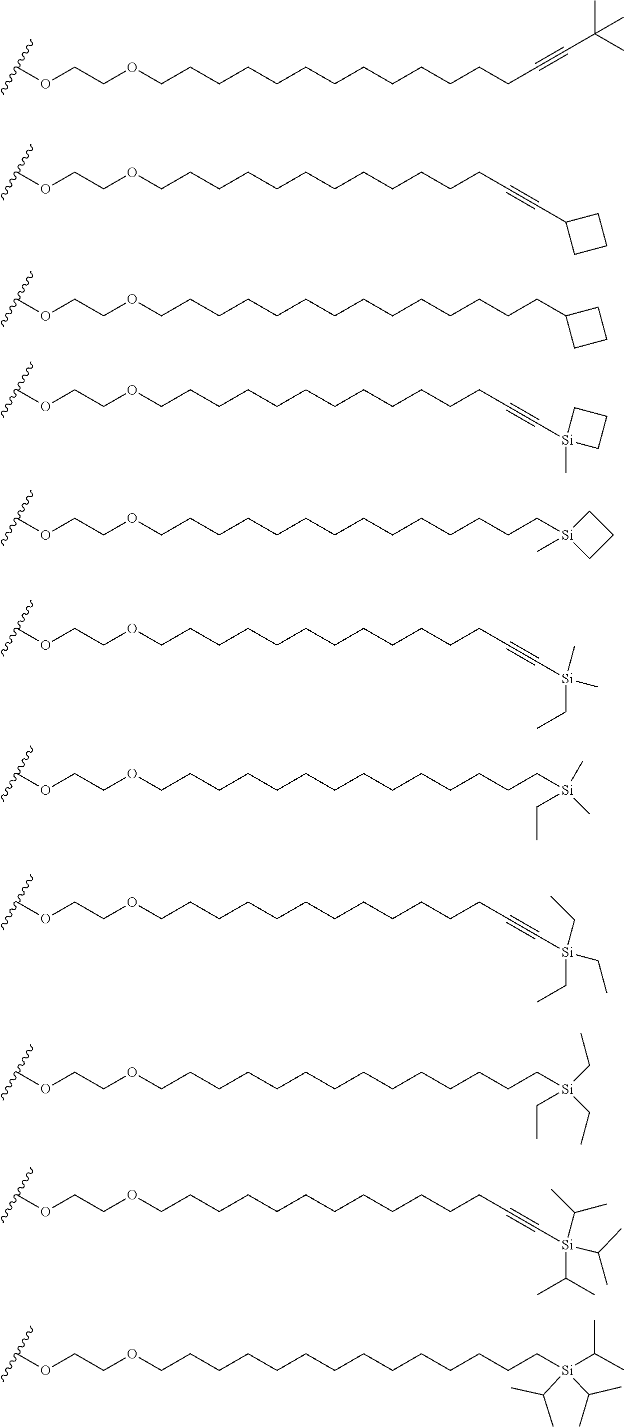

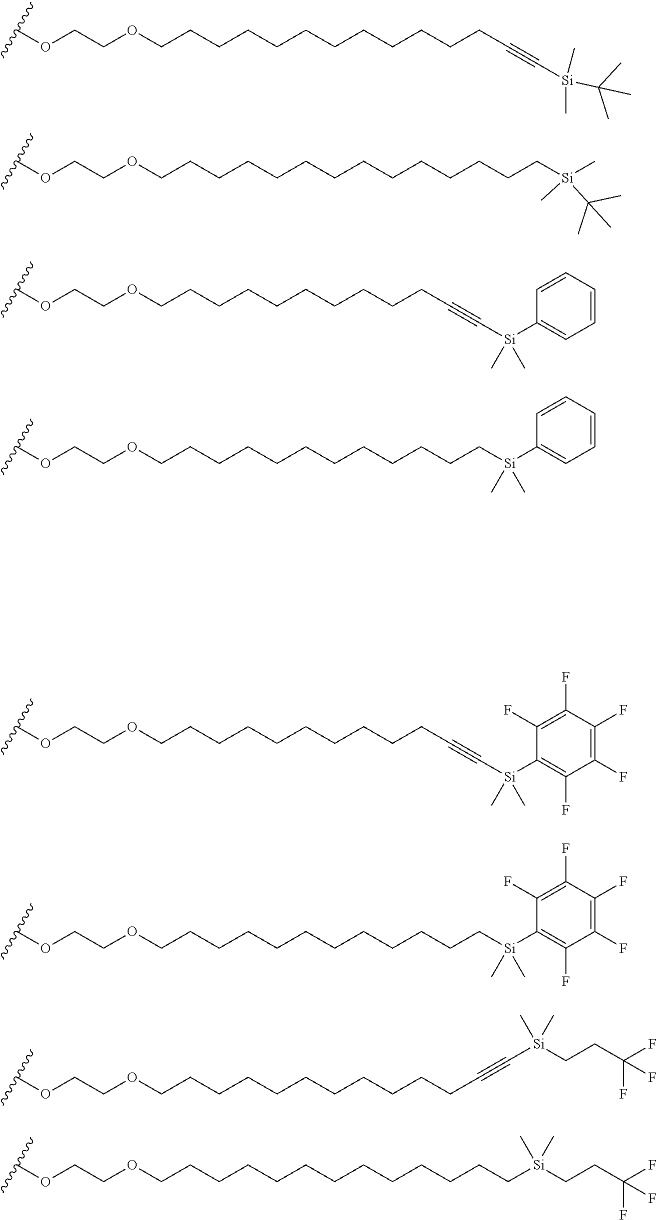

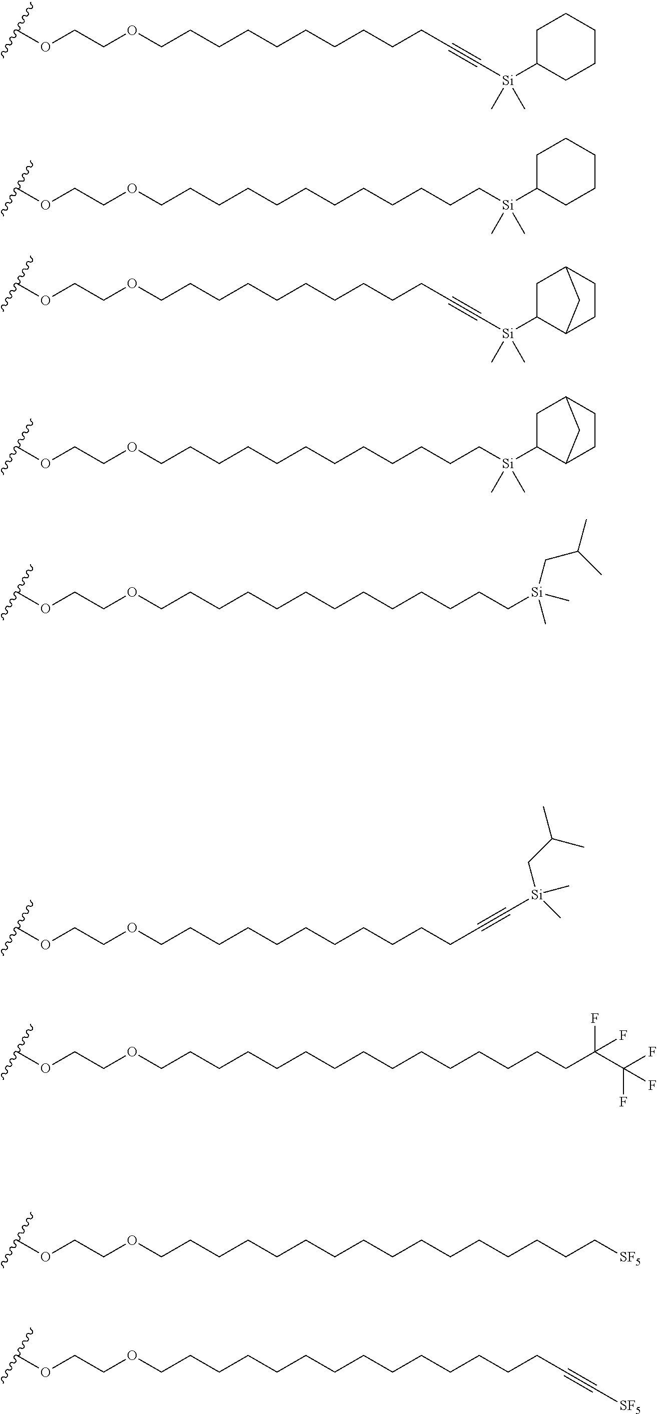

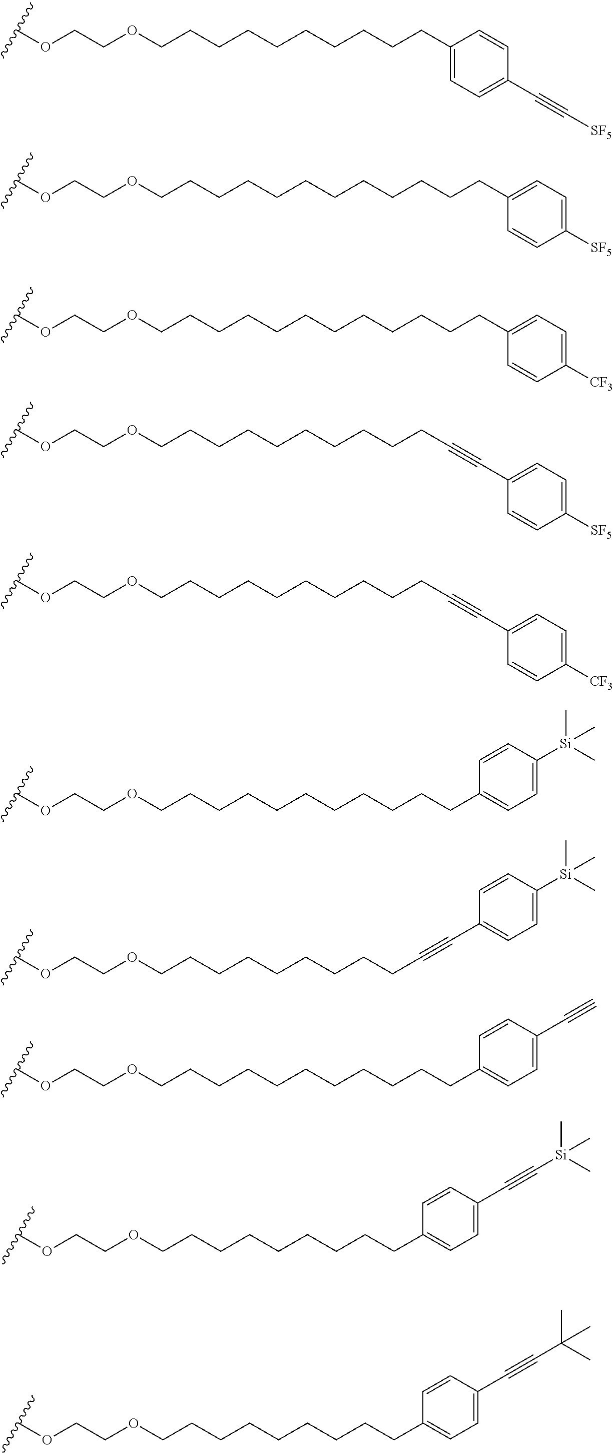

In some examples, Z is selected from:

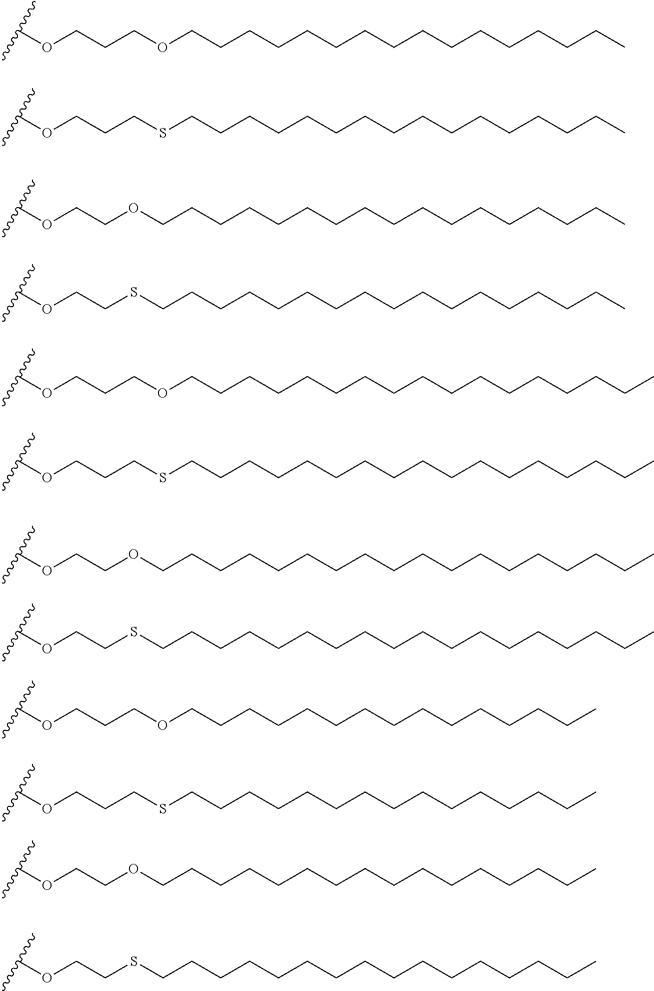

In some examples, Z is selected from:

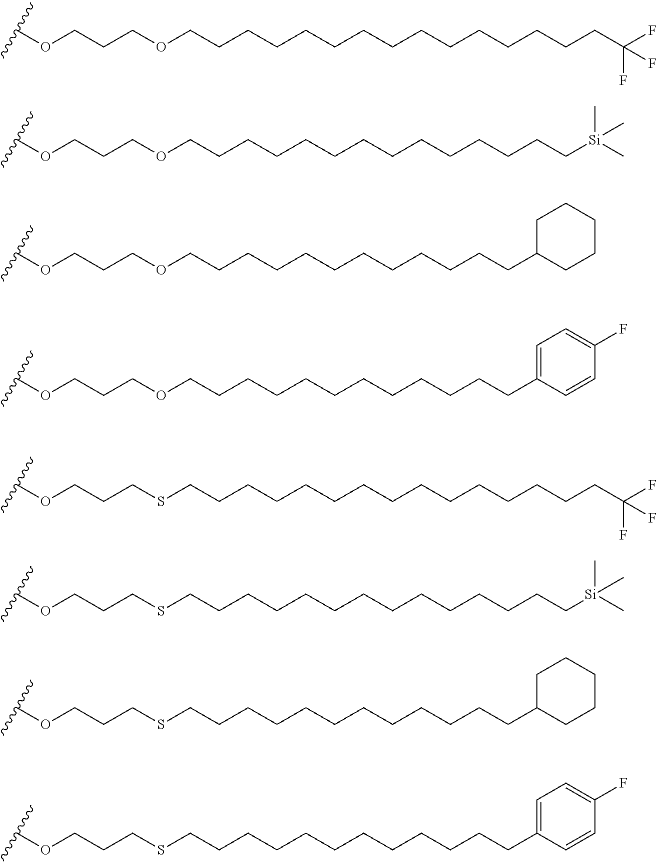

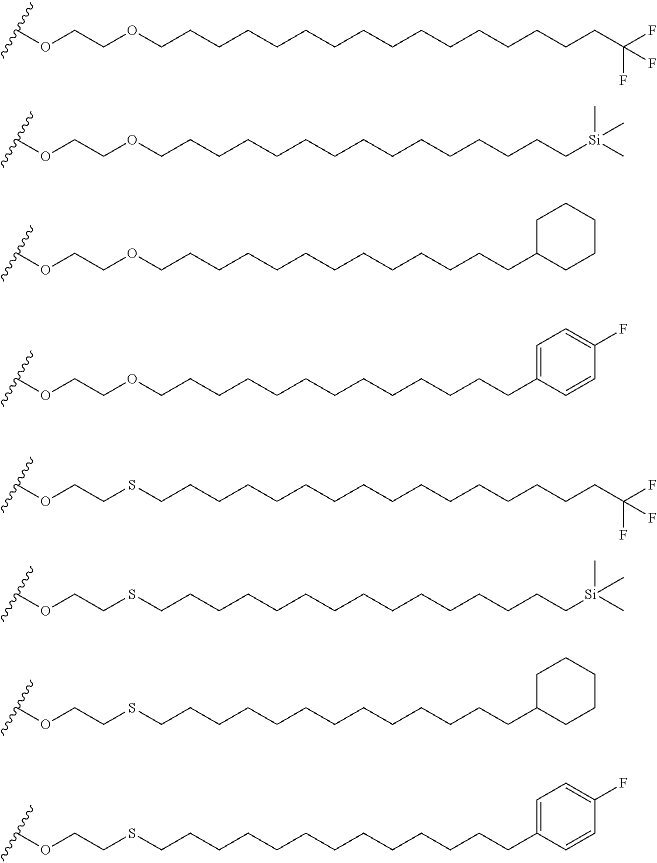

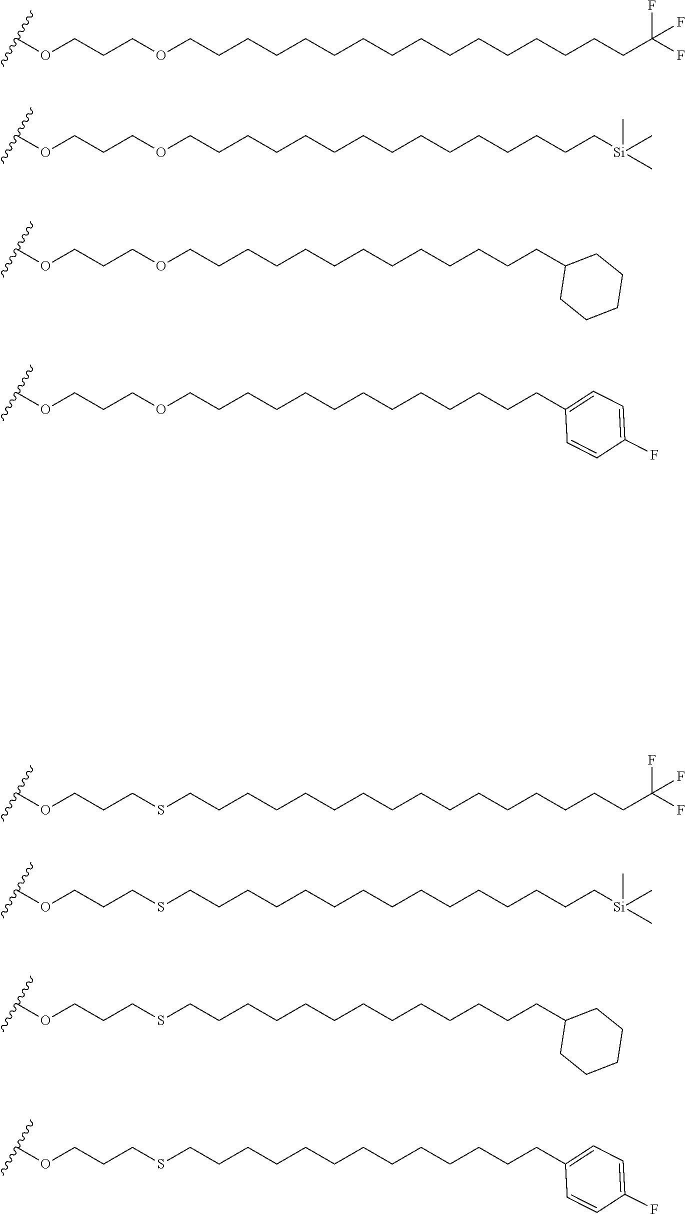

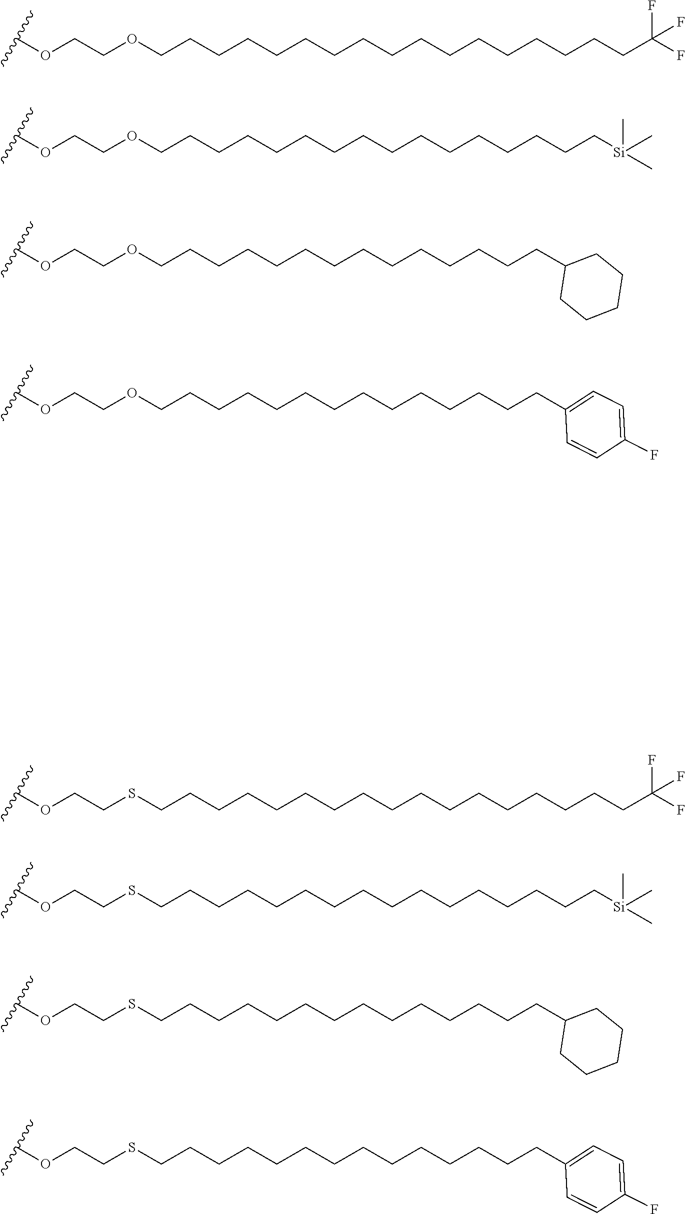

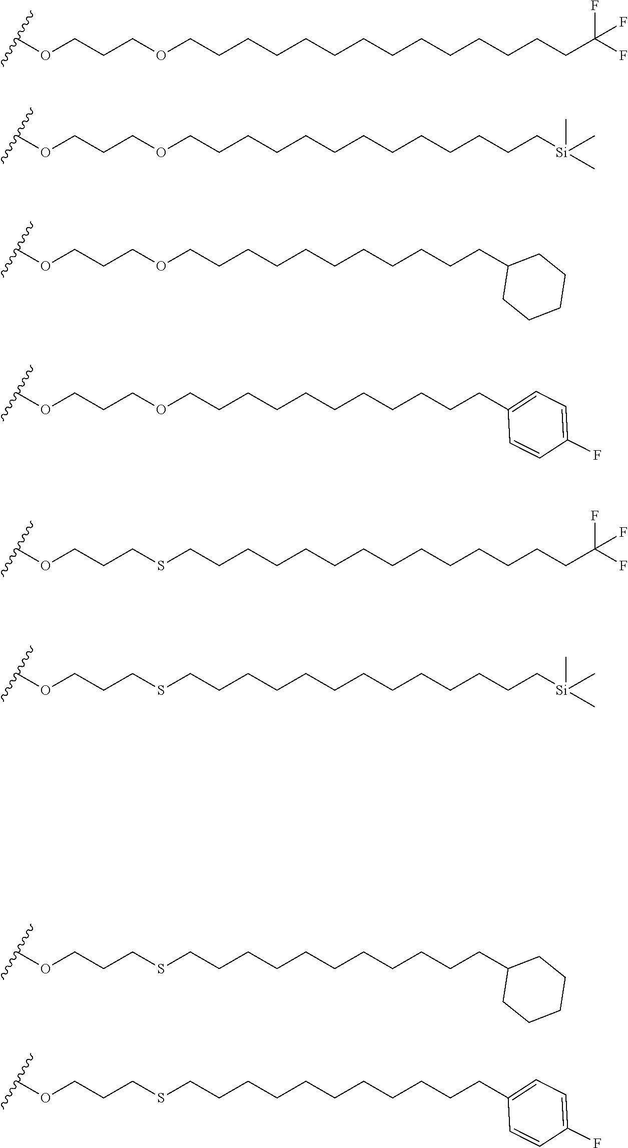

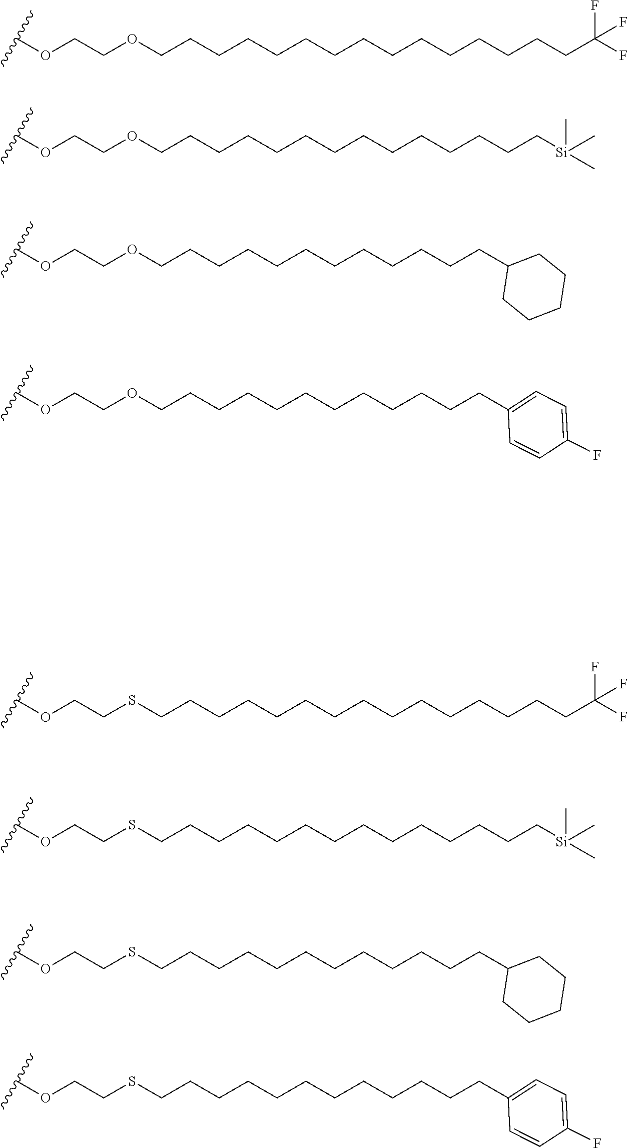

In some examples, the lipid-derived prodrugs have a structure selected from the following structures or pharmaceutically acceptable salts (such as ammonium and lithium salts) thereof:

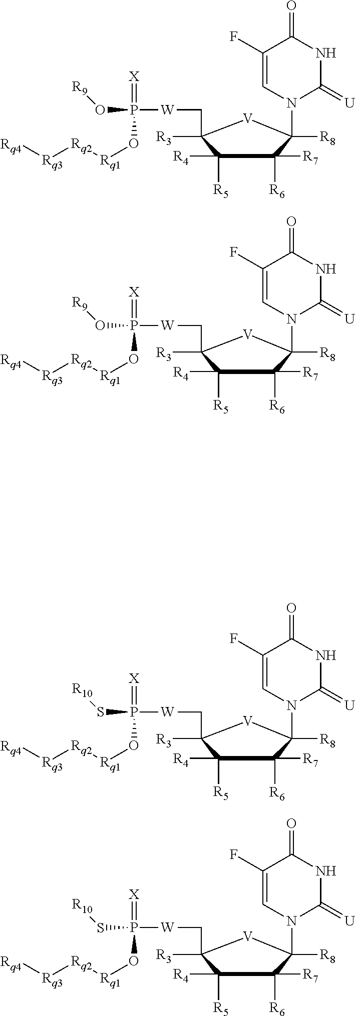

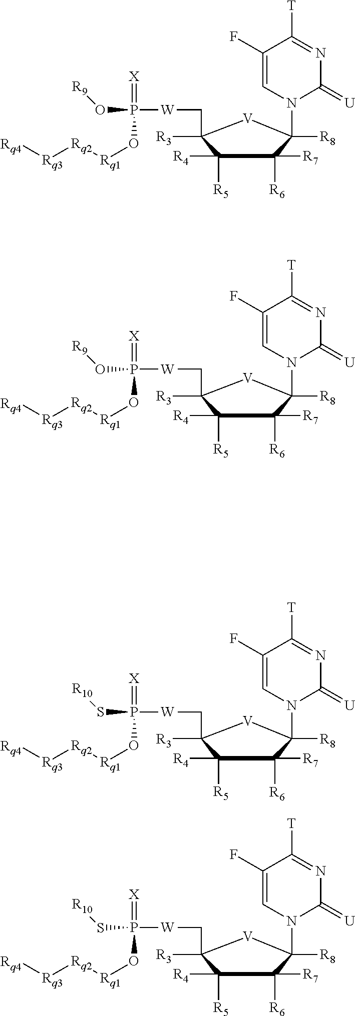

In some examples, the lipid-derived prodrugs have a structure selected from the following structures or pharmaceutically acceptable salts (such as ammonium and lithium salts) thereof:

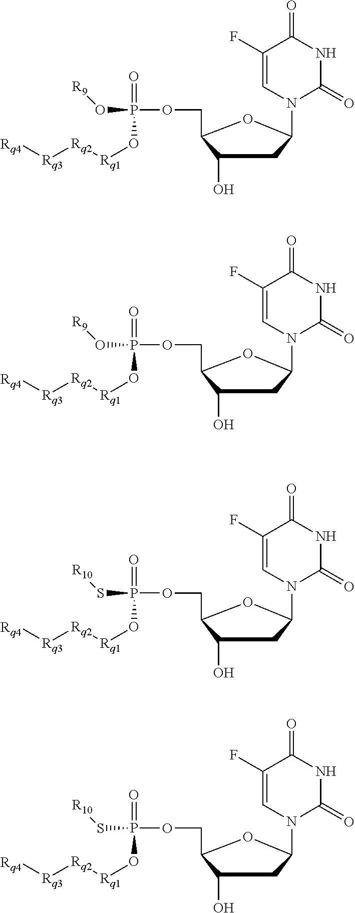

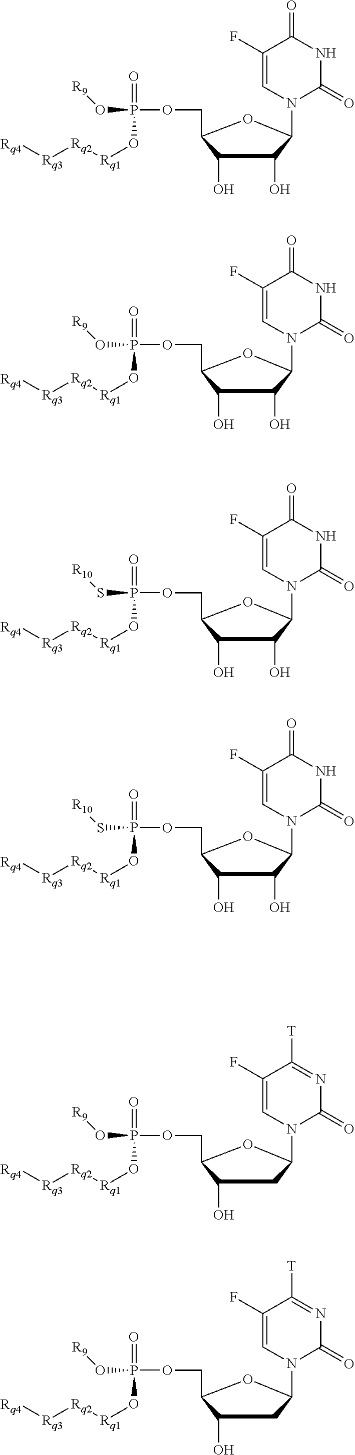

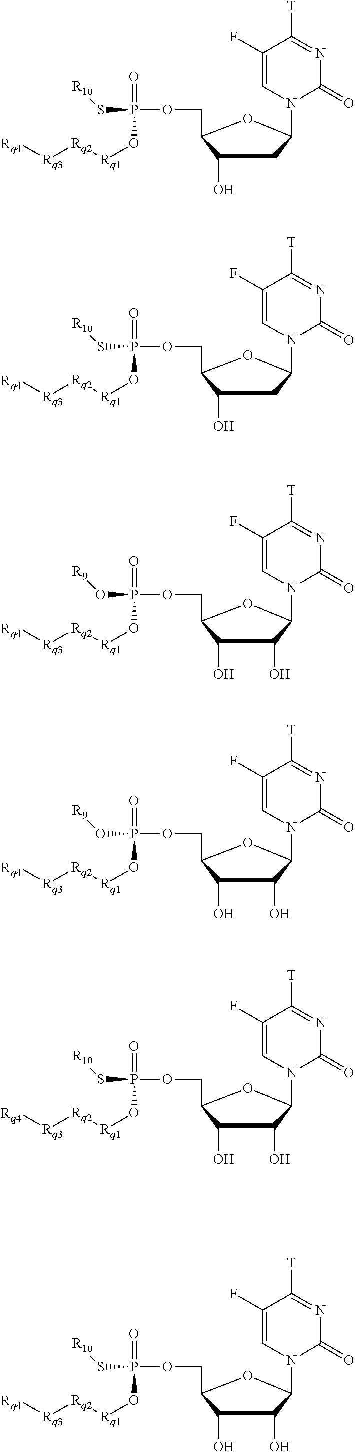

Exemplary lipid-derived prodrugs include, but are not limited to, the following structures and pharmaceutically acceptable salts (such as ammonium and lithium salts) thereof:

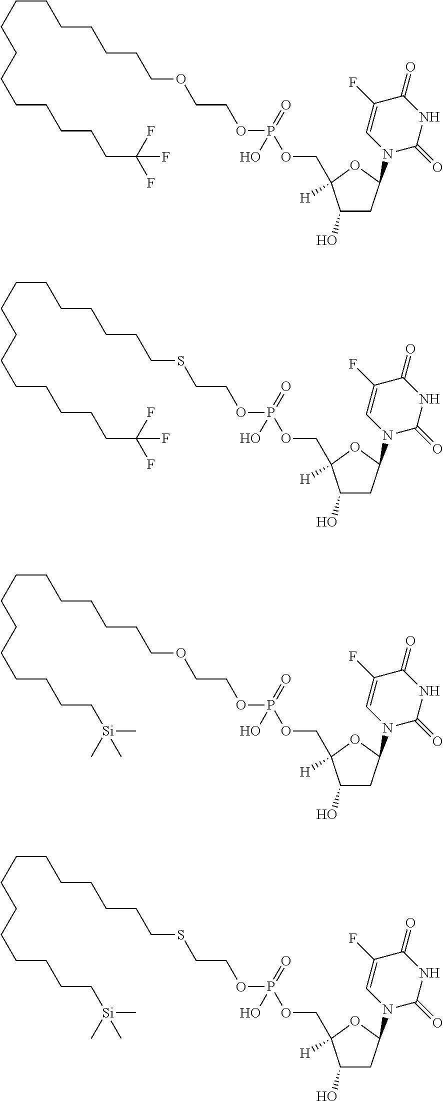 [SMILES: O=c1[nH]c(=O)n([C@H]2C[C@H](O)[C@@H](COP(=O)(O)OCCOCCCCCCCCCCCCCCCC(F)(F)F)O2)cc1F ; O=c1[nH]c(=O)n([C@H]2C[C@H](O)[C@@H](COP(=O)(O)OCCSCCCCCCCCCCCCCCCC(F)(F)F)O2)cc1F ; C[Si](C)(C)CCCCCCCCCCCCCCOCCOP(=O)(O)OC[C@H]1O[C@@H](n2cc(F)c(=O)[nH]c2=O)C[C@@H]1O ; C[Si](C)(C)CCCCCCCCCCCCCCSCCOP(=O)(O)OC[C@H]1O[C@@H](n2cc(F)c(=O)[nH]c2=O)C[C@@H]1O]

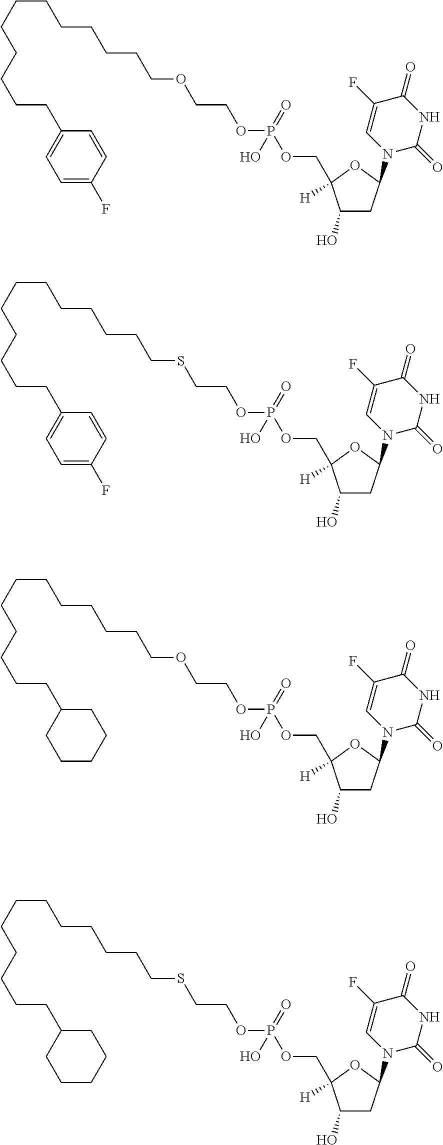 [SMILES: O=c1[nH]c(=O)n([C@H]2C[C@H](O)[C@@H](COP(=O)(O)OCCOCCCCCCCCCCCCc3ccc(F)cc3)O2)cc1F ; O=c1[nH]c(=O)n([C@H]2C[C@H](O)[C@@H](COP(=O)(O)OCCSCCCCCCCCCCCCc3ccc(F)cc3)O2)cc1F ; O=c1[nH]c(=O)n([C@H]2C[C@H](O)[C@@H](COP(=O)(O)OCCOCCCCCCCCCCCCC3CCCCC3)O2)cc1F ; O=c1[nH]c(=O)n([C@H]2C[C@H](O)[C@@H](COP(=O)(O)OCCSCCCCCCCCCCCCC3CCCCC3)O2)cc1F]

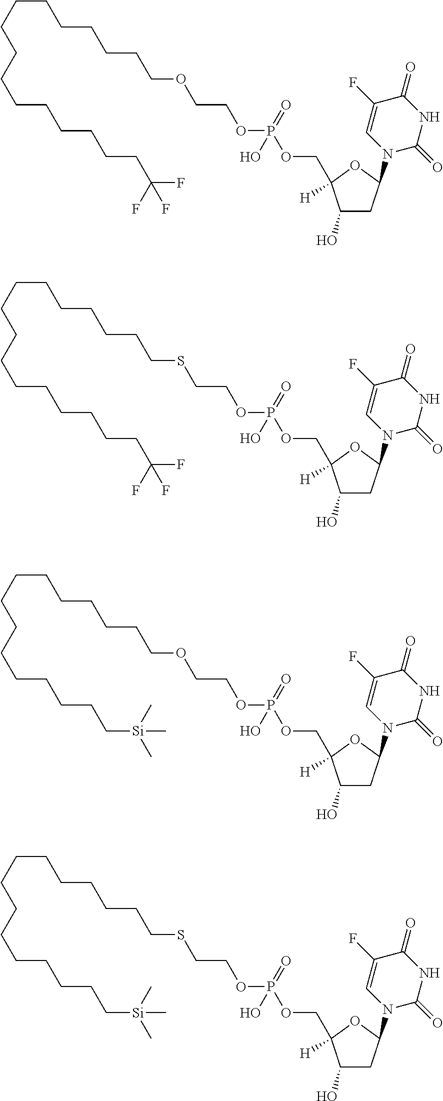 [SMILES: O=c1[nH]c(=O)n([C@H]2C[C@H](O)[C@@H](COP(=O)(O)OCCOCCCCCCCCCCCCCCCCC(F)(F)F)O2)cc1F ; O=c1[nH]c(=O)n([C@H]2C[C@H](O)[C@@H](COP(=O)(O)OCCSCCCCCCCCCCCCCCCCC(F)(F)F)O2)cc1F ; C[Si](C)(C)CCCCCCCCCCCCCCCOCCOP(=O)(O)OC[C@H]1O[C@@H](n2cc(F)c(=O)[nH]c2=O)C[C@@H]1O ; C[Si](C)(C)CCCCCCCCCCCCCCCSCCOP(=O)(O)OC[C@H]1O[C@@H](n2cc(F)c(=O)[nH]c2=O)C[C@@H]1O]

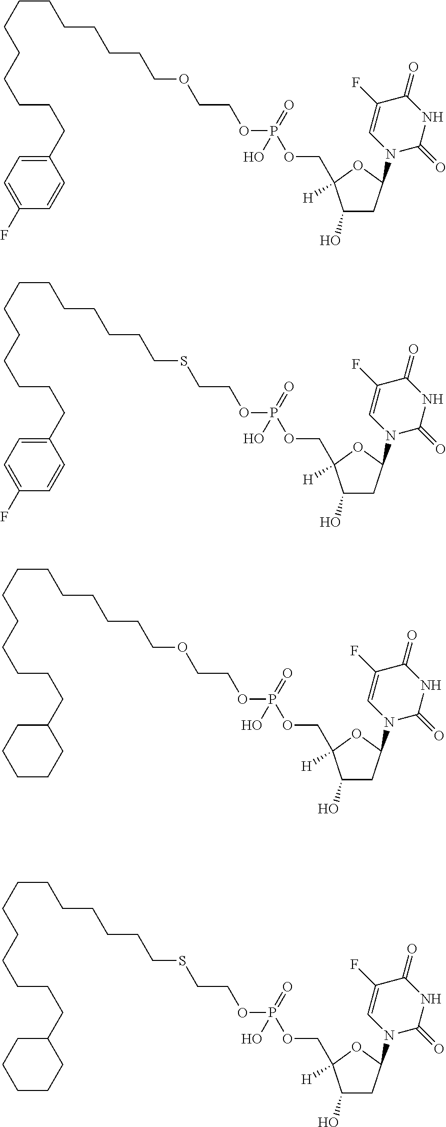 [SMILES: O=c1[nH]c(=O)n([C@H]2C[C@H](O)[C@@H](COP(=O)(O)OCCOCCCCCCCCCCCCCc3ccc(F)cc3)O2)cc1F ; O=c1[nH]c(=O)n([C@H]2C[C@H](O)[C@@H](COP(=O)(O)OCCSCCCCCCCCCCCCCc3ccc(F)cc3)O2)cc1F ; O=c1[nH]c(=O)n([C@H]2C[C@H](O)[C@@H](COP(=O)(O)OCCOCCCCCCCCCCCCCC3CCCCC3)O2)cc1F ; O=c1[nH]c(=O)n([C@H]2C[C@H](O)[C@@H](COP(=O)(O)OCCSCCCCCCCCCCCCCC3CCCCC3)O2)cc1F]

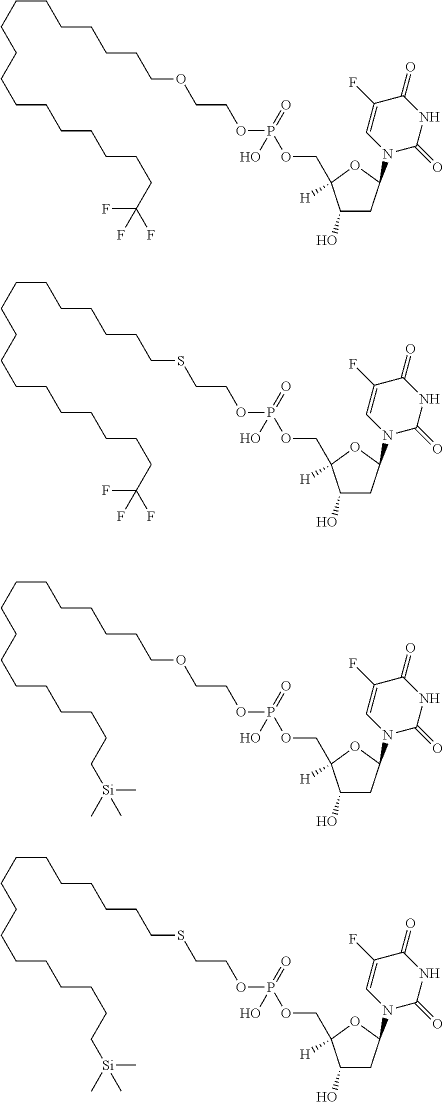 [SMILES: O=c1[nH]c(=O)n([C@H]2C[C@H](O)[C@@H](COP(=O)(O)OCCOCCCCCCCCCCCCCCCCCC(F)(F)F)O2)cc1F ; O=c1[nH]c(=O)n([C@H]2C[C@H](O)[C@@H](COP(=O)(O)OCCSCCCCCCCCCCCCCCCCCC(F)(F)F)O2)cc1F ; C[Si](C)(C)CCCCCCCCCCCCCCCCOCCOP(=O)(O)OC[C@H]1O[C@@H](n2cc(F)c(=O)[nH]c2=O)C[C@@H]1O ; C[Si](C)(C)CCCCCCCCCCCCCCCCSCCOP(=O)(O)OC[C@H]1O[C@@H](n2cc(F)c(=O)[nH]c2=O)C[C@@H]1O]

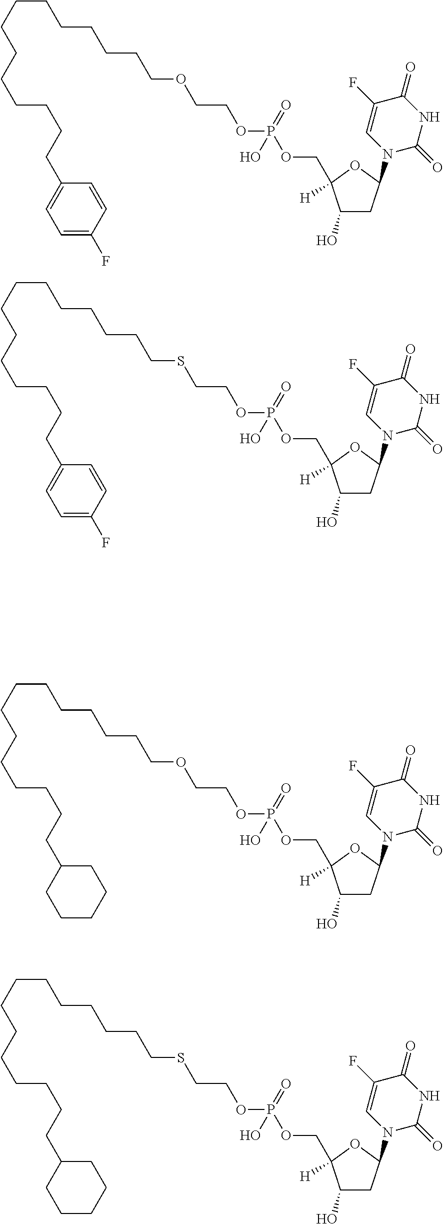 [SMILES: O=c1[nH]c(=O)n([C@H]2C[C@H](O)[C@@H](COP(=O)(O)OCCOCCCCCCCCCCCCCCc3ccc(F)cc3)O2)cc1F ; O=c1[nH]c(=O)n([C@H]2C[C@H](O)[C@@H](COP(=O)(O)OCCSCCCCCCCCCCCCCCc3ccc(F)cc3)O2)cc1F ; O=c1[nH]c(=O)n([C@H]2C[C@H](O)[C@@H](COP(=O)(O)OCCOCCCCCCCCCCCCCCC3CCCCC3)O2)cc1F ; O=c1[nH]c(=O)n([C@H]2C[C@H](O)[C@@H](COP(=O)(O)OCCSCCCCCCCCCCCCCCC3CCCCC3)O2)cc1F]

2. Additional Prodrugs

Additional prodrugs have a structure of Formulas IV, V, IVa, IVb, Va, Vb, IVa′, IVb′, Va′, Vb′, IVa″, IVb″, Va″, or Vb″, or a pharmaceutically acceptable salt thereof, wherein Y is —O—R₉ or —S—R₁₀ and Z is —O—Rᵣ₁—Rᵣ₂, and wherein the other groups in the foregoing formulas are the same as described above.

In some examples, Y is —O—R₉. In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and —Rᵣ₁—Rᵣ₂.

In some examples, R₉ is selected from hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl. In some examples, R₉ is hydrogen. In some examples, R₉ is optionally substituted alkyl, such as benzyl, isopropyl, and 2-ethylbutyl. In some examples, R₉ is optionally substituted aryl, such as phenyl and naphthyl.

In some examples, Y is —S—R₁₀. In some examples, R₁₀ is optionally substituted alkyl, such as benzyl, isopropyl, and 2-ethylbutyl. In some examples, R₁₀ is optionally substituted aryl, such as phenyl and naphthyl.

In some examples, Rᵣ₁ is independently optionally substituted C₁-C₄ bridging alkylene, such as optionally substituted, linear, C₁-C₄ bridging alkylene, e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—. In some examples, Rᵣ₁ is —CH₂CH₂— or —CH₂CH₂CH₂CH₂—.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5. In some examples, Q is —C₆H₄—, such as

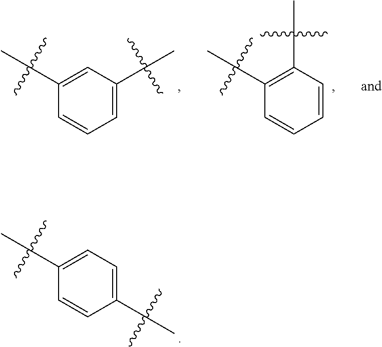 [SMILES: *c1cccc(*)c1 ; *c1ccccc1* ; *c1ccc(*)cc1]

In some examples, Q is —CH₂—C₆H₄—, such as

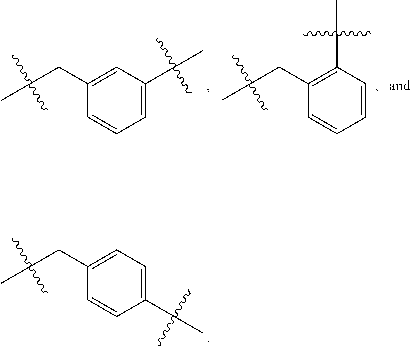 [SMILES: *Cc1cccc(*)c1 ; *Cc1ccccc1* ; *Cc1ccc(*)cc1]

Rᵣ₂ is selected from ester, thioester, amide, acylamino, carbonate ester, carbomate, disulfide, optionally substituted (4-acylamino)phenyl, and optionally substituted (4-acyloxy)phenyl. In some examples, Rᵣ₂ is selected from —C(═O)OR_{c1}, —OC(═O)Rₑ2, —C(═O)SRₒ₁, —SC(═O)Rₒ₂, —C(═O)NR_{f1}R_{f2}, —NR_{g}[C(═O)Rₕ], —OC(═O)ORᵢ, —OC(═O)NRⱼ₁Rⱼ₂, —NRₖ[(C═O)ORᵢ], —S—S—R_{z}, optionally substituted (4-acylamino)phenyl, and optionally substituted (4-acyloxy)phenyl, wherein R_{c1}, R_{c2}, R_{f1}, R_{f2}, R_{g}, Rₕ, Rᵢ, Rⱼ₁, Rⱼ₂, Rₖ, Rₗ, Rₒ₁, Rₒ₂, and R_{z} are the same as described above. In some examples, R_{c1}, R_{c2}, R_{f1}, R_{f2}, Rₕ, Rᵢ, Rⱼ₁, Rⱼ₂, Rₖ, Rₗ, Rₒ₁, Rₒ₂, and R_{z} are independently selected from optionally substituted aryl (such as phenyl or naphthyl) and optionally substituted alkyl (such as benzyl, isopropyl, and 2-ethylbutyl); in this case, “optionally substituted” refers to optionally substituted by one or more R groups described herein. In some examples, R_{c1}, R_{c2}, Rₙ, Rₐ, Rₕ, Rᵢ, Rⱼ₁, Rⱼ₂, Rₖ, Rₗ, Rₒ₁, Rₒ₂, and R_{z} are independently phenyl or naphthyl. In some examples, Rei, Rₑ2, Rₙ, Rₐ, Rₕ, Rᵢ, Rⱼ₁, Rⱼ₂, Rₖ, Rₗ, Rₒ₁, Rₒ₂, and R_{z} are independently benzyl, isopropyl, or 2-ethylbutyl. In some examples, R_{g} and Rₖ are independently hydrogen or optionally substituted alkyl such as methyl; in this case, “optionally substituted” refers to optionally substituted by one or more R groups described herein. In some examples, R_{g} and Rₖ are hydrogen. In some examples, R_{g} and Rₖ are methyl. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or a C₂-C₂₂ alkyl. In some examples, R_{z} is a linear C₁-C₂₀ alkyl or a linear C₂-C₂₂ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₈-C₂₂ alkyl or a linear C₁₁-C₂₀ alkyl.

In some examples, Rᵣ₂ is —S—S—R_{z}. In some examples, R_{z} is alkyl, alkenyl, alkynyl, carbocyclyl, heterocyclyl, aryl, or heteroaryl, optionally substituted by one or more R groups described herein. In some examples, R_{z} is alkyl, optionally substituted by one or more R groups described herein. In some examples, R_{z} is a C₁-C₂₀ alkyl or a C₂-C₂₂ alkyl. In some examples, R_{z} is a linear C₂-C₂₂ alkyl or a linear C₁-C₂₀ alkyl, such as ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), heptylene (—CH₂CH₂CH₂CH₂CH₂CH₂CH₂—), octylene (—CH₂CH₂CH₂CH₂CH₂CH₂CH₂CH₂—), nonylene [—(CH₂)₉—], decylene [—(CH₂)₁₀—], undecylene [—(CH₂)₁₁—], dodecylene [—(CH₂)₁₂—], tridecylene [—(CH₂)₁₃—], tetradecylene [—(CH₂)₁₄—], pentadecylene [—(CH₂)₁₅—], hexadecylene [—(CH₂)₁₆—], heptadecylene [—(CH₂)₁₇—], octadecylene [—(CH₂)₁₈—], nonadecylene [—(CH₂)₁₉—], and icosylene [—(CH₂)₂₀—]. In some examples, R_{z} is a linear C₁-C₁₀ alkyl or a linear C₂-C₇ alkyl. In some examples, R_{z} is a linear C₈-C₂₂ alkyl or a linear C₁₁-C₂₀ alkyl.

In some examples, Rᵣ₂ is —NR_{g}[C(═O)Rₕ]; in some examples, R_{g} is hydrogen or methyl. In some examples, Rᵣ₂ is —OC(═O)ORᵢ. In some examples, Rᵣ₂ is optionally substituted (4-acylamino)phenyl or optionally substituted (4-acyloxy)phenyl; in some examples, Rᵣ₂ is (4-acylamino)phenyl and (4-acyloxy)phenyl.

Exemplary —Rᵣ₁—Rᵣ₂ includes, but is not limited to, —CH₂—OC(═O)ORᵢ, —CH₂CH₂CH₂—NHC(═O)ORₕ, —CH₂CH₂CH₂—S—S—R_{z}, —CH₂CH₂CH₂CH₂—S—S—R_{z}, —CH₂CH₂CH(CH₃)—S—S—R_{z}, —CH₂CH₂CH₂CH(CH₃)—S—S—R_{z}, —CH₂CH₂CH₂—NH[C(═O)Rₕ],

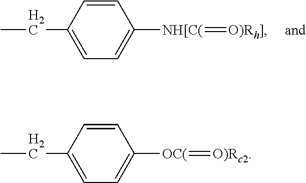

In some examples, Rᵣ₁ is optionally substituted C₁-C₄ bridging alkylene, and Rᵣ₂ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is optionally substituted, linear, C₁-C₄ bridging alkylene (e.g. —CH₂—, —CH₂CH₂—, —CH₂CH₂CH₂—, —CH(CH₃)CH₂—, —CH₂CH(CH₃)—, —CH₂CH₂CH₂CH₂—, —CH(CH₃)CH₂CH₂—, —CH₂CH₂CH(CH₃)—, —CH(CH₃)CH₂CH₂CH₂—, and —CH₂CH₂CH₂CH(CH₃)—), and Rᵣ₂ is —S—S—R_{z}, wherein R_{z} is alkyl, optionally substituted by one or more R groups described herein. In some examples, Rᵣ₁ is —CH₂CH₂— or —CH₂CH₂CH₂CH₂—, and Rᵣ₂ is —S—S—R_{z}, wherein R_{z} is a linear C₂-C₂₂ alkyl or a linear C₁-C₂₀ alkyl.

In some examples, Rᵣ₁ is -Q-CH₂—, wherein Q is —C₆H₄—, —(CH₂)ₙ—, —CH₂—C₆H₄—, an optionally substituted bridging alkylene, an optionally substituted bridging arylene, an optionally substituted bridging carbocyclyl, or an optionally substituted bridging heterocyclyl, wherein n is 1, 2, 3, 4, or 5; and Rₐ is —S—S—R_{z}, wherein R_{z} is alkyl, alkenyl, alkynyl, carbocyclyl, heterocyclyl, aryl, or heteroaryl, optionally substituted by one or more R groups described herein. In some examples, Rᵣ₁ is -Q-CH₂—, wherein Q is —C₆H₄— (such as

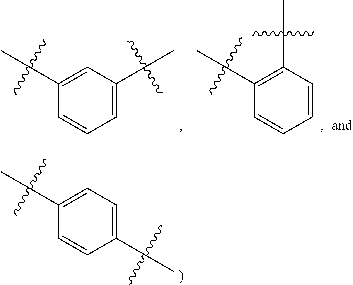 [SMILES: *c1cccc(*)c1 ; *c1ccccc1* ; *c1ccc(*)cc1]

or —CH₂—C₆H₄-(such as

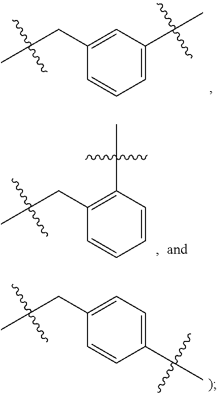

and and Rₐ is —S—S—R_{z}, wherein R_{z} is a linear C₂-C₂₂ alkyl or a linear C₁-C₂₀ alkyl.

When Y is also —O—Rᵣ₁—Rᵣ₂, Y may be the same as or different from Z. In other words, each occurrence of Rᵣ₁ or Rᵣ₂ is independent.

### III. Compositions

The disclosed compounds may be present in a mixture of stereoisomers. In some examples, the compounds in the mixture of stereoisomers may be in greater than 60%, 70%, 80%, 90%, 95%, or 98% diastereomeric or enantiomeric excess. In some examples, the compounds in the mixture of stereoisomers may be in greater than 90% diastereomeric or enantiomeric excess.

The disclosed compounds may be present in a mixture of a salt form and a non-salt form. In some examples, more than 50%, 60%, 70%, 80%, 90%, 95%, or 98% of the compound in the mixture may be in the salt form, calculated as the ratio of the weight of the salt form to the total weight of the salt form and the non-salt form. In some examples, more than 90% of the compound in the mixture may be in the salt form. In some examples, more than 50%, 60%, 70%, 80%, 90%, 95%, or 98% of the compound in the mixture may be in the ammonium salt form, calculated as the ratio of the weight of the ammonium salt form to the total weight of the ammonium salt form and the non-salt form. In some examples, more than 90% of the compound in the mixture may be in the ammonium salt form. In some examples, more than 50%, 60%, 70%, 80%, 90%, 95%, or 98% of the compound in the mixture may be in the lithium salt form, calculated as the ratio of the weight of the lithium salt form to the total weight of the lithium salt form and the non-salt form. In some examples, more than 90% of the compound in the mixture may be in the lithium salt form. In some examples, more than 50%, 60%, 70%, 80%, 90%, 95%, or 98% of the compound in the mixture may be in the non-salt form, calculated as the ratio of the weight of the salt form to the total weight of the salt form and the non-salt form.

### IV. Formulations

Disclosed are pharmaceutical formulations containing a compound disclosed herein. In some examples, the compound is a 5′-substituted nucleoside monophosphate, as disclosed herein. In some examples, the compound is a prodrug of 5′-substituted nucleoside monophosphates, as disclosed herein. In some examples, the compound is a prodrug of a nucleoside monophosphates that is unsubstituted at the 5′ position, as disclosed herein. Generally, the pharmaceutical formulations also contain a pharmaceutically acceptable excipient. Optionally, the pharmaceutical formulations may also contain one or more pharmaceutically active agent, such as other anti-cancer agents.

The pharmaceutical formulations can be in the form of tablet, capsule, pill, caplet, powder, bead, granule, particle, cream, gel, solution (such as aqueous solution, e.g., saline or buffered saline), emulsion, suspension, nanoparticle formulation, etc. In some examples, the pharmaceutical formulations are oral formulations. In some examples, the pharmaceutical formulations are intravenous formulations. In some examples, the pharmaceutical formulations are topical formulations.

In some examples, the 5′-substituted nucleoside monophosphates, as disclosed herein, are formulated in intravenous formulations. In some examples, the prodrugs of 5′-substituted nucleoside monophosphates, as disclosed herein, are formulated in oral formulations. In some examples, the prodrugs of nucleoside monophosphates that are unsubstituted at the 5′ position, as disclosed herein, are formulated in oral formulations.

The pharmaceutical formulations may be prepared in a manner known per se, which usually involves mixing a compound according to the disclosure with the pharmaceutically acceptable excipient, and, if desired, in combination with other pharmaceutical active agent(s), when necessary under aseptic conditions.

As used herein, “emulsion” refers to a composition containing a mixture of non-miscible components homogenously blended together. In some forms, the non-miscible components include a lipophilic component and an aqueous component. For example, an emulsion is a preparation of one liquid distributed in small globules throughout the body of a second liquid. The dispersed liquid is the discontinuous phase, and the dispersion medium is the continuous phase. When oil or oleaginous substance is the dispersed liquid and water or an aqueous solution is the continuous phase, it is known as an oil-in-water emulsion, whereas when water or an aqueous solution is the dispersed phase and oil or oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to degradation or breakdown of a material into its component subunits, or digestion, e.g., by a biochemical process, of the material into smaller (e.g., non-polymeric) subunits.

As used herein, “enteric polymers” refers to polymers that become soluble in the higher pH environment of the lower gastrointestinal tract or slowly erode as the dosage form passes through the gastrointestinal tract.

As used herein, “enzymatically degradable polymers” refer to polymers that are degraded by bacterial enzymes present in the lower gastrointestinal tract, particularly in the colon.

As used herein, “pharmaceutically acceptable” refers to compounds, materials, compositions, and/or formulations which are, within the scope of sound medical judgment, suitable for use in contact with the tissues of human beings and animals without excessive toxicity, irritation, allergic response, or other problems or complications that commensurate with a reasonable benefit/risk ratio, in accordance with the guidelines of agencies such as the Food and Drug Administration.

As used herein, “nanoparticle” generally refers to particles having a diameter from about 1 nm to 1000 nm, preferably from about 10 nm to 1000 nm, more preferably from about 100 nm to 1000 nm, most preferably from about 250 nm to 1000 nm. In some examples, “nanoparticles” can also refer to “microparticles,” which are particles having a diameter from about 1 micron to about 100 microns, preferably from about 1 to about 50 microns, more preferably from about 1 to about 30 microns, most preferably from about 1 micron to about 10 microns. In some examples, the nanoparticles can be a mixture of nanoparticles, as defined above, and microparticles, as defined above.

As used herein, the term “surfactant” refers to any agent which preferentially absorbs to an interface between two immiscible phases, such as the interface between water and an organic polymer solution, a water/air interface, or organic solvent/air interface. Surfactants generally possess a hydrophilic moiety and a lipophilic moiety.

As used herein, “gel” is a semisolid system containing a dispersion of the active agent, i.e., a compound disclosed herein, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely-dispersed polymer chains that are water-insoluble, where the polymeric molecules are in the external or dispersion phase and water (or an aqueous solution) forms the internal or dispersed phase. The polymer chains can be chemically cross-linked (chemical gels) or physically cross-linked (physical gels). Chemical gels possess polymer chains that are connected through covalent bonds, whereas physical gels have polymer chains linked by non-covalent interactions, such as van der Waals interactions, ionic interactions, hydrogen bonding interactions, or hydrophobic interactions.

As used herein, drug-containing “beads” refer to beads made with drug and one or more excipients. Drug-containing beads can be produced by applying drug to an inert support, e.g., inert sugar beads coated with drug or by creating a “core” comprising both drug and the one or more excipients. As is also known, drug-containing “granules” and “particles” comprise drug particles that may or may not include one or more additional excipients. Typically, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

A. Physical Forms and Unit Dosages

Depending upon the manner of introduction, the compounds described herein may be formulated in a variety of ways. The pharmaceutical formulations can be prepared in various forms, such as granules, tablets, capsules, pills, caplets, suppositories, powders, controlled release formulations, nanoparticle formulations, solutions (such as aqueous solutions, e.g., saline, buffered saline), suspensions, emulsions, creams, gels, ointments, salves, lotions, aerosols, and the like.

In some examples, the pharmaceutical formulations are in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspensions (including nano- or microsuspensions), shakes, emulsions, etc. can also be utilized. Intravenous formulations are usually in liquid dosage forms, including solutions, emulsions, and suspensions. Suitable topical formulations include, but are not limited to, lotions, ointments, creams, and gels. In some examples, the topical formulations are in the form of gels or creams.

In some examples, the pharmaceutical formulations are in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages contain between 1 and 1000 mg, and usually between 5 and 500 mg, of at least one compound from the disclosure, e.g., about 10, 25, 50, 100, 200, 300 or 400 mg per unit dosage.

The concentration of the compound to the pharmaceutically acceptable excipient may vary from about 0.5 to about 100 wt %. For oral use, the pharmaceutical formulations generally contain from about 5 to about 100 wt % of the compound. For other uses, the pharmaceutical formulations generally have from about 0.5 to about 50 wt % of the compound.

B. Pharmaceutically Acceptable Excipients

As used herein, “excipient” refers to all components present in the pharmaceutical formulations other than the active ingredient(s). Pharmaceutically acceptable excipients are composed of materials that are considered safe and may be administered to an individual without causing undesirable biological side effects or unwanted interactions. For example, the pharmaceutically acceptable excipients can be compounds or materials recognized by the U.S. Food & Drug Administration as “generally recognized as safe” or “GRAS”.

Generally, excipients include, but are not limited to, diluents (fillers), binders, lubricants, disintegrants, pH-modifying or buffering agents, preservatives, antioxidants, solubility enhancers, wetting or emulsifying agents, plasticizers, colorants (such as pigments and dyes), flavoring or sweetening agents, thickening agents, emollients, humectants, stabilizers, glidants, solvent or dispersion medium, surfactants, pore formers, and coating or matrix materials.

In some examples, drug-containing tablets, beads, granules or particles contain one or more of the following excipients: diluents, binders, lubricants, disintegrants, pigments, stabilizers, and surfactants. If desired, the tablets, beads, granules, or particles may also contain minor amount of nontoxic auxiliary substances such as wetting or emulsifying agents, dyes, pH-buffering agents, or preservatives.

Examples of the coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate, ethyl methacrylate, and other methacrylic resins that are commercially available under the tradename EUDRAGIT®), enzymatically degradable polymers (such as azo polymers, pectin, chitosan, amylose and guar gum), zein, shellac, and polysaccharides. In some examples, the coating or matrix materials may contain one or more conventional excipients such as plasticizers, colorants, glidants, stabilizers, pore formers, and surfactants.

In some examples, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above, as a result of a higher degree of esterification.

In some examples, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some examples, the coating or matrix materials are selected such that the pharmaceutical formulations can survive exposure to gastric acid and release the compound in the intestines after oral administration.

Diluents, also referred to as “fillers,” can increase the bulk of a solid dosage formulation so that a practical size is provided for compression of tablets or formation of beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a tablet, bead, granule, or particle remains intact after the formation of the solid dosage formulation. Suitable binder materials include, but are not limited to, starch, pregelatinized starch, gelatin, sugars (such as sucrose, glucose, dextrose, lactose and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, and synthetic polymers (such as acrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked polyvinylpyrrolidone (e.g., POLYPLASDONE® XL from GAF Chemical Corp.).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active agents in the formulations or stabilize particles in a dispersion. For example, when the decomposition reactions involve an oxidation reaction of an active agent in the formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidants include, but are not limited to talc, magnesium stearate, and glycerol monostearates.

Pigments such as titanium dioxide may also be used.

Preservatives can inhibit the deterioration and/or decomposition of a pharmaceutical formulation. Deterioration or decomposition can be brought about by any of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, propionate salts (e.g., sodium propionate), chlorobutanol, benzyl alcohol, and combinations thereof.

Surfactants may be anionic, cationic, amphoteric or nonionic surface-active agents. Exemplary anionic surfactants include, but are not limited to, those containing a carboxylate, sulfonate or sulfate ion. Examples of anionic surfactants include sodium, potassium, ammonium of long chain (e.g., 13-21) alkyl sulfonates (such as sodium lauryl sulfate), alkyl aryl sulfonates (such as sodium dodecylbenzene sulfonate), and dialkyl sodium sulfosuccinates (such as sodium bis-(2-ethylthioxyl)-sulfosuccinate). Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, poloxamers (such as poloxamer 401), stearoyl monoisopropanolamide, and polyoxyethylene hydrogenated tallow amide. Examples of amphoteric surfactants include, but are not limited to, sodium N-dodecyl-β-alanine, sodium N-lauryl-β-iminodipropionate, myristoamphoacetate, lauryl betaine, and lauryl sulfobetaine.

Pharmaceutical formulations in liquid forms typically contain a solvent or dispersion medium such as water, aqueous solution (such as saline and buffered saline), ethanol, polyol (such as glycerol, propylene glycol, and liquid polyethylene glycol), oil (such as vegetable oil, e.g., peanut oil, corn oil, sesame oil, etc.), and combinations thereof. Preferably, the pharmaceutical formulations in liquid forms are aqueous formulations. Suitable solvent or dispersion medium for intravenous formulations include, but are not limited to, water, saline, buffered saline (such as phosphate-buffered saline), and Ringer's solution.

C. Pharmaceutical Acceptable Carriers

In some examples, the pharmaceutical formulations are prepared using a pharmaceutically acceptable carrier, which encapsulates, embeds, entraps, dissolves, disperses, absorbs, and/or binds to a compound disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound in performing its function. The pharmaceutically acceptable carrier can be formed of biodegradable materials, non-biodegradable materials, or combinations thereof. The pharmaceutically acceptable excipient described above may be partially or entirely present in the pharmaceutical acceptable carrier.

In some examples, the pharmaceutical acceptable carrier is a controlled-release carrier, such as delayed-release carriers, sustained-release (extended-release) carriers, and pulsatile-release carriers.

In some examples, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to, nanoparticles, liposomes, hydrogels, polymer matrices, and solvent systems.

In some examples, the pharmaceutical acceptable carrier is nanoparticles. In some forms, the compound is embedded in the matrix formed by materials of the nanoparticles.

The nanoparticles can be biodegradable, and preferably are capable of biodegrading at a controlled rate for delivery of the compound. The nanoparticles can be made of a variety of materials. Both inorganic and organic materials can be used. Both polymeric and non-polymeric materials can be used.

Preferably, the nanoparticles are polymeric nanoparticles formed of one or more biocompatible polymers, copolymers, or blends thereof. In some forms, the biocompatible polymers are biodegradable. In some forms, the biocompatible polymers are non-biodegradable. In some forms, the nanoparticles are formed of a mixture of biodegradable and non-biodegradable polymers. The polymers may be tailored to optimize different characteristics of the nanoparticles including: (i) interactions between the compound and the polymer to provide stabilization of the compound and retention of activity upon delivery; (ii) rate of polymer degradation and, thereby, rate of release; (iii) surface characteristics and targeting capabilities via chemical modification; and (iv) particle porosity.

Exemplary polymers include, but are not limited to, polymers prepared from lactones such as poly(caprolactone) (PCL), polyhydroxy acids and copolymers thereof such as poly(lactic acid) (PLA), poly(glycolic acid) (PGA), poly(lactic acid-co-glycolic acid)(PLGA), and blends thereof, polyalkyl cyanoacralate, polyurethanes, polyamino acids such as poly-L-lysine (PLL), poly(valeric acid), and poly-L-glutamic acid, hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), celluloses including derivatized celluloses such as alkyl celluloses, hydroxyalkyl celluloses, cellulose ethers, cellulose esters, nitro celluloses, hydroxypropylcellulose, and carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”), polydioxanone and its copolymers, polyhydroxyalkanoates, polypropylene fumarate, polyoxymethylene, poloxamers, poly(butyric acid), trimethylene carbonate, polyphosphazenes, polysaccharides, peptides or proteins, and copolymers or blends thereof.

Preferably, the polymer is an FDA approved biodegradable polymer such as polyhydroxy acids (e.g., PLA, PLGA, PGA), polyanhydride, polyhydroxyalkanoate such as poly(3-butyrate) or poly(4-butyrate), and copolymer or blends thereof.

Materials other than polymers may be used to form the nanoparticles. Suitable materials include excipients such as surfactants.

The use of surfactants in the nanoparticles may improve surface properties by, for example, reducing particle-particle interactions, and render the surface of the particles less adhesive. Both naturally occurring surfactants and synthetic surfactants can be incorporated into the nanoparticles. Exemplary surfactants include, but are not limited to, phosphoglycerides such as phosphatidylcholines (e.g., L-α-phosphatidylcholine dipalmitoyl), diphosphatidyl glycerol, hexadecanol, fatty alcohols, polyoxyethylene-9-lauryl ether, fatty acids such as palmitic acid or oleic acid, sorbitan trioleate, glycocholate, surfactin, poloxomers, sorbitan fatty acid esters such as sorbitan trioleate, tyloxapol, and phospholipids.

The nanoparticles can contain a plurality of layers. The layers can have similar or different release kinetic profiles for the compound. For example, the nanoparticles can have a controlled-release core surrounded by one or more additional layers. The one or more additional layers can include an instant-release layer, preferably on the surface of the nanoparticles. The instant-release layer can provide a bolus of the compound shortly after administration.

The composition and structure of the nanoparticles can be selected such that the nanoparticles are pH-sensitive or pH-responsive. In some forms, the particles are formed of pH-sensitive or pH-responsive polymers such as the enteric polymers commercially available under the tradename EUDRAGIT®, as described above. Depending on the particle materials, the decomposition/degradation or structural change of the nanoparticles may occur at different locations of the gastrointestinal tract. In some examples, the particle materials are selected such that the pharmaceutical formulations can survive exposure to gastric acid and release the compound in the intestines after oral administration.

D. Controlled Release

In some examples, the pharmaceutical formulations can be controlled release formulations. Examples of controlled release formulations include extended release formulations, delayed release formulations, pulsatile release formulations, and combinations thereof. In some examples, each dosage unit in capsule may contain a plurality of drug-containing beads, granules or particles, having different release profiles.

1. Extended Release

In some examples, the extended release formulations are prepared as diffusion or osmotic systems, for example, as described in “Remington—The science and practice of pharmacy” (20th Ed., Lippincott Williams & Wilkins, 2000).

A diffusion system is typically in the form of a matrix, generally prepared by compressing the drug with a slowly dissolving carrier, optionally into a tablet form. The three major types of materials used in the preparation of the matrix are insoluble plastics, hydrophilic polymers, and fatty compounds. Plastic matrices include, but are not limited to, methyl acrylate-methyl methacrylate copolymer, polyvinyl chloride, and polyethylene. Hydrophilic polymers include, but are not limited to, cellulosic polymers such as methyl ethyl cellulose, hydroxyalkylcelluloses (such as hydroxypropylcellulose, hydroxypropylmethylcellulose), sodium carboxymethylcellulose, CARBOPOL® 934, polyethylene oxides, and mixtures thereof. Fatty compounds include, but are not limited to, various waxes such as carnauba wax and glyceryl tristearate, wax-type substances including hydrogenated castor oil and hydrogenated vegetable oil, and mixtures thereof.

In some examples, the plastic is a pharmaceutically acceptable acrylic polymer, including but not limited to, acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid)(anhydride), polymethacrylate, polyacrylamide, poly(methacrylic acid anhydride), and glycidyl methacrylate copolymers.

In some examples, the acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some examples, the acrylic polymer is an acrylic resin lacquer such as those commercially available under the tradename EUDRAGIT®. In some examples, the acrylic polymer contains a mixture of two acrylic resin lacquers, EUDRAGIT® RL30D and EUDRAGIT® RS30D. EUDRAGIT® RL30D and EUDRAGIT® RS30D are copolymers of acrylic and methacrylic acid esters with a low content of quaternary ammonium groups, the molar ratio of ammonium groups to the remaining neutral methacrylic esters being 1:20 in EUDRAGIT® RL30D and 1:40 in EUDRAGIT® RS30D. In some examples, the mean molecular weight for both copolymers is about 150,000. The code designations RL (high permeability) and RS (low permeability) refer to the permeability properties of these polymers. EUDRAGIT® RL/RS mixtures are insoluble in water and in digestive fluids. However, multiparticulate systems formed to include the same are swellable and permeable in aqueous solutions and digestive fluids. In some examples, the acrylic polymer can also be or include other EUDRAGIT® acrylic resin lacquers, such as EUDRAGIT® S—100, EUDRAGIT® L-100, or a mixture thereof.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, to 50% EUDRAGIT® RL+50% EUDRAGIT® RS, and to 10% EUDRAGIT® RL+90% EUDRAGIT® RS.

Matrices with different drug release mechanisms described above can be combined in a final dosage form containing single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Alternatively, extended release formulations can be prepared using osmotic systems or by applying a semi-permeable coating to a solid dosage form. In the latter case, the desired drug release profile can be achieved by combining low permeable and high permeable coating materials in suitable proportions.

2. Delayed Release

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a coating material. The drug-containing composition may be a tablet for incorporation into a capsule, a tablet for use as an inner core in a “coated core” dosage form, or a plurality of drug-containing beads, particles or granules, for incorporation into either a tablet or capsule. Suitable coating materials include bioerodible, gradually hydrolyzable, gradually water-soluble, and/or enzymatically degradable polymers, such as those described above. In some examples, the coating material is or contains enteric polymers. Combinations of different coating materials may also be used. Multilayer coatings using different polymers may also be applied.

The preferred coating weights for particular coating materials may be readily determined by those skilled in the art by evaluating individual release profiles for tablets, beads and granules prepared with different quantities of the coating materials.

The coating materials may contain conventional additives, such as plasticizers (generally represent about 10 wt % to 50 wt % relative to the dry weight of the coating material), colorants, stabilizers, glidants, etc., such as those described above.

3. Pulsatile Release

Pulsatile-release formulations release a plurality of drug doses at spaced-apart time intervals. Generally, upon administration, such as ingestion, of the pulsatile-release formulations, release of the initial dose is substantially immediate, e.g., the first drug release “pulse” occurs within about one hour of administration. This initial pulse is followed by a first time-interval (lag time) during which very little or no drug is released from the formulations, after which a second dose is then released. Similarly, a second lag time (nearly drug release-free interval) between the second and third drug release pulses may be designed. The duration of the lag times will vary depending on the formulation design, especially on the length of the desired drug administration interval, e.g., a twice daily dosing profile, a three times daily dosing profile, etc.

For pulsatile-release formulations providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In some forms, the pulsatile-release formulations contain a plurality of drug carriers with different drug-release kinetics.

In some forms, the pulsatile-release formulations contain a drug carrier with a plurality of drug-loaded layers. The drug-loaded layers may have different drug release kinetics. The layers may be separated by a delayed-release coating. For example, the carrier may have a drug-loaded layer on the surface for the first pulse and a drug-loaded core for the second pulse; the drug-loaded core may be surrounded by a delayed-release coating, which creates a lag time between the two pulses.

In some examples, the pulsatile release profile is achieved with formulations that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the drug in a controlled release polymer matrix. Each dosage unit may comprise a compressed or molded tablet, wherein each tablet within the capsule provides a different drug release profile.

E. Exemplary Formulations for Different Routes of Administration

A subject suffering from a condition, disorder or disease as described herein, can be treated by either targeted or systemic administration, via oral, inhalation, topical, trans- or sub-mucosal, subcutaneous, parenteral, intramuscular, intravenous, or transdermal administration of a pharmaceutical formulation containing a compound or composition described herein. In some examples, the pharmaceutical formulation is suitable for oral administration. In some examples, the pharmaceutical formulation is suitable for inhalation or intranasal administration. In some examples, the pharmaceutical formulation is suitable for transdermal or topical administration. In some examples, the pharmaceutical formulation is suitable for subcutaneous, intravenous, intraperitoneal, intramuscular, parenteral, or submucosal administration.

In some examples, the pharmaceutical formulation is an oral pharmaceutical formulation. In some examples, the active ingredient may be incorporated with one or more pharmaceutically acceptable excipients as described above and used in the form of tablets, pills, caplets, or capsules. For example, the corresponding oral pharmaceutical formulation may contain one or more of the following pharmaceutically acceptable excipients or those of a similar nature: a binder as described above, a disintegrant as described above, a lubricant as described above, a glidant as described above, a sweetening agent (such as sucrose and saccharin), and a flavoring agent (such as methyl salicylate and fruit flavorings). In some examples, when the oral pharmaceutical formulation is in the form of capsules, it may contain, in addition to the material(s) listed above, a liquid carrier (such as a fatty oil). In some examples, when the oral pharmaceutical formulation is in the form of capsules, each capsule may contain a plurality of beads, granules, and/or particles of the active ingredient. In some examples, the oral pharmaceutical formulation may contain one or more other materials which modify the physical form or one or more pharmaceutical properties of the dosage unit, for example, coatings of polysaccharides, shellac, or enteric polymers as described in previous sections.

In some examples, the oral pharmaceutical formulation can be in the form of an elixir, suspension, syrup, wafer, chewing gum or the like. A syrup may contain, in addition to the active ingredient, one or more sweetening agents (such as sucrose and saccharine), one or more flavoring agents, one or more preservatives, and/or one or more dyes or colorings.

In some examples, the pharmaceutical formulation is a parenteral pharmaceutical formulation. In some examples, the parenteral pharmaceutical formulation can be enclosed in an ampoule, syringe, or a single or multiple dose vial made of glass or plastic. In some examples, the parenteral pharmaceutical formulation is an intravenous pharmaceutical formulation. In some examples, the intravenous pharmaceutical formulation contains a liquid, pharmaceutically acceptable carrier for the active ingredient. Suitable liquid, pharmaceutically acceptable carriers include, but are not limited to, physiological saline, bacteriostatic water, Cremophor EL™ (BASF, Parsippany, NJ), phosphate buffered saline (PBS), and combinations thereof.

In some examples, the pharmaceutical formulation is a topical pharmaceutical formulation. Suitable forms of the topical pharmaceutical formulation include lotions, suspensions, ointments, creams, gels, tinctures, sprays, powders, pastes, slow-release transdermal patches, and suppositories for application to rectal, vaginal, nasal or oral mucosa.

In some examples, thickening agents, emollients (such as mineral oil, lanolin and its derivatives, and squalene), humectants (such as sorbitol), and/or stabilizers can be used to prepare the topical pharmaceutical formulations. Examples of thickening agents include petrolatum, beeswax, xanthan gum, and polyethylene.

In some examples, the active ingredient is prepared with a pharmaceutically acceptable carrier that will protect it against rapid degradation or elimination from the body of the subject after administration, such as the controlled-release formulations as described in previous sections.

### V. Methods of Making

Methods of making exemplary compounds are disclosed. The methods are compatible with a wide variety of functional groups and compounds, and thus a wide variety of compounds can be obtained from the disclosed methods.

For example, methods for introducing substituents at the 5′ position, with different types of stereochemistry, to nucleoside monophosphates, are described in the examples.

Methods for making ProTide prodrugs are well known in the art (e.g., Mehellou, et al., J. Med. Chem., 2018, 61, 6, 2211-2226) and described in the examples.

Methods for making lipid-derived prodrugs of nucleoside monophosphates, especially the 5′-substituted nucleoside monophosphates, typically involve phosphate mono-esterification. Exemplary methods of performing such mono-esterification reactions are described in the examples and PCT Patent Application No. PCT/US2020/047631. For example, depending on the properties of the lipid-like moiety (i.e., —R_{Q}—R_{q2}—R_{q3}—R₄) to be incorporated to the nucleoside monophosphates, the synthetic method may involve the use of DCC or EDC as the coupling agent in the presence of triethylamine (TEA) and DMAP. The reaction can be conducted under high heat (e.g., 90-105° C.) for 18-24 hours, and the product can be purified immediately after a quench with water. The purification of the product can be performed using a sequential normal (DCM:MeOH:NH₄Cl) and reverse (H₂O:MeOH) phase column chromatography approach. In some examples, a deprotection step either in NH₃/MeOH or AcOH/MeOH can be performed to produce the unprotected product. Alternatively, the coupling reactions between the nucleoside monophosphates and the lipid-like moiety can be performed via microwave-assisted synthesis using cyanotrichloromethane as a coupling agent, as demonstrated in the examples. Methods for making lipid disulfide prodrugs are described in U.S. Patent Application Publication No. 2020/0306272.

Introducing the “T” group in the nucleobase in Formulas III, IIIa, IIIa′, IIIb, IIIb′, V, Va, Va′, Va″, Vb, Vb′, and Vb″ can be performed using methods described in U.S. Pat. No. 5,472,949.

Methods of making pharmaceutical formulations are generally known in the art. Exemplary methods can be found in the following references and references cited therein: Lieberman, et al., Pharmaceutical Dosage Forms: Tablets, Marcel Dekker, Inc., New York, 1989; Ansel, et al., Pharmaceutical Dosage Forms and Drug Delivery Systems, 6th Ed., Williams & Wilkins, Media, P A, 1995; Remington—The Science and Practice of Pharmacy, 20th Ed., Lippincott Williams & Wilkins, Baltimore, M D, 2000. Delayed release, extended release, and/or pulsatile release formulations may be prepared as described in the references described above. These references provide information on carriers, materials, equipments, and processes for preparing tablets, capsules, and granules, as well as controlled release forms of the tablets, capsules, and granules.

Techniques for making nanoparticles are known in the art and include, but are not limited to, solvent evaporation, solvent removal, spray drying, phase inversion, low temperature casting, and nanoprecipitation, for example, as described in WO/2013/110028. In some forms, the compound, other pharmaceutically active agent(s), and/or pharmaceutically acceptable excipient(s) can be incorporated into the nanoparticles during particle formation. Methods for making nanoparticles for delivery of encapsulated agents are described in the literature, for example, as described in Doubrow, Ed., Microcapsules and Nanoparticles in Medicine and Pharmacy, CRC Press, Boca Raton, 1992. Methods are also described in Mathiowitz and Langer, J. Controlled Release, 5, 13-22 (1987); Mathiowitz, et al., Reactive Polymers, 6, 275-283 (1987); and Mathiowitz, et al., J. Appl. Polymer Sci., 35, 755-774 (1988). Selection of the method depends on the polymer structure, size of the nanoparticles, external morphology, and crystallinity that is desired, as described, for example, by Mathiowitz, et al., Scanning Microscopy, 4, 329-340 (1990); Mathiowitz, et al., J. Appl. Polymer Sci., 45, 125-134 (1992); and Benita, et al., J. Pharm. Sci., 73, 1721-1724 (1984).

Techniques for making liposomes and hydrogels are also known in the art, for example, as described in U.S. Patent Application Publication Nos. 2017/0281541, 2017/0100342, and 2018/0021435.

### VI. Methods of Using

Methods for treating cancer in a subject in need thereof are disclosed.

The methods generally include administering an effective amount of a 5′-substituted nucleoside monophosphate, as disclosed herein, or a prodrug thereof, also as disclosed herein, to the subject. In some examples, the 5′-substituted nucleoside monophosphate or the prodrug thereof can be administered in the form of a pharmaceutical formulation, such as those described above. The 5′-substituted nucleoside monophosphate or the prodrug thereof can be administered in a variety of manners, depending on whether local or systemic administration is desired.

Alternatively, the methods include administering an effective amount of a prodrug of nucleoside monophosphates that is unsubstituted at the 5′ position, as disclosed herein, to the subject. In some examples, the prodrug can be administered in the form of a pharmaceutical formulation, such as those described above. The prodrug can be administered in a variety of manners, depending on whether local or systemic administration is desired.

In some examples, the compounds mentioned above are directly administered to a specific bodily location of the subject, e.g., topical administration. In some examples, the compounds are administered in a systemic manner, such as enteral administration (e.g., oral administration) or parenteral administration (e.g., injection, infusion, and implantation). Exemplary administration routes include oral administration, intravenous administration such as intravenous injection or infusion, and topical administration.

In some examples, the 5′-substituted nucleoside monophosphate is administered, optionally via intravenous administration. In some examples, the prodrug of 5′-substituted nucleoside monophosphates is administered, optionally via oral administration. In some examples, the prodrug of nucleoside monophosphates that is unsubstituted at the 5′ position is administered, optionally via oral administration.

The cancer to be treated can be selected from breast cancers, head and neck cancers, anal cancers, stomach cancers, skin cancers (e.g., melanoma), colon and rectal cancers, pancreatic cancers, esophageal cancers, gastrointestinal cancers, thymic cancers, cervical cancers, bladder cancers, hepatobiliary cancers, thyroid cancers, ovarian cancers, prostate cancers, endometrial cancers, small cell and non-small cell lung cancers, gallbladder cancers, testicular cancers, neuroendocrine tumors, leukemias, lymphomas, hepatocellular carcinomas, renal cell carcinomas, sarcomas, mesotheliomas, multiple myelomas, glioblastomas, neuroblastomas, and gliomas. In some examples, the cancer is a hepatobiliary cancer. In some examples, the cancer is a colorectal cancer. Optionally, the methods described herein can include selecting a subject having cancer.

The compound can be administered during a period before, during, or after onset of one or more symptoms of the cancer, or any combination of periods before, during or after onset of the symptoms.

The efficacy of administration of the compound according to the methods described herein can be determined by evaluating different aspects of the medical history, signs, symptoms, and objective laboratory tests that are known to be useful in evaluating the status of the subject in need of treatment. These signs, symptoms, and objective laboratory tests may vary, depending upon the cancer being treated. For example, if, based on a comparison with an appropriate control group and/or knowledge of the normal progression of the cancer in the general population: (1) the subject's physical condition is shown to be improved, (2) the progression of the cancer is shown to be stabilized, slowed, or reversed, and/or (3) the need for other medications for treating the cancer is lessened or obviated, then a particular treatment regimen is considered efficacious. In some forms, the efficacy of the compound in treating the cancer can be determined by monitoring the progression of the cancer, such as monitoring tumor size or aggression using invasive (e.g., biopsy) and/or non-invasive (e.g., MRI) methods.

As used herein, “effective amount” of a material refers to a nontoxic but sufficient amount of the material to provide the desired result. The exact amount required may vary from subject to subject, depending on the species, age, and general condition of the subject, the severity of the disease, disorder, or condition that is being treated, the particular drug or therapy used, its mode of administration, and the like.

In some examples, the 5′-substituted nucleoside monophosphate, the prodrug of 5′-substituted nucleoside monophosphates, or the prodrug of nucleoside monophosphates that is unsubstituted at the 5′ position is administered at a dosage that is lower than the lower limit of the FDA recommended dosage of 5-fluorouracil for gastric adenocarcinoma, i.e., 1.54 mM per m². In some examples, the 5′-substituted nucleoside monophosphate, the prodrug of 5′-substituted nucleoside monophosphates, or the prodrug of nucleoside monophosphates that is unsubstituted at the 5′ position is administered orally as a dosage that is lower than 1.50 mM per m², 1.20 mM per m², 1.00 mM per m², or 0.80 mM per m².

In some examples, the 5′-substituted nucleoside monophosphate, the prodrug of 5′-substituted nucleoside monophosphates, or the prodrug of nucleoside monophosphates that is unsubstituted at the 5′ position is administered at a dosage that is lower than the lower limit of the FDA recommended dosage of 5-fluorouracil for the same indication.

In some examples, the 5′-substituted nucleoside monophosphate, the prodrug of 5′-substituted nucleoside monophosphates, or the prodrug of nucleoside monophosphates that is unsubstituted at the 5′ position is administered at a dosage that is higher than the upper limit of the FDA recommended dosage of 5-fluorouracil for the same indication.

In some examples, the 5′-substituted nucleoside monophosphate, the prodrug of 5′-substituted nucleoside monophosphates, or the prodrug of nucleoside monophosphates that is unsubstituted at the 5′ position can be administered in combination with one or more additional pharmaceutically active agents, such as other anti-cancer agents. The one or more additional pharmaceutically active agents can be formulated in the same pharmaceutical formulation as the 5′-substituted nucleoside monophosphate, the prodrug of 5′-substituted nucleoside monophosphates, or the prodrug of nucleoside monophosphates that is unsubstituted at the 5′ position. Alternatively, the one or more additional pharmaceutically active agents can be formulated in separate pharmaceutical formulation(s). As used herein, “combination with” means that the compound may be administered prior to, together with, or after the additional pharmaceutically active agents, or a combination thereof.

In some examples, the one or more additional pharmaceutically active agents are selected from, but not limited to: leucovorin; docetaxel, paclitaxel, cabazitaxel, etoposide, ixabepilone, vinorelbine, vinblastine, teniposide, vincristine, and eribulin; doxorubicin, daunorubicin, epirubicin, idarubicin, valrubicin, and mitoxantrone; cisplatin, oxaliplatin, and carboplatin; ifosfamide, busulfan, cyclophosphamide, carmustine, bendamustine, melphalan, lomustine, chloromethine, thiotepa, and mitotane; irinotecan, camptothecin, SN-38, and topotecan; gemcitabine, cytarabine, decitabine, azacytidine, cladribine, fludarabine, nelarabine, clofarabine, tioguanine, azathioprine, mercaptopurine, and prodrugs thereof; methotrexate, pemetrexed, pralatrexate, proguanil, pyrimethamine, and trimethoprim; ribociclib, palbociclib, and abemaciclib; pazopanib, sunitinib, sorafenib, axitinib, regorafenib, ponatinib, afatinib, cabozantinib, vandetanib, lenvatinib, gefitinib, erlotinib, lapatinib, neratinib, osimertinib, imatinib, dasatinib, nilotinib, bosutinib, nintedanib, crizotinib, trametinib, dabrafenib, midostaurin, vemurafenib, ruxolitinib, baricitinib, ibrutinib, brigatinib, alectinib, encorafenib, acalabrutinib, vandetanib, cobimetinib, binimetinib, and ceritinib; temozolamide, dacarbazine, altretamine, procarbazine, miltefosine, and hydroxycarbamide; trabectedin, streptozotocin, venetoclax, omacetaxine mepesuccinate, dactinomycin, and bleomycin; bortezomib, ixazomib, and carfilzomib; copanlisib and idelalisib; niraparib, olaparib, rucaparib, and talazoparib; sirolimus, temsirolimus, everolimus, tacrolimus, ciclosporin, mycophenolic acid, levamisole, fingolimod, and NSAIDs; romidepsin, belinostat, vorinostat, and panobinostat; vismodegib and sonidegib; megestral acetate, medroxyprogesterone acetate, abiraterone acetate, bicalutamide, raloxifene, letrazole, anastrozole, tamoxifen, fulvestrant, exemestane, enzalutamide, fluoxymesteone, estramustine, apalutamide, flutamide, toremifene, nilutamide, testolactone, teriflunamide; thalidomide, lenalidomide, and pomalidomide; isotretinoin, tretinoin, bexarotene, and pentostatin; enasidenib and ivosidenib; plerixafor and mavorixafor; nivolumab, pembrolizumab, atezolizumab, avelumab, durvalumab, cemiplimab, dostarlimab, and ipilimumab; alemtuzumab, elotuzumab, ofatumumab, rituximab, bevacizumab, brentuximab vedotin, gemtuzumab, daratumumab, and isatuximab; and tisagenlecleucel, axicabtagene ciloleucel, sipuleucel-T, brexucabtagene, and autoleucel.

In some examples, at least one of the one or more additional pharmaceutically active agents is an immuno-oncology agent.

In some examples, the immuno-oncology agent is a cellular immunotherapy agent. In some examples, the cellular immunotherapy agent is a dendritic cell therapy agent such as sipuleucel-T. In some examples, the cellular immunotherapy agent is a CAR-T cell therapy agent such as tisagenlecleucel and axicabtagene ciloleucel.

In some examples, the immuno-oncology agent is an antibody. In some examples, the antibody is a monoclonal antibody. In some examples, the antibody is selected from rituximab, ofatumumab, elotuzumab, alemtuzumab, atezolizumab, avelumab, durvalumab, ipilimumab, nivolumab, and pembrolizumab.

In some examples, the immuno-oncology agent is an immune checkpoint inhibitor. Suitable immune checkpoint inhibitors include, but are not limited to, CTLA-4 inhibitors, PD-1 inhibitors, and PD-L1 inhibitors. In some examples, the immune checkpoint inhibitor is a CTLA-4 inhibitor, such as ipilimumab. In some examples, the immune checkpoint inhibitor is a PD-1 inhibitor, such as nivolumab and pembrolizumab. In some examples, the immune checkpoint inhibitor is a PD-L1 inhibitor, such as atezolizumab, avelumab, and durvalumab.

### EXAMPLES

### General Chemical Synthesis and Characterization:

Automated flash column chromatography was performed using a Teledyne ISCO CombiFlash Companion system with silica gel-packed columns (SiliCycle Inc.). Analytical thin-layer chromatography (TLC, commercially available from Sigma Aldrich) was carried out on aluminum-supported silica gel plates (thickness: 200 μm) with fluorescent indicator (F-254). Visualization of compounds on TLC plates was accomplished with UV light (254 nm) and/or with phosphomolybdic acid or ceric ammonium molybdate. NMR spectra (¹H, ¹³C, ¹⁹F, and ³¹P) were obtained using either a Bruker 400 MHz spectrometer, a Bruker 600 MHz spectrometer, a Varian INOVA 600 MHz spectrometer, a Varian INOVA 500 MHz spectrometer, a Varian INOVA 400 MHz spectrometer, or a Varian VNMR 400 MHz. NMR samples were prepared in deuterated chloroform (CDCl₃) using the residual solvent peak (CDCl₃: ¹H=7.26 ppm, ¹³C═77.16 ppm) as an internal reference. The residual chloroform peak in ¹H NMR was used as an absolute reference for ³¹P NMR and ¹⁹F NMR, unless otherwise specified. MestReNova software was used to process all NMR spectra. NMR data are reported to include chemical shifts (S) reported in ppm, multiplicities indicated as s (singlet), d (doublet), t (triplet), q (quartet), m (multiplet), br (broad), or app (apparent), coupling constants (J) reported in Hz, and integration normalized to 1 atom (H, C, F, or P). High resolution mass spectrometry (HRMS) was performed by the Emory University Mass Spectrometry Center, directed by Dr. Fred Strobel.

### Example 1. Synthesis of isopropyl ((S)-((R)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate

Scheme 1 below illustrates the synthetic procedures involved in Example 1.

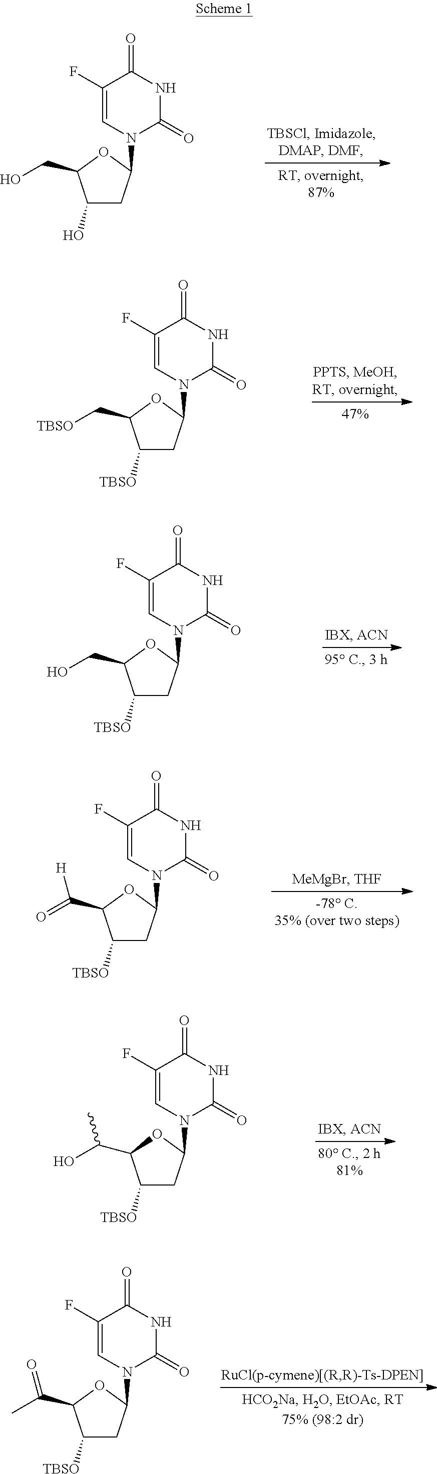 [SMILES: O=c1[nH]c(=O)n([C@H]2C[C@H](O)[C@@H](CO)O2)cc1F ; CC(C)(C)[Si](C)(C)OC[C@H]1O[C@@H](n2cc(F)c(=O)[nH]c2=O)C[C@@H]1O[Si](C)(C)C(C)(C)C ; CC(C)(C)[Si](C)(C)O[C@H]1C[C@H](n2cc(F)c(=O)[nH]c2=O)O[C@@H]1CO ; CC(C)(C)[Si](C)(C)O[C@H]1C[C@H](n2cc(F)c(=O)[nH]c2=O)O[C@@H]1C=O ; CC(O)[C@H]1O[C@@H](n2cc(F)c(=O)[nH]c2=O)C[C@@H]1O[Si](C)(C)C(C)(C)C ; CC(=O)[C@H]1O[C@@H](n2cc(F)c(=O)[nH]c2=O)C[C@@H]1O[Si](C)(C)C(C)(C)C]

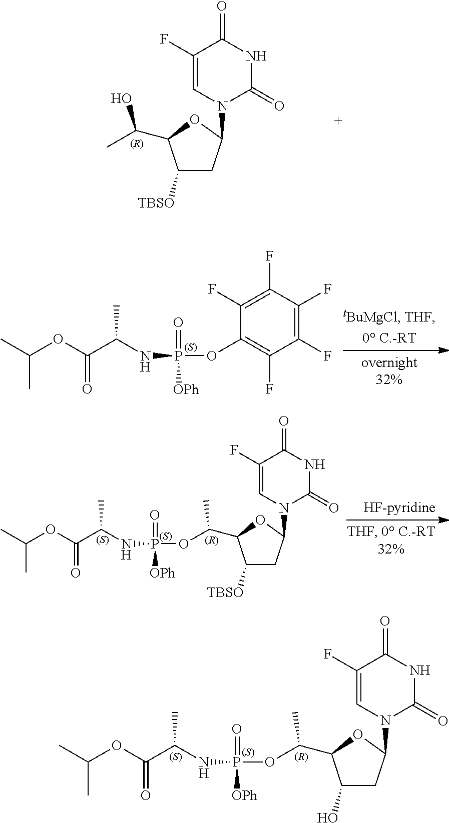

A. Synthesis of 1-((2R,4S,5R)-4-((tert-butyldimethylsilyl)oxy)-5-(((tert-butyldimethylsilyl)oxy)methyl)tetrahydrofuran-2-yl)-5-fluoropyrimidine-2,4(1H,3H)-dione (MD-7-28)

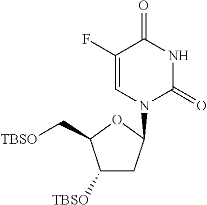 [SMILES: CC(C)(C)[Si](C)(C)OC[C@H]1O[C@@H](n2cc(F)c(=O)[nH]c2=O)C[C@@H]1O[Si](C)(C)C(C)(C)C]

To a solution of 5-fluoro-2′-deoxyuridine (5.0 g, 20.31 mmol, 1.0 eq) in anhydrous DMF (45 mL) was added imidazole (6.91 g, 101.55 mmol, 5 eq), and 4-dimethylaminopyridine (248.12 mg, 2.03 mmol, 0.1 eq). To this mixture tert-butyldimethylchlorosilane (7.65 g, 50.77 mmol, 2.5 eq) was added in portions and stirred at room temperature for 3 h. The reaction mixture was quenched with saturated NaHCO₃ solution (100 mL) and extracted with CH₂Cl₂ (×3). The organic layer was rewashed with water (×2) followed by brine solution. The organic layers were dried over anhydrous Na₂SO₄, filtered, and concentrated to get a crude mixture. Purification of the crude mixture by silica gel chromatography using 0-50% EtOAc/hexanes eluted the product at ˜20-35% gradient as a white solid (8.9 g, 17.623 mmol, 87% yield). ¹H NMR (600 MHz, CDCl₃) δ 9.82 (s, 1H), 8.02 (d, J=6.2 Hz, 1H), 6.28 (d, J=1.7 Hz, 1H), 4.40 (dt, J=6.7, 3.6 Hz, 1H), 3.94-3.89 (m, 2H), 3.80-3.65 (m, 1H), 2.31 (ddd, J=13.3, 6.1, 3.9 Hz, 1H), 2.08-2.01 (m, 1H), 0.91 (s, 9H), 0.87 (s, 9H), 0.11 (d, J=4.2 Hz, 6H), 0.06 (d, J=3.6 Hz, 6H). ¹³C NMR (151 MHz, CDCl₃) δ 157.28 (d, J=26.6 Hz), 149.18, 140.64 (d, J=236.6 Hz), 124.35 (d, J=34.1 Hz), 88.16, 85.62, 77.37, 77.16, 76.95, 71.57, 62.74, 41.90, 25.99, 25.82, 25.80, 18.51, 18.08, −4.52, −4.77, −5.47, −5.50. ¹F NMR (565 MHz, CDCl₃) δ −164.27 (t, J=5.6 Hz). HRMS (APCI) m/z calculated for C₂₁H₄₀O₅N₂FSi₂ [M+H]⁺: 475.24543, found 475.24563.

B. Synthesis of 1-((2R,4S,5R)-4-((tert-butyldimethylsilyl)oxy)-5-(hydroxymethyl)tetrahydrofuran-2-yl)-5-fluoropyrimidine-2,4(1H,3H)-dione (MD-7-30)

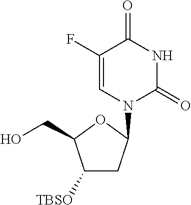 [SMILES: CC(C)(C)[Si](C)(C)O[C@H]1C[C@H](n2cc(F)c(=O)[nH]c2=O)O[C@@H]1CO]

To a solution of 1-((2R,4S,5R)-4-((tert-butyldimethylsilyl)oxy)-5-(((tert-butyldimethylsilyl)oxy)methyl)tetrahydrofuran-2-yl)-5-fluoropyrimidine-2,4(1H,3H)-dione (8.90 g, 18.75 mmol, 1.0 eq) in methanol (100 mL) was added pyridinium p-toluenesulfonate (6.12 g, 24.37 mmol, 1.3 eq) and stirred at room temperature overnight. After 17 h, the reaction mixture was concentrated under reduced pressure and redissolved in EtOAc and washed with water followed by brine solution. The organic layer was dried over anhydrous Na₂SO₄, filtered, and concentrated in vacuo. Purification by silica gel flash chromatography using 10-100% EtOAc/hexanes eluted product in ˜50% gradient as a white solid (3.14 g, 8.711 mmol, 47% yield). ¹H NMR (600 MHz, CDCl₃) δ 9.13 (d, J=3.8 Hz, 1H), 7.96 (dd, J=6.4, 1.4 Hz, 1H), 6.42-5.91 (m, 1H), 4.48 (dd, J=4.9, 2.1 Hz, 1H), 4.10-3.88 (m, 2H), 3.80 (dt, J=11.2, 2.1 Hz, 1H), 2.34-2.26 (m, 1H), 2.26-2.15 (m, 1H), 0.89 (d, J=1.1 Hz, 9H), 0.08 (s, 6H). ¹³C NMR (151 MHz, CDCl₃) δ 157.07 (d, J=26.8 Hz), 148.93, 140.60 (d, J=236.8 Hz), 125.19 (d, J=34.3 Hz), 87.74, 86.37, 77.37, 77.16, 76.95, 71.48, 61.90, 41.35, 25.84, 18.10, −4.56, −4.74. ¹⁹F NMR (565 MHz, CDCl₃) δ −164.55-−164.69 (m). HRMS (APCI) m/z calculated for C₁₅H₂₆O₅N₂FSi [M+H]⁺: 361.15895, found 361.15853.

C. Synthesis of (2S,3S,5R)-3-((tert-butyldimethylsilyl)oxy)-5-(5-fluoro-2,4-dioxo-3,4-dihydropyrimidin-1(2H)-yl)tetrahydrofuran-2-carbaldehyde (MD-7-31)

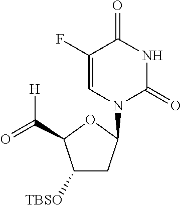 [SMILES: CC(C)(C)[Si](C)(C)O[C@H]1C[C@H](n2cc(F)c(=O)[nH]c2=O)O[C@@H]1C=O]

To a solution of 1-((2R,4S,5R)-4-((tert-butyldimethylsilyl)oxy)-5-(hydroxymethyl)tetrahydrofuran-2-yl)-5-fluoropyrimidine-2,4(1H,3H)-dione (3.00 g, 8.32 mmol, 1.0 eq) in anhydrous MeCN (80 mL) was added IBX (4.66 g, 16.65 mmol, 2.0 eq) and refluxed at 95° C. for 2.5 h. The reaction mixture was allowed to cool to room temperature and filtered using sintered glass funnel and rinsed with EtOAc. The filtrate was concentrated and vacuum dried to obtain the product as a crude solid (3.35 g, 9.346 mmol, 112% crude yield). HRMS (APCI) m/z calculated for C₁₅H₂₄O₅N₂FSi [M+H]⁺: 359.1433, found 359.14327.

D. Synthesis of 1-((2R,4S,5R)-4-((tert-butyldimethylsilyl)oxy)-5-(1-hydroxyethyl)tetrahydrofuran-2-yl)-5-fluoropyrimidine-2,4(1H,3H)-dione (MD-7-32)

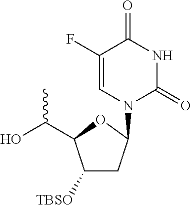 [SMILES: CC(O)[C@H]1O[C@@H](n2cc(F)c(=O)[nH]c2=O)C[C@@H]1O[Si](C)(C)C(C)(C)C]

To a solution of crude (2S,3S,5R)-3-((tert-butyldimethylsilyl)oxy)-5-(5-fluoro-2,4-dioxo-3,4-dihydropyrimidin-1(2H)-yl)tetrahydrofuran-2-carbaldehyde (1.00 g, 2.79 mmol, 1.0 eq) in anhydrous THF (24 mL) was cooled to −78° C. and added methylmagnesium bromide (2.79 mL, 8.37 mmol, 3 eq) dropwise over 10 min. After 2.5 h, aliquot analyzed by ¹H-NMR indicated 70% conversion with 3:1 d/r ratio. Reaction continued stirring at −78° C. for another 3.5 h. After 7 h in total, ¹H-NMR analysis indicated no further increase in conversion. The reaction mixture was quenched with saturated aqueous NH₄Cl (25 mL) and allowed to warm to room temperature and extracted with EtOAc (×2). The combined organic extracts were washed with water followed by brine. The organic layer was dried over anhydrous Na₂SO₄, filtered, and concentrated under reduced pressure to obtain a crude mixture (2.6:1 diastereomeric ratio (dr)). Purification of the crude mixture by silica gel chromatography using 0-100% EtOAc/hexanes eluted the product as a solid (0.330 g, 0.881 mmol, 35% yield over two steps). ¹H NMR (600 MHz, CDCl₃); mixture of diastereomers at 4:1 ratio. S 8.65 (s, 1H), 8.04 (d, J=6.4 Hz, 1H), 7.94 (d, J=6.4 Hz, 0.25H), 6.28-6.19 (m, 1.25H), 4.53 (dt, J=5.4, 2.6 Hz, 0.25H), 4.42 (dt, J=6.1, 3.7 Hz, 1H), 4.14-4.10 (m, 0.25H), 3.99 (qd, J=6.5, 2.4 Hz, 1H), 3.82 (t, J=2.5 Hz, 0.25H), 3.73 (dd, J=3.4, 2.3 Hz, 1H), 2.31-2.16 (m, 2.5H), 1.34 (d, J=6.5 Hz, 3H), 1.29 (d, J=6.6 Hz, 0.75H), 0.89 (s, 2.25H), 0.89 (s, 9H), 0.10 (s, 1.25H), 0.08 (s, 3H), 0.08 (s, 3H). ¹³C NMR (151 MHz, CDCl₃, major isomer) S 157.12 (d, J=26.7 Hz), 148.99, 140.61 (d, J=236.6 Hz), 125.30 (d, J=34.3 Hz), 90.73, 86.20, 72.67, 67.32, 40.98, 25.82, 20.89, 18.08, −4.48, −4.70. ¹⁹F NMR (376 MHz, CDCl₃, major isomer) δ −164.60 (td, J=5.9, 5.3, 1.6 Hz). HRMS (APCI) m/z calculated for C₁₆H₂SO₅N₂FSi [M+H]⁺: 375.1746, found 375.17444.

E. Synthesis of 1-((2R,4S,5S)-5-acetyl-4-((tert-butyldimethylsilyl)oxy)tetrahydrofuran-2-yl)-5-fluoropyrimidine-2,4(1H,3H)-dione (MD-7-36)

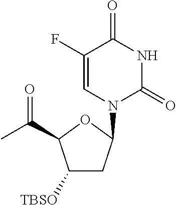 [SMILES: CC(=O)[C@H]1O[C@@H](n2cc(F)c(=O)[nH]c2=O)C[C@@H]1O[Si](C)(C)C(C)(C)C]

To 1-((2R,4S,5R)-4-((tert-butyldimethylsilyl)oxy)-5-(1-hydroxyethyl)tetrahydrofuran-2-yl)-5-fluoropyrimidine-2,4(1H,3H)-dione (245.0 mg, 0.650 mmol, 1.0 eq) in anhydrous MeCN (8 mL) was added IBX (458.0 mg, 1.64 mmol, 2.5 eq) and the reaction mixture was heated at 80° C. for 2 h. The reaction mixture was allowed to cool to room temperature and filtered through sintered glass funnel and rinsed with EtOAc. The filtrate was concentrated and purified by silica gel chromatography eluted with 0-70% EtOAc/hexanes to obtain the product as a solid (0.198 g, 0.531 mmol, 81% yield). ¹H NMR (600 MHz, CDCl₃) δ 8.96 (s, 1H), 8.50 (d, J=6.5 Hz, 1H), 6.39 (ddd, J=8.2, 5.3, 1.6 Hz, 1H), 4.56 (d, J=1.9 Hz, 1H), 4.50-4.36 (m, 1H), 2.33 (ddd, J=13.4, 5.4, 2.2 Hz, 1H), 2.27 (s, 3H), 1.88 (ddd, J=12.1, 8.2, 5.0 Hz, 1H), 0.93 (s, 9H), 0.15 (s, 3H), 0.14 (s, 3H). ¹³C NMR (151 MHz, CDCl₃) δ 205.71, 156.91 (d, J=27.1 Hz), 148.94, 140.73 (d, J=237.3 Hz), 125.00 (d, J=34.9 Hz), 91.10, 86.91, 77.37, 77.31, 77.16, 76.95, 73.64, 39.94, 27.58, 25.78, 18.06, −4.49, −4.71. ¹F NMR (565 MHz, CDCl₃) δ −163.71-−163.82 (m). HRMS (APCI) m/z calculated for C₁₆H₂₆O₅N₂FSi [M+H]⁺: 373.15895, found 373.15888.

F. Synthesis of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (MD-7-29)

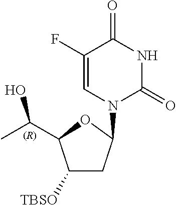

An oven dried three neck flask was charged with 1-((2R,4S,5S)-5-acetyl-4-((tert-butyldimethylsilyl)oxy)tetrahydrofuran-2-yl)-5-fluoropyrimidine-2,4(1H,3H)-dione (160.0 mg, 0.430 mmol, 1.0 eq) and RuCl(p-cymene)[(R,R)-Ts-DPEN] (2.73 mg, 0.004 mmol, 0.1 eq) and flushed with argon. A solution of sodium formate (1.21 g, 17.8 mmol) in H₂O (7 mL) was added, followed by ethyl acetate (1.76 mL). The resulting two-phase mixture was stirred overnight at room temperature. After 17 h, the reaction mixture was diluted with EtOAc (10 mL). Organic layer was separated, and aqueous layer was again extracted with EtOAc. The combined organic layers were washed with water followed by brine. The organic layer was dried over anhydrous Na₂SO₄, filtered, and concentrated under reduced pressure to get a crude solid. Purification of the crude solid by silica gel chromatography eluted with 0-70% EtOAc/hexanes afforded the product as a solid (120 mg, 0.320 mmol, 75% yield, 98:2 dr). ¹H NMR (400 MHz, CDCl₃) δ 9.14 (d, J=4.7 Hz, 1H), 7.94 (d, J=6.4 Hz, 1H), 6.22 (ddd, J=7.8, 6.1, 1.6 Hz, 1H), 4.53 (dt, J=5.6, 2.8 Hz, 1H), 4.11 (qd, J=6.7, 2.6 Hz, 1H), 3.81 (t, J=2.6 Hz, 1H), 2.34 (s, 1H), 2.31-2.05 (m, 2H), 1.28 (d, J=6.7 Hz, 3H), 0.89 (s, 9H), 0.09 (s, 6H). ¹C NMR (101 MHz, CDCl₃) δ 157.05 (d, J=26.9 Hz), 149.00, 140.61 (d, J=236.7 Hz), 125.46 (d, J=34.2 Hz), 91.53, 86.47, 77.48, 77.36, 77.16, 76.84, 70.66, 67.98, 41.40, 25.82, 20.10, 17.96, −4.30, −4.69. ¹⁹F NMR (376 MHz, CDCl₃) δ −164.55 (ddd, J=6.4, 4.7, 1.6 Hz). HRMS (APCI) m/z calculated for C₁₆H₂₈O₅N₂FSi [M+H]⁺: 375.1746, found 375.17445.

G. Synthesis of isopropyl ((S)-((R)-1-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)ethoxy)(phenoxy)phosphoryl)-L-alaninate (MD-7-33)

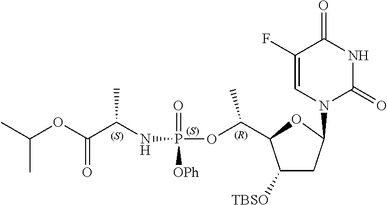

To a solution of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (120.0 mg, 0.320 mmol, 1.0 eq) in a mixture of anhydrous THF (2.5 mL) and anhydrous NMP (0.1 mL) at 0° C. was added tert-butylmagnesium chloride (1 M in THF, 801.1 μL, 0.800 mmol, 2.5 eq) dropwise. Ice bath was removed, and the reaction mixture was stirred at room temperature for 1 h and 30 min. The mixture was again cooled to 0° C. and a solution of isopropyl rac-(2S)-2-[[(2,3,4,5,6-pentafluorophenoxy)-phenoxy-phosphoryl] amino]propanoate (217.89 mg, 0.480 mmol, 1.5 eq) in anhydrous THF (1.5 mL) was added dropwise. The mixture was allowed to warm to room temperature and stirred overnight. After 16 h, the reaction mixture was cooled to 0° C. and quenched with MeOH, concentrated to a crude solid and purified by silica gel chromatography eluted with 5-70% EtOAc/hexanes to obtain the product as a solid (67 mg, 0.104 mmol, 32% yield). ¹H NMR (600 MHz, CDCl₃) δ 9.35 (s, 1H), 7.90 (d, J=6.3 Hz, 1H), 7.32-7.27 (m, 2H), 7.23-7.18 (m, 2H), 7.16-7.11 (m, 1H), 6.16 (ddd, J=8.7, 5.3, 1.6 Hz, 1H), 5.12-4.92 (m, 1H), 4.86-4.70 (m, 1H), 4.46 (dt, J=6.1, 2.3 Hz, 1H), 4.05-3.88 (m, 1H), 3.82 (q, J=2.8 Hz, 1H), 3.78 (d, J=11.5 Hz, 1H), 2.13 (ddd, J=13.2, 5.3, 2.0 Hz, 1H), 1.66 (ddd, J=13.2, 8.7, 6.1 Hz, 1H), 1.44 (d, J=6.6 Hz, 3H), 1.34 (d, J=7.0 Hz, 3H), 1.22 (d, J=6.4 Hz, 6H), 0.88 (s, 9H), 0.08 (s, 3H), 0.08 (s, 3H). ¹³C NMR (151 MHz, CDCl₃) δ 172.89 (d, J=7.8 Hz), 156.91 (d, J=26.8 Hz), 150.44 (d, J=6.6 Hz), 148.77, 140.51 (d, J=237.1 Hz), 129.72, 125.24, 124.50 (d, J=34.2 Hz), 120.42 (d, J=4.7 Hz), 89.56 (d, J=6.6 Hz), 84.78, 73.96 (d, J=5.4 Hz), 70.51, 69.40, 50.51, 40.76, 25.64, 21.67, 21.60, 21.23 (d, J=4.3 Hz), 18.38 (d, J=2.1 Hz), 17.77, −4.43, −4.86. ¹⁹F NMR (565 MHz, CDCl₃) δ −164.18 (t, J=5.8 Hz). ³¹P NMR (162 MHz, CDCl₃) δ 2.17 (m). HRMS (ESI) m/z calculated for C₂₈H₄₃O₉N₃FNaPSi [M+Na]⁺: 666.23824, found 666.2385.

H. Synthesis of isopropyl ((S)-((R)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate (MD-7-41)

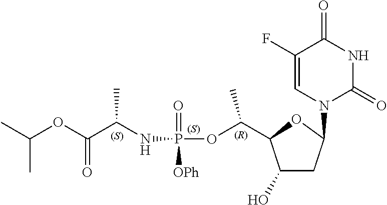

To a solution of isopropyl ((S)-((R)-1-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)ethoxy)(phenoxy)phosphoryl)-L-alaninate (60.0 mg, 0.090 mmol, 1.0 eq) in anhydrous THF (1 mL) at 0° C. was added hydrogen fluoride-pyridine (71.98 μL, 0.560 mmol, 6.0 eq) dropwise. The reaction mixture was allowed to warm to room temperature and stirred for 5 h. The reaction mixture was then quenched with saturated aqueous NaHCO₃ until neutral pH and extracted with EtOAc (×3). The combined organic layers were dried over anhydrous Na₂SO₄, filtered, and concentrated under reduced pressure to obtain a crude mixture. Purification of the crude mixture by silica gel chromatography using 0-100% EtOAc/hexanes eluted the product in 100% EtOAc as a solid (16 mg, 0.030 mmol, 32% yield). ¹H NMR (600 MHz, CDCl₃) δ 9.62-9.56 (m, 1H), 7.67 (d, J=6.0 Hz, 1H), 7.31 (t, J=7.8 Hz, 2H), 7.20 (d, J=7.6 Hz, 2H), 7.15 (t, J=7.4 Hz, 1H), 6.16 (ddd, J=7.7, 5.8, 1.6 Hz, 1H), 5.02 (hept, J=6.3 Hz, 1H), 4.81-4.61 (m, 1H), 4.46 (dd, J=6.9, 3.4 Hz, 1H), 4.13-3.84 (m, 2H), 3.84-3.68 (m, 1H), 2.29 (ddd, J=13.8, 5.9, 3.1 Hz, 1H), 1.86 (dt, J=14.3, 7.4 Hz, 1H), 1.44 (d, J=6.5 Hz, 3H), 1.37 (d, J=6.7 Hz, 3H), 1.24 (d, J=6.3 Hz, 6H). ¹³C NMR (151 MHz, CDCl₃) δ 173.18 (d, J=7.6 Hz), 157.06 (d, J=26.6 Hz), 150.51 (d, J=6.7 Hz), 148.98, 140.75 (d, J=237.7 Hz), 129.92, 125.47, 124.41 (d, J=34.0 Hz), 120.59 (d, J=4.5 Hz), 88.60 (d, J=6.5 Hz), 84.81, 74.71 (d, J=5.4 Hz), 71.01, 69.69, 50.69, 40.08, 21.81, 21.75, 21.22 (d, J=4.5 Hz), 18.70 (d, J=2.6 Hz). HRMS (APCI) m/z calculated for C₂₂H₃₀O₉N₃FP [M+H]⁺: 530.16982, found 530.1698.

### Example 2. Prophetic synthesis of isopropyl ((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate

Isopropyl ((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate can be synthesized according to the procedures described in Scheme 2.

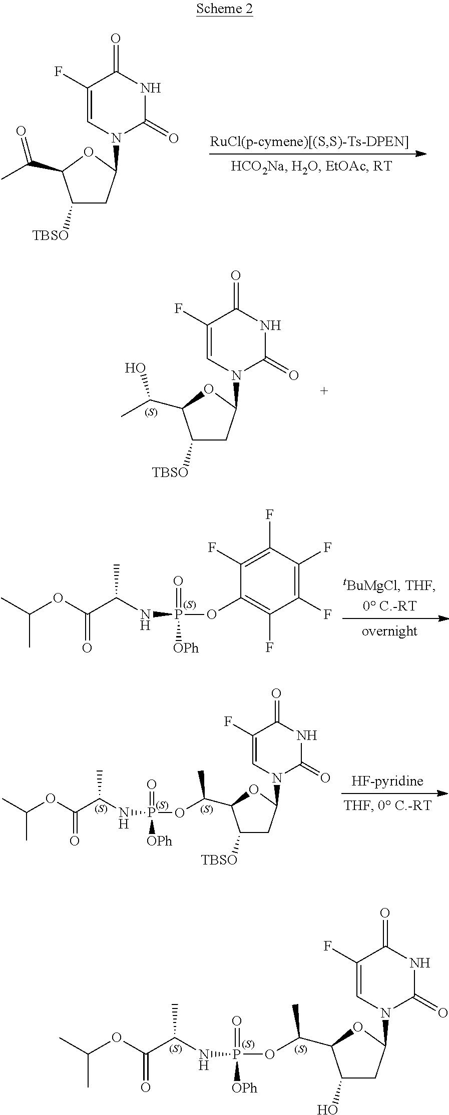

A. Synthesis of 1-((2R,4S,5R)-4-((tert-butyldimethylsilyl)oxy)-5-((S)-J-hydroxyethyl)tetrahydrofuran-2-yl)-5-fluoropyrimidine-2,4(1H,3H)-dione (MD-7-42)

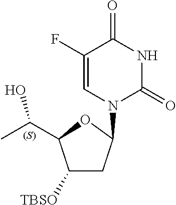

An oven dried three neck flask was charged with 1-((2R,4S,5R)-4-((tert-butyldimethylsilyl)oxy)-5-(1-hydroxyethyl)tetrahydrofuran-2-yl)-5-fluoropyrimidine-2,4(1H,3H)-dione (150.0 mg, 0.40 mmol, 1.0 eq) and RuCl(p-cymene)[(S,S)-Ts-DPEN] (2.56 mg, 0.004 mmol, 0.1 eq) and flushed with argon. A solution of sodium formate (1.14 g, 16.76 mmol, 42 eq) in H₂O (6.5 mL) was added, followed by ethyl acetate (1.6 mL). The resulting two-phase mixture was stirred overnight at room temperature. After 19 h, the reaction mixture was diluted with CH₂Cl₂ (10 mL). The organic layer was separated, and the aqueous layer was again extracted with CH₂Cl₂. The combined organic layers were washed with water followed by brine. The organic layer was dried over anhydrous Na₂SO₄, filtered, and concentrated under reduced pressure to get a crude solid. Purification of the crude solid by silica gel chromatography eluted with 0-60% EtOAc/hexanes afforded the product as a solid (120 mg, 0.336 mmol, 83% yield, 70:30 dr). The product was further subjected to silica gel chromatography eluted with 20-60% EtOAc/hexanes. Fractions that contained pure (S)-isomer (TLC analysis) were pooled and concentrated to obtain the product as a white solid with 94:6 dr. ¹H NMR (600 MHz, CDCl₃) δ 9.17 (d, J=4.8 Hz, 1H), 8.05 (d, J=6.4 Hz, 1H), 6.25 (td, J=6.5, 1.6 Hz, 1H), 4.42 (dt, J=6.1, 3.7 Hz, 1H), 3.99 (qd, J=6.5, 2.4 Hz, 1H), 3.73 (dd, J=3.4, 2.4 Hz, 1H), 2.38-2.10 (m, 2H), 1.33 (d, J=6.5 Hz, 3H), 0.89 (s, 9H), 0.08 (d, J=1.1 Hz, 6H). ¹³C NMR (151 MHz, CDCl₃) δ 157.06 (d, J=25.8 Hz), 148.95, 140.60 (d, J=236.5 Hz), 125.29 (d, J=34.3 Hz), 90.71, 86.20, 72.67, 67.33, 40.97, 25.85, 20.91, 18.08, −4.49, −4.70. HRMS (APCI) m/z calculated for C₁₆H₂₆O₅N₂FSi [M−H]⁻: 373.16005, found 373.15971.

1-((2R,4S,5R)-4-((tert-butyldimethylsilyl)oxy)-5-((S)-1-hydroxyethyl)tetrahydrofuran-2-yl)-5-fluoropyrimidine-2,4(1H,3H)-dione (MD-7-42) can be used to synthesis the final product, isopropyl ((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate, according to the rest of the procedures shown Scheme 2.

### Example 3. Alternative synthetic route of isopropyl ((S)-((R)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate

Scheme 3 below illustrates the synthetic procedures involved in Example 3. Scheme 3 is an alternative synthetic route to the one shown in Scheme 1, for making isopropyl ((S)-((R)-1-((2S,3S,5R)-5-(5-fluoro-2,4-dioxo-3,4-dihydropyrimidin-1 (2H)-yl)-3-hydroxytetrahydrofuran-2-yl)ethoxy)(phenoxy)phosphoryl)-L-alaninate.

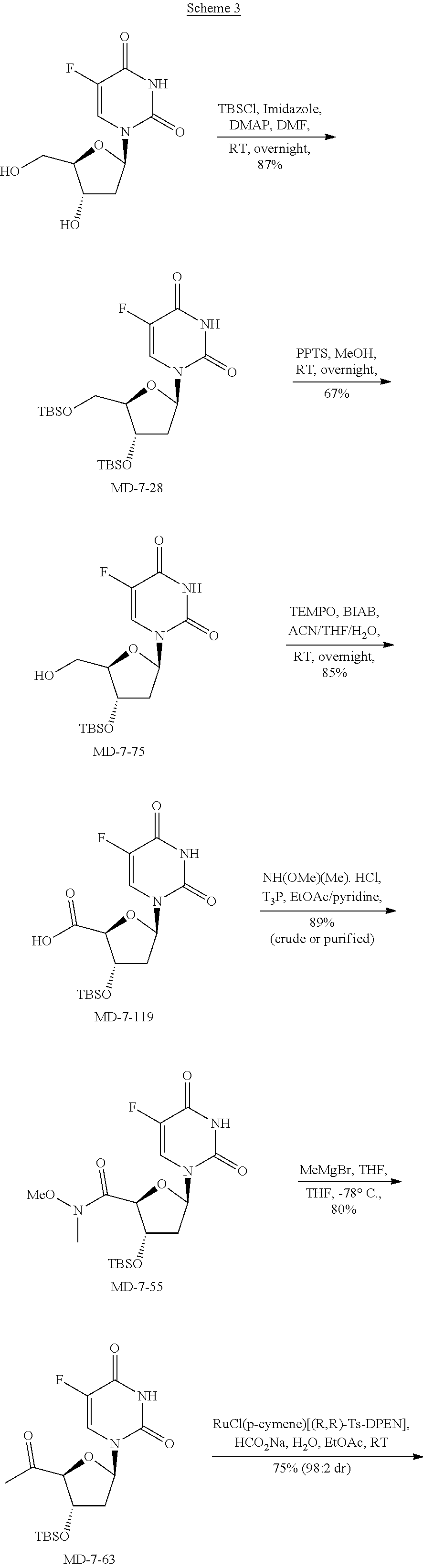 [SMILES: O=c1[nH]c(=O)n([C@H]2C[C@H](O)[C@@H](CO)O2)cc1F ; CC(C)(C)[Si](C)(C)OC[C@H]1O[C@@H](n2cc(F)c(=O)[nH]c2=O)C[C@@H]1O[Si](C)(C)C(C)(C)C ; CC(C)(C)[Si](C)(C)O[C@H]1C[C@H](n2cc(F)c(=O)[nH]c2=O)O[C@@H]1CO ; CC(C)(C)[Si](C)(C)O[C@H]1C[C@H](n2cc(F)c(=O)[nH]c2=O)O[C@@H]1C(=O)O ; CON(C)C(=O)[C@H]1O[C@@H](n2cc(F)c(=O)[nH]c2=O)C[C@@H]1O[Si](C)(C)C(C)(C)C ; CC(=O)[C@H]1O[C@@H](n2cc(F)c(=O)[nH]c2=O)C[C@@H]1O[Si](C)(C)C(C)(C)C]

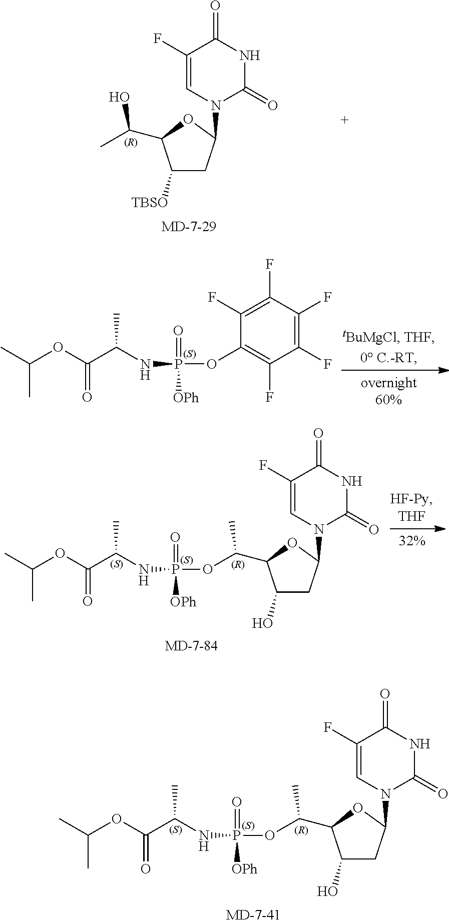

A. Synthesis of 1-((2R,4S,5R)-4-((tert-butyldimethylsilyl)oxy)-5-(hydroxymethyl)tetrahydrofuran-2-yl)-5-fluoropyrimidine-2,4(1H,3H)-dione (MD-7-75)

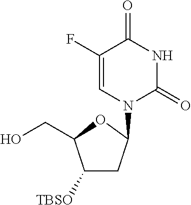 [SMILES: CC(C)(C)[Si](C)(C)O[C@H]1C[C@H](n2cc(F)c(=O)[nH]c2=O)O[C@@H]1CO]

To a solution of 1-((2R,4S,5R)-4-((tert-butyldimethylsilyl)oxy)-5-(((tert-butyldimethylsilyl)oxy)methyl)tetrahydrofuran-2-yl)-5-fluoro pyrimidine-2,4(1H,3H)-dione (19.0 g, 34.02 mmol, 1.0 eq) in methanol (400 mL) was added pyridinium p-toluenesulfonate (11.11 g, 44.23 mmol, 1.3 eq) and stirred at room temperature overnight with the exclusion of light. After 17 h, the reaction mixture was concentrated under reduced pressure and redissolved in EtOAc and washed with water followed by brine solution. The organic layer was dried over anhydrous Na₂SO₄, filtered, and concentrated in vacuo. Purification by silica gel flash chromatography using 10-100% EtOAc/hexanes eluted MD-7-75 at ˜50% gradient as a white solid (8.3 g, 23.027 mmol, 67% yield). ¹H NMR (600 MHz, CDCl₃) δ 9.13 (d, J=3.8 Hz, 1H), 7.96 (dd, J=6.4, 1.4 Hz, 1H), 6.42-5.91 (m, 1H), 4.48 (dd, J=4.9, 2.1 Hz, 1H), 4.10-3.88 (m, 2H), 3.80 (dt, J=11.2, 2.1 Hz, 1H), 2.34-2.26 (m, 1H), 2.26-2.15 (m, 1H), 0.89 (d, J=1.1 Hz, 9H), 0.08 (s, 6H). ¹³C NMR (151 MHz, CDCl₃) δ 157.07 (d, J=26.8 Hz), 148.93, 140.60 (d, J=236.8 Hz), 125.19 (d, J=34.3 Hz), 87.74, 86.37, 77.37, 77.16, 76.95, 71.48, 61.90, 41.35, 25.84, 18.10, −4.56, −4.74. ¹⁹F NMR (565 MHz, CDCl₃) δ −164.62 (t, J=3.9 Hz). HRMS (APCI) m/z calculated for C₁₅H₂₆O₅N₂FSi [M+H]⁺: 361.15895, found 361.15853.

B. Synthesis of (2S,3S,5R)-3-((tert-butyldimethylsilyl)oxy)-5-(5-fluoro-2,4-dioxo-3,4-dihydropyrimidin-1(2H)-yl)tetrahydrofuran-2-carboxylic acid (MD-7-119)

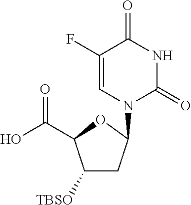 [SMILES: CC(C)(C)[Si](C)(C)O[C@H]1C[C@H](n2cc(F)c(=O)[nH]c2=O)O[C@@H]1C(=O)O]

To a solution of 1-((2R,4S,5R)-4-((tert-butyldimethylsilyl)oxy)-5-(hydroxymethyl)tetrahydrofuran-2-yl)-5-fluoropyrimidine-2,4(1H,3H)-dione (12.32 g, 34.18 mmol, 1.0 eq) in a mixture of MeCN (55 mL), water (55 mL), and THF (55 mL) was added iodobenzene diacetate (24.22 g, 75.19 mmol, 2.2 eq) followed by TEMPO (1.07 g, 6.84 mmol, 0.2 eq) and stirred at room temperature overnight. After 17 h, the reaction mixture was concentrated under reduced pressure to a crude solid. The solid was suspended in a mixture of CH₂Cl₂/hexanes (1:1) and filtered and rinsed thoroughly with excess solvent mixture to remove excess reagents and byproducts. The resulting solid was freeze-dried to obtain MD-7-119 as a solid (11 g, 29.37 mmol, 85%). ¹H NMR (400 MHz, CDCl₃) δ 8.60 (d, J=6.6 Hz, 1H), 6.43 (ddd, J=9.2, 5.0, 1.7 Hz, 1H), 4.65-4.49 (m, 1H), 4.44 (s, 1H), 2.32 (dd, J=13.3, 5.1 Hz, 1H), 1.88 (ddd, J=12.0, 9.2, 4.5 Hz, 1H), 0.88 (d, J=1.5 Hz, 9H), 0.11 (d, J=4.7 Hz, 6H). ¹³C NMR (151 MHz, CDCl₃) δ 173.51, 172.62, 149.20, 140.65 (d, J=235.7 Hz), 125.61, 125.38, 87.56, 85.63, 77.37, 77.16, 76.95, 75.96, 49.67, 49.53, 49.38, 39.93, 25.70, 18.02, −4.90, −4.95. ¹⁹F NMR (376 MHz, CDCl₃) δ −164.62 (d, J=6.6 Hz). HRMS (APCI) m/z calculated for C₁₅H₂₂O₆N₂FSi [M−H]⁻: 373.12366, found 373.12431. LC-MS (ESI) 7595% MeOH/H₂O (0.1% HCO₂H), 3 min, 1.00 mL/min, t_{R}=0.79 min, m/z=373 [M−H]⁻.

C. Synthesis of (2S,3S,5R)-3-((tert-butyldimethylsilyl)oxy)-5-(5-fluoro-2,4-dioxo-3,4-dihydropyrimidin-1(2H)-yl)-N-methoxy-N-methyltetrahydrofuran-2-carboxamide (MD-7-55)

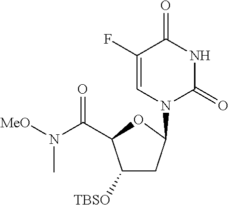 [SMILES: CON(C)C(=O)[C@H]1O[C@@H](n2cc(F)c(=O)[nH]c2=O)C[C@@H]1O[Si](C)(C)C(C)(C)C]

To a mixture of (2S,3S,5R)-3-((tert-butyldimethylsilyl)oxy)-5-(5-fluoro-2,4-dioxo-3,4-dihydropyrimidin-1(2H)-yl)tetrahydrofuran-2-carboxylic acid (0.13 g, 0.330 mmol, 1.0 eq) and N,O-dimethylhydroxylamine hydrochloride (37.45 mg, 0.380 mmol, 1.15 eq) in ethyl acetate (0.9 mL) and anhydrous pyridine (0.3 mL) at 0° C. was added propylphosphoric anhydride solution (414.98 μL, 0.670 mmol, 2.0 eq) dropwise and stirred for 2 h at 0° C. TLC analysis (5% MeOH/CH₂Cl₂) indicated complete consumption of the starting material. The reaction was quenched with aqueous citric acid (1.5 mL) and extracted with EtOAc (×2). The combined organic layers were again washed with saturated NaHCO₃ solution followed by water and brine. The organic layer was dried over anhydrous Na₂SO₄, filtered, and concentrated under reduced pressure to get a crude mixture. The mixture was azeotropically distilled under reduced pressure by the addition of toluene (×2) followed by CH₂Cl₂ to obtain crude MD-7-55 as a solid (124 mg, 0.29 mmol, 89% crude yield), which was used in the next reaction without further purification. ¹H NMR (600 MHz, CDCl₃) δ 8.95 (d, J=6.8 Hz, 1H), 8.16 (s, 1H), 6.51 (ddd, J=9.3, 5.1, 1.7 Hz, 1H), 4.81 (s, 1H), 4.47 (d, J=4.3 Hz, 1H), 3.75 (t, J=0.7 Hz, 3H), 3.25 (s, 3H), 2.28 (dd, J=13.2, 5.2 Hz, 1H), 2.00 (ddd, J=13.3, 9.2, 4.3 Hz, 1H), 0.91 (d, J=0.6 Hz, 9H), 0.11 (s, 3H), 0.10 (s, 3H). HRMS (APCI) m/z calculated for Cl₇H₂₉O₆N₃FSi [M+H]⁺: 418.18042, found 418.18057. LC-MS: 95% ISO MeOH/H₂O, 3 min, t_{R}=1.5 min, [M+H]⁺=418; [M−H]⁻=416.

D. Synthesis of 1-((2R,4S,5S)-5-acetyl-4-((tert-butyldimethylsilyl)oxy)tetrahydrofuran-2-yl)-5-fluoropyrimidine-2,4(1H,3H)-dione (MD-7-63)

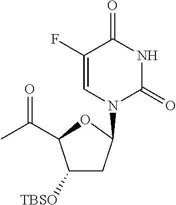 [SMILES: CC(=O)[C@H]1O[C@@H](n2cc(F)c(=O)[nH]c2=O)C[C@@H]1O[Si](C)(C)C(C)(C)C]

To a solution of (2S,3S,5R)-3-((tert-butyldimethylsilyl)oxy)-5-(5-fluoro-2,4-dioxo-3,4-dihydropyrimidin-1(2H)-yl)-N-methoxy-N-methyltetrahydrofuran-2-carboxamide (3.90 g, 9.34 mmol, 1.0 eq) in anhydrous THF (60 mL) at −22° C. (ice+NaCl) was added methylmagnesium bromide (9.34 mL, 28.02 mmol, 3.0 eq, 3.0 M in ether) dropwise and stirred for 1.5 h (during the time, the temperature raised to −18° C.). TLC analysis (50% EtOAc/hexanes) indicated complete conversion. The reaction mixture was quenched with careful addition of saturated aqueous NH₄Cl (55 mL) and allowed to warm to room temperature. The reaction mixture was extracted with EtOAc (×2). The combined organic layers were dried over anhydrous Na₂SO₄, filtered, and concentrated under reduced pressure to obtain a crude solid. The crude solid was purified by silica gel chromatography using 1060% EtOAc/hexanes to elute MD-7-63 at˜35-42% gradient as a solid (2.8 g, 7.51 mmol, 80% yield).

¹H NMR (600 MHz, CDCl₃) δ 8.96 (s, 1H), 8.50 (d, J=6.5 Hz, 1H), 6.39 (ddd, J=8.2, 5.3, 1.6 Hz, 1H), 4.56 (d, J=1.9 Hz, 1H), 4.50-4.36 (m, 1H), 2.33 (ddd, J=13.4, 5.4, 2.2 Hz, 1H), 2.27 (s, 3H), 1.88 (ddd, J=12.1, 8.2, 5.0 Hz, 1H), 0.93 (s, 9H), 0.15 (s, 3H), 0.14 (s, 3H). ¹³C NMR (151 MHz, CDCl₃) δ 205.71, 156.91 (d, J=27.1 Hz), 148.94, 140.73 (d, J=237.3 Hz), 125.00 (d, J=34.9 Hz), 91.10, 86.91, 77.37, 77.31, 77.16, 76.95, 73.64, 39.94, 27.58, 25.78, 18.06, −4.49, −4.71. ¹⁹F NMR (565 MHz, CDCl₃) δ −163.71-−163.82 (m). HRMS (APCI) m/z calculated for C₁₆H₂₄O₅N₂FSi [M−H]⁻: 371.1444, found 371.14466.

E. Synthesis of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (MD-7-29)

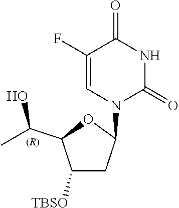

An oven dried three neck flask was charged with 1-((2R,4S,5S)-5-acetyl-4-((tert-butyldimethylsilyl)oxy)tetrahydrofuran-2-yl)-5-fluoropyrimidine-2,4(1H,3H)-dione (160.0 mg, 0.430 mmol, 1.0 eq) and RuCl(p-cymene)[(R,R)-Ts-DPEN] (2.73 mg, 0.004 mmol, 0.01 eq) and flushed with argon. A solution of sodium formate (1.21 g, 17.8 mmol) in H₂O (7 mL) was added, followed by ethyl acetate (1.76 mL). The resulting two-phase mixture was stirred overnight at room temperature. After 17 h, the reaction mixture was diluted with CH₂Cl₂. Organic layer was separated, and the aqueous layer was again extracted with CH₂Cl₂. The combined organic layers were dried over anhydrous Na₂SO₄, filtered, and concentrated under reduced pressure to get a crude solid. Purification of the crude solid by silica gel chromatography eluting with 0-70% EtOAc/hexanes afforded MD-7-29 as a solid (120 mg, 0.32 mmol, 75% yield, 98:2 dr). ¹H NMR (400 MHz, CDCl₃) δ 9.14 (d, J=4.7 Hz, 1H), 7.94 (d, J=6.4 Hz, 1H), 6.22 (ddd, J=7.8, 6.1, 1.6 Hz, 1H), 4.53 (dt, J=5.6, 2.8 Hz, 1H), 4.11 (qd, J=6.7, 2.6 Hz, 1H), 3.81 (t, J=2.6 Hz, 1H), 2.34 (s, 1H), 2.31-2.05 (m, 2H), 1.28 (d, J=6.7 Hz, 3H), 0.89 (s, 9H), 0.09 (s, 6H). ¹³C NMR (101 MHz, CDCl₃) δ 157.05 (d, J=26.9 Hz), 149.00, 140.61 (d, J=236.7 Hz), 125.46 (d, J=34.2 Hz), 91.53, 86.47, 77.48, 70.66, 67.98, 41.40, 25.82, 20.10, 17.96, −4.30, −4.69. ¹F NMR (376 MHz, CDCl₃) δ −164.55 (ddd, J=6.4, 4.7, 1.6 Hz). HRMS (APCI) m/z calculated for C₁₆H₂₈O₅N₂FSi [M+H]⁺: 375.1746, found 375.17445.

F. Synthesis of isopropyl ((S)-((R)-1-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)ethoxy)(phenoxy)phosphoryl)-L-alaninate (MD-7-84)

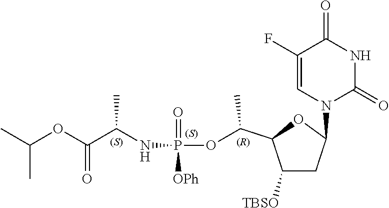

To a solution of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (300.0 mg, 0.8 mmol, 1.0 eq) in anhydrous THF (7.5 mL) at 0° C. was added tert-butylmagnesium chloride (1 M in THF, 2.0 mL, 2.0 mmol, 2.5 eq) dropwise. Ice bath was removed, and the reaction was stirred at room temperature for 1 h and 30 min. The mixture was again cooled to 0° C. and a solution of isopropyl rac-(2S)-2-[[(2,3,4,5,6-pentafluorophenoxy)-phenoxy-phosphoryl]amino]propanoate (544.72 mg, 1.2 mmol, 1.5 eq) in anhydrous THF (5 mL) was added dropwise. The mixture was allowed to warm to room temperature and stirred overnight. After 16 h, the reaction mixture was cooled to 0° C. and quenched with MeOH, concentrated to a crude solid, and purified by silica gel chromatography eluted with 5-70% EtOAc/hexanes to obtain MD-7-84 as a solid (310 mg, 0.48 mmol, 60% yield). 1H NMR (400 MHz, CDCl₃) δ 8.27 (d, J=4.9 Hz, 1H), 7.93 (d, J=6.3 Hz, 1H), 7.35-7.26 (m, 2H), 7.25-7.17 (m, 2H), 7.14 (dd, J=7.3, 1.1 Hz, 1H), 6.15 (ddd, J=8.7, 5.2, 1.7 Hz, 1H), 5.01 (hept, J=6.3 Hz, 1H), 4.83-4.70 (m, 1H), 4.47 (dt, J=6.2, 2.3 Hz, 1H), 4.03-3.89 (m, 1H), 3.83 (q, J=2.7 Hz, 1H), 3.64 (dd, J=10.9, 9.4 Hz, 1H), 2.13 (ddd, J=13.2, 5.3, 2.0 Hz, 1H), 1.67-1.60 (m, 1H), 1.44 (d, J=6.7 Hz, 3H), 1.36 (d, J=7.0 Hz, 3H), 1.23 (d, J=6.3 Hz, 6H), 0.89 (s, 9H), 0.10 (s, 3H), 0.09 (s, 3H). ¹³C NMR (151 MHz, CDCl₃) δ 172.89 (d, J=7.8 Hz), 156.91 (d, J=26.8 Hz), 150.44 (d, J=6.6 Hz), 148.77, 140.51 (d, J=237.1 Hz), 129.72, 125.24, 124.50 (d, J=34.2 Hz), 120.42 (d, J=4.7 Hz), 89.56 (d, J=6.6 Hz), 84.78, 73.96 (d, J=5.4 Hz), 70.51, 69.40, 50.51, 40.76, 25.64, 21.67, 21.60, 21.23 (d, J=4.3 Hz), 18.38 (d, J=2.1 Hz), 17.77, −4.43, −4.86. ¹F NMR (376 MHz, CDCl₃) δ −164.19 (ddd, J=6.6, 4.9, 2.1 Hz). ³¹P NMR (162 MHz, CDCl₃) δ 2.14. HRMS (ESI) m/z calculated for C₂₈H₄₄O₉N₃FPSi [M+H]⁺: 644.2563, found 644.25513.

G. Synthesis of isopropyl ((S)-((R)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate (MD-7-41)

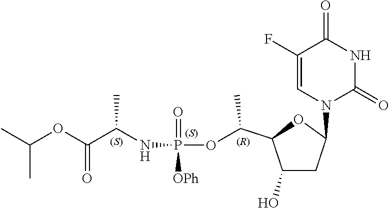

To a solution of isopropyl ((S)-((R)-1-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)ethoxy)(phenoxy)phosphoryl)-L-alaninate (60.0 mg, 0.090 mmol, 1.0 eq) in anhydrous THF (1 mL) at 0° C. was added hydrogen fluoride-pyridine (71.98 μL, 0.560 mmol, 6.0 eq) dropwise. The reaction mixture was allowed to warm to room temperature and stirred for 5 h. The reaction mixture was then quenched with saturated aqueous NaHCO₃ until neutral pH and extracted with EtOAc (×3). The combined organic layers were dried over anhydrous Na₂SO₄, filtered, and concentrated under reduced pressure to obtain a crude mixture. Purification of the crude mixture by silica gel chromatography using 0-100% EtOAc/hexanes eluted MD-7-41 in 100% EtOAc as a solid (16 mg, 0.030 mmol, 32% yield). ¹H NMR (600 MHz, CDCl₃) δ 9.62-9.56 (m, 1H), 7.67 (d, J=6.0 Hz, 1H), 7.31 (t, J=7.8 Hz, 2H), 7.20 (d, J=7.6 Hz, 2H), 7.15 (t, J=7.4 Hz, 1H), 6.16 (ddd, J=7.7, 5.8, 1.6 Hz, 1H), 5.02 (hept, J=6.3 Hz, 1H), 4.81-4.61 (m, 1H), 4.46 (dd, J=6.9, 3.4 Hz, 1H), 4.13-3.84 (m, 2H), 3.84-3.68 (m, 1H), 2.29 (ddd, J=13.8, 5.9, 3.1 Hz, 1H), 1.86 (dt, J=14.3, 7.4 Hz, 1H), 1.44 (d, J=6.5 Hz, 3H), 1.37 (d, J=6.7 Hz, 3H), 1.24 (d, J=6.3 Hz, 6H). ¹³C NMR (151 MHz, CDCl₃) δ 173.18 (d, J=7.6 Hz), 157.06 (d, J=26.6 Hz), 150.51 (d, J=6.7 Hz), 148.98, 140.75 (d, J=237.7 Hz), 129.92, 125.47, 124.41 (d, J=34.0 Hz), 120.59 (d, J=4.5 Hz), 88.60 (d, J=6.5 Hz), 84.81, 74.71 (d, J=5.4 Hz), 71.01, 69.69, 50.69, 40.08, 21.81, 21.75, 21.22 (d, J=4.5 Hz), 18.70 (d, J=2.6 Hz). ¹⁹F NMR (376 MHz, CDCl₃) δ −163.82 (t, J=5.7 Hz). ³¹P NMR (162 MHz, CDCl₃) δ 2.22 (q, J=9.8 Hz). HRMS (APCI) m/z calculated for C₂₂H₃₀O₉N₃FP [M+H]⁺: 530.16982, found 530.1698.

### Example 4. Synthesis of isopropyl ((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate

Scheme 4 below illustrates the synthetic procedures involved in Example 4. Scheme 4 is an alternative synthetic route to the one shown in Scheme 2, for making isopropyl ((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate.

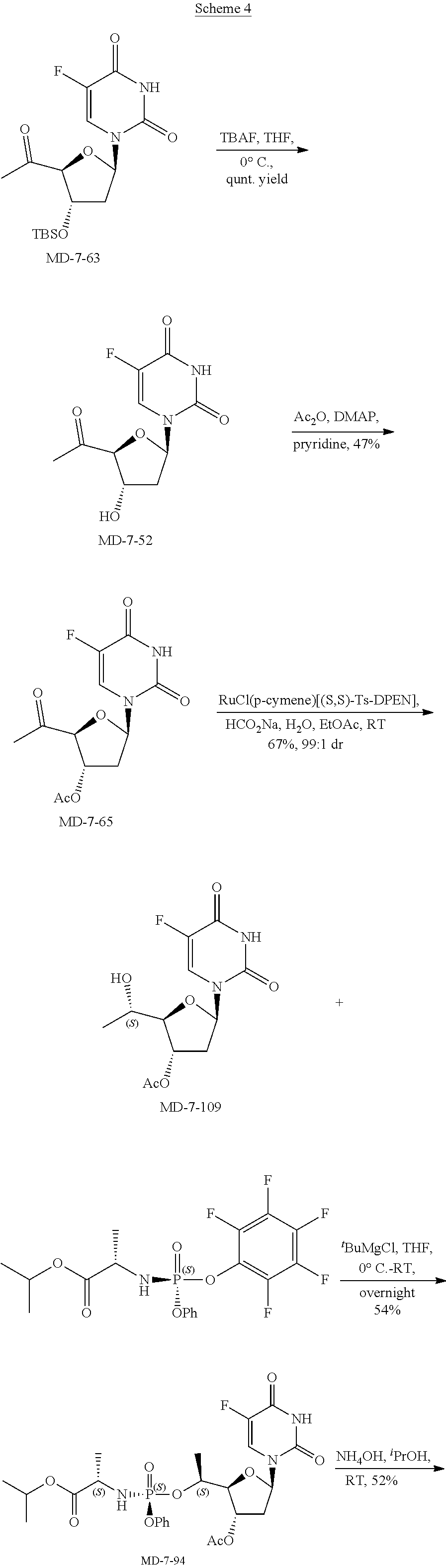

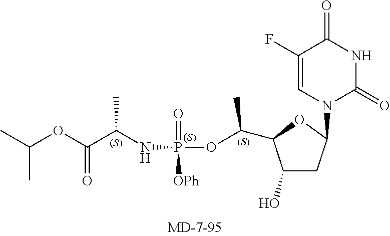

A. Synthesis of 1-((2R,4S,5S)-5-acetyl-4-hydroxytetrahydrofuran-2-yl)-5-fluoropyrimidine-2,4(1H,3H)-dione (MD-7-52)

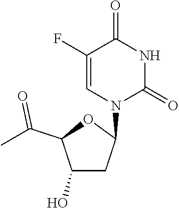 [SMILES: CC(=O)[C@H]1O[C@@H](n2cc(F)c(=O)[nH]c2=O)C[C@@H]1O]

To a solution of 1-((2R,4S,5S)-5-acetyl-4-((tert-butyldimethylsilyl)oxy)tetrahydrofuran-2-yl)-5-fluoropyrimidine-2,4(1H,3H)-dione (100.00 mg, 0.270 mmol, 1.0 eq) in anhydrous THF (3 mL) at 0° C. was added tetra-n-butylammonium fluoride (402.73 μL, 0.400 mmol, 1.5 eq) dropwise and stirred at 0° C. for 1 h. TLC (5% MeOH/CH₂Cl₂) analysis indicated complete conversion. The reaction mixture was concentrated and purified by silica gel chromatography eluted with 0-10% MeOH/CH₂Cl₂ to obtain MD-7-52 at 4-5% gradient as a solid (71 mg, 0.275 mmol, quant. yield). ¹H NMR (400 MHz, DMSO) δ 11.87 (s, 1H), 8.45 (d, J=7.3 Hz, 1H), 6.22 (ddd, J=7.8, 5.6, 1.8 Hz, 1H), 5.78 (d, J=4.2 Hz, 1H), 4.54-4.49 (m, 1H), 4.49-4.44 (m, 1H), 2.20 (s, 3H), 2.13 (ddd, J=13.6, 5.7, 2.3 Hz, 1H), 1.93 (ddd, J=13.7, 8.4, 5.4 Hz, 1H). ¹³C NMR (151 MHz, DMSO) δ 206.79, 157.02 (d, J=26.4 Hz), 149.04, 139.98 (d, J=230.1 Hz), 124.92 (d, J=35.1 Hz). 90.70, 85.83, 71.61, 40.06, 39.94, 39.80, 39.66, 39.52, 39.38, 39.24, 39.10, 38.19, 26.74. ¹⁹F NMR (376 MHz, DMSO) δ −167.56 (d, J=7.3 Hz). HRMS (APCI) m/z calculated for C₁₀H₁₀O₅N₂F [M−H]⁻: 257.05792, found 257.05799.

B. Synthesis of (2S,3S,5R)-2-acetyl-5-(5-fluoro-2,4-dioxo-3,4-dihydropyrimidin-1(2H)-yl)tetrahydrofuran-3-yl acetate (MD-7-65)

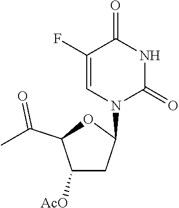 [SMILES: CC(=O)O[C@H]1C[C@H](n2cc(F)c(=O)[nH]c2=O)O[C@@H]1C(C)=O]

To a solution of 1-((2R,4S,5S)-5-acetyl-4-hydroxytetrahydrofuran-2-yl)-5-fluoropyrimidine-2,4(1H,3H)-dione (585.00 mg, 2.06 mmol, 1.0 eq) in anhydrous pyridine (12 mL) was added 4-dimethylaminopyridine (25.19 mg, 0.2100 mmol, 0.1 eq) followed by acetic anhydride (389.79 μL, 4.12 mmol, 2.0 eq) and stirred at room temperature. After 4 h, LC-MS analysis (25-95% MeOH/H₂O; 6 min) indicated complete conversion. The mixture was then concentrated under reduced pressure and co-concentrated with toluene (×2) to obtain a residue. Purification by silica gel chromatography using 20-100% EtOAc/hexanes eluted MD-7-65 at 70-85% gradient as a white solid (290 mg, 0.9659 mmol, 47% yield). ¹H NMR (600 MHz, CDCl₃) δ 9.26 (d, J=4.7 Hz, 1H), 8.62 (d, J=6.3 Hz, 1H), 6.47 (ddd, J=9.5, 5.1, 1.7 Hz, 1H), 5.25 (d, J=5.1 Hz, 1H), 4.72 (s, 1H), 2.54 (dd, J=14.1, 5.1 Hz, 1H), 2.39 (s, 3H), 2.18 (s, 3H), 1.92 (ddd, J=14.4, 9.4, 5.2 Hz, 1H). ¹³C NMR (151 MHz, CDCl₃) δ 205.28, 170.75, 156.86 (d, J=27.1 Hz), 149.25, 140.93 (d, J=237.9 Hz), 124.70 (d, J=35.3 Hz), 88.04, 86.61, 77.37, 77.16, 76.95, 74.71, 35.65, 27.45, 21.06. ¹⁹F NMR (565 MHz, CDCl₃) δ −162.82-−162.97 (m). HRMS (ESI) m/z calculated for C₁₂H₁₄O₆N₂F [M+H]⁺: 301.08304, found 301.08264.

C. Synthesis of (2R,3S,5R)-5-(5-fluoro-2,4-dioxo-3,4-dihydropyrimidin-1(2H)-yl)-2-((S)-1-hydroxyethyl)tetrahydrofuran-3-yl acetate (MD-7-109)

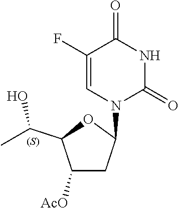

An oven dried three neck flask was charged with (2S,3S,5R)-2-acetyl-5-(5-fluoro-2,4-dioxo-3,4-dihydropyrimidin-1(2H)-yl)tetrahydrofuran-3-yl acetate (350 mg, 1.17 mmol, 1.0 eq) and RuCl(p-cymene)[(S,S)-Ts-DPEN] (7.42 mg, 0.01 mmol, 0.01 eq) and flushed with argon. A solution of sodium formate (3.30 g, 48.49 mmol, 42 eq) in H₂O (20 mL) was added, followed by ethyl acetate (4 mL). The resulting two-phase mixture was stirred overnight at room temperature. After 16 h, TLC analysis (5% MeOH/CH₂Cl₂) indicated complete conversion. The reaction mixture was diluted with EtOAc (15 mL). Organic layer was separated, and aqueous layer was reextracted with EtOAc (10 mL). The combined organic layers were dried over anhydrous Na₂SO₄, filtered, and concentrated under reduced to get a crude solid. Purification of the crude mixture by silica gel chromatography using 0-100% EtOAc/hexanes eluted MD-7-109 in 70-85% gradient as a solid (238 mg, 0.787 mmol, 67% yield). ¹H NMR indicated product with 99:1 dr at 5′(S/R)-carbon. ¹H NMR (600 MHz, CDCl₃) δ 8.25 (d, J=6.4 Hz, 1H), 6.32 (ddd, J=8.8, 5.7, 1.8 Hz, 1H), 5.25 (d, J=6.0 Hz, 1H), 4.04 (qd, J=6.5, 2.0 Hz, 1H), 3.86 (t, J=1.8 Hz, 1H), 2.33 (ddd, J=14.1, 5.7, 1.5 Hz, 1H), 2.24 (ddd, J=14.3, 8.8, 6.1 Hz, 1H), 2.06 (s, 3H), 1.24 (d, J=6.4 Hz, 3H), 1.21 (s, 1H). ¹³C NMR (151 MHz, CDCl₃+few drops MeOD) δ 171.00, 157.45 (d, J=26.5 Hz), 149.25, 140.78 (d, J=236.1 Hz), 124.87 (d, J=34.7 Hz), 88.37, 85.51, 76.37, 67.34, 37.26, 21.09, 20.17. ¹⁹F NMR (565 MHz, CDCl₃) δ −164.43, −164.57. HRMS (APCI) m/z calculated for C₁₂H₁₄O₆N₂F [M−H]⁻: 301.08414, found 301.08407.

D. Synthesis of isopropyl((S)-((S)-1-((2S,3S,5R)-3-acetoxy-5-(5-fluoro-2,4-dioxo-3,4-dihydropyrimidin-1(2H)-yl)tetrahydrofuran-2-yl)ethoxy)(phenoxy)phosphoryl)-L-alaninate (MD-7-94)

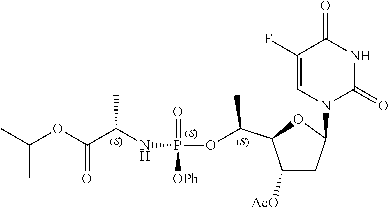

To a solution of (2R,3S,5R)-5-(5-fluoro-2,4-dioxo-3,4-dihydropyrimidin-1(2H)-yl)-2-((S)-1-hydroxyethyl)tetrahydrofuran-3-yl acetate (90.00 mg, 0.300 mmol, 1.0 eq) in anhydrous THF (3.5 mL) at 0° C. was added tert-butylmagnesium chloride (744.39 μL, 0.740 mmol, 2.5 eq, 1 M in THF) dropwise. Ice bath was removed and the reaction was stirred at room temperature for 30 min. The resulting turbid solution was recooled to 0° C. and a solution of isopropyl rac-(2S)-2-[[(2,3,4,5,6-pentafluorophenoxy)-phenoxy-phosphoryl]amino]propanoate (202.46 mg, 0.450 mmol) in anhydrous THF (1.5 mL) was added dropwise. The reaction mixture was allowed to warm to room temperature and stirred overnight. TLC (70% EtOAc/hexanes) indicated complete conversion with the product spot being slightly polar to the starting material. The reaction mixture was concentrated to a crude mixture and purified by silica gel chromatography eluted with 20-100% EtOAc/hexanes to obtain MD-7-94 as a solid (92 mg, 0.161 mmol, 54% yield). ¹H NMR (600 MHz, CDCl₃) δ 9.01 (d, J=4.6 Hz, 1H), 7.85 (d, J=6.1 Hz, 1H), 7.33 (t, J=7.9 Hz, 2H), 7.25-7.22 (m, 2H), 7.16 (t, J=7.4 Hz, 1H), 6.37 (ddd, J=9.3, 5.4, 1.8 Hz, 1H), 5.45-5.36 (m, 1H), 4.99 (p, J=6.3 Hz, 1H), 4.92 (ddt, J=10.7, 6.6, 3.3 Hz, 1H), 4.04-3.93 (m, 2H), 3.85-3.77 (m, 1H), 2.42 (ddd, J=14.1, 5.4, 1.3 Hz, 1H), 2.13 (ddd, J=14.1, 9.2, 6.5 Hz, 1H), 2.10 (s, 3H), 1.45 (d, J=6.5 Hz, 3H), 1.36 (d, J=7.0 Hz, 3H), 1.21 (d, J=6.2 Hz, 6H). ¹³C NMR (151 MHz, CDCl₃) δ 172.95 (d, J=7.8 Hz), 170.58, 156.70 (d, J=27.0 Hz), 150.71 (d, J=6.7 Hz), 148.93, 140.94 (d, J=238.2 Hz), 129.95, 125.26, 123.93 (d, J=34.3 Hz), 120.13 (d, J=4.9 Hz), 87.23 (d, J=6.6 Hz), 84.97, 75.50, 74.81 (d, J=5.9 Hz), 69.69, 50.66, 37.13, 21.78, 21.73, 21.35 (d, J=4.4 Hz), 21.04, 18.46 (d, J=2.2 Hz). ³¹P NMR (162 MHz, CDCl₃) δ 1.98. ¹⁹F NMR (376 MHz, CDCl₃) δ −163.00-−163.18 (m). HRMS (APCI) m/z calculated for C₂₄H₃₂O₁₀N₃FP [M+H]⁺: 572.18039, found 572.18079.

E. Synthesis of isopropyl((S)-((S)-1-((2S,3S,5R)-5-(5-fluoro-2,4-dioxo-3,4-dihydropyrimidin-1(2H)-yl)-3-hydroxytetrahydrofuran-2-yl)ethoxy)(phenoxy)phosphoryl)-L-alaninate (MD-7-95)

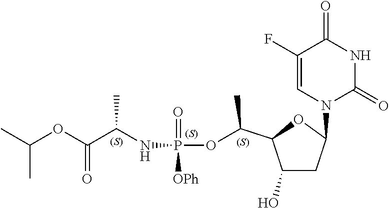

To a solution of isopropyl((S)-((S)-1-((2S,3S,5R)-3-acetoxy-5-(5-fluoro-2,4-dioxo-3,4-dihydropyrimidin-1(2H)-yl)tetrahydrofuran-2-yl)ethoxy)(phenoxy)phosphoryl)-L-alaninate (72.00 mg, 0.130 mmol) in isopropanol (2 mL) was added excess 25% aqueous ammonia (1.6 mL, 18.48 mmol) and stirred at room temperature for 5 h. The reaction was concentrated and purified by silica gel chromatography using 0-20% MeOH/CH₂Cl₂ to obtain MD-7-95 as a solid (35 mg, 0.066 mmol, 52% yield). ¹H NMR (400 MHz, CDCl₃) δ 9.73 (d, J=4.8 Hz, 1H), 7.81 (d, J=6.2 Hz, 1H), 7.37-7.26 (m, 2H), 7.22 (d, J=8.8 Hz, 2H), 7.16 (t, J=7.6 Hz, 1H), 6.25 (td, J=6.1, 1.7 Hz, 1H), 4.98 (hept, J=6.2 Hz, 1H), 4.89-4.81 (m, 1H), 4.56 (q, J=5.6 Hz, 1H), 4.25 (t, J=10.5 Hz, 1H), 3.93 (h, J=7.8 Hz, 1H), 3.82 (dt, J=4.5, 2.0 Hz, 1H), 3.39 (s, 1H), 2.39 (dt, J=13.7, 5.9 Hz, 1H), 2.16 (dt, J=13.2, 6.4 Hz, 1H), 1.44 (d, J=6.5 Hz, 3H), 1.33 (d, J=7.1 Hz, 3H), 1.20 (d, J=6.2 Hz, 6H). ¹³C NMR (151 MHz, CDCl₃) δ 173.06 (d, J=7.7 Hz), 157.11 (d, J=26.6 Hz), 150.70 (d, J=6.6 Hz), 149.03, 140.74 (d, J=236.7 Hz), 129.96, 125.24, 124.29 (d, J=34.2 Hz), 120.02 (d, J=5.0 Hz), 88.19 (d, J=5.6 Hz), 84.73, 77.37, 77.16, 76.95, 73.69 (d, J=6.0 Hz), 70.26, 69.74, 50.55, 40.06, 21.79, 21.71, 21.00 (d, J=4.8 Hz), 18.38 (d, J=2.8 Hz). ³¹P NMR (162 MHz, CDCl₃) δ 2.83. ¹⁹F NMR (376 MHz, CDCl₃) δ −164.27 (t, J=5.9 Hz). HRMS (ESI) m/z calculated for C₂₂H₃₀O₉N₃FP [M+H]⁺: 530.16982, found 530.17108.

### Example 5. Synthesis of isopropyl ((R)-((2-((2S,3S,5R)-5-(5-fluoro-2,4-dioxo-3,4-dihydropyrimidin-1(2H)-yl)-3-hydroxytetrahydrofuran-2-yl)propan-2-yl)oxy)(phenoxy)phosphoryl)-L-alaninate

Scheme 5 below illustrates the synthetic procedures involved in Example 5.

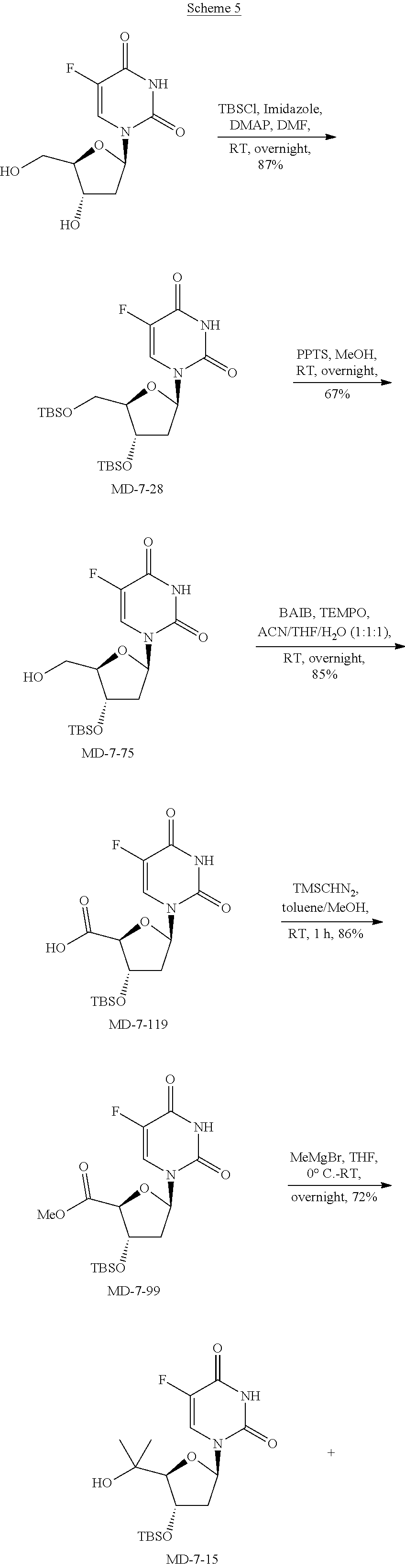 [SMILES: O=c1[nH]c(=O)n([C@H]2C[C@H](O)[C@@H](CO)O2)cc1F ; CC(C)(C)[Si](C)(C)OC[C@H]1O[C@@H](n2cc(F)c(=O)[nH]c2=O)C[C@@H]1O[Si](C)(C)C(C)(C)C ; CC(C)(C)[Si](C)(C)O[C@H]1C[C@H](n2cc(F)c(=O)[nH]c2=O)O[C@@H]1CO ; CC(C)(C)[Si](C)(C)O[C@H]1C[C@H](n2cc(F)c(=O)[nH]c2=O)O[C@@H]1C(=O)O ; COC(=O)[C@H]1O[C@@H](n2cc(F)c(=O)[nH]c2=O)C[C@@H]1O[Si](C)(C)C(C)(C)C ; CC(C)(O)[C@H]1O[C@@H](n2cc(F)c(=O)[nH]c2=O)C[C@@H]1O[Si](C)(C)C(C)(C)C]

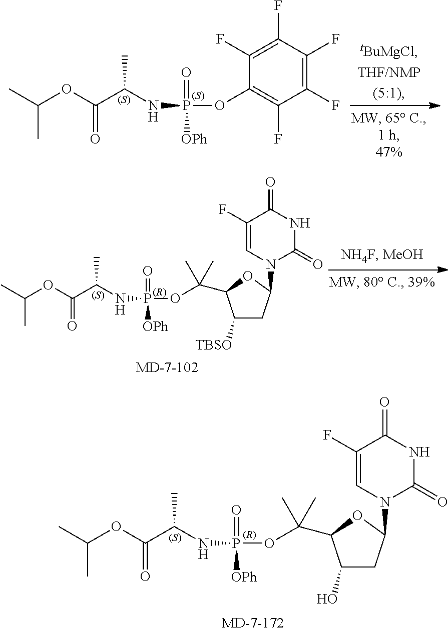

A. Synthesis of methyl (2S,3S,5R)-3-((tert-butyldimethylsilyl)oxy)-5-(5-fluoro-2,4-dioxo-3,4-dihydropyrimidin-1(2H)-yl)tetrahydrofuran-2-carboxylate (MD-7-99)

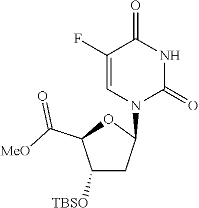 [SMILES: COC(=O)[C@H]1O[C@@H](n2cc(F)c(=O)[nH]c2=O)C[C@@H]1O[Si](C)(C)C(C)(C)C]

To a stirred solution of (2S,3S,5R)-3-((tert-butyldimethylsilyl)oxy)-5-(5-fluoro-2,4-dioxo-3,4-dihydropyrimidin-1(2H)-yl)tetrahydrofuran-2-carboxylic acid (0.73 g, 1.95 mmol, 1.0 eq) in anhydrous methanol (4 mL) and anhydrous toluene (6 mL) was added trimethylsilyldiazomethane (3.56 mL, 3.11 mmol, 1.6 eq) dropwise at room temperature for 1 h. The reaction mixture was quenched with acetic acid and concentrated in vacuo. The crude mixture was purified by silica gel chromatography using 0-100% EtOAc/hexanes to elute MD-7-99 at ˜45-50% gradient as a solid (0.65 g, 1.73 mmol, 86% yield). ¹H NMR (400 MHz, CDCl₃) δ 9.30 (d, J=4.7 Hz, 1H), 8.53 (d, J=6.5 Hz, 1H), 6.48 (ddd, J=9.1, 5.2, 1.8 Hz, 1H), 4.50 (d, J=4.4 Hz, 1H), 4.47 (s, 1H), 2.37 (ddd, J=13.3, 5.2, 1.4 Hz, 1H), 1.91 (ddd, J=13.5, 9.0, 4.6 Hz, 1H), 0.90 (s, 92 (s, 3H), 0.12 (s, 3H). ¹F NMR (376 MHz, CDCl₃) δ −163.69 (ddd, J=6.5, 4.6, 1.8 Hz). ¹³C NMR (151 MHz, CDCl₃) δ 171.96, 157.07 (d, J=26.9 Hz), 149.11, 140.80 (d, J=237.1 Hz), 125.01 (d, J=35.1 Hz), 87.44, 85.51, 75.79, 52.87, 39.99, 25.77, 18.11, −4.82. HRMS (APCI) m/z calculated for C₁₆H₂₄O₆N₂FSi [M−H]⁻: 387.13931, found 387.13974. LC-MS: 75-95% MeOH/H₂O, 3 min, t_{R}=2.27 min, [M+Na]⁺=411, [M−H]⁻=387.

B. Synthesis of 1-((2R,4S,5S)-4-((tert-butyldimethylsilyl)oxy)-5-(2-hydroxypropan-2-yl)tetrahydrofuran-2-yl)-5-fluoropyrimidine-2,4(1H,3H)-dione (MD-7-15)

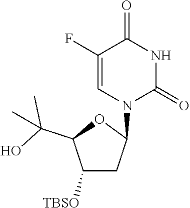 [SMILES: CC(C)(O)[C@H]1O[C@@H](n2cc(F)c(=O)[nH]c2=O)C[C@@H]1O[Si](C)(C)C(C)(C)C]

To a solution of methyl (2S,3S,5R)-3-((tert-butyldimethylsilyl)oxy)-5-(5-fluoro-2,4-dioxo-3,4-dihydropyrimidin-1(2H)-yl)tetrahydrofuran-2-carboxylate (0.84 g, 2.16 mmol, 1.0 eq) in anhydrous THF (15 mL) at 0° C. was added methylmagnesium bromide (3.60 mL, 10.81 mmol, 5.0 eq, 3.0 M in diethylether) dropwise. After 15 min, ice bath was removed, and the reaction was allowed to warm to room temperature and stirred overnight. After 20 h, TLC analysis (50% EtOAc/hexanes) indicated the product with very close R_{f} to the starting material. The reaction mixture was cooled to 0° C. and quenched with saturated aqueous NH₄Cl and extracted with EtOAc (×3). The combined organic layers were dried over anhydrous Na₂SO₄, filtered, and concentrated under reduced pressure, purified by silica gel chromatography using 20-100% EtOAc/hexanes to elute MD-7-15 in 40-50% gradient as a white solid (620 mg, 1.563 mmol, 72% yield). ¹H NMR (400 MHz, CDCl₃) δ 8.32 (s, 1H), 8.00 (d, J=6.4 Hz, 1H), 6.24 (td, J=6.9, 1.6 Hz, 1H), 4.58-4.44 (m, 1H), 3.68 (d, J=2.6 Hz, 1H), 2.23-2.13 (m, 2H), 1.71 (brs, 1H), 1.33 (s, 3H), 1.31 (s, 3H), 0.89 (s, 9H), 0.10 (s, 6H). ¹⁹F NMR (376 MHz, CDCl₃) δ −164.41 (ddd, J=6.5, 4.8, 1.6 Hz). ¹¹C NMR (151 MHz, cdcl₃) S 172.65, 157.05 (d, J=26.7 Hz), 149.02, 140.64 (d, J=236.8 Hz), 125.55 (d, J=34.5 Hz), 93.75, 86.28, 77.37, 77.16, 76.95, 71.75, 71.52, 41.11, 27.71, 27.11, 25.81, 17.92, −4.18, −4.68. HRMS (APCI) m/z calculated for C₁₇H₂₈O₅N₂FSi [M−H]⁻: 387.1757, found 387.17616. LC-MS (ESI) 85% MeOH/H₂O (0.1% HCO₂H), 3 min, 1.00 mL/min, t_{R}=0.72 min, m/z=387 [M−H]⁻.

C. Synthesis of isopropyl((R)-((2-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)propan-2-yl)oxy)(phenoxy)phosphoryl)-L-alaninate (MD-7-102)

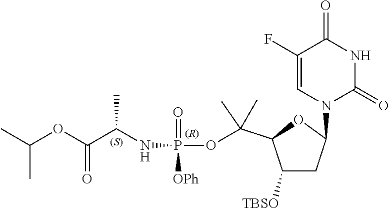

In a flame dried microwave vial, 1-((2R,4S,5S)-4-((tert-butyldimethylsilyl)oxy)-5-(2-hydroxypropan-2-yl)tetrahydrofuran-2-yl)-5-fluoropyrimidine-2,4(1H,3H)-dione (100.00 mg, 0.260 mmol, 1.0 eq) was suspended and dissolved in a mixture of anhydrous THF (2 mL) and anhydrous NMP (0.4 mL). tert-Butylmagnesium chloride (514.79 μL, 0.510 mmol, 1 M in THF, 2.0 eq) was then added dropwise at room temperature. After stirring for 10 min, a solution of isopropyl rac-(2S)-2-[[(2,3,4,5,6-pentafluorophenoxy)-phenoxy-phosphoryl]amino]propanoate (233 mg, 0.510 mmol, 2.0 eq) in anhydrous THF (1 mL) was added dropwise. The vial was sealed and subjected to microwave irradiation at 65° C. for 1 h. The reaction mixture was quenched with aqueous saturated NH₄Cl solution and extracted with CH₂Cl₂ (×3). The combined organic layers were again washed with water followed by brine solution. The organic layer was dried over anhydrous Na₂SO₄, filtered, and concentrated to get a crude mixture. Purification by silica gel chromatography using 0-70% EtOAc/hexanes afforded MD-7-102 at 50% gradient as a solid (85 mg, 0.120 mmol, 47% yield). ¹H NMR (600 MHz, CDCl₃) δ 8.98 (d, J=4.8 Hz, 1H), 8.01 (d, J=6.3 Hz, 1H), 7.30 (t, J=7.9 Hz, 2H), 7.25-7.21 (m, 2H), 7.14 (t, J=7.3 Hz, 1H), 6.24 (ddd, J=8.4, 5.5, 1.7 Hz, 1H), 5.02-4.95 (m, 1H), 4.51-4.47 (m, 1H), 3.94 (ddt, J=16.2, 9.2, 7.0 Hz, 1H), 3.73 (t, J=10.0 Hz, 1H), 3.65 (dd, J=5.0, 2.6 Hz, 1H), 2.12 (ddd, J=13.2, 5.5, 2.0 Hz, 1H), 1.70 (s, 3H), 1.66 (dd, J=8.6, 2.2 Hz, 1H), 1.63 (s, 3H), 1.33 (d, J=7.0 Hz, 3H), 1.21 (m, 6H), 0.89 (s, 9H), 0.10 (d, J=5.5 Hz, 6H). ¹³C NMR (151 MHz, CDCl₃) δ 173.01 (d, J=7.7 Hz), 156.88 (d, J=27.0 Hz), 150.81 (d, J=6.6 Hz), 148.79, 140.74 (d, J=236.8 Hz), 129.83, 125.15, 124.70 (d, J=33.9 Hz), 120.44 (d, J=4.7 Hz), 93.69 (d, J=7.3 Hz), 84.70, 84.26 (d, J=7.2 Hz), 71.54, 69.50, 60.54, 50.70, 41.30, 25.78, 25.31 (d, J=2.1 Hz), 24.85, 21.80, 21.74, 21.35 (d, J=4.4 Hz) 17.86, −4.14, −4.69. ¹⁹F NMR (376 MHz, CDCl₃) δ −163.96 (ddd, J=6.5, 4.9, 1.8 Hz). ³¹P NMR (162 MHz, CDCl₃) δ −1.28. HRMS (ESI) m/z calculated for C₂₉H₄₆O₉N₃FPSi [M+H]⁺: 658.27195, found 658.27122. LC-MS: 85-95% MeOH/H₂O, 6 min, t_{R}=3.5, [M+Na]+=680; [M−H]⁻=656.

D. Synthesis of isopropyl ((R)-((2-((2S,3S,5R)-5-(5-fluoro-2,4-dioxo-3,4-dihydropyrimidin-1(2H)-yl)-3-hydroxytetrahydrofuran-2-yl)propan-2-yl)oxy)(phenoxy)phosphoryl)-L-alaninate (MD-7-172)

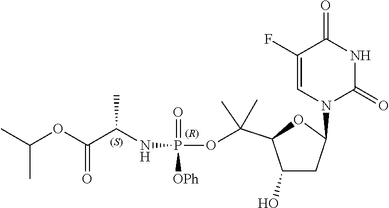

In a microwave vial, to a solution of isopropyl((R)-((2-((2S,3S,5R)-3-((tert-butyldimethylsilyl)oxy)-5-(5-fluoro-2,4-dioxo-3,4-dihydropyrimidin-1(2H)-yl)tetrahydrofuran-2-yl)propan-2-yl)oxy)(phenoxy)phosphoryl)-L-alaninate (50.00 mg, 0.07 mmol, 1.0 eq) in methanol (0.7 mL) was added ammonium fluoride (36.81 mg, 0.71 mmol, 10 eq) and irradiated at 80° C. for 30 min. TLC analysis (70% EtOAc/hexanes) indicated incomplete conversion. Additional ammonium fluoride (36.81 mg, 0.71 mmol, 10 eq) was added and irradiated at 80° C. for another 30 min. TLC indicated increase in conversion. Additional ammonium fluoride (18.5 mg, 0.35 mmol, 5 eq) was added and irradiated at 80° C. for another 30 min. The contents were concentrated and purified by silica gel chromatography using 30-100% EtOAc/hexanes to elute MD-7-172 in 100% EtOAc as a solid (15 mg, 0.927 mmol, 39% yield). ¹H NMR (600 MHz, CDCl₃) δ 9.51 (s, 1H), 7.73 (d, J=6.0 Hz, 1H), 7.31 (t, J=7.9 Hz, 2H), 7.22 (d, J=8.0 Hz, 2H), 7.15 (t, J=7.4 Hz, 1H), 6.27 (td, J=7.4, 6.6, 3.5 Hz, 1H), 5.05-4.97 (m, 1H), 4.57 (dt, J=6.5, 2.8 Hz, 1H), 3.94-3.88 (m, 2H), 3.83 (t, J=3.3 Hz, 1H), 2.34 (ddd, J=13.6, 5.7, 2.4 Hz, 1H), 1.88 (dt, J=14.4, 7.6 Hz, 1H), 1.67 (s, 3H), 1.61 (s, 3H), 1.32 (d, J=5.7 Hz, 3H), 1.22 (d, J=6.2 Hz, 6H). ¹³C NMR (151 MHz, CDCl₃) δ 173.35 (d, J=6.7 Hz), 156.95 (d, J=26.7 Hz), 150.75 (d, J=6.6 Hz), 149.00, 140.85 (d, J=237.8 Hz), 129.87, 125.18, 124.15 (d, J=33.8 Hz), 120.31 (d, J=4.9 Hz), 92.03 (d, J=6.3 Hz), 84.41 (d, J=7.7 Hz), 84.38, 70.51, 69.63, 50.72, 40.51, 25.47, 24.40 (d, J=3.3 Hz), 21.82, 21.74, 21.09, 21.06. ¹⁹F NMR (565 MHz, CDCl₃) δ −163.53. ³¹P NMR (243 MHz, CDCl₃) δ −1.26. LC-MS (ESI) 50-95% MeOH/H₂O (0.1% HCO₂H), 6 min, 1.00 mL/min, t_{R}=2.81 min, m/z=542.2 [M−H]⁻; LC-MS (ESI) 50-95% MeOH/H₂O (0.1% HCO₂H), 3 min, t_{R}=2.98 min, m/z=542.2 [M−H]⁻.

### Example 6. Synthesis of lithium (R)-1-((2S,3S,5R)-5-(5-fluoro-2,4-dioxo-3,4-dihydropyrimidin-1(2H)-yl)-3-hydroxytetrahydrofuran-2-yl)ethyl phosphate

Scheme 6 below illustrates the synthetic procedures involved in Example 6.

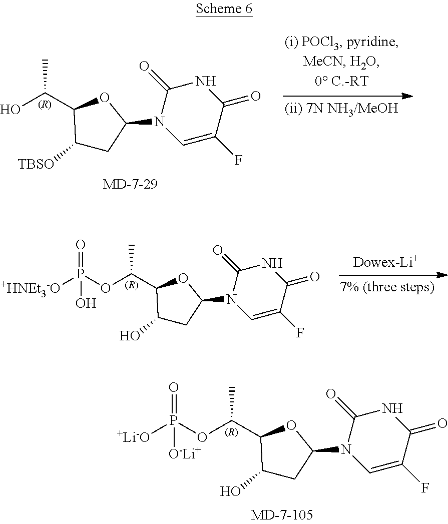

To a solution of 1-((2R,4S,5R)-4-((tert-butyldimethylsilyl)oxy)-5-((R)-1-hydroxyethyl)tetrahydrofuran-2-yl)-5-fluoropyrimidine-2,4(1H,3H)-dione (150 mg, 0.4000 mmol, 1.0 eq) in anhydrous MeCN (0.9 mL) at 0° C. was added water (15.88 mg, 0.880 mmol, 2.2 eq) followed by pyridine (0.14 mL, 1.76 mmol, 4.4 eq). After 10 min, phosphorus oxychloride (0.17 mL, 1.76 mmol, 4.4 eq) was added dropwise and stirred overnight by allowing the reaction mixture to be gradually warmed to room temperature. After 19 h, the reaction was cooled to 0° C. and quenched with ice cold water (0.5 mL) and stirred for 1 h. The contents were concentrated and co-concentrated with methanol (×2) to give a green colored crude product.

The crude product was dissolved in methanol (2 mL) and transferred to a falcon tube. The pH of the resulting solution was acidic (pH<3). The solution was neutralized to pH=7 using 7 N NH₃/MeOH (˜600 μL). The precipitated solids were dissolved in water (2 mL), and to the resulting solution, ammonium fluoride (148.37 mg, 4.01 mmol) was added at room temperature and stirred overnight. Reaction progress was monitored by LC-MS. After 18 h, The reaction mixture was concentrated and purified by RP C₁₈ flash chromatography using 0-10% MeOH/H₂O. The product fractions were concentrated and repurified by DEAE resin flash chromatography using freshly prepared 1 M triethylammonium bicarbonate (TEAB) with a gradient of 50 mM TEAB-400 mM TEAB. The purified fractions were pooled, concentrated, and lyophilized to obtain a solid. The solid was then subjected to RP C₁₈ flash chromatography eluted with 100% H₂O followed by 0-100% MeOH/H₂O to obtain the product as 0.8 mol % Et₃N salt (15 mg, 0.0340 mmol, 8% yield).

A solution of the product as a triethylammonium salt in DI water (1 mL) was slowly added to the Dowex-Li⁺ resin and eluted with DI water (5-6 column volumes). The eluant was collected in small fractions and analyzed by nanodrop for UV absorption. The product fractions were pooled and lyophilized to obtain MD-7-105 as a white fluffy solid (10 mg, 0.0284 mmol, 7.1% yield over three steps). ¹H NMR (600 MHz, D₂O) S 8.07 (d, J=6.2 Hz, 1H), 6.32 (ddd, J=7.8, 6.0, 1.7 Hz, 1H), 4.70-4.56 (m, 1H), 4.42-4.17 (m, 1H), 3.87 (ddd, J=5.4, 2.9, 1.3 Hz, 1H), 2.43-2.25 (m, 2H), 1.36 (d, J=6.4 Hz, 3H). ¹³C NMR (151 MHz, D₂O) S 160.15 (d, J=25.1 Hz), 150.72, 141.04 (d, J=233.9 Hz), 125.82 (d, J=34.2 Hz), 89.59 (d, J=7.8 Hz), 85.14, 71.20 (d, J=5.3 Hz), 70.67, 38.40, 17.75. ³¹P NMR (243 MHz, D₂O) S 1.20. HRMS (ESI) m/z calculated for C₁₀H₁₃O₅N₂FP [M−H]⁻: 339.0399, found 339.04038.

### Example 7. Synthesis of lithium (S)-1-((2S,35R)-5-(5-fluoro-2,4-dioxo-3,4-dihydropyrimidin-1(2H)-yl)-3-hydroxytetrahydrofuran-2-yl)ethyl phosphate

Scheme 7 below illustrates the synthetic procedures involved in Example 7.

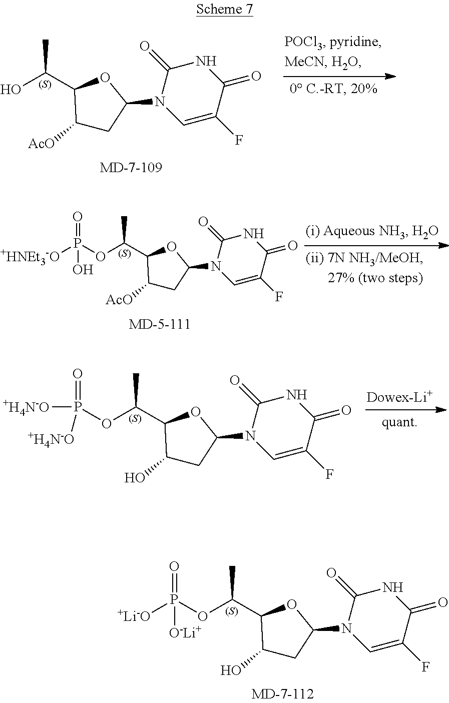

A. Synthesis of triethylammonium (S)-1-((2S,3S,5R)-3-acetoxy-5-(5-fluoro-2,4-dioxo-3,4-dihydropyrimidin-1(2H)-yl)tetrahydrofuran-2-yl)ethyl hydrogen phosphate (MD-7-111)

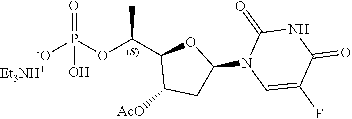

To a solution of (2R,3S,5R)-5-(5-fluoro-2,4-dioxo-3,4-dihydropyrimidin-1(2H)-yl)-2-((S)-1-hydroxyethyl)tetrahydrofuran-3-yl acetate (125.00 mg, 0.410 mmol, 1.0 eq) in anhydrous MeCN (1.5 mL) at 0° C. was added pyridine (0.15 mL, 1.82 mmol, 4.4 eq) followed by water (0.02 mL, 0.9100 mmol, 2.2 eq). After 10 min, phosphorus oxychloride (0.17 mL, 1.82 mmol, 4.4 eq) was added dropwise and stirred at 0° C. overnight (cold room). After 17 h, the reaction was cooled to 0° C. and quenched with water (1 mL) followed by 0.5 M TEAB (10 mL) and then 1 M TEAB (10 mL) until pH >7. The contents were concentrated and co-concentrated with methanol (×2) to yield a crude solid. The reaction mixture was purified by RP C₁₈ flash chromatography using 0-100% MeOH/H₂O to elute the product at 25-30% gradient. The product fractions were pooled and concentrated and lyophilized to get MD-7-111 as a solid (40 mg, 0.0827 mmol, 20% yield). ¹H NMR (400 MHz, D₂O) S 8.27 (d, J=6.2 Hz, 1H), 6.58-6.23 (m, 1H), 5.43 (d, J=5.2 Hz, 1H), 4.52 (s, 1H), 4.19 (s, 1H), 2.69-2.31 (m, 2H), 2.14 (s, 3H), 1.37 (d, J=5.3 Hz, 3H). ¹⁹F NMR (376 MHz, D₂O) δ −164.34 (d, J=6.1 Hz). HRMS (ESI) m/z calculated for C₁₂H₁₅FN₂O₉P [M−H]⁻: 381.05047, found 381.0503.

B. Synthesis of lithium (S)-1-((2S,3S,5R)-5-(5-fluoro-2,4-dioxo-3,4-dihydropyrimidin-1(2H)-yl)-3-hydroxytetrahydrofuran-2-yl)ethyl phosphate (MD-7-112)

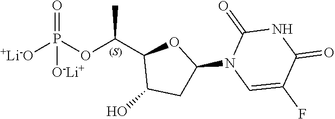

To a solution of triethylammonium (S)-1-((2S,3S,5R)-3-acetoxy-5-(5-fluoro-2,4-dioxo-3,4-dihydropyrimidin-1(2H)-yl)tetrahydrofuran-2-yl)ethyl hydrogen phosphate (40.00 mg, 0.0800 mmol) in water (2 mL) was added aqueous ammonia (0.50 mL, 5.79 mmol) at room temperature and stirred overnight. Reaction progress was monitored by LC-MS (50-95% MeOH/H₂O, 3 min). After 22 h, the reaction mixture was purified by RP C₁₈ flash chromatography eluted with 100% H₂O. Product fractions were pooled and lyophilized to get the product (acid form) as a white solid (29 mg, 103% yield).

To the product (acid form) in methanol (2 mL) was added ammonia solution (7 N in methanol) and stirred for 1 h. The mixture was concentrated and lyophilized to obtain the product (ammonium salt) as a solid. ¹H NMR (400 MHz, D₂O) δ 8.24 (d, J=6.5 Hz, 1H), 6.39 (ddd, J=8.0, 6.2, 1.9 Hz, 1H), 4.59 (dt, J=5.7, 2.8 Hz, 1H), 4.51-4.44 (m, 1H), 4.00 (q, J=2.3 Hz, 1H), 2.45-2.25 (m, 2H), 1.39 (d, J=6.5 Hz, 3H). ¹F NMR (376 MHz, D₂O) δ −164.91 (dd, J=6.4, 1.9 Hz). ³¹P NMR (162 MHz, D₂O) δ −0.42. ¹C NMR (151 MHz, D₂O) δ 159.52 (d, J=26.1 Hz), 150.20, 140.81 (d, J=233.1 Hz), 125.89, 125.78 (d, J=34.8 Hz), 89.76 (d, J=7.5 Hz), 85.29, 71.95, 71.66 (d, J=5.3 Hz), 38.72, 17.86. HRMS (ESI) m/z calculated for C₁₀H₁₃O₅N₂FP [M−H]⁻: 339.0399, found 339.03984.

A solution of the triethylammonium salt in DI water (1 mL) was slowly added to the Dowex-Li⁺ resin and eluted with DI water (5-6 column volumes). The eluant was collected in small fractions and analyzed by nanodrop for UV absorption. The product fractions were pooled and lyophilized to obtain MD-7-112 as a white fluffy solid (8 mg, 0.0227 mmol, 27% yield). ¹H NMR (600 MHz, D₂O) δ 8.26 (d, J=6.4 Hz, 1H), 6.39 (t, J=7.1 Hz, 1H), 4.64-4.53 (m, 1H), 4.47 (t, J=7.8 Hz, 1H), 4.00 (t, J=2.4 Hz, 1H), 2.62-1.93 (m, 2H), 1.39 (d, J=6.5 Hz, 3H). ¹³C NMR (151 MHz, D₂O) 6159.69 (d, J=25.9 Hz), 150.36, 140.91 (d, J=233.1 Hz), 125.90 (d, J=34.6 Hz), 89.91 (d, J=7.4 Hz), 85.34, 72.04, 71.52 (d, J=5.4 Hz), 38.79, 17.96. ³¹P NMR (243 MHz, D₂O) δ −0.07. HRMS (ESI) m/z calculated for C₁₀H₁₃O₅N₂FP [M−H]⁻: 339.0399, found 339.03967.

### Example 8. Synthesis of lithium 2-((2S,3S,5R)-5-(5-fluoro-2,4-dioxo-3,4-dihydropyrimidin-1(2H)-yl)-3-hydroxytetrahydrofuran-2-yl)propan-2-yl phosphate

Scheme 8 below illustrates the synthetic procedures involved in Example 8.

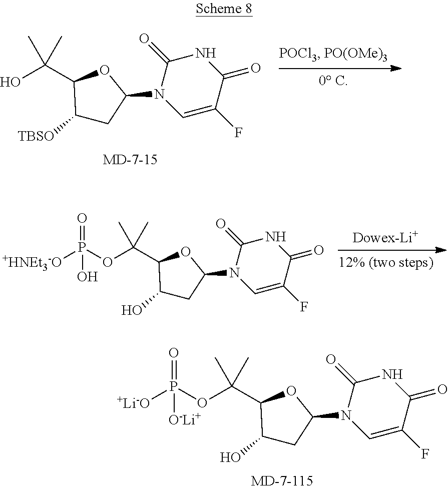

A solution of 1-((2R,4S,5S)-4-((tert-butyldimethylsilyl)oxy)-5-(2-hydroxypropan-2-yl)tetrahydrofuran-2-yl)-5-fluoropyrimidine-2,4(1H,3H)-dione (50.00 mg, 0.1300 mmol, 1.0 eq) in trimethyl phosphate (0.500 mL, stored over activated molecular sieves) was heated at 50° C. for 15 mins and then cooled to 0° C. and then added phosphorus oxychloride (0.02 mL, 0.260 mmol, 2.0 eq) dropwise and stirred overnight at 0° C. (cold room). After 4 days, the reaction was quenched with 100 mM TEAB (40 mL) at 0° C. and stirred at room temperature for 1 h. The mixture was extracted with ether (3×7 mL). The aqueous layer was concentrated to a crude mixture. LC-MS analysis (25-95% MeOH/H₂O, 3 min) indicated product (TBS deprotected). The mixture was purified by RP C₁₈ flash chromatography using 100% H₂O to elute TBS deprotected product. The product fractions were pooled and lyophilized to get a fluffy white solid. ¹H NMR indicated product as a triethylammonium salt (8.1 mg, 0.0178 mmol, 14% yield). ¹H NMR (600 MHz, D₂O) δ 8.14 (d, J=6.8 Hz, 1H), 6.34 (t, J=6.9 Hz, 1H), 4.87 (m, 1H), 3.95 (d, J=2.7 Hz, 1H), 3.22 (q, J=7.3 Hz, 6H), 2.58 (dd, J=14.5, 6.4 Hz, 1H), 2.33 (dt, J=14.3, 7.1 Hz, 1H), 1.35 (s, 3H), 1.32-1.27 (m, 12H). ¹³C NMR (151 MHz, D₂O) δ 159.65 (d, J=25.9 Hz), 150.38, 140.84 (d, J=233.3 Hz), 126.02 (d, J=34.4 Hz), 91.64 (d, J=6.9 Hz), 84.96, 74.00, 71.15, 46.74, 38.10, 25.44, 25.01, 8.29. ³¹P NMR (243 MHz, D₂O) δ −0.03. ¹F NMR (376 MHz, D₂O) δ −165.57 (d, J=6.3 Hz). HRMS [ESI]m/z [M−H]⁻ calculated for C₁₁H₁₅O₈N₂FP: 353.05555; found: 353.05539.

A solution of the triethylammonium salt in DI water (1 mL) was slowly added to the Dowex-Li⁺ resin and eluted with DI water (5-6 column volumes). The eluant was collected in small fractions and analyzed by nanodrop for UV absorption. The product fractions were pooled and lyophilized to obtain MD-7-115 as a white fluffy solid (5.8 mg, 0.0158 mmol, 12% yield). ¹H NMR (600 MHz, D₂O) δ 8.14 (d, J=6.4 Hz, 1H), 6.34 (t, J=7.1 Hz, 1H), 4.84 (t, J=3.5 Hz, 1H), 3.93 (d, J=3.2 Hz, 1H), 2.58 (ddd, J=14.2, 6.2, 2.9 Hz, 1H), 2.33 (dt, J=14.4, 7.3 Hz, 1H), 1.35 (s, 3H), 1.31 (s, 3H). ¹³C NMR (151 MHz, D₂O) δ 159.83 (d, J 25.6 Hz), 150.54, 140.87 (d, J=233.3 Hz), 126.04 (d, J=34.4 Hz), 91.64 (d, J=7.3 Hz), 84.97, 73.55 (d, J=4.8 Hz), 38.15, 71.18, 38.15 (d, J=2.2 Hz), 25.37, 25.09. ³¹P NMR (243 MHz, D₂O) δ 0.95. HRMS [ESI] nm/z [M−H]⁻ calculated for C₁₁H₁₅O₈N₂FP: 353.05555; found: 353.05584.

### Example 9. Synthesis of (R/S)-2,2,2-trifluoro-1-((2S,3S,5R)-5-(5-fluoro-2,4-d oxo-3,4-dihydropyrimidin-1(2H)-yl)-3-hydroxytetrahydrofuran-2-yl)ethyl dihydrogen phosphate

Scheme 9 below illustrates the synthetic procedures involved in Example 9.

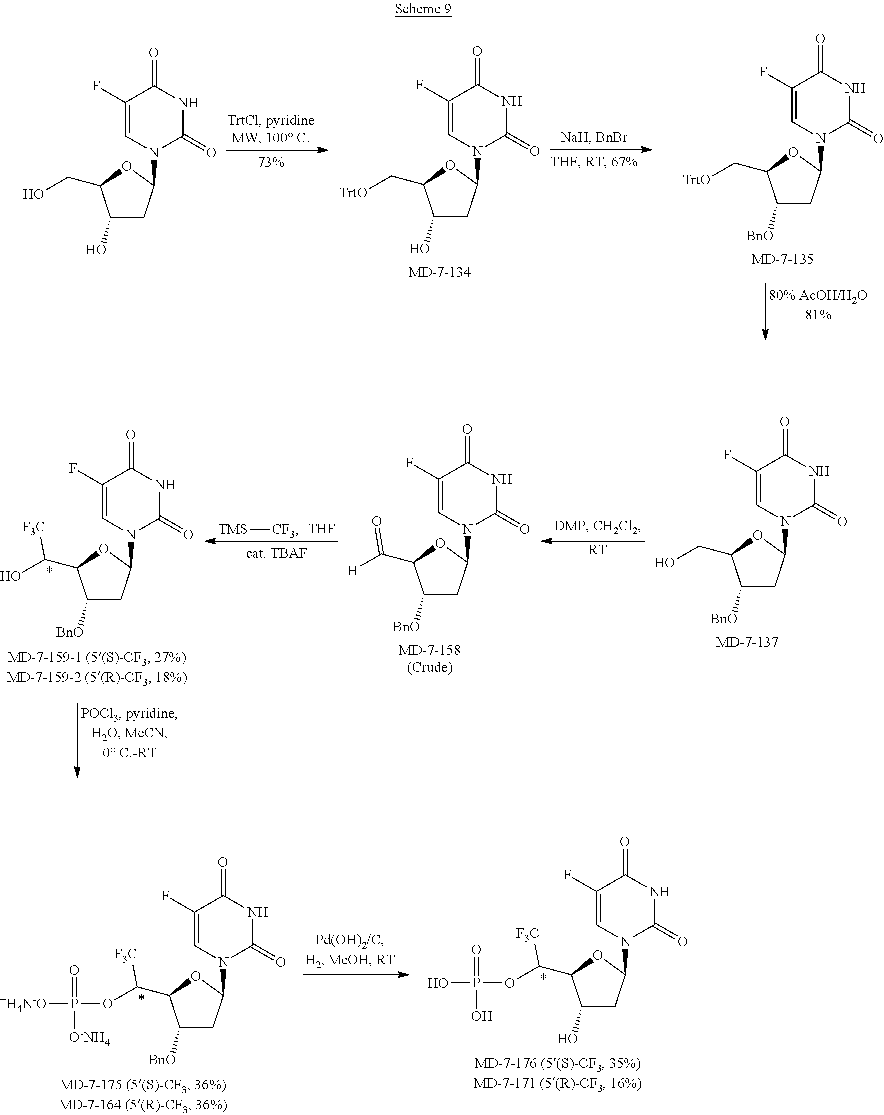

A. Synthesis of 5-fluoro-1-((2R,4S,5R)-4-hydroxy-5-((trityloxy)methyl)tetrahydrofuran-2-yl)pyrimidine-2,4(1H,3H)-dione (MD-7-134)

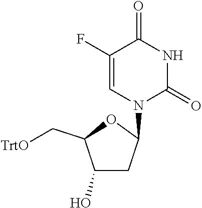

In a microwave vial, a mixture of 5-fluoro-2′-deoxyuridine (2.00 g, 8.12 mmol, 1.0 eq) and trityl chloride (2.29 g, 8.21 mmol, 1.01) in anhydrous pyridine (20 mL) was subjected to microwave irradiation at 100° C. for 10 min. The mixture was then poured into 1 N HCl (100 mL) and extracted with CHCl₃ (100 mL). The organic layer was again washed with water, followed by saturated NaHCO₃ solution. The organic layer was dried over anhydrous Na₂SO₄, filtered, and concentrated to give a crude solid. Purification of the crude solid by silica gel chromatography using 20-100% EtOAc/hexanes eluted MD-7-141 at 80% gradient as a solid (2.9 g, 5.93 mmol, 73% yield). ¹H NMR (600 MHz, CDCl₃) δ 8.52-8.48 (m, 1H), 7.80 (d, J=6.0 Hz, 1H), 7.44-7.40 (m, 6H), 7.35-7.26 (m, 6H), 7.26 (d, J=3.4 Hz, 3H), 6.27 (td, J=6.5, 1.6 Hz, 1H), 4.54 (dq, J=6.8, 3.3 Hz, 1H), 4.04 (q, J=3.4 Hz, 1H), 3.49-3.41 (m, 2H), 2.47 (ddd, J=13.8, 6.1, 3.7 Hz, 1H), 2.26 (dt, J=13.4, 6.5 Hz, 1H), 1.96 (d, J=3.9 Hz, 1H). ¹⁹F NMR (565 MHz, CDCl₃) δ −164.34. ¹³C NMR (151 MHz, CDCl₃) δ 156.64 (d, J=27.1 Hz), 148.56, 143.29, 140.60 (d, J=238.2 Hz), 128.68, 128.26, 128.07, 127.63, 124.19 (d, J=33.9 Hz), 87.97, 86.26, 85.50, 71.89, 63.35, 41.10.

B. Synthesis of 1-((2R,4S,5R)-4-(benzyloxy)-5-((trityloxy)methyl)tetrahydrofuran-2-yl)-5-fluoropyrimidine-2,4(1H,3H)-dione (MD-7-135)

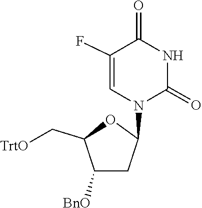

To a suspension of sodium hydride (614.09 mg, 15.35 mmol, 3.0 eq, 60% dispersion in mineral oil) in anhydrous THF (5 mL) was added a solution of 5-fluoro-1-((2R,4S,5R)-4-hydroxy-5-((trityloxy)methyl)tetrahydrofuran-2-yl)pyrimidine-2,4(1H,3H)-dione (2.50 g, 5.12 mmol, 1.0 eq) in anhydrous THF (12 mL) dropwise at room temperature. After 1 h, benzyl bromide (0.91 mL, 7.68 mmol, 1.5 eq) was added and stirred overnight. After 17 h, the reaction mixture was cooled to 0° C. and quenched with ice cold water slowly. The mixture was extracted with EtOAc (×2). The combined organic layers were washed with water followed by brine solution. The organic layer was dried over anhydrous Na₂SO₄, filtered, and concentrated to give a crude mixture. Purification by silica gel chromatography using 0-80% EtOAc/hexanes eluted MD-7-135 at 40-70% gradient as a solid (2.0 g, 3.45 mmol, 67% yield).

¹H NMR (600 MHz, cdcl₃) δ 8.16 (d, J=4.6 Hz, 1H), 7.82 (d, J=6.0 Hz, 1H), 7.38-7.24 (m, 20H), 6.26 (ddd, J=7.6, 5.7, 1.7 Hz, 1H), 4.58-4.43 (m, 2H), 4.28 (dt, J=5.9, 2.8 Hz, 1H), 4.20 (q, J=3.1 Hz, 1H), 3.39 (qd, J=10.8, 3.2 Hz, 2H), 2.60 (ddd, J=13.7, 5.9, 2.7 Hz, 1H), 2.15 (ddd, J=13.6, 7.6, 6.2 Hz, 1H). ¹⁹F NMR (565 MHz, CDCl₃) δ −164.31, −164.31. ¹³C NMR (151 MHz, CDCl₃) δ 156.87 (d, J=26.7 Hz), 148.77, 147.01, 143.27, 140.62 (d, J=238.1 Hz), 137.35, 128.74, 128.65, 128.20, 128.17, 128.06, 127.86, 127.56, 127.39, 124.15 (d, J=34.1 Hz), 87.82, 85.78, 84.42, 78.35, 71.56, 63.62, 38.47.

C. Synthesis of 1-((2R,4S,5R)-4-(benzyloxy)-5-(hydroxymethyl)tetrahydrofuran-2-yl)-5-fluoropyrimidine-2,4(1H,3H)-dione (MD-7-137)

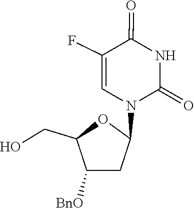 [SMILES: O=c1[nH]c(=O)n([C@H]2C[C@H](OCc3ccccc3)[C@@H](CO)O2)cc1F]

A solution of 1-((2R,4S,5R)-4-(benzyloxy)-5-((trityloxy)methyl)tetrahydrofuran-2-yl)-5-fluoropyrimidine-2,4(1H,3H)-dione (1.90 g, 3.28 mmol) in 80% AcOH in H₂O (15 mL) was heated at 60° C. for 1 h followed by stirring at 0° C. for 2 days. The reaction mixture was concentrated and purified by silica gel chromatography eluted with 20-100% EtOAc/hexanes to obtain MD-7-137 at ˜80% gradient as a solid (0.9 g, 2.67 mmol, 81% yield). ¹H NMR (600 MHz, CDCl₃) δ 8.03 (d, J=6.4 Hz, 1H), 7.35-7.23 (m, 5H), 6.22 (ddd, J=7.6, 5.9, 1.6 Hz, 1H), 4.57-4.43 (m, 2H), 4.22 (dt, J=5.9, 2.7 Hz, 1H), 4.13 (q, J=2.7 Hz, 1H), 3.84 (dd, J=11.9, 2.7 Hz, 1H), 3.69 (dd, J=11.9, 2.6 Hz, 1H), 2.73 (d, J=5.8 Hz, 2H), 2.45 (ddd, J=13.7, 6.0, 2.7 Hz, 1H), 2.10 (ddd, J=13.7, 7.6, 6.2 Hz, 1H). ¹⁹F NMR (565 MHz, CDCl₃) δ −165.34 (d, J=6.6 Hz). ¹³C NMR (151 MHz, CDCl₃) δ 157.65 (d, J=26.4 Hz), 149.14, 140.59 (d, J=235.5 Hz), 137.48, 128.60, 128.03, 127.77, 127.75, 124.98 (d, J=34.6 Hz), 86.14, 85.49, 78.80, 71.54, 62.21, 38.02.

D. Synthesis of (2S,3S,5R)-3-(benzyloxy)-5-(5-fluoro-2,4-dioxo-3,4-dihydropyrimidin-1(2H)-yl)tetrahydrofuran-2-carbaldehyde (MD-7-158)

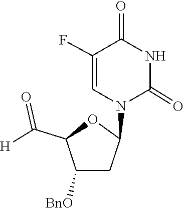 [SMILES: O=C[C@H]1O[C@@H](n2cc(F)c(=O)[nH]c2=O)C[C@@H]1OCc1ccccc1]

To a turbid solution of 1-((2R,4S,5R)-4-(benzyloxy)-5-(hydroxymethyl)tetrahydrofuran-2-yl)-5-fluoropyrimidine-2,4(1H,3H)-dione (0.65 g, 1.93 mmol, 1.0 eq) in anhydrous DCM (25 mL) was added dess-martin periodinane (2.45 g, 5.79 mmol, 3.0 eq) at room temperature and stirred for 3.5 h. The reaction mixture was then filtered through celite pad and rinsed thoroughly with DCM. The filtrate appeared cloudy. The filtrate was again filtered through celite pad and rinsed with DCM. The clear filtrate was concentrated and vacuum dried to obtain a white solid. Crude MD-7-158 was used in the next reaction without further purification (1.51 g, 1.35 mmol, 70% yield).

E. Synthesis of 1-((2R,4S,5R)-4-(benzyloxy)-5-(2,2,2-trifluoro-1-hydroxyethyl)tetrahydrofuran-2-yl)-5-fluoropyrimidine-2,4(1H,3H)-dione (MD-7-159)

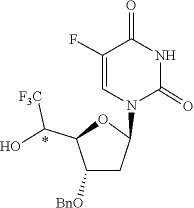 [SMILES: O=c1[nH]c(=O)n([C@H]2C[C@H](OCc3ccccc3)[C@@H](C(O)C(F)(F)F)O2)cc1F]

To a white suspension of crude (2S,3S,5R)-3-(benzyloxy)-5-(5-fluoro-2,4-dioxo-3,4-dihydropyrimidin-1(2H)-yl)tetrahydrofuran-2-carbaldehyde (1.50 g, 4.49 mmol, 1.0 eq, 30% pure) and (trifluoromethyl)trimethylsilane (3.31 mL, 22.43 mmol, 17 eq) in anhydrous THF (20 mL) was added tetra-n-butylammonium fluoride (117.32 mg, 0.45 mmol, 0.3 eq) at 0° C. After addition, ice bath was removed, and the reaction was stirred at room temperature for 1 h. Reaction progress was monitored by LC-MS indicated two diastereomers of the product as TMS adducts ([M−H]⁻=475). To the reaction mixture, 0.5 N HCl (30 mL) was added and stirred overnight. LC-MS analysis indicated TMS hydrolyzed product ([M−H]⁻=403). The reaction mixture was extracted with EtOAc and washed with saturated NaHCO₃ solution followed by brine solution. The organic layer was dried over anhydrous Na₂SO₄, filtered, and concentrated to a solid. Purification by silica gel chromatography using 0-80% EtOAc/hexanes eluted the two isomers of the product consecutively in 50-60% gradient. The fractions corresponding to each isomer were concentrated separately to obtain both isomers as solids. MD-7-159-1 (the 5′(S)—CF₃ isomer): 1-[(2R,4S,5R)-4-benzyloxy-5-[(1S)-2,2,2-trifluoro-1-hydroxy-ethyl]tetrahydrofuran-2-yl]-5-fluoro-pyrimidine-2,4-dione (150 mg, 0.37 mmol, 27% yield). MD-7-159-2 (the 5′(R)—CF₃ isomer): 1-[(2R,4S,5R)-4-benzyloxy-5-[(1R)-2,2,2-trifluoro-1-hydroxy-ethyl]tetrahydrofuran-2-yl]-5-fluoro-pyrimidine-2,4-dione (100 mg, 0.24 mmol, 18% yield). MD-7-159-1: ¹H NMR (400 MHz, CDCl₃) δ 7.82 (dd, J=6.4, 1.0 Hz, 1H), 7.38-7.23 (m, 5H), 6.32 (ddd, J=9.3, 5.6, 1.7 Hz, 1H), 4.49 (d, J=2.2 Hz, 2H), 4.46-4.44 (m, 1H), 4.37 (t, J=1.5 Hz, 1H), 4.19 (qd, J=7.7, 1.9 Hz, 1H), 2.46 (ddd, J=13.6, 5.6, 1.1 Hz, 1H), 2.10 (d, J=5.8 Hz, 5H), 2.09-2.00 (m, 1H). ¹F NMR (376 MHz, CDCl₃) δ −76.61 (d, J=7.9 Hz), −164.78 (d, J=6.2 Hz). 13C NMR (151 MHz, CDCl₃) δ 157.84 (d, J=26.1 Hz), 149.39, 140.79 (d, J=236.7 Hz), 137.31, 128.52, 128.00, 127.77, 125.14, 124.33 (d, J=34.3 Hz), 124.20 (d, J=283.1 Hz), 123.27, 85.67, 83.25, 78.03, 71.41, 70.65 (q, J=29.3, 28.7 Hz), 38.13. HRMS (ESI) m/z calculated for C₁₇H₁₅O₅N₂F₄ [M−H]⁻: 403.09226, found 403.09174. LC-MS (ESI) 75-95% MeOH/H₂O (0.1% HCO₂H), 3 min, 1.00 mL/min, t_{R}=1.61 min, m/z=403.0 [M−H]⁻. MD-7-159-2: ¹H NMR (400 MHz, CDCl₃) δ 8.08 (d, J=6.5 Hz, 1H), 7.47-7.24 (m, 5H), 6.21 (ddd, J=7.7, 5.9, 1.5 Hz, 1H), 4.71-4.38 (m, 2H), 4.34 (dd, J=2.6, 1.4 Hz, 1H), 4.22 (dt, J=5.5, 2.6 Hz, 1H), 3.98 (qd, J=7.3, 1.5 Hz, 1H), 2.48-2.39 (m, 1H), 2.17 (ddd, J=13.8, 7.8, 6.0 Hz, 1H). ¹⁹F NMR (376 MHz, CDCl₃) δ −76.35 (d, J=7.5 Hz), −165.32-−165.37 (m). ¹³C NMR (151 MHz, CDCl₃) δ 157.79 (d, J=26.1 Hz), 149.17, 140.59 (d, J=235.4 Hz), 137.10, 128.64, 128.17, 127.74, 125.12 (d, J=34.9 Hz), 124.17 (d, J=250.9), 87.04, 82.20, 80.08, 71.63, 69.52 (q, J=29.8 Hz), 37.23. HRMS (ESI) m/z calculated for C₁₇H₁₅O₅N₂F₄ [M−H]⁻: 403.09226, found 403.09207. LC-MS (ESI) 50-95% MeOH/H₂O (0.1% HCO₂H), 6 min, 1.00 mL/min, t_{R}=3.449 min, m/z=403.0 [M−H]⁻; 75-95% MeOH/H₂O (0.1% HCO₂H), 6 min, t_{R}=1.237 min, m/z=403.0 [M−H]⁻.

F. Synthesis of ammonium (R)-1-((2S,3S,5R)-3-(benzyloxy)-5-(5-fluoro-2,4-dioxo-3,4-dihydropyrimidin-1(2H)-yl)tetrahydrofuran-2-yl)-2,2,2-trifluoroethyl phosphate (MD-7-164)

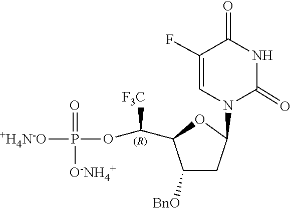

To a solution of phosphorus oxychloride (0.05 mL, 0.54 mmol, 4.4 eq) in anhydrous MeCN (0.5 mL) at 0° C. was added pyridine (0.04 mL, 0.54 mmol, 4.4 eq) and water (0.004 mL, 0.27 mmol, 2.2 eq). After 10 min, 1-[(2R,4S,5R)-4-benzyloxy-5-[(1R)-2,2,2-trifluoro-1-hydroxy-ethyl]tetrahydrofuran-2-yl]-5-fluoro-pyrimidine-2,4-dione (50.00 mg, 0.12 mmol, 1.0 eq) in anhydrous MeCN (0.7 mL) was added. After the addition, the mixture was stirred at room temperature overnight. After 15 h, LC-MS analysis didn't show any conversion. Additional pyridine (0.04 mL, 0.54 mmol, 4.4 eq) and phosphorus oxychloride (0.05 mL, 0.54 mmol, 4.4 eq) were added at 0° C. and continued to stir at room temperature. Reaction progress was monitored by LC-MS. After 5 h, the reaction was cooled to 0° C., quenched with water (15 mL), and stirred overnight at 0° C. The mixture was then neutralized with ammonium bicarbonate solid until pH >7. The contents were concentrated in vacuo and resuspended in methanol to precipitate the inorganic salts. The solids were removed by filtration, and the filtrate was concentrated. The process of precipitation and filtration was repeated one more time. Then, the filtrate was concentrated and purified by RP C₁₈ flash chromatography eluted with 0-50% MeOH/H₂O. Product fractions were pooled, concentrated, and freeze dried to obtain MD-7-164 as a solid (23 mg, 0.04 mmol, 36% yield). ¹H NMR (400 MHz, MeOD) δ 8.41-8.03 (m, 1H), 7.59-7.09 (m, 5H), 6.55-6.10 (m, 1H), 4.87 (m, 1H), 4.70 (d, J=5.4 Hz, 1H), 4.67-4.41 (m, 2H), 4.36 (m, 1H), 2.51-2.37 (m, 1H), 2.31 (tt, J=9.4, 5.4 Hz, 1H). ¹⁹F NMR (376 MHz, MeOD) δ −75.86 (d, J=7.3 Hz), −168.30 (d, J=6.7 Hz). ³¹P NMR (162 MHz, MeOD) δ −1.35. ¹C NMR (151 MHz, MeOD) δ 159.50 (d, J=26.4 Hz), 150.89, 141.93 (d, J=233.4 Hz), 139.42, 129.44, 128.99, 128.80, 126.10 (d, J=35.3 Hz), 125.05 (d, J=279.7), 87.38, 83.97-83.70 (m), 82.43, 74.37-72.77 (m), 37.84. HRMS (ESI) m/z calculated for C₁₇H₁₆O₈N₂F₄P [M−H]⁻: 483.05899, found 483.05899. LC-MS (ESI) 25-95% MeOH/H₂O (0.1% HCO₂H), 6 min, 1.00 mL/min, t_{R}=4.804 min, m/z=483.0 [M−H]⁻.

G. Synthesis of ammonium (S)-1-((2S,3S,5R)-3-(benzyloxy)-5-(5-fluoro-2,4-dioxo-3,4-dihydropyrimidin-1(2H)-yl)tetrahydrofuran-2-yl)-2,2,2-trifluoroethyl phosphate (MD-7-175)

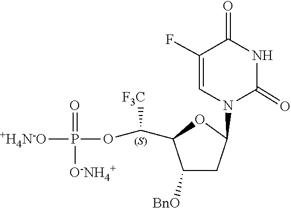

To a solution of phosphorus oxychloride (87.25 μL, 0.93 mmol, 15 eq) in anhydrous MeCN (1.5 mL) at 0° C. was added pyridine (74.71 μL, 0.93 mmol, 15 eq) and water (8.38 μL, 0.46 mmol, 7.5 eq). After 10 min, 1-[(2R,4S,5R)-4-benzyloxy-5-[(1S)-2,2,2-trifluoro-1-hydroxy-ethyl]tetrahydrofuran-2-yl]-5-fluoro-pyrimidine-2,4-dione (25.00 mg, 0.06 mmol, 1.0 eq) was added (in THF, 1 mL) and stirred at room temperature. The reaction progress was monitored by LC-MS. After 4.5 h, the reaction was cooled to 0° C., quenched with water (15 mL), and stirred for 30 min at 0° C. The mixture was then slowly quenched with ammonium bicarbonate solid until pH >7 and stirred at room temperature overnight. After 17 h, the mixture was concentrated in vacuo and reconstituted in methanol. The precipitated solids were filtered off and rinsed with methanol. The filtrate was concentrated to a crude solid and purified by RP C₁₈ flash chromatography using 0-50% MeOH/H₂O to afford the product at 30% gradient. The product fractions were pooled, concentrated, and freeze dried to obtain MD-7-175 as a white solid (11.5 mg, 0.02 mmol, 36% yield). ¹H NMR (400 MHz, MeOD) δ 8.34 (d, J=6.8 Hz, 1H), 7.42-7.22 (m, 5H), 6.26 (ddd, J=10.1, 4.7, 1.8 Hz, 1H), 4.83-4.76 (m, 1H), 4.64 (d, J=5.5 Hz, 1H), 4.64-4.52 (m, 2H), 4.28 (dt, J=4.0, 1.4 Hz, 1H), 2.40 (dd, J=13.5, 4.8 Hz, 1H), 2.19-2.06 (m, 1H). ¹⁹F NMR (376 MHz, MeOD) δ −76.77 (d, J=7.2 Hz), −168.57 (d, J=6.5 Hz). ³¹P NMR (162 MHz, MeOD) δ −1.28. ¹C NMR (151 MHz, MeOD) δ 159.47 (d, J=26.5 Hz), 150.97, 142.03 (d, J=233.7 Hz), 139.29, 129.36, 128.95, 128.74, 126.74 (d, J=34.8 Hz), 125.25 (d, J=282.3 Hz), 86.19, 83.97 (d, J=3.2 Hz), 79.79, 74.32-73.93 (m), 72.46, 37.27. HRMS (ESI) m/z calculated for C₁₇H₁₆O₈N₂F₄P [M−H]⁻: 483.05859, found 483.05869. LC-MS (ESI) 25-95% MeOH/H₂O (0.1% HCO₂H), 6 min, 1.00 mL/min, t_{R}=4.975 min, m/z=483.0 [M−H]⁻.

H. Synthesis of (R)-2,2,2-trifluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate (MD-7-171)

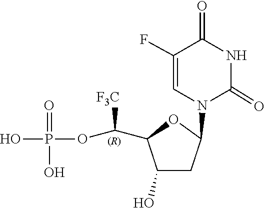

A solution of ammonium (R)-1-((2S,3S,5R)-3-(benzyloxy)-5-(5-fluoro-2,4-dioxo-3,4-dihydropyrimidin-1(2H)-yl)tetrahydrofuran-2-yl)-2,2,2-trifluoroethyl phosphate (15.00 mg, 0.03 mmol, 1.0 eq) in methanol (1 mL) was purged with argon and added palladium hydroxide on carbon (4.50 mg, 0.09 mmol, 3.0 eq, 20 wt. %) and stirred at room temperature under hydrogen balloon. The reaction progress was monitored by LC-MS. After 5 h, the reaction mixture was filtered through celite pad and rinsed with methanol. The filtrate was concentrated and purified by RP C₁₈ flash chromatography eluted with 100% H₂O. The product fractions were freeze dried to obtain MD-7-171 as a fluffy solid (1.9 mg, 0.0048 mmol, 16% yield). ¹H NMR (400 MHz, MeOD) δ 8.21 (d, J=6.7 Hz, 1H), 6.36 (ddd, J=8.1, 5.7, 1.9 Hz, 1H), 4.79-4.65 (m, 2H), 4.20 (s, 1H), 2.35 (ddd, J=13.9, 8.5, 5.6 Hz, 1H), 2.23 (ddd, J=13.6, 5.8, 2.2 Hz, 1H). ¹⁹F NMR (376 MHz, MeOD) δ −75.97 (d, J=7.1 Hz), −168.45 (d, J=6.9 Hz). ³¹P NMR (162 MHz, MeOD) δ −1.21. LC-MS (ESI) 50-95% MeOH/H₂O (0.1% HCO₂H), 3 min, 1.00 mL/min, t_{R}=1.027 min, m/z=393.0 [M−H]⁻; LC-MS (ESI) 25-95% MeOH/H₂O (0.1% HCO₂H), 6 min, t_{R}=1.42 min, m/z=393.0 [M−H]⁻.

I. Synthesis of (S)-2,2,2-trifluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate (MD-7-176)

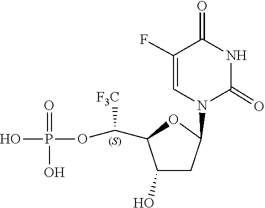

A solution of ammonium (S)-1-((2S,3S,5R)-3-(benzyloxy)-5-(5-fluoro-2,4-dioxo-3,4-dihydropyrimidin-1(2H)-yl)tetrahydrofuran-2-yl)-2,2,2-trifluoroethyl phosphate (14.00 mg, 0.03 mmol, 1.0 eq) in methanol (1.5 mL) was purged with argon and added palladium hydroxide on carbon (7.15 mg, 0.14 mmol, 20 wt. %, 5.3 eq) and stirred at room temperature under hydrogen balloon. The reaction progress was monitored by LC-MS. After 8 h, the mixture was filtered through celite pad, and rinsed with methanol. The filtrate was concentrated and purified by RP C₁₈ chromatography eluting with 100% H₂O. The product fractions were pooled and freeze dried to obtain MD-7-176 as a white fluffy solid (3.7 mg, 0.009 mmol, 35% yield). ¹H NMR (400 MHz, MeOD) δ 8.25 (d, J=6.8 Hz, 1H), 6.29 (td, J=7.9, 7.3, 1.8 Hz, 1H), 4.75 (dt, J=4.9, 2.8 Hz, 2H), 4.07 (dd, J=4.7, 1.9 Hz, 1H), 2.23-2.14 (m, 2H). ¹⁹F NMR (376 MHz, MeOD) δ −76.94 (d, J=7.0 Hz), −168.74 (d, J=6.6 Hz). ³¹P NMR (162 MHz, MeOD) δ −1.16. HRMS (ESI) m/z calculated for C₁₀H₁₀O₈N₂F₄P [M−H]⁻: 393.01164, found 393.01136. LC-MS (ESI) 25-95% MeOH/H₂O (0.1% HCO₂H), 3 min, 1.00 mL/min, t_{R}=0.854 min, m/z=393.0 [M−H]⁻; LC-MS (ESI) 10-95% MeOH/H₂O (0.1% HCO₂H), 6 min, t_{R}=1.379 min, m/z=393.0 [M−H]⁻.

### Example 10. Synthesis of Lipid Prodrugs of FdUMP

Scheme 10 below illustrates the synthetic procedures involved in Example 10.

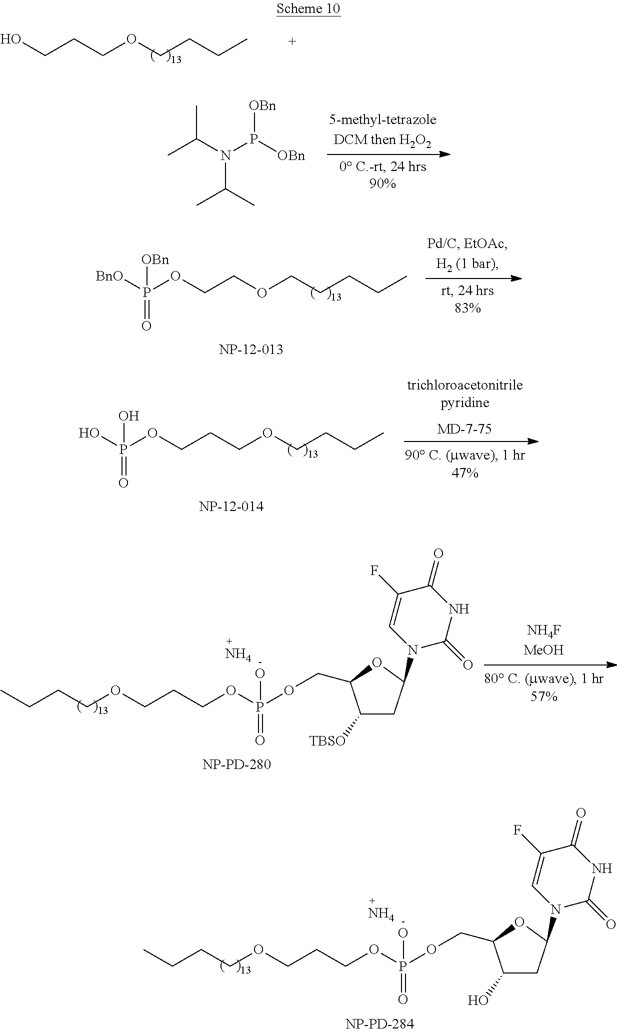

A. Synthesis of dibenzyl 3-hexadecoxypropyl phosphate (NP-12-013)

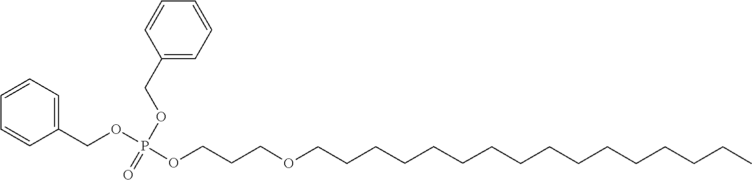 [SMILES: CCCCCCCCCCCCCCCCOCCCOP(=O)(OCc1ccccc1)OCc1ccccc1]

In a 100 mL Schlenk flask equipped with a stir bar and under an argon atmosphere, a solution of 3-hexadecoxypropan-1-ol (500 mg, 1.66 mmol, 1.0 eq) and 5-methyl-1H-tetrazole (839 mg, 9.98 mmol, 6.0 eq) in DCM (15 mL) was cooled in an ice-water bath to 0° C., and then treated dropwise with dibenzyl diisopropylphosphoramidite (1.64 mL, 4.99 mmol, 3.0 eq). The resulting reaction mixture was stirred at 0° C. for approximately 5 min, and then at room temperature for 24 h, at which point TLC indicated that all of the starting material had been consumed. The mixture was cooled again in an ice-water bath to 0° C. and treated with 30% hydrogen peroxide (1.9 mL, 18 mmol, 11 eq). After 1 h, the mixture was partitioned between DCM and saturated aqueous NaHCO₃. The organic phase was washed with brine, dried, and concentrated in vacuo. The resulting crude residue was then purified by column chromatography on silica gel (100% hexanes-20% EtOAc in hexanes), to provide dibenzyl 3-hexadecoxypropyl phosphate (841 mg, 1.50 mmol, 90% yield) as a clear oil. ¹H NMR (600 MHz, CDCl₃) δ 7.38-7.28 (m, 10H), 5.09-4.99 (m, 4H), 4.11 (q, J=6.5 Hz, 2H), 3.43 (t, J=6.1 Hz, 2H), 3.35 (t, J=6.7 Hz, 2H), 1.87 (p, J=6.2 Hz, 2H), 1.52 (p, J=6.8 Hz, 2H), 1.33-1.24 (m, 26H), 0.88 (t, J=6.9 Hz, 3H). ¹³C NMR (151 MHz, CDCl₃) δ 136.0 (d, J_{CP}=6.7 Hz, 2C), 128.7 (4C), 128.6 (2C), 128.0 (4C), 71.3, 69.3 (d, J_{CP}=5.5 Hz, 2C), 66.5, 65.3 (d, J_{CP}=5.9 Hz), 32.0, 30.7 (d, J_{CP}=6.9 Hz), 29.8 (4C), 29.8 (2C), 29.8, 29.7, 29.7, 29.6, 29.5, 26.3, 22.8, 14.2. ³¹P NMR (243 MHz, CDCl₃) δ −0.83.

B. Synthesis of 3-hexadecoxypropyl dihydrogen phosphate (NP-12-014)

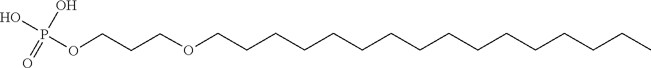 [SMILES: CCCCCCCCCCCCCCCCOCCCOP(=O)(O)O]

Dibenzyl 3-hexadecoxypropyl phosphate (1.23 g, 2.19 mmol, 1.0 eq) and EtOH (10 mL) were added to an oven-dried flask equipped with a magnetic stir bar. The solution was subsequently degassed under gentle vacuum for approximately 10 min and then the reaction flask was purged with argon. This cycle was repeated twice more before the addition of 10% palladium on carbon (233 mg, 0.219 mmol, 0.1 eq). Once more, the reaction flask was placed under vacuum before a final purge using an H₂ balloon. The resulting reaction mixture was subsequently stirred vigorously under an atmosphere of H₂ at room temperature for 24 h. After this time, the heterogeneous reaction mixture was filtered over a bed of celite, and the mother liquor was concentrated under reduced pressure to yield 3-hexadecoxypropyl dihydrogen phosphate (694 mg, 1.82 mmol, 83% yield) as an off-white solid. ¹H NMR (400 MHz, CD₃OD) δ 4.03 (q, J=6.4 Hz, 2H), 3.53 (t, J=6.3 Hz, 2H), 3.43 (t, J=6.6 Hz, 2H), 1.90 (p, J=6.3 Hz, 2H), 1.54 (q, J=6.9 Hz, 2H), 1.35-1.26 (m, 26H), 0.88 (t, 3H). ¹³C NMR (101 MHz, CD₃OD) δ 72.0, 67.9, 64.4 (d, J_{CP}=5.4 Hz), 32.9, 31.7 (d, J_{CP}=7.3 Hz), 30.7 (4C), 30.6 (3C), 30.6 (2C), 30.5, 30.3, 27.1, 23.6, 14.5. ³¹P NMR (162 MHz, CD₃OD) δ −0.42.

C. Synthesis of ammonium [3-[tert-butyl(dimethyl)silyl]oxy-5-(5-fluoro-2,4-dioxo-pyrimidin-1-yl)tetrahydrofuran-2-yl]methyl 3-hexadecoxypropylphosphate (NP-PD-280)

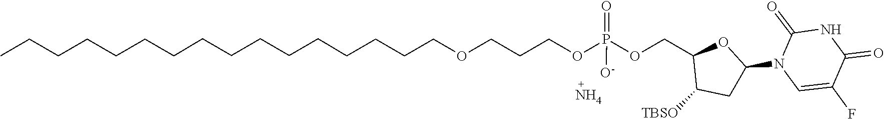

1-[4-[tert-Butyl(dimethyl)silyl]oxy-5-(hydroxymethyl)tetrahydrofuran-2-yl]-5-fluoro-pyrimidine-2,4-dione (71 mg, 0.20 mmol, 1.5 eq), 3-hexadecoxypropyl dihydrogen phosphate (50 mg, 0.13 mmol, 1.0 eq), cyanotrichloromethane (0.02 mL, 0.2 mmol, 1.5 eq), and pyridine (0.7 mL) were placed in a 0.5-2.0 mL microwave vial, fitted with a stir bar. The vial was sealed and irradiated in a microwave reactor for 1 h at 90° C. The reaction mixture was then concentrated in vacuo and the resulting crude material was purified by column chromatography (0-100% 80:20:3 DCM:MeOH:NH₄OH in DCM) to afford the product NP-PD-280 (46 mg, 0.062 mmol, 47% yield) as a pale-yellow oil. ¹H NMR (400 MHz, CD₃OD) δ 8.08 (d, J=6.6 Hz, 1H), 6.34-6.25 (m, 1H), 4.61-4.55 (m, 1H), 4.03 (t, J=3.0 Hz, 3H), 3.97 (q, J=6.4 Hz, 2H), 3.53 (t, J=6.5 Hz, 2H), 3.41 (t, J=6.6 Hz, 2H), 2.30-2.16 (m, 2H), 1.88 (p, J=6.3 Hz, 2H), 1.54 (p, J=6.8 Hz, 2H), 1.36-1.28 (m, 26H), 0.96-0.86 (m, 12H), 0.14 (s, 6H). LC-MS (ESI, C₈, 0.6 mL/min) 65-95% MeCN in H₂O, 6 min, t_{R}=4.106, m/z=721.5 [M−H]⁻.

D. Synthesis of ammonium [5-(5-fluoro-2,4-dioxo-pyrimidin-1-yl)-3-hydroxy-tetrahydrofuran-2-yl]methyl 3-hexadecoxypropyl phosphate (NP-PD-284)

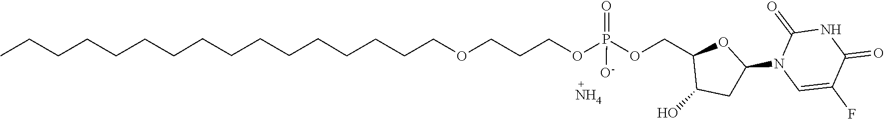

In a 0.5-2.0 mL microwave vial fitted with a stirrer bar, ammonium [3-[tert-butyl(dimethyl)silyl]oxy-5-(5-fluoro-2,4-dioxo-pyrimidin-1-yl)tetrahydrofuran-2-yl]methyl 3-hexadecoxypropyl phosphate (46 mg, 0.062 mmol, 1.0 eq) was treated with ammonium fluoride (23 mg, 0.62 mmol, 10 eq) and MeOH (0.6 mL). The vial was then sealed and irradiated in a microwave reactor for 1 h at 80° C. The reaction mixture was then concentrated in vacuo and the resulting crude material was purified using reverse phase (Cis) column chromatography (10-100% MeOH in H₂O) to afford NP-PD-284 (22 mg, 0.035 mmol, 57% yield) as a white solid. ¹H NMR (400 MHz, CD₃OD) S 8.10 (d, J=6.6 Hz, 1H), 6.30 (td, J=6.9, 1.8 Hz, 1H), 4.52-4.45 (m, 1H), 4.06-4.03 (m, 3H), 3.97 (q, J=6.3 Hz, 2H), 3.53 (t, J=6.3 Hz, 2H), 3.41 (t, J=6.6 Hz, 2H), 2.30-2.22 (m, 2H), 1.88 (p, J=6.3 Hz, 2H), 1.53 (q, J=6.9 Hz, 2H), 1.34-1.26 (m, 26H), 0.89 (t, J=6.7 Hz, 3H). ¹C NMR (101 MHz, CD₃OD) δ 159.5 (d, J_{CF}=26.2 Hz), 150.8, 141.9 (d, J_{CF}=233.3 Hz), 126.2 (d, J_{CF}=34.4 Hz), 87.8 (d, J_{CP}=8.6 Hz), 86.8, 72.7, 72.1, 68.3, 66.2 (d, J_{CP}=5.4 Hz), 63.8 (d, J_{CP}=5.7 Hz), 41.0, 33.1, 32.1 (d, J_{CP}=7.6 Hz), 30.8 (6C), 30.8 (3C), 30.6, 30.5, 27.3, 23.7, 14.5. ³¹P NMR (162 MHz, CD₃OD) δ −0.34. ¹F NMR (376 MHz, CD₃OD) δ −168.38 (dd, J=6.4, 2.0 Hz). HRMS (ESI) m/z calculated for C₂₈H₄₉O₉N₂FP [M−H]⁻ 607.31652, found 607.31729. LC-MS (ESI, C₈, 0.6 mL/min) 55-95% MeCN in H₂O, 6 min, t_{R}=1.469 min, m/z=607.5 [M−H]⁻; 40-95% MeCN in H₂O, 6 min, t_{R}=3.760 min, m/z=607.5 [M−H]⁻.

### Example 11. Synthesis of Terminally Modified Lipid Prodrugs of FdUMP and its 5′-(R)-Methyl Analog

Scheme 11 below illustrates the synthetic procedures involved in Example 11.

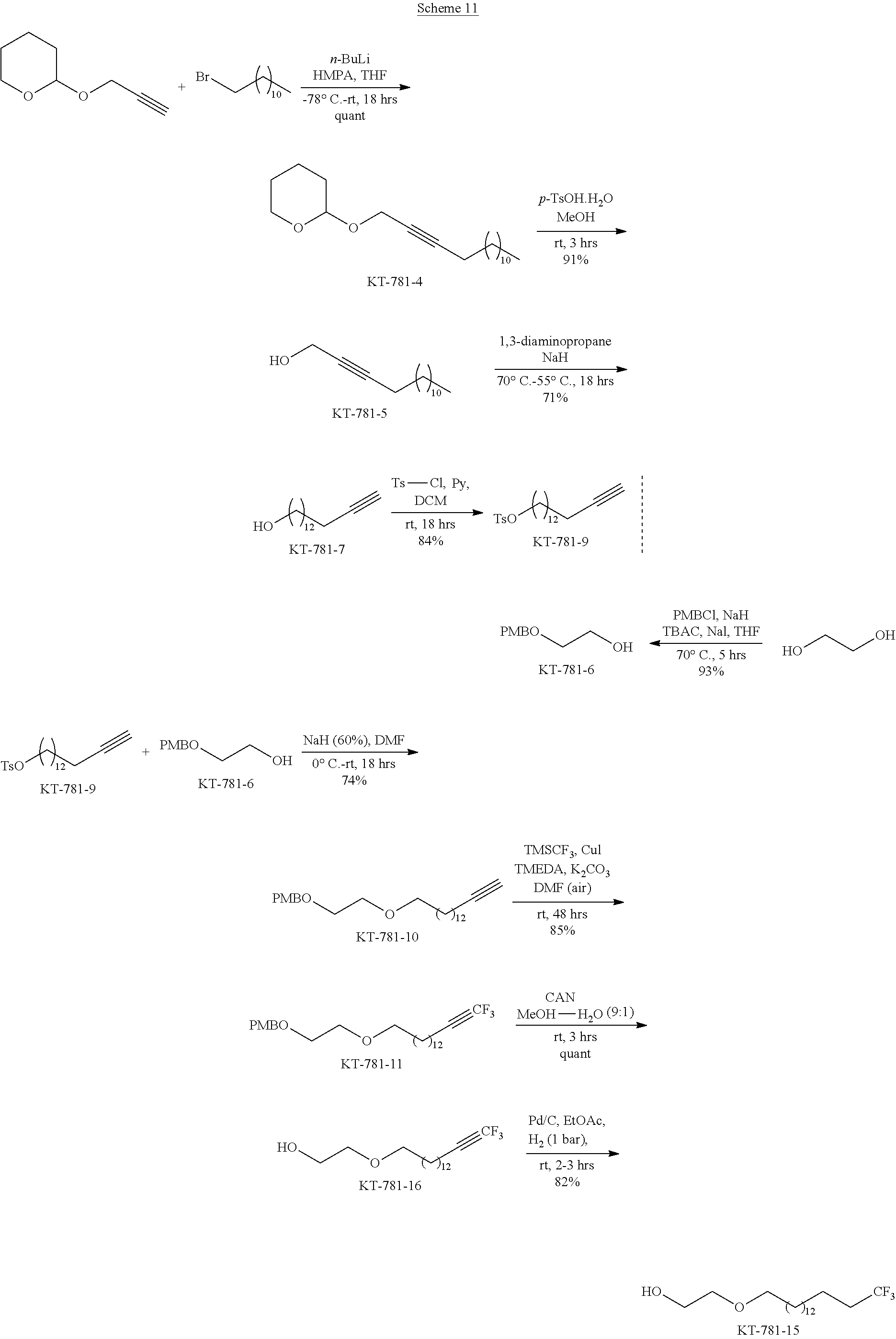

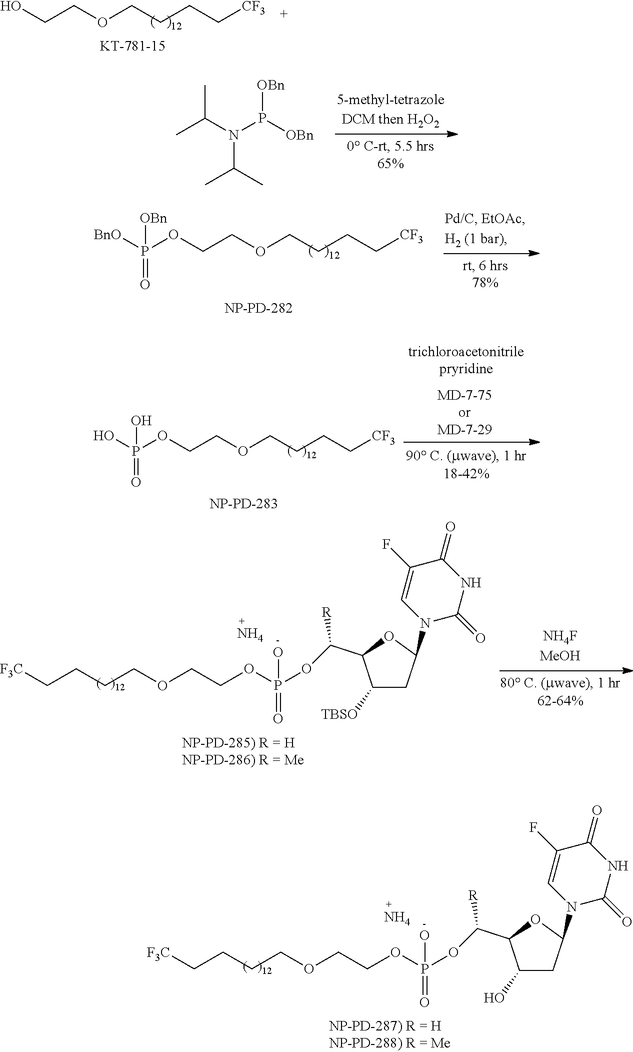

A. Synthesis of 2-(pentadec-2-yn-1-yloxy)tetrahydro-2H-pyran (KT-781-4)

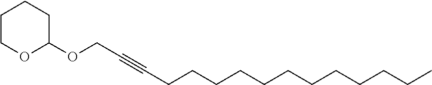 [SMILES: CCCCCCCCCCCCC#CCOC1CCCCO1]

To a 250 mL RB-flask equipped with a stir bar was added 2-(2-propynyloxy)tetrahydro-2H-pyran (2.0 mL, 14 mmol, 1.0 eq), hexamethylphosphoramide (8.7 mL, 50 mmol, 3.5 eq), and THF (20 mL) under argon atmosphere. The reaction mixture was cooled to −78° C. and n-butyllithium (2.5 M in hexanes, 7.4 mL, 19 mmol, 1.3 eq) was added dropwise. The reaction was stirred at −78° C. for approximately 1 hour and subsequently added 1-iodododecane (4.6 mL, 19 mmol, 1.3 eq) dropwise with vigorous stirring. The resulting reaction mixture was allowed to warm to room temperature overnight while stirring vigorously. The reaction mixture was quenched with a saturated solution of ammonium chloride and then extracted three times into EtOAc. The organic phases were then combined, washed with brine, dried over anhydrous sodium sulfate, and concentrated in vacuo. Subsequent purification of the resulting crude material by flash chromatography (2-10% EtOAc/hexane) afforded a clear oil (4.60 g, 14.9 mmol, quant). ¹H NMR (400 MHz, CDCl₃) δ 4.78 (t, J=3.2 Hz, 1H), 4.30-4.15 (m, 2H), 3.85-3.75 (m, 1H), 3.55-3.45 (m, 1H), 2.25-2.15 (m, 2H), 1.84-1.77 (m, 1H), 1.73-1.67 (m, 1H), 1.63-1.1.43 (m, 6H), 1.39-1.21 (m, 18H), 0.84 (t, J=6.8 Hz, 3H). HRMS (ESI) m/z calculated for C₂₀H₃₆O₂Na [M+Na]⁺331.26130, found 331.26105.

B. Synthesis of pentadec-2-yn-1-ol (KT-781-5)

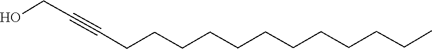 [SMILES: CCCCCCCCCCCCC#CCO]

To a solution of 2-(pentadec-2-yn-1-yloxy)tetrahydro-2H-pyran (KT-781-4, 4.60 g, 14.9 mmol, 1.0 eq) in methanol (50 mL) in a 100 mL flask equipped with a stir bar was added p-toluenesulfonic acid monohydrate (284 mg, 1.49 mmol, 0.1 eq), and the reaction mixture was stirred vigorously for 3 hours at room temperature. TLC had confirmed full consumption of the starting material. The reaction mixture was subsequently concentrated in vacuo and the resulting crude material was purified by flash chromatography (2-10% EtOAc/hexane) to afford a white solid (3.04 g, 13.6 mmol, 91%). ¹H NMR (400 MHz, CDCl₃) δ 4.22 (t, J=0.8 Hz, 2H), 2.20-2.15 (m, 2H), 1.76-1.40 (m, 1H), 1.48 (p, J=6.8 Hz, 2H), 1.36-1.30 (m, 2H), 1.30-1.20 (m, 16H), 0.851 (t, J=6.8 Hz, 3H). ¹³C NMR (100 MHz, CDCl₃) δ 86.6, 78.2, 51.3, 31.9, 29.6, 29.6 (2C), 29.5, 29.3, 29.1, 28.9, 28.6, 22.7, 18.7, 14.1. HRMS (APCI) m/z calculated for C₁₅H₂₉O [M+H]⁺225.22129, found 225.22171.

C. Synthesis of pentadec-14-yn-1-ol (KT-781-7)

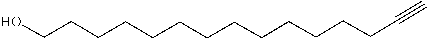 [SMILES: C#CCCCCCCCCCCCCCO]

250 mL RB-flask equipped with a stir bar was charged with 1,3-diaminopropane (20 mL) under an argon atmosphere. Sodium hydride (60% in mineral oil, 1.43 g, 35.7 mmol, 8.0 eq) was added with vigorous stirring. The flask was placed in an oil bath preheated to 70° C. After stirring for 1 hour at this temperature, the reaction temperature was lowered to 55° C. A solution of pentadec-2-yn-1-ol (1.0 g, 4.5 mmol, 1.0 eq) in 1,3-diaminopropane (6.0 mL) was added dropwise to the reaction mixture, which was then left stirring vigorously overnight at 55° C. The following morning the reaction was cooled to 0° C., quenched with ice, and acidified with a 1 N aqueous HCl solution to a pH of 2. The resulting aqueous phase was then extracted three times with hexane. The combined organic phases were then dried over sodium sulfate and concentrated in vacuo. The resulting crude material was then purified by column chromatography (5-20% EtOAc/hexane) to afford a white solid (0.7 g, 3.1 mmol, 71%). ¹H NMR (400 MHz, CDCl₃) δ 3.60 (t, J=6.4 Hz, 2H), 2.14 (td, J=7.2, 2.8 Hz, 2H), 1.91 (t, J=2.8 Hz, 1H), 1.84 (s, 1H), 1.55-1.45 (m, 5H), 1.40-1.20 (m, 20H). ¹³C NMR (100 MHz, CDCl₃) δ 84.8, 68.0, 63.0, 32.7, 29.6 (2C), 29.5 (2C), 29.5, 29.4, 29.1, 28.7, 28.5, 25.7, 18.4. HRMS (APCI) m/z calculated for C₁₅H₂₉O [M+H]⁺225.22129, found 225.22156.

D. Synthesis of pentadec-14-ynyl 4-methylbenzenesulfonate (KT-781-9)

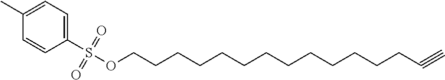 [SMILES: C#CCCCCCCCCCCCCCOS(=O)(=O)c1ccc(C)cc1]

To a solution of pentadec-14-yn-1-ol (1.70 g, 7.58 mmol, 1.0 eq) in DCM (50 mL) at 0° C. and under an atmosphere of argon was added pyridine (1.2 mL, 15 mmol, 2.0 eq) and then p-toluenesulfonyl chloride (2.17 g, 11.4 mmol, 1.5 eq). The reaction mixture was stirred at room temperature overnight. The reaction mixture was subsequently diluted with DCM and then quenched with water. The phases were separated, and the organic layer was washed with 2 M HCl, followed by a saturated NaHCO₃ solution, water, and brine, and then dried over sodium sulfate. The solvent was concentrated in vacuo and the resulting crude material was then purified by way of silica gel flash column chromatography (100% hexanes-10% EtOAc/hexanes) to yield KT-781-9 as a white solid (2.41 g, 6.37 mmol, 84%). ¹H NMR (CDCl₃, 400 MHz) δ 7.76 (d, J=6.4 Hz, 2H), 7.32 (d, J=8.0 Hz, 2H), 3.99 (t, J=6.5 Hz, 2H), 2.42 (s, 3H), 2.16 (td, J=7.2, 2.7 Hz, 2H), 1.91 (t, J=2.6 Hz, 1H), 1.63-1.56 (m, 2H), 1.52-1.45 (m, 2H), 1.40-1.32 (m, 2H), 1.30-1.15 (m, 16H). HRMS (APCI) m/z calculated for C₂₂H₃₅O₃³²S [M+H]⁺379.23014, found 379.23053.

E. Synthesis of 2-[(4-methoxybenzyl)oxy]ethan-1-ol (KT-781-6)

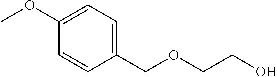

Sodium hydride (60% in mineral oil, 1.4 g, 35 mmol, 1.1 eq) was added to a solution of ethylene glycol (5.3 mL, 96 mmol, 3.0 eq) in THF (50 mL). After stirring for 30 min at room temperature, 4-methoxybenzyl chloride (5.0 g, 32 mmol, 1.0 eq), Bu₄NCl (887 mg, 3.19 mmol, 0.1 eq), and KI (530 mg, 3.19 mmol, 0.1 eq) were added sequentially, and the reaction mixture was heated to reflux. After 5 h, the reaction mixture was cooled to room temperature, the reaction was quenched with saturated NH₄Cl (30 mL) and extracted with Et₂O (3×60 mL). The combined organic layers were washed with brine (30 mL), dried over sodium sulfate, and the solvent was removed under reduced pressure. The crude product was purified by flash column chromatography to yield protected alcohol KT-781-6 as a yellow oil (5.43 g, 29.8 mmol, 93%). ¹H NMR (CDCl₃, 400 MHz) S 7.21 (d, J=8.4 Hz, 2H), 6.8r3 (d, J=8.6 Hz, 2H), 4.42 (s, 2H), 3.73 (s, 3H), 3.66 (t, J=4.0 Hz, 2H), 3.49 (t, J=4.0 Hz, 2H), 3.00 (br.s, 1H). ¹H-NMR in agreement with the literature values. HRMS (ESI) m/z calculated for C₁₀H₁₄O₃Na [M+Na]⁺205.08406, found 205.08397.

F. Synthesis of 1-(2-pentadec-14-ynoxyethoxymethyl)-4-methoxy-benzene (KT-781-10)

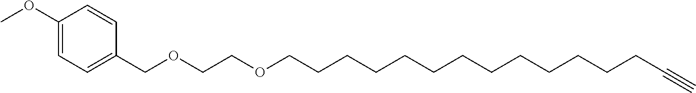 [SMILES: C#CCCCCCCCCCCCCCOCCOCc1ccc(OC)cc1]

To a solution of 2-[(4-methoxyphenyl)methoxy]ethanol (1.39 g, 7.61 mmol, 1.2 eq) in anhydrous DMF (25 mL) under argon atmosphere at 0° C. was added sodium hydride (60% in mineral oil, 304 mg, 7.61 mmol, 1.2 eq) portionwise. After approximately 30 minutes at this temperature, pentadec-14-ynyl 4-methylbenzenesulfonate (2.40 g, 6.34 mmol, 1.0 eq) was added portionwise and the resulting suspension was allowed to warm to room temperature and stirred vigorously at this temperature overnight. The following morning TLC indicated consumption of the starting material. The reaction was subsequently cooled to room temperature, quenched with a saturated ammonium chloride solution, and extracted three times into DCM. The resulting organic phases were combined, dried over sodium sulfate, and then concentrated in vacuo. The crude material was then purified by silica gel column chromatography (2080% DCM/hexanes) to yield 1-(2-pentadec-14-ynoxyethoxymethyl)-4-methoxy-benzene (1.82 g, 4.67 mmol, 74%) as a white solid. ¹H NMR (600 MHz, CDCl₃) δ 7.29 (d, J=8.7 Hz, 2H), 6.89 (d, J=8.6 Hz, 2H), 4.52 (s, 2H), 3.82 (s, 3H), 3.61 (s, 4H), 3.47 (t, J=6.8 Hz, 2H), 2.19 (td, J=7.2, 2.7 Hz, 2H), 1.95 (t, J=2.6 Hz, 1H), 1.64-1.57 (m, 2H), 1.54 (p, J=7.2 Hz, 2H), 1.44-1.37 (m, 2H), 1.37-1.24 (m, 20H). ¹³C NMR (151 MHz, CDCl₃) δ 159.2, 130.5, 129.4 (2C), 113.8 (2C), 84.8, 72.9, 71.6, 70.2, 69.1, 68.1, 55.3, 29.7, 29.6, 29.6, 29.6 (2C), 29.5 (2C), 29.1, 28.8, 28.5, 26.1, 18.4. HRMS (APCI) m/z calculated for C₂₅H₄₀O₃ [M]⁺388.29775, found 388.29665.

G. Synthesis of 1-methoxy-4-[2-(16,16,16-trfluorohexadec-14-ynoxy)ethoxymethyl]benzene (KT-781-11)

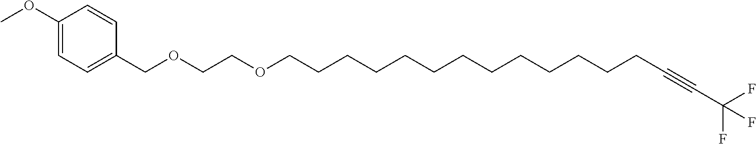 [SMILES: COc1ccc(COCCOCCCCCCCCCCCCCC#CC(F)(F)F)cc1]

A 100 mL RB-flask equipped with a stir bar was charged with copper(I)iodide (1.32 g, 6.95 mmol, 1.5 eq), potassium carbonate (1.92 g, 13.9 mmol, 3.0 eq) and N,N,N′,N′-tetramethylethylenediamine (1.1 mL, 7.0 mmol, 1.5 eq) in DMF (10 mL) under an atmosphere of air (balloon). The resulting blue mixture was stirred vigorously at room temperature for 15 minutes. (Trifluoromethyl)trimethylsilane (1.4 mL, 9.3 mmol, 2.0 eq) was added to the reaction mixture and the reaction was stirred for an additional 5 minutes before cooling to 0° C. To the reaction mixture was added (in one portion) a solution of 1-(2-pentadec-14-ynoxyethoxymethyl)-4-methoxy-benzene (1.80 g, 4.63 mmol, 1.0 eq) and (trifluoromethyl)trimethylsilane (1.4 mL, 9.3 mmol, 2.0 eq) in DMF (10 mL). The reaction was left to warm to room temperature and stirred vigorously for 48 hours. For the workup, the reaction was quenched with H₂O and extracted three times with DCM. The organic phases were combined, dried over Na₂SO₄, filtered, and concentrated in vacuo. The crude material was subsequently purified by column chromatography (5-20% EtOAc/Hexane) to yield KT-781-11 as a white solid (1.80 g, 3.94 mmol, 85%). ¹H NMR (600 MHz, CDCl₃) δ 7.29 (d, J=8.6 Hz, 2H), 6.89 (d, J=8.6 Hz, 2H), 4.53 (s, 2H), 3.82 (s, 3H), 3.61 (s, 4H), 3.47 (t, J=6.8 Hz, 2H), 2.31 (tq, J=7.4, 3.8 Hz, 2H), 1.64-1.56 (m, 4H), 1.44-1.24 (m, 18H). ¹¹C NMR (151 MHz, CDCl₃) δ 159.2, 130.5, 129.4 (2C), 114.2 (q, J_{CF}=256.7 Hz), 113.7 (2C), 89.4 (q, J_{CF}=6.3 Hz), 72.9, 71.6, 70.2, 69.1, 68.3 (q, J_{CF}=51.7 Hz), 55.2, 29.7, 29.6 (2C), 26.6, 29.5, 29.5, 29.4, 29.0, 28.7, 27.2 (app d, J_{CF}=1.7 Hz), 26.1, 18.1 (q, J_{CF}=1.6 Hz). ¹⁹F NMR (565 MHz, CDCl₃) δ −49.33 (t, J=3.9 Hz). HRMS (ESI) m/z calculated for C₂₆H₃₉O₃F₃Na [M+Na]⁺479.27435, found 479.27423.

H. Synthesis of 2-(16,16,16-trifluorohexadec-14-ynoxy)ethanol (KT-781-16)

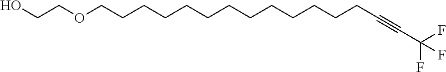 [SMILES: OCCOCCCCCCCCCCCCCC#CC(F)(F)F]

In a 25 mL flask equipped with a stir bar, KT-781-11 (0.90 g, 1.97 mmol, 1.0 eq) was dissolved in a mixture of methanol (10 mL) and water (1.0 mL). The reaction was cooled to 0° C. and ceric ammonium nitrate (3.24 g, 5.91 mmol, 3.0 eq) was added portionwise. The reaction mixture was then warmed to room temperature and stirred vigorously for 3 hours or until TLC confirmed the consumption of the starting material. Subsequent quenching with water was followed by three extractions with DCM. The organic phase was dried over sodium sulfate and concentrated in vacuo. The resulting crude material was purified by column chromatography (100% hexanes-20% EtOAc/hexanes) to yield 2-(16,16,16-trifluorohexadec-14-ynoxy)ethanol as a light brown solid (0.70 g, 2.08 mmol, quant). ¹H NMR (600 MHz, CDCl₃) δ 3.86-3.81 (m, 2H), 3.63-3.59 (m, 2H), 3.53 (t, J=6.8 Hz, 2H), 2.32 (tq, J=7.5, 3.8 Hz, 2H), 1.65-1.55 (m, 4H), 1.40 (p, J=7.1 Hz, 2H), 1.37-1.27 (m, 18H). ¹³C NMR (151 MHz, CDCl₃) δ 114.2 (q, J_{CF}=255.9 Hz), 89.4 (q, J_{CF}=6.2 Hz), 71.7, 71.1, 68.3 (q, J_{CF}=51.1 Hz), 61.8, 29.6, 29.6, 29.5, 29.5, 29.4, 29.4, 29.4, 28.9, 28.7, 27.2, 26.0, 18.1. ¹⁹F NMR (565 MHz, CDCl₃) δ −49.35 (t, J=4.5 Hz).

I. Synthesis of 2-(16,16,16-trifluorohexadecoxy)ethanol (KT-781-15)

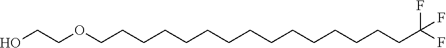 [SMILES: OCCOCCCCCCCCCCCCCCCC(F)(F)F]

1-Methoxy-4-[2-(16,16,16-trifluorohexadec-14-ynoxy)ethoxymethyl]benzene (900 mg, 1.97 mmol, 1.0 eq), ethyl acetate (30 mL), 10% Pd—C (500 mg) were added to a Parr flask and hydrogenated at 15 psi/1 bar for 2-3 h. After this time, the heterogeneous reaction mixture was filtered over a bed of celite, and the filtrate was collected and concentrated in vacuo. The resulting crude product was purified by column chromatography (5-20% EtOAc/hexane) to yield KT-781-15 as a white solid (550 mg, 1.62 mmol, 82% yield). ¹H NMR (600 MHz, CDCl₃) δ 3.74 (dd, J=5.3, 4.0 Hz, 2H), 3.56-3.53 (m, 2H), 3.48 (t, J=6.7 Hz, 2H), 2.12-2.01 (m, 3H), 1.64-1.52 (m, 4H), 1.42-1.22 (m, 23H). ¹³C NMR (151 MHz, CDCl₃) δ 127.3 (q, J_{CF}=276.3 Hz), 71.7, 71.4, 61.9, 33.7 (q, J_{CF}=28.2 Hz), 29.7, 29.6, 29.6, 29.6 (3C), 29.5, 29.5, 29.4, 29.2, 28.7, 26.1, 21.8 (q, J_{CF}=2.9 Hz). ¹⁹F NMR (565 MHz, CDCl₃) δ −66.44 (t, J=11.0 Hz).
J. Synthesis of dibenzyl 2-(16,16,16-trifluorohexadecoxy)ethyl phosphate (NP-PD-282)

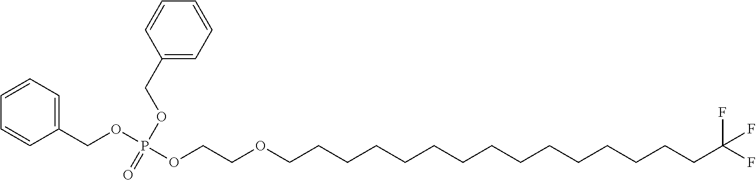 [SMILES: O=P(OCCOCCCCCCCCCCCCCCCC(F)(F)F)(OCc1ccccc1)OCc1ccccc1]

In a 100 mL Schlenk flask equipped with a stir bar and under an argon atmosphere, a solution of 2-(16,16,16-trifluorohexadecoxy)ethanol (520 mg, 1.53 mmol, 1.0 eq) and 5-methyl-1H-tetrazole (771 mg, 9.16 mmol, 6.0 eq) in DCM (20 mL) was cooled in an ice-water bath to 0° C., and then treated dropwise with dibenzyl N,N-diisopropylphosphoramidite (0.75 mL, 2.29 mmol, 1.5 eq). The resulting reaction mixture was stirred at 0° C. for approximately 5 min, and then at room temperature for 2 h, at which point TLC indicated that all of the starting material had been consumed. The mixture was cooled again in an ice-water bath to 0° C. and treated with 30% hydrogen peroxide (4.3 mL, 42 mmol, 11 eq). After 1 h, the mixture was partitioned between DCM and saturated aqueous NaHCO₃. The organic phase was washed with brine, dried, and concentrated in vacuo. The resulting crude residue was then purified by column chromatography on silica gel (100% hexanes-20% EtOAc in hexanes) to yield dibenzyl 2-(16,16,16-trifluorohexadecoxy)ethyl phosphate (599 mg, 0.997 mmol, 65% yield) as a clear oil. ¹H NMR (600 MHz, Chloroform-d) δ 7.42-7.28 (m, 10H), 5.05 (dd, J=7.9, 3.5 Hz, 4H), 4.16-4.10 (m, 2H), 3.58 (td, J=4.7, 1.1 Hz, 2H), 3.42 (t, J=6.7 Hz, 2H), 2.12-1.99 (m, 2H), 1.59-1.49 (m, 4H), 1.39-1.33 (m, 2H), 1.32-1.21 (m, 20H). ¹¹C NMR (151 MHz, CDCl₃) δ 136.1 (d, J=7.1 Hz, 2C), 128.7 (4C), 128.6 (2C), 128.1 (4C), 127.5 (q, J_{CF}=276.2 Hz), 71.7, 69.5 (d, J_{CP}=7.1 Hz), 69.4 (d, J_{CP}=5.5 Hz), 67.0 (d, J_{CP}=6.0 Hz, 2C), 33.9 (q, J_{CF}=28.2 Hz), 29.8 (2C), 29.8, 29.8 (3C), 29.7, 29.6, 29.5, 29.3, 28.8, 26.2, 22.0 (q, J_{CF}=2.9 Hz). ³¹P NMR (243 MHz, CDCl₃) δ −0.96. ¹F NMR (565 MHz, CDCl₃) δ −66.43 (t, J=11.1 Hz). HRMS(ESI) m/z calculated for C₃₂H₄₉O₅F₃P [M+H]⁺601.32642, found 601.32797.

K. Synthesis of 2-(16,16,16-trifluorohexadecoxy)ethyl dihydrogen phosphate (NP-PD-283)

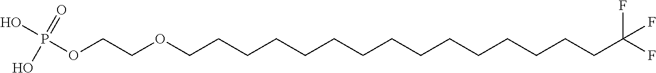 [SMILES: O=P(O)(O)OCCOCCCCCCCCCCCCCCCC(F)(F)F]

Dibenzyl 2-(16,16,16-trifluorohexadecoxy)ethyl phosphate (599 mg, 0.997 mmol, 1.0 eq) and EtOAc (5.0 mL) were added to an oven-dried flask equipped with a magnetic stir bar. The solution was subsequently degassed under gentle vacuum for approximately 10 min and then the reaction flask was purged with argon. This cycle was repeated twice more before the addition of 10% palladium on carbon (106 mg, 0.0997 mmol, 0.1 eq). Once more, the reaction flask was placed under vacuum before a final purge using an H₂ balloon. The resulting reaction mixture was subsequently stirred vigorously under an atmosphere of H₂ at room temperature for 6 h. After this time, the heterogeneous reaction mixture was filtered over a bed of celite, and the mother liquor was concentrated under reduced pressure to yield 2-(16,16,16-trifluorohexadecoxy)ethyl dihydrogen phosphate (328 mg, 0.780 mmol, 78% yield) as an off-white solid. ¹H NMR (600 MHz, DMSO-d₆) δ 3.87 (q, J=5.6 Hz, 2H), 3.51 (t, J=4.9 Hz, 2H), 3.37 (t, J=13.3 Hz, 2H), 2.27-2.15 (m, 2H), 1.51-1.42 (m, 4H), 1.35-1.30 (m, 2H), 1.30-1.19 (m, 22H). ¹³C NMR (151 MHz, DMSO-d₆) δ 127.7 (q, J_{CF}=276.6 Hz), 70.3, 69.3 (d, J_{CP}=7.7 Hz), 64.3 (d, J_{CP}=5.2 Hz), 32.4 (q, J_{CF}=27.2 Hz), 29.2, 29.0, 29.0 (4C), 28.9, 28.9, 28.8, 28.5, 27.9, 25.6, 21.4 (q, J_{CF}=3.0 Hz). ³¹P NMR (243 MHz, DMSO-d₆) δ −1.52. ¹⁹F NMR (565 MHz, DMSO-d₆) δ −65.34 (t, J=11.7 Hz). HRMS (ESI) m/z calculated for C₁₈H₃₅O5F₃P [M−H]⁻ 419.21797, found 419.21814.

L. Synthesis of ammonium [3-[tert-butyl(dimethyl)silyl]oxy-5-(5-fluoro-2,4-dioxo-pyrimidin-1-yl)tetrahydrofuran-2-yl]methyl 2-(16,16,16-trifluorohexadecoxy)ethyl phosphate (NP-PD-285)

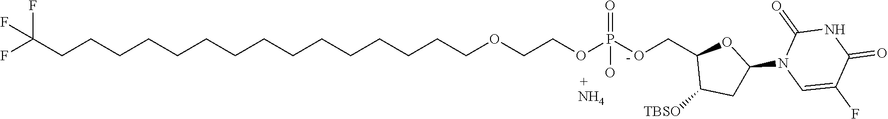

1-[4-[tert-Butyl(dimethyl)silyl]oxy-5-(hydroxymethyl)tetrahydrofuran-2-yl]-5-fluoro-pyrimidine-2,4-dione (129 mg, 0.357 mmol, 1.5 eq), 2-(16,16,16-trifluorohexadecoxy)ethyl dihydrogen phosphate (100 mg, 0.238 mmol, 1.0 eq), cyanotrichloromethane (36 μL, 0.36 mmol), and pyridine (1.0 mL) were placed in a 0.5-2.0 mL microwave vial, fitted with a stir bar. The vial was sealed and irradiated in a microwave reactor for 1 h at 90° C. The reaction mixture was then concentrated in vacuo and the resulting crude material was purified by column chromatography (0-100% 80:20:3 DCM:MeOH:NH₄₀H in DCM) to afford the product NP-PD-285 (78.4 mg, 0.101 mmol, 42% yield) as a pale-yellow oil. ¹H NMR (400 MHz, CD₃OD) δ 8.10 (d, J=6.6 Hz, 1H), 6.33-6.25 (m, 1H), 4.62-4.56 (m, 1H), 4.08-4.03 (m, 3H), 4.03-3.95 (m, 2H), 3.60 (t, J=5.0 Hz, 2H), 3.46 (t, J=6.6 Hz, 2H), 2.32-2.05 (m, 4H), 1.60-1.48 (m, 4H), 1.41-1.27 (m, 22H), 0.93 (s, 9H), 0.14 (d, J=0.9 Hz, 6H). ¹³C NMR (151 MHz, CD₃OD) δ 159.5 (d, J_{CF}=26.4 Hz), 150.9, 141.9 (d, J_{CF}=233.4 Hz), 128.9 (q, J_{CF}=275.3 Hz), 126.3 (d, J_{CF}=34.4 Hz), 88.5 (d, J_{CP}=8.8 Hz), 86.9, 74.7, 72.4, 71.4 (d, J_{CP}=7.8 Hz), 66.2 (d, J_{CP}=5.5 Hz), 66.0 (d, J_{CP}=5.8 Hz), 41.7, 34.4 (q, J_{CF}=28.2 Hz), 30.9, 30.8, 30.8 (2C), 30.8 (2C), 30.7, 30.6, 30.5, 30.3, 29.8, 27.3, 26.3 (3C), 23.0 (q, J_{CF}=2.9 Hz), 18.8,−4.6,—4.6. ³¹P NMR (162 MHz, CD₃OD) δ −0.46. ¹⁹F NMR (376 MHz, CD₃OD) δ −68.75 (t, J=11.1 Hz), −168.37 (dd, J=6.6, 1.7 Hz). HRMS (ESI) m/z calculated for C₃₃H₅₈O₉N₂F₄P^{2S}Si [M−H]⁻ 761.35908, found 761.35847.

M. Synthesis of ammonium [(JR)-1-[3-[tert-butyl(dimethyl)silyl]oxy-5-(5-fluoro-2,4-dioxo-pyrimidin-1-yl)tetrahydrofuran-2-yl]ethyl] 3-(15,15,15-trifluoropentadecoxy)propyl phosphate (NP-PD-286)

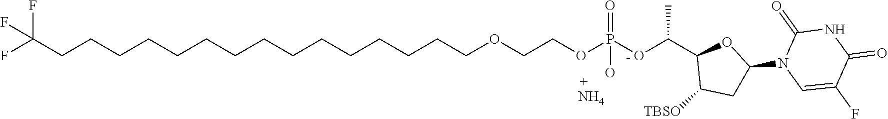

1-[4-[tert-Butyl(dimethyl)silyl]oxy-5-[(1R)-1-hydroxyethyl]tetrahydrofuran-2-yl]-5-fluoro-pyrimidine-2,4-dione (134 mg, 0.357 mmol, 1.5 eq), 3-(15,15,15-trifluoropentadecoxy)propyl dihydrogen phosphate (100 mg, 0.238 mmol, 1.0 eq), cyanotrichloromethane (36 μL, 0.36 mmol), and pyridine (1.0 mL) were placed in a 0.5-2.0 mL microwave vial, fitted with a stir bar. The vial was sealed and irradiated in a microwave reactor for 1 h at 90° C. The reaction mixture was then concentrated in vacuo and the resulting crude material was purified by column chromatography (0-100% 80:20:3 DCM:MeOH:NH₄OH in DCM) to afford the product NP-PD-286 (33 mg, 0.042 mmol, 18% yield) as a pale yellow oil. ¹H NMR (600 MHz, CD₃OD) δ 8.12 (d, J=6.5 Hz, 1H), 6.30-6.25 (m, 1H), 4.68 (d, J=5.1 Hz, 1H), 4.43-4.34 (m, 1H), 4.04-3.96 (m, 2H), 3.79-3.75 (m, 1H), 3.61 (t, J=5.1 Hz, 2H), 3.46 (t, J=6.6 Hz, 2H), 2.22-2.08 (m, 4H), 1.57-1.50 (m, 4H), 1.42-1.36 (m, 5H), 1.36-1.27 (m, 20H), 0.93 (s, 9H), 0.15 (s, 6H). ¹³C NMR (151 MHz, CD₃OD) δ 159.5 (d, J_{CF}=26.3 Hz), 151.0, 142.0 (d, J_{CF}=233.9 Hz), 128.9 (q, J_{CF}=275.3 Hz), 126.5 (d, J_{CF}=34.3 Hz), 92.4 (d, J_{CP}=8.2 Hz), 86.4, 73.2 (app s), 73.1, 72.4, 71.5 (d, J_{CP}=8.3 Hz), 65.8 (d, J_{CP}=5.5 Hz), 41.2, 34.4 (q, J_{CF}=28.2 Hz), 30.9, 30.8, 30.8 (2C), 30.7, 30.7, 30.7, 30.6, 30.5, 30.3, 29.8, 27.3, 26.3 (3C), 23.0 (q, J_{CF}=3.1 Hz), 19.0, 18.7, −4.4,-4.5. ³¹P NMR (243 MHz, CD₃OD) δ −0.94 (app q, ³J_{PH}=6.9 Hz). ¹F NMR (565 MHz, CD₃OD) δ −68.75 (t, J=11.2 Hz), −168.05 (d, J=6.7 Hz). HRMS (ESI) m/z calculated for C₃₄H₆₀O₉N₂F₄P²⁸Si [M−H]⁻ 775.37473, found 775.37421.

N. Synthesis of ammonium [5-(5-fluoro-2,4-dioxo-pyrimidin-1-yl)-3-hydroxy-tetrahydrofuran-2-yl]methyl 2-(16,16,16-trifluorohexadecoxy)ethyl phosphate (NP-PD-287)

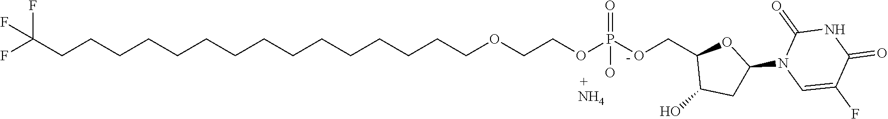

In a 0.5-2.0 mL microwave vial fitted with a stir bar, ammonium [3-[tert-butyl(dimethyl)silyl]oxy-5-(5-fluoro-2,4-dioxo-pyrimidin-1-yl)tetrahydrofuran-2-yl]methyl 2-(16,16,16-trifluorohexadecoxy)ethyl phosphate (78 mg, 0.10 mmol, 1.0 eq) was treated with ammonium fluoride (37 mg, 1.0 mmol, 10 eq) and MeOH (1.0 mL). The vial was then sealed and irradiated in a microwave reactor for 1 h at 80° C. The reaction mixture was then concentrated in vacuo and the resulting crude material was purified using reverse phase (Cis) column chromatography (10-100% MeOH in H₂O) to afford NP-PD-287 (43 mg, 0.065 mmol, 64% yield) as a white solid. ¹H NMR (400 MHz, CD₃OD) δ 8.11 (d, J=6.6 Hz, 1H), 6.30 (td, J=6.9, 1.8 Hz, 1H), 4.49 (td, J=4.2, 2.1 Hz, 1H), 4.11-4.02 (m, 3H), 4.02-3.95 (m, 2H), 3.61 (t, J=5.0 Hz, 2H), 3.47 (t, J=6.6 Hz, 2H), 2.26 (dd, J=7.0, 4.2 Hz, 2H), 2.21-2.04 (m, 2H), 1.61-1.48 (m, 4H), 1.43-1.26 (m, 22H). ¹³C NMR (101 MHz, CD₃OD) δ 159.5 (d, J_{CF}=26.2 Hz), 150.8, 141.9 (d, J_{CF}=233.1 Hz), 128.9 (q, J_{CF}=275.4 Hz), 126.3 (d, J_{CF}=34.5 Hz), 87.8 (d, J_{CP}=8.7 Hz), 86.8, 72.8, 72.4, 71.4 (d, J_{CP}=8.0 Hz), 66.3 (d, J_{CP}=5.4 Hz), 66.0 (d, J_{CP}=5.6 Hz), 41.0, 34.4 (q, J_{CF}=28.2 Hz), 30.8, 30.8 (2C), 30.8 (3C), 30.7, 30.6, 30.5, 30.3, 29.8, 27.2, 23.0 (q, J_{CF}=3.0 Hz). ³¹P NMR (162 MHz, CD₃OD) δ −0.44. ¹F NMR (376 MHz, CD₃OD) δ −68.71 (t, J=11.2 Hz), −168.50 (dd, J=6.9, 2.0 Hz). HRMS (ESI) m/z calculated for C₂₇H₄₄O₉N₂F₄P [M−H]⁻ 647.27260, found 647.27262. LC-MS (ESI, C₈, 0.6 mL/min) 55-95% MeCN in H₂O, 6 min, t_{R}=1.285 min, m/z=647.5 [M−H]⁻; 40-95% MeCN in H₂O, 6 min, t_{R}=3.555 min, m/z=647.4 [M−H]⁻.

O. Synthesis of ammonium [(JR)-1-[5-(5-fluoro-2,4-dioxo-pyrimidin-1-yl)-3-hydroxy-tetrahydrofuran-2-yl]ethyl] 3-(15,15,15-trifluoropentadecoxy)propyl phosphate (NP-PD-288)

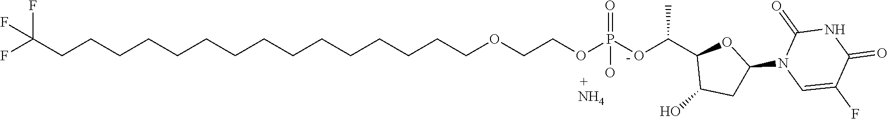

In a 0.5-2.0 mL microwave vial fitted with a stirrer bar, ammonium [(1R)-1-[3-[tert-butyl(dimethyl)silyl]oxy-5-(5-fluoro-2,4-dioxo-pyrimidin-1-yl)tetrahydrofuran-2-yl]ethyl] 3-(15,15,15-trifluoropentadecoxy)propyl phosphate (27 mg, 0.034 mmol, 1.0 eq) was treated with ammonium fluoride (13 mg, 0.34 mmol, 10 eq) and MeOH (0.5 mL). The vial was then sealed and irradiated in a microwave reactor for 1 h at 80° C. The reaction mixture was then concentrated in vacuo and the resulting crude material was purified using reverse phase (Cis) column chromatography (10-100% MeOH in H₂O) to afford NP-PD-288 (14 mg, 0.021 mmol, 62% yield) as a white solid. ¹H NMR (400 MHz, CD₃OD) δ 8.09 (d, J=6.5 Hz, 1H), 6.29 (td, J=7.2, 1.9 Hz, 1H), 4.57 (td, J=4.5, 4.0, 2.1 Hz, 1H), 4.46-4.33 (m, 1H), 4.06-3.94 (m, 2H), 3.78-3.71 (m, 1H), 3.61 (t, J=5.1 Hz, 2H), 3.47 (t, J=6.6 Hz, 2H), 2.24-2.06 (m, 4H), 1.59-1.48 (m, 4H), 1.40-1.27 (m, 25H). ¹³C NMR (101 MHz, CD₃OD) δ 159.5 (d, J_{CF}=26.2 Hz), 150.9, 142.0 (d, J_{CF}=233.7 Hz), 128.9 (app t, J_{CF}=275.4 Hz), 126.5 (d, J_{CF}=34.3 Hz), 91.5 (d, J_{CP}=8.2 Hz), 86.2, 73.4 (d, J_{CP}=5.8 Hz), 72.4, 71.8, 71.5 (d, J_{CP}=8.3 Hz), 65.8 (d, J_{CP}=5.5 Hz), 40.5, 34.4 (q, J_{CF}=28.2 Hz), 30.8, 30.8 (2C), 30.8 (3C), 30.7, 30.6, 30.5, 30.3, 29.8, 27.2, 23.0 (q, J_{CF}=2.9 Hz), 18.8. ³¹P NMR (162 MHz, CD₃OD) δ −0.91 (app d, ³J_{PH}=7.2 Hz). ¹⁹F NMR (376 MHz, CD₃OD) δ −68.74 (t, J=11.2 Hz), −168.19 (dd, J=6.6, 2.1 Hz). HRMS (ESI) m/z calculated for C₂₈H₄₆O₉N₂F₄P [M−H]⁻ 661.28825, found 661.28838. LC-MS (ESI, C₈, 0.6 mL/min) 55-95% MeCN in H₂O, 6 min, t_{R}=1.137 min, m/z=661.5 [M−H]⁻; 40-95% MeCN in H₂O, 6 min, t_{R}=3.754 min, m/z=661.5 [M−H]⁻.

### Example 12. Crystallography Analysis

### A. Methods

Single colorless needle crystals of MD-7-29, MD-7-42, and MD-7-159-1 were obtained via recrystallization from either DCM or ethanol by slow evaporation. A suitable crystal with dimensions 0.43×0.07×0.03 mm³ for MD-7-29, 0.51×0.37×0.11 mm³ for MD-7-42, and 0.12×0.04×0.03 mm³ for MD-7-159-1 was selected and mounted on a loop with paratone on a XtaLAB Synergy-S diffractometer. The crystals were kept at a steady temperature (T=ca. 100 K) during data collection. Data were measured using a scans using Cu Kₐ radiation. The diffraction pattern was indexed and the total number of runs and images was based on the strategy calculation from the program CrysAlisPro (Rigaku, V1.171.40.82a, 2020). The maximum resolution that was achieved was Θ=68.25° (0.83 Å) for MD-7-29, Θ=65.088° (0.85 Å) for MD-7-42, and Θ=44.48° (1.10 Å) for MD-7-159-1. The unit cell was refined using CrysAlisPro.

Data reduction, scaling and absorption corrections were performed using CrysAlisPro. The final completeness was 99.67% out to 68.25° in Θ for MD-7-29, 99.70% out to 65.088° in Θ for MD-7-42, and 79.42% out to 44.48° in Θ for MD-7-159-1. A numerical absorption correction based on a Gaussian integration over a multifaceted crystal model absorption correction was performed using CrysAlisPro 1.171.40.79a (Rigaku Oxford Diffraction, 2020). An empirical absorption correction using spherical harmonics, implemented in SCALE3 ABSPACK scaling algorithm was also applied. For MD-7-29, the absorption coefficient μ of this material was 0.924 mm⁻¹ at this wavelength (λ=1.54184 Å) and the minimum and maximum transmissions were 0.455 and 1.000. For MD-7-42, the absorption coefficient μ of this material was 1.380 mm⁻¹ at this wavelength (λ=1.54184 Å) and the minimum and maximum transmissions were 0.216 and 1.000. For MD-7-159-1, the absorption coefficient μ of this material was 1.162 mm⁻¹ at this wavelength (λ=1.54184 Å) and the minimum and maximum transmissions were 0.850 and 1.000.

The structures of MD-7-29 and MD-7-42 were solved in the space group P2₁ (#4) and the structure of MD-7-159-1 was solved in the space group P2₁2₁2₁ (#19), with the ShelXT (Sheldrick, Acta Cryst., 2015, A71, 3-8) solution program or ShelXT 2018/2, using dual methods and using Olex2 (Dolomanov, et al., J. Appl. Crystallogr, 2009, 42, 339-341) as the graphical interface. The model was refined with olex2.refine 1.3-dev (Bourhis, et al., Acta Cryst, 2015, A71, 59-75) or ShelXT 2018/3 using full matrix least squares minimization on F². For MD-7-29, all atoms including hydrogens were refined anisotropically; hydrogen atom positions were freely refined. For MD-7-42 and MD-7-159-1, all non-hydrogen atoms were refined anisotropically; hydrogen atom positions were calculated geometrically and refined using the riding model.

### B. Results

Representative crystallization data for MD-7-29 are summarized below: C₁₆H₂₇FN₂O₅Si, Mr=374.487, monoclinic, P2₁ (No. 4), a=18.4223(5) Å, b=7.8844(2) Å, c=21.7189(6) Å, β=109.490(3)°, α=γ=90°, V=2973.88(15) Å³, T=100(2) K, Z=6, Z′=3, μ(Cu K_{α})=0.924 mm-1, 46958 reflections measured, 10712 unique (Rᵢₙₜ=0.1129) which were used in all calculations. The final wR₂ was 0.2126 (all data) and R₁ was 0.0777 (I≥2σ(I)).

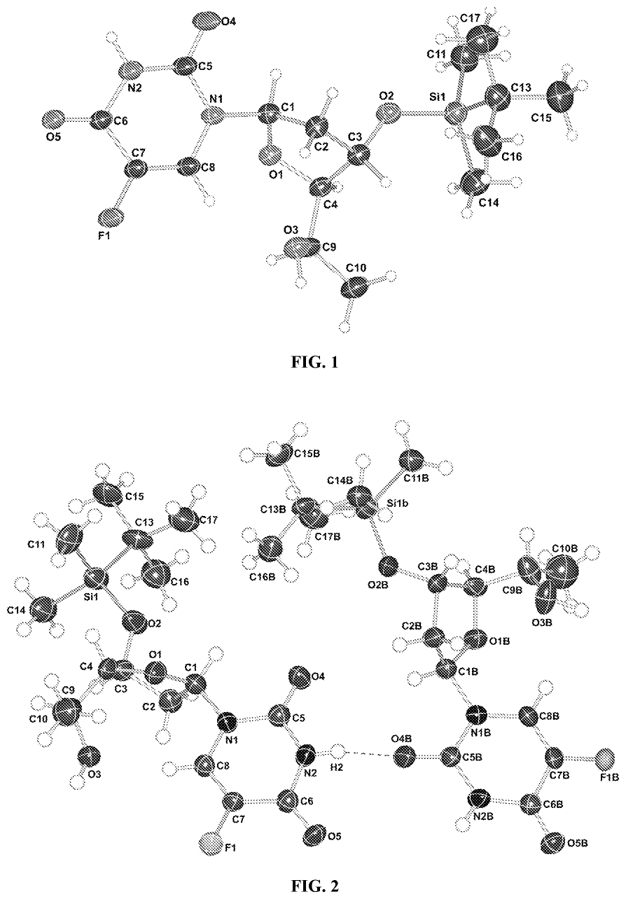

The results showed that there were three molecules in the asymmetric unit. The molecules formed strong hydrogen bonds to each other. FIG. 1 shows a representative molecule of MD-7-29 in the asymmetric unit. The chirality was R, S, R, and R at the atoms C1, C3, C4, and C9 (according to the numbering of atoms shown in FIG. 1), respectively. This result was consistent with the anticipated stereochemistry of MD-7-29.

Representative crystallization data for MD-7-42 are summarized below:

C₁₆H₂₇FN₂O₅Si, Mr=374.48, monoclinic, P2₁ (No. 4), a=7.97810(10) Å, b=10.9340(2) Å, c=22.5705(4) A, =91.837(2)°, α=γ=90°, V=1967.87(6) Å³, T=99.99(10) K, Z=4, Z′=2, μ(Cu K_{α})=1.380 mm-1, 24163 reflections measured, 6684 unique (Rᵢₙₜ=0.0365) which were used in all calculations. The final wR₂ was 0.1634 (all data) and R₁ was 0.0605 (I≥2σ(I)).

The results showed that there were two independent molecules of MD-7-42 in the asymmetric unit (FIG. 2). The chirality was R, S, R, and S at the atoms C1, C3, C4, and C9 (according to the numbering of atoms shown in FIG. 2), respectively. This result was consistent with the anticipated stereochemistry of MD-7-42.

Representative crystallization data for MD-7-159-1 are summarized below:

C₁₉H₂₂F₄N₂O₆, Mr=450.390, orthorhombic, P2₁2₁2₁ (No. 19), a=4.9274(9) A, b=8.8511(13) Å, c=46.430(8) Å, α=β=γ=90°, V=2025.0(6) Å³, T=100.00(10) K, Z=4, Z′=1, μ(Cu K_{α})=1.162, 3688 reflections measured, 1158 unique (Rᵢₙₜ=0.1114) which were used in all calculations. The final wR₂ was 0.3367 (all data) and R₁ was 0.1281 (1≥2σ(I))).

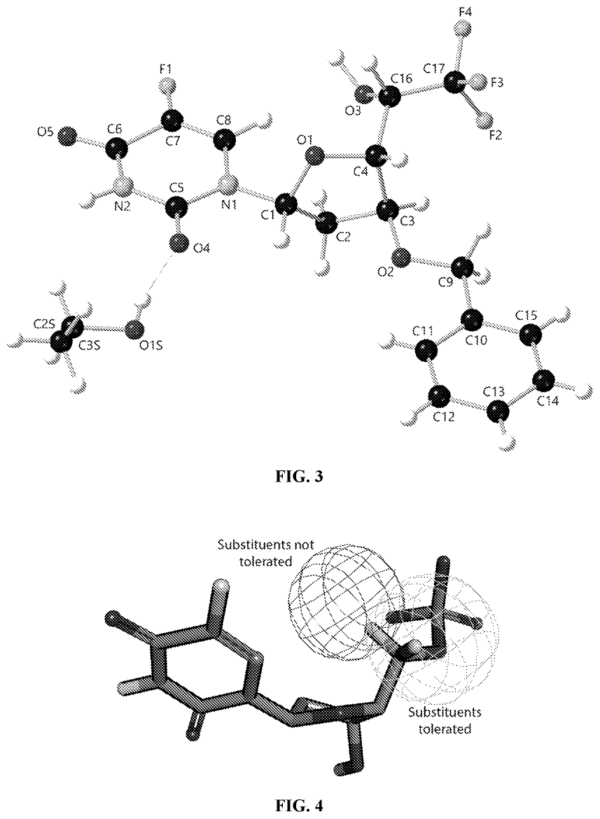

The results showed that there was one independent molecule of MD-7-159-1 and one molecule of ethanol in the asymmetric unit (FIG. 3). The chirality was R, S, R at the atoms C1, C3, and C4, respectively, of MD-7-159-1 (according to the numbering of atoms shown in FIG. 3). This result was consistent with the anticipated stereochemistry of MD-7-159-1.

### Example 13. Computational Analysis

### A. Methods

Docking studies of FdUMP derivatives with one or more substitutions at the 5′ position was perform using Glide (Schrödinger Release 2020-3: Glide, Schrödinger, LLC, New York, NY, 2020). A crystal structure of human TS co-crystalized with FdUMP was used (PDB ID 6QXG, 2.08 Å, chain A).

Protein preparation was carried out using the Protein Preparation wizard in the Schrödinger 2020-3 suite, including adding side chains if missing. All water molecules were deleted after the preparation workflow and the FdUMP ligand in chain A was used to identify the docking binding pocket. Docking studies were carried out using GlideXP with expanded sampling. Following this, binding energy calculations were carried out using the Prime MM-GBSA tool, with a 10 Å minimization radius. GlideXP results are unitless, more negative values represent better docking scores. Prime MM-GBSA units are kcal/mol.

Since docking is generally very sensitive to steric clashes, it was performed as a first pass. The native ligand FdUMP was docked first to see if the crystal structure pose could be accurately recapitulated. The docking structure of the FdUMP-human TS complex highly resembled the co-crystal structure.

FdUMP was then modified to introduce different substitutions at the 5′ position. These FdUMP derivatives was docked with the afore-identified structure of human TS. All FdUMP derivatives were visually inspected to compare their docked poses to that of the co-crystallized FdUMP ligand. Following the docking, analysis of the binding energies by Prime MM-GBSA was carried out as described above.

Selected FdUMP derivatives were further analyzed by free energy perturbation (FEP) methods (D. E. Shaw Research, 2021) to compare the change in free binding energy (AAG) as compared to the co-crystalized FdUMP ligand.

### B. Results

The docking results of the FdUMP derivatives are summarized in Table 1.

TABLE 1
Summary of docking studies on FdUMP derivatives

| | | Binding | |
|---|---|---|---|
| | Glide | Energy (Prime | |
| | XP | MM-GBSA, | FEP ΔΔG |
| Structure | Score | kcal/mol) | (kcal/mol) |
| 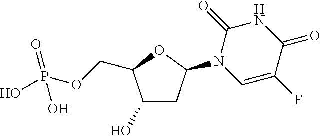 [SMILES: O=c1[nH]c(=O)n([C@H]2C[C@H](O)[C@@H](COP(=O)(O)O)O2)cc1F] | −13.0 | −61.1 | Reference |
| FdUMP | | | |
| 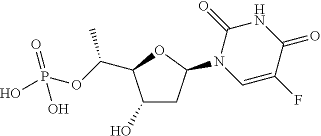 [SMILES: C[C@@H](OP(=O)(O)O)[C@H]1O[C@@H](n2cc(F)c(=O)[nH]c2=O)C[C@@H]1O] | −12.9 | −62.6 | −0.62 |
| 5′-R-methyl FdUMP | | | |
| 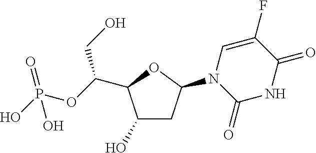 [SMILES: O=c1[nH]c(=O)n([C@H]2C[C@H](O)[C@@H]([C@@H](CO)OP(=O)(O)O)O2)cc1F] | −13.2 | −62.8 | ND |
| 5′-R-hydroxymethyl FdUMP | | | |
| 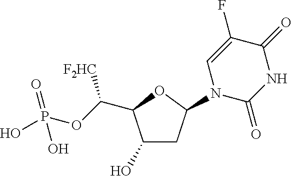 [SMILES: O=c1[nH]c(=O)n([C@H]2C[C@H](O)[C@@H]([C@H](OP(=O)(O)O)C(F)F)O2)cc1F] | −12.8 | −63.1 | 1.19 |
| 5′-S-difluoromethyl FdUMP | | | |
| 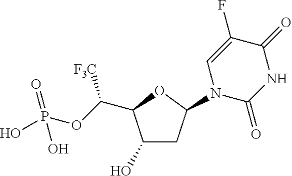 [SMILES: O=c1[nH]c(=O)n([C@H]2C[C@H](O)[C@@H]([C@H](OP(=O)(O)O)C(F)(F)F)O2)cc1F] | −12.8 | −60.6 | −5.24 |
| 5′-S-trifluoromethyl FdUMP | | | |
| 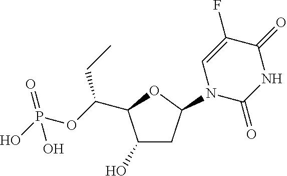 [SMILES: CC[C@@H](OP(=O)(O)O)[C@H]1O[C@@H](n2cc(F)c(=O)[nH]c2=O)C[C@@H]1O] | −12.8 | −57.7 | 1.49 |
| 5′-R-ethyl FdUMP | | | |
| 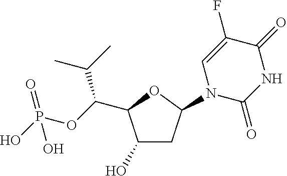 [SMILES: CC(C)[C@@H](OP(=O)(O)O)[C@H]1O[C@@H](n2cc(F)c(=O)[nH]c2=O)C[C@@H]1O] | −12.9 | −51.4 | ND |
| 5′-R-isopropyl FdUMP | | | |
| 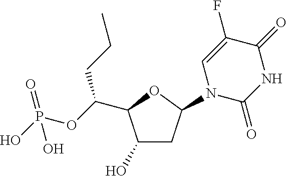 [SMILES: CCC[C@@H](OP(=O)(O)O)[C@H]1O[C@@H](n2cc(F)c(=O)[nH]c2=O)C[C@@H]1O] | −12.8 | −50.4 | ND |
| 5′-R-propyl FdUMP | | | |
| 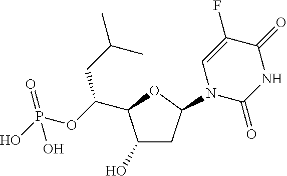 [SMILES: CC(C)C[C@@H](OP(=O)(O)O)[C@H]1O[C@@H](n2cc(F)c(=O)[nH]c2=O)C[C@@H]1O] | −9.7 | −45.8 | ND |
| 5′-R-isobutyl FdUMP | | | |
| 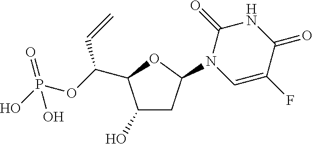 [SMILES: C=C[C@@H](OP(=O)(O)O)[C@H]1O[C@@H](n2cc(F)c(=O)[nH]c2=O)C[C@@H]1O] | −12.8 | −56.8 | ND |
| 5′-R-ethenyl FdUMP | | | |
| 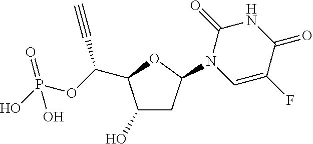 [SMILES: C#C[C@@H](OP(=O)(O)O)[C@H]1O[C@@H](n2cc(F)c(=O)[nH]c2=O)C[C@@H]1O] | −12.9 | −51.7 | ND |
| 5′-R-ethynyl FdUMP | | | |
| 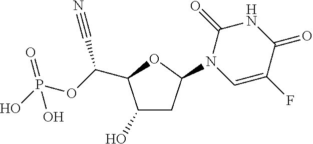 [SMILES: N#C[C@@H](OP(=O)(O)O)[C@H]1O[C@@H](n2cc(F)c(=O)[nH]c2=O)C[C@@H]1O] | −12.7 | −51.7 | ND |
| 5′-R-cyano FdUMP | | | |
| 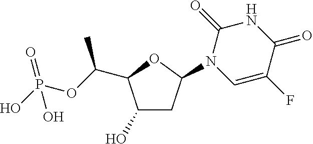 [SMILES: C[C@H](OP(=O)(O)O)[C@H]1O[C@@H](n2cc(F)c(=O)[nH]c2=O)C[C@@H]1O] | −12.3 | −52.3 | 4.46 |
| 5′-S-methyl FdUMP | | | |
| 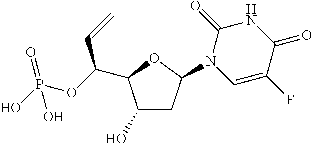 [SMILES: C=C[C@H](OP(=O)(O)O)[C@H]1O[C@@H](n2cc(F)c(=O)[nH]c2=O)C[C@@H]1O] | −12.5 | −53.3 | ND |
| 5′-S-ethenyl FdUMP | | | |
| 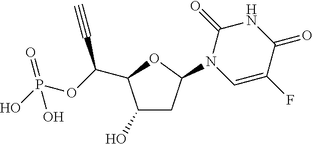 [SMILES: C#C[C@H](OP(=O)(O)O)[C@H]1O[C@@H](n2cc(F)c(=O)[nH]c2=O)C[C@@H]1O] | −12.2 | −45.8 | ND |
| 5′-S-ethynyl FdUMP | | | |
| 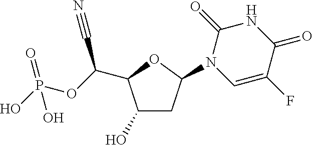 [SMILES: N#C[C@H](OP(=O)(O)O)[C@H]1O[C@@H](n2cc(F)c(=O)[nH]c2=O)C[C@@H]1O] | −12.4 | −45.9 | ND |
| 5′-S-cyano FdUMP | | | |
| 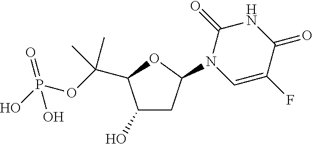 [SMILES: CC(C)(OP(=O)(O)O)[C@H]1O[C@@H](n2cc(F)c(=O)[nH]c2=O)C[C@@H]1O] | −12.5 | −51.9 | 3.60 |
| 5′-gem-dimethyl FdUMP | | | |
| 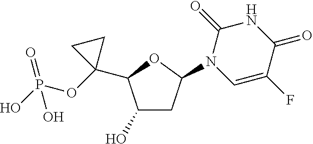 [SMILES: O=c1[nH]c(=O)n([C@H]2C[C@H](O)[C@@H](C3(OP(=O)(O)O)CC3)O2)cc1F] | −13.1 | −51.9 | ND |
| 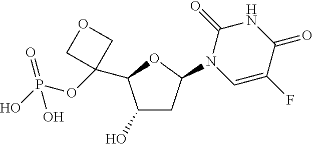 [SMILES: O=c1[nH]c(=O)n([C@H]2C[C@H](O)[C@@H](C3(OP(=O)(O)O)COC3)O2)cc1F] | −12.3 | −51.6 | ND |

ND: not determined

5′-R-methyl FdUMP and 5′-R-hydroxymethyl FdUMP scored very similarly to FdUMP in both Glide XP and binding energy. There was a drop in both parameters when the substituent became larger. Therefore, larger substituents performed poorly. There was a drop in both parameters when the substituent was placed on the other side of the 5′ position, such as the scenario in 5′-S-methyl HdUMP. Therefore, substituents placed on the other side of the 5′ position performed poorly. The docking results revealed that the active site of human TS has more room for the R-methyl than the S-methyl. 5′-R-methyl FdUMP would be as well accommodated as the native crystal ligand, HdUMP. For 5′-S-methyl HdUMP, to retain the original co-crystal pose, the S-methyl would clash with the cysteine residue that acts as the enzyme's catalytic nucleophile.

5′-S-trifluoromethyl FdUMP and 5′-S-difluoromethyl FdUMP also performed well in both Glide XP and binding energy. In addition, 5′-S-trifluoromethyl FdUMP performed well in the FEP analysis. It should be noted that the trifluoromethyl group in 5′-R-trifluoromethyl FdUMP and the difluoromethyl group in 5′-S-difluoromethyl FdUMP are on the same side of the methyl group in 5′-R-methyl FdUMP.

Taken together, the docking study showed that there is space for small substituents on the 5′ position of FdUMP. Substitution on one side of the 5′ position, i.e., the same side as the R-methyl in 5′-R-methyl FdUMP, would not cause steric clashes. Substitution on one side of the 5′ position, i.e., the same side as the S-methyl in 5′—S-methyl FdUMP, would induce conformational changes of FdUMP to avoid steric clashes with residues in the active site of human TS, thereby causing a deviation from the original crystal structure pose. See FIG. 4 for illustration.

### Example 14. Human Thymidylate Synthase Inhibition Assays

### A. Materials and Methods

Recombinant human thymidylate synthase (hTS) was purchased from Abcam. The protein was >95% pure according to the vendor and contained a His₆-tag. FdUMP (disodium salt) was purchased from Sigma Aldrich and used without further purification. 5,10-Methylenetetrahydrofolate (calcium salt) was purchased from Boc Sciences and used without further purification.

The hTS inhibition assays were performed on a BioTek Synergy Neo2 plate reader equipped with a Xenon flash lamp and a photodiode array detector. The hTS reaction was conducted at room temperature in a buffer solution (pH 7.5) containing 50 mM Tris-HCl, 1 mM EDTA, 25 mM MgCl₂, and 5 mM HCHO. The reaction system (175 μL total volume, 1 cm optical path length) consisted of 50 μM dUMP (primary substrate), 250 μM 5,10-methylenetetrahydrofolate (co-substrate and enzyme cofactor), FdUMP or a 5′-substituted analog thereof (inhibitor, at various concentrations), and 0.84 μM hTS. 5,10-Methylenetetrahydrofolate was added last to initiate the reaction. Formation of the co-product, dihydrofolate, was monitored at 340 nm. The absorbance at 340 nm was plotted against the reaction time to calculate the reaction rate (i.e., the slope of the linear fitting). The reaction rates at different inhibitor concentrations were normalized to the reaction rate determined in the absence of inhibitor (100% enzymatic activity). The resulting % enzymatic activity values were plotted against the corresponding log value of the inhibitor concentration to construct concentration-response curves. IC₅₀ values were then calculated using non-linear regression (four-parameter logistic equation, see Equation 1 below) in the GraphPad Prism v.9 software.

Y=Yₘᵢₙ+(Yₘₐₓ−Yₘᵢₙ)/[1+10^{((logIC50-X)×Hill Slope)}] Equation 1

### B. Results

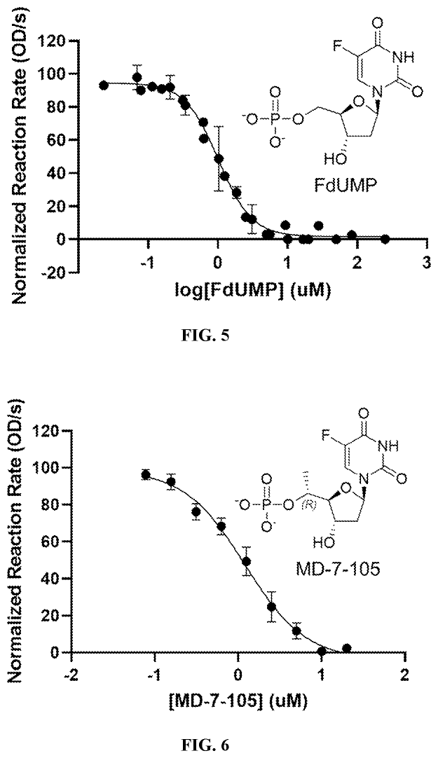

FdUMP inhibited hTS in a concentration-dependent manner (FIG. 5). The IC₅₀ value of FdUMP was determined as 1.02 μM with a confidence interval of 0.87-1.19 μM (from five repeats) in the presence of 50 μM dUMP and 0.84 μM hTS.

5′-(R)-methyl-FdUMP (MD-7-105) also inhibited hTS in a concentration-dependent manner (FIG. 6). The potency of this compound against hTS was comparable to FdUMP, as shown by an IC₅₀ value of 1.21 μM with a confidence interval of 0.81-1.72 μM (from five repeats).

In contrast, 5′-(S)-methyl-FdUMP (MD-7-112) and 5′-gem-dimethyl-FdUMP (MD-7-115) were inactive against hTS. Neither compound was able to inhibit hTS at the highest concentration tested (20 μM).

1. A compound having a structure of Formula I or a pharmaceutically acceptable salt thereof: 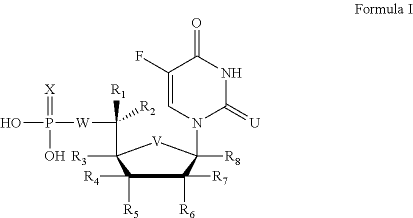
wherein:
(1) R₁ and R₂ are independently selected from the group consisting of hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen,
(2) R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ, or
(3) one of R₁ and R₂ is selected from the group consisting of hydrogen, deuterium, halogen, cyano, carboxyl, C₁-C₃ alkyl optionally substituted by one or more Rₐ, ethenyl optionally substituted by one or more Rₑ, ethynyl optionally substituted by Rₐ, 3- or 4-membered carbocyclyl optionally substituted by one or more Rₐ, and 3- or 4-membered heterocyclyl optionally substituted by one or more Rₐ, and the other one of R₁ and R₂ joins with the 4′ carbon and the 5′ carbon to form a 3- or 4-membered carbocycle optionally substituted by one or more Rₐ;
wherein U is O or S;
wherein V is O or S;
wherein W is O or optionally substituted methylene;
wherein X is O or S;
wherein R₃ is absent or selected from the group consisting of hydrogen, deuterium, halogen, cyano, ethynyl, optionally substituted alkyl, optionally O-substituted hydroxyl, and ester;
wherein R₄ is hydrogen or deuterium;
wherein R₅ is selected from the group consisting of fluorine, optionally O-substituted hydroxyl, amino, acyl, ester, amide, acylamino, and substituted alkyl comprising a substituent selected from the group consisting of fluorine, optionally O-substituted hydroxyl, and amino;
wherein R₆, R₇, and R₈ are independently selected from the group consisting of hydrogen, deuterium, halogen, cyano, ethynyl, optionally substituted alkyl, optionally O-substituted hydroxyl, and ester; and
wherein Rₐ is selected from the group consisting of deuterium, halogen, and hydroxyl.

2. The compound of claim 1, wherein R₁ and R₂ are independently selected from the group consisting of hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen.

3. The compound of claim 2, wherein R₁ is hydrogen, deuterium, halogen, methyl optionally substituted by one or more Rₐ, or ethenyl optionally substituted by one or more Rₐ.

4. The compound of claim 3, wherein R₁ is hydrogen, methyl, or ethenyl.

5. The compound of claim 4, wherein R₁ is hydrogen.

6. The compound of claim 1, wherein R₂ is selected from the group consisting of cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ.

7. The compound of claim 6, wherein R₂ is methyl optionally substituted by one or more Rₐ, ethyl optionally substituted by one or more Rₐ, ethenyl optionally substituted by one or more Rₐ, or 3- or 4-membered carbocyclyl optionally substituted by one or more Rₐ.

8. The compound of claim 7, wherein R₂ is methyl, —CF₃, —CH₂OH, ethenyl, cyclopropyl, or cyclobutyl.

9. The compound of claim 8, wherein R₂ is methyl, —CF₃, or —CH₂OH.

10. The compound of claim 2, wherein R₁ is hydrogen and R₂ is selected from the group consisting of cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ.

11. The compound of claim 10, wherein R₁ is hydrogen and R₂ is methyl, —CF₃, —CH₂OH, ethenyl, cyclopropyl, or cyclobutyl.

12. The compound of claim 11, wherein R₁ is hydrogen and R₂ is methyl, —CF₃, or —CH₂OH.

13.-21. (canceled)

22. The compound of claim 1, wherein U, V, W, and X are O.

23.-28. (canceled)

29. The compound of claim 1, wherein R₃, R₄, R₆, R₇, and R₈ are hydrogen and R₅ is hydroxyl.

30. The compound of claim 1, wherein the compound is selected from the group consisting of:
(R)-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(S)-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
2-((2S,3S,5R)-5-(5-fluoro-2,4-dioxo-3,4-dihydropyrimidin-1(2H)-yl)-3-hydroxytetrahydrofuran-2-yl)propan-2-yl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)-2-hydroxyethyl dihydrogen phosphate,
(S)-1-((2S,3S,5R)-5-(5-fluoro-2,4-dioxo-3,4-dihydropyrimidin-1(2H)-yl)-3-hydroxytetrahydrofuran-2-yl)-2-hydroxyethyl dihydrogen phosphate,
(S)-2,2,2-trifluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(R)-2,2,2-trifluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(S)-2,2-difluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(R)-2,2-difluoro-1-((2S,3S,5R)-5-(5-fluoro-2,4-dioxo-3,4-dihydropyrimidin-1(2H)-yl)-3-hydroxytetrahydrofuran-2-yl)ethyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)propyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)-2-methylpropyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)butyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)allyl dihydrogen phosphate,
(S)-1-((2S,3S,5R)-5-(5-fluoro-2,4-dioxo-3,4-dihydropyrimidin-1(2H)-yl)-3-hydroxytetrahydrofuran-2-yl)allyl dihydrogen phosphate,
(R)-1-((2S,3S,5R)-5-(5-fluoro-2,4-dioxo-3,4-dihydropyrimidin-1(2H)-yl)-3-hydroxytetrahydrofuran-2-yl)prop-2-yn-1-yl dihydrogen phosphate,
(S)-1-((2S,3S,5R)-5-(5-fluoro-2,4-dioxo-3,4-dihydropyrimidin-1(2H)-yl)-3-hydroxytetrahydrofuran-2-yl)prop-2-yn-1-yl dihydrogen phosphate,
(R)-cyano((2S,3S,5R)-5-(5-fluoro-2,4-dioxo-3,4-dihydropyrimidin-1(2H)-yl)-3-hydroxytetrahydrofuran-2-yl)methyl dihydrogen phosphate,
(S)-cyano((2S,3S,5R)-5-(5-fluoro-2,4-dioxo-3,4-dihydropyrimidin-1(2H)-yl)-3-hydroxytetrahydrofuran-2-yl)methyl dihydrogen phosphate,
1-((2S,3S,5R)-5-(5-fluoro-2,4-dioxo-3,4-dihydropyrimidin-1(2H)-yl)-3-hydroxytetrahydrofuran-2-yl)cyclopropyl dihydrogen phosphate,
1-((2S,3S,5R)-5-(5-fluoro-2,4-dioxo-3,4-dihydropyrimidin-1(2H)-yl)-3-hydroxytetrahydrofuran-2-yl)cyclobutyl dihydrogen phosphate,
3-((2S,3S,5R)-5-(5-fluoro-2,4-dioxo-3,4-dihydropyrimidin-1(2H)-yl)-3-hydroxytetrahydrofuran-2-yl)oxetan-3-yl dihydrogen phosphate,
3-((2S,3S,5R)-5-(5-fluoro-2,4-dioxo-3,4-dihydropyrimidin-1(2H)-yl)-3-hydroxytetrahydrofuran-2-yl)thietan-3-yl dihydrogen phosphate,
1-((2S,3S,5R)-5-(5-fluoro-2,4-dioxo-3,4-dihydropyrimidin-1(2H)-yl)-3-hydroxytetrahydrofuran-2-yl)vinyl dihydrogen phosphate,
(1R,3S,5R,7S)-5-(5-fluoro-2,4-dioxo-3,4-dihydropyrimidin-1(2H)-yl)-7-hydroxy-4-oxaspiro[2.4]heptan-1-yl dihydrogen phosphate,
(1R,4S,6R,8S)-6-(5-fluoro-2,4-dioxo-3,4-dihydropyrimidin-1(2H)-yl)-8-hydroxy-5-oxaspiro[3.4]octan-1-yl dihydrogen phosphate, and
pharmaceutically acceptable salts thereof.

31. (canceled)

32. A prodrug having a structure of Formula II or Formula III, or a pharmaceutically acceptable salt thereof, 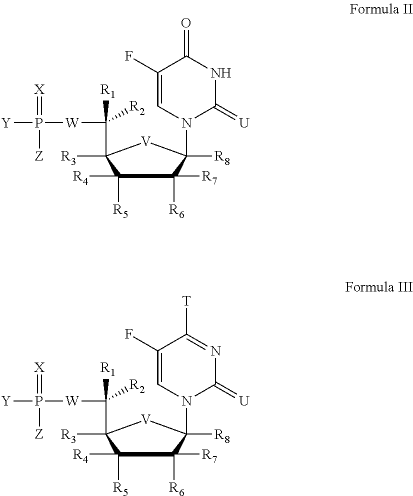
wherein:
(1) R₁ and R₂ are independently selected from the group consisting of hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, with the proviso that at least one of R₁ and R₂ is not hydrogen, deuterium, or halogen,
(2) R₁ and R₂ join with the 5′ carbon to form a 3- or 4-membered carbocycle optionally substituted by one or more Rₐ, a 3- or 4-membered heterocycle optionally substituted by one or more Rₐ, or an ethene moiety optionally substituted by one or more Rₐ, or
(3) one of R₁ and R₂ is selected from the group consisting of hydrogen, deuterium, halogen, cyano, carboxyl, C₁-C₃ alkyl optionally substituted by one or more Rₐ, ethenyl optionally substituted by one or more Rₐ, ethynyl optionally substituted by Rₐ, 3- or 4-membered carbocyclyl optionally substituted by one or more Rₐ, and 3- or 4-membered heterocyclyl optionally substituted by one or more Rₐ, and the other one of R₁ and R₂ joins with the 4′ carbon and the 5′ carbon to form a 3- or 4-membered carbocycle optionally substituted by one or more Rₐ;
wherein U is O or S;
wherein V is O or S;
wherein W is O or optionally substituted methylene;
wherein X is O or S;
wherein R₃ is absent or selected from the group consisting of hydrogen, deuterium, halogen, cyano, ethynyl, optionally substituted alkyl, optionally O-substituted hydroxyl, and ester;
wherein R₄ is hydrogen or deuterium;
wherein R₅ is selected from the group consisting of fluorine, optionally O-substituted hydroxyl, amino, acyl, ester, amide, acylamino, and substituted alkyl comprising a substituent selected from the group consisting of fluorine, optionally O-substituted hydroxyl, and amino;
wherein R₆, R₇, and R₈ are independently selected from the group consisting of hydrogen, deuterium, halogen, cyano, ethynyl, optionally substituted alkyl, optionally O-substituted hydroxyl, and ester;
wherein Rₐ is selected from the group consisting of deuterium, halogen, and hydroxyl;
wherein Y and Z are independently selected from the group consisting of —O—R₉, —S—R₁₀, and 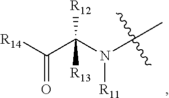
with the proviso that Y and Z are not both hydroxyl;
wherein T is —NR₁₅R₁₆ or —OR₁₇;
wherein R₉ is selected from the group consisting of hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, —R_{q1}—R_{q2}—R_{q3}—R_{q4}, and Rᵣ₁—Rᵣ₂;
wherein R₁₀ is selected from the group consisting of optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₁ is selected from the group consisting of hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl;
wherein R₁₂ and R₁₃ are independently selected from the group consisting of hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, and standard amino acid side chain,
wherein when the standard amino acid side chain is a proline side chain, one of R₁₂ and R₁₃ is hydrogen, and the other one of R₁₂ and R₁₃ forms a pyrrolidine ring together with R₁₁, the nitrogen atom connected to R₁₁, and the carbon atom connected to R₁₂ and R₁₃;
wherein R₁₄ is —NRₛ₁Rₛ₂ or —ORₜ;
wherein R₁₅ and R₁₆ are independently selected from the group consisting of hydrogen, acyl, ester, thioester, and amide;
wherein R₁₇ is acyl, ester, thioester, or amide;
wherein:
R_{q1} is absent or a C₁-C₉ alkyl chain (i.e., C₁-C₉ bridging alkylene),
R_{q2} is absent or is selected from the group consisting of substituted methylene or ethylene, —O—, —S—, —S(═O)—, —S—S—, and —S(O)₂—,
R_{q3} is a C₂-C₂₀ alkyl chain (i.e., C₂-C₂₀ bridging alkylene), and
R_{q4} is selected from the group consisting of hydrogen, optionally substituted methyl or ethyl, optionally substituted C₂-C₃ alkenyl or alkynyl, optionally substituted carbocyclyl, optionally substituted heterocyclyl, optionally substituted aryl, optionally substituted heteroaryl, Si-substituted silyl, S-substituted thiol, O-substituted hydroxyl, ester, and —SF₅;
wherein:
Rᵣ₁ is optionally substituted C₁-C₄ bridging alkylene, and
Rᵣ₂ is selected from the group consisting of ester, thioester, amide, acylamino, carbonate ester, carbomate, disulfide, optionally substituted (4-acylamino)phenyl, and optionally substituted (4-acyloxy)phenyl; and
wherein:
Rₛ₁ and Rₛ₂ are independently selected from the group consisting of hydrogen, optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl, and
Rₜ is selected from the group consisting of optionally substituted alkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, and optionally substituted heteroaryl.

33.-54. (canceled)

55. A composition comprising the compound claim 1, wherein the compound is in greater than 60%, 70%, 80%, 90%, 95%, 98% diastereomeric or enantiomeric excess.

56. (canceled)

57. A pharmaceutical formulation comprising the compound of claim 1, wherein the pharmaceutical formulation further comprises a pharmaceutically acceptable excipient.

58. The pharmaceutical formulation of claim 57, wherein the pharmaceutical formulation is in the form of tablet, capsule, pill, caplet, gel, cream, granule, solution, emulsion, suspension, or nanoparticulate formulation.

59. (canceled)

60. A method for treating cancer in a subject in need thereof, comprising administering an effective amount of the compound of claim 1.

61.-64. (canceled)
